SEQUENCE LISTING (476 sequences)

SEQ ID NO: 1 - DNA, 29903 bp, Unknown
  Wuhan-Hu-1
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - protein, 7096 aa, Unknown [sequence omitted]

SEQ ID NO: 3 - protein, 1273 aa, Unknown
  SARS-CoV-2 surface glycoprotein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 275 aa, Unknown
  SARS-CoV-2 ORF3A
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 5 - protein, 75 aa, Unknown
  SARS-CoV-2 envelope
MYSFVSEETGTLIVNSVLLFLAFVVFLLVTLAILTALRLCAYCCNIVNVSLVKPSFYVYSRVKNLNSSRVPDLLV

SEQ ID NO: 6 - protein, 218 aa, Unknown
  SARS-CoV-2 membrane glycoprotein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIA

SEQ ID NO: 7 - protein, 61 aa, Unknown
  SARS-CoV-2 ORF6
MFHLVDFQVTIAEILLIIMRTFKVSIWNLDYIINLIIKNLSKSLTENKYSQLDEEQPMEID

SEQ ID NO: 8 - protein, 121 aa, Unknown
  SARS-CoV-2 ORF7a
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 9 - protein, 121 aa, Unknown
  SARS-CoV-2 ORF8
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 10 - protein, 419 aa, Unknown
  SARS-CoV-2 nucleocapsid phosphoprotein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 38 aa, Unknown
  SARS-CoV-2 ORF10
MGYINVFAFPFTIYSLLLCRMNSRNYIAQVDVVNFNLT

SEQ ID NO: 12 - protein, 1285 aa, Unknown
  SARS-CoV-2 spike protein
MDAMKRGLCCVLLLCGAVFVSPSGTGSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 697 aa, Unknown
  SARS-CoV-2 spike protein
MDAMKRGLCCVLLLCGAVFVSPSGTGSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 14 - protein, 246 aa, Unknown
  SARS-CoV-2 spike protein
MDAMKRGLCCVLLLCGAVFVSPSGTGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 15 - protein, 269 aa, Unknown
  SARS-CoV-2 spike protein
MDAMKRGLCCVLLLCGAVFVSPSGTGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNF

SEQ ID NO: 16 - protein, 192 aa, Unknown
  SARS-CoV-2 spike protein
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRFRKSNLKPFERDISTEIYQAGSTPCNGVEGNCYFPLYGFQPTGVGYQPYRVVVLSFELLHAPATVCGP

SEQ ID NO: 17 - protein, 86 aa, Unknown
  SARS-CoV-2 spike protein
NSNNLDSKVGGNYNYLYRFRKSNLKPFERDISTEIYQAGSTPCNGVEGNCYFPLYGFQPTGVGYQPYRVVVLSFELLHAPATVCGP

SEQ ID NO: 18 - protein, 1275 aa, Unknown
  SARS-CoV-2 spike protein
MDAMKRGLCCVLLLCGAVFVSPSGTGSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 1285 aa, Unknown
  SARS-CoV-2 spike protein
MDAMKRGLCCVLLLCGAVFVSPSGTGSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKQSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 1285 aa, Unknown
  SARS-CoV-2 spike protein
MDAMKRGLCCVLLLCGAVFVSPSGTGSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKQSFIEDPLFNKVTLADPGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVALAYT

SEQ ID NO: 21 - protein, 615 aa, Unknown
  SARS-CoV-2 spike protein
MDAMKRGLCCVLLLCGAVFVSPSGTGSSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 485 aa, Unknown
  SARS-CoV-2 spike protein
MDAMKRGLCCVLLLCGAVFVSPSGTGSSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 393 aa, Unknown
  SARS-CoV-2 spike protein
MDAMKRGLCCVLLLCGAVFVSPSGTGSGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 444 aa, Unknown
  SARS-CoV-2 spike protein
GPPLSSSLGLALLLLLLALLFWLYIVMGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 16 aa, Unknown
  SARS-CoV-2 spike protein
QTQTNSPRRARSVASQ

SEQ ID NO: 26 - protein, 9 aa, Unknown
  SARS-CoV-2 spike protein
PSKPSKRSF

SEQ ID NO: 27 - protein, 8 aa, Unknown
  peptide
ILLNKHID

SEQ ID NO: 28 - protein, 18 aa, Unknown
  peptide
AFFGMSRIGMEVTPSGTW

SEQ ID NO: 29 - protein, 10 aa, Unknown
  peptide
MEVTPSGTWL

SEQ ID NO: 30 - protein, 9 aa, Unknown
  peptide
GMSRIGMEV

SEQ ID NO: 31 - protein, 9 aa, Unknown
  peptide
ILLNKHIDA

SEQ ID NO: 32 - protein, 9 aa, Unknown
  peptide
ALNTPKDHI

SEQ ID NO: 33 - protein, 17 aa, Unknown
  peptide
IRQGTDYKHWPQIAQFA

SEQ ID NO: 34 - protein, 17 aa, Unknown
  peptide
KHWPQIAQFAPSASAFF

SEQ ID NO: 35 - protein, 9 aa, Unknown
  peptide
LALLLLDRL

SEQ ID NO: 36 - protein, 9 aa, Unknown
  pepdite
LLLDRLNQL

SEQ ID NO: 37 - protein, 18 aa, Unknown
  peptide
LLNKHIDAYKTFPPTEPK

SEQ ID NO: 38 - protein, 9 aa, Unknown
  peptide
LQLPQGTTL

SEQ ID NO: 39 - protein, 15 aa, Unknown
  peptide
AQFAPSASAFFGMSR

SEQ ID NO: 40 - protein, 18 aa, Unknown
  peptide
AQFAPSASAFFGMSRIGM

SEQ ID NO: 41 - protein, 15 aa, Unknown
  peptide
RRPQGLPNNTASWFT

SEQ ID NO: 42 - protein, 16 aa, Unknown
  peptide
YKTFPPTEPKKDKKKK

SEQ ID NO: 43 - protein, 16 aa, Unknown
  peptide
GAALQIPFAMQMAYRF

SEQ ID NO: 44 - protein, 16 aa, Unknown
  peptide
MAYRFNGIGVTQNVLY

SEQ ID NO: 45 - protein, 18 aa, Unknown
  peptide
QLIRAAEIRASANLAATK

SEQ ID NO: 46 - protein, 16 aa, Unknown
  peptide
PEPTIDEFIAGLIAIV

SEQ ID NO: 47 - protein, 9 aa, Unknown
  peptide
ALNTLVKQL

SEQ ID NO: 48 - protein, 9 aa, Unknown
  peptide
LITGRLQSL

SEQ ID NO: 49 - protein, 9 aa, Unknown
  peptide
NLNESLIDL

SEQ ID NO: 50 - protein, 17 aa, Unknown
  peptide
QALNTLVKQLSSNFGAI

SEQ ID NO: 51 - protein, 9 aa, Unknown
  peptide
RLNEVAKNL

SEQ ID NO: 52 - protein, 9 aa, Unknown
  peptide
VLNDILSRL

SEQ ID NO: 53 - protein, 9 aa, Unknown
  peptide
VVFLHVTYV

SEQ ID NO: 54 - protein, 9 aa, Unknown
  peptide
SEETGTLIV

SEQ ID NO: 55 - protein, 9 aa, Unknown
  peptide
FLWLLWPVT

SEQ ID NO: 56 - protein, 10 aa, Unknown
  peptide
FLWLLWPVTL

SEQ ID NO: 57 - protein, 15 aa, Unknown
  peptide
FLWLLWPVTLACFVL

SEQ ID NO: 58 - protein, 15 aa, Unknown
  peptide
IKDLPKEITVATSRT

SEQ ID NO: 59 - protein, 10 aa, Unknown
  peptide
LEQWNLVIGF

SEQ ID NO: 60 - protein, 9 aa, Unknown
  peptide
LFARTRSMW

SEQ ID NO: 61 - protein, 9 aa, Unknown
  peptide
LWLLWPVTL

SEQ ID NO: 62 - protein, 9 aa, Unknown
  peptide
LWPVTLACF

SEQ ID NO: 63 - protein, 9 aa, Unknown
  peptide
SEETGTLIV

SEQ ID NO: 64 - protein, 10 aa, Unknown
  peptide
MWSFNPETNI

SEQ ID NO: 65 - protein, 9 aa, Unknown
  peptide
NLVIGFLFL

SEQ ID NO: 66 - protein, 15 aa, Unknown
  peptide
PKEITVATSRTLSYY

SEQ ID NO: 67 - protein, 9 aa, Unknown
  peptide
ATSRTLSYY

SEQ ID NO: 68 - protein, 10 aa, Unknown
  peptide
ATSRTLSYYK

SEQ ID NO: 69 - protein, 10 aa, Unknown
  peptide
QWNLVIGFLF

SEQ ID NO: 70 - protein, 9 aa, Unknown
  peptide
RYRIGNYKL

SEQ ID NO: 71 - protein, 9 aa, Unknown
  peptide
SELVIGAVI

SEQ ID NO: 72 - protein, 9 aa, Unknown
  peptide
SFNPETNIL

SEQ ID NO: 73 - protein, 9 aa, Unknown
  peptide
SMWSFNPET

SEQ ID NO: 74 - protein, 9 aa, Unknown
  peptide
TSRTLSYYK

SEQ ID NO: 75 - protein, 10 aa, Unknown
  peptide
TVATSRTLSY

SEQ ID NO: 76 - protein, 9 aa, Unknown
  peptide
WLLWPVTLA

SEQ ID NO: 77 - protein, 10 aa, Unknown
  peptide
WPVTLACFVL

SEQ ID NO: 78 - protein, 8 aa, Unknown
  peptide
ILLNKHID

SEQ ID NO: 79 - protein, 9 aa, Unknown
  peptide
FPRGQGVPI

SEQ ID NO: 80 - protein, 10 aa, Unknown
  peptide
MEVTPSGTWL

SEQ ID NO: 81 - protein, 11 aa, Unknown
  peptide
GMEVTPSGTWL

SEQ ID NO: 82 - protein, 9 aa, Unknown
  peptide
LLLLDRLNQ

SEQ ID NO: 83 - protein, 9 aa, Unknown
  peptide
GMSRIGMEV

SEQ ID NO: 84 - protein, 9 aa, Unknown
  peptide
GTTLPKGFY

SEQ ID NO: 85 - protein, 9 aa, Unknown
  peptide
ALALLLLDR

SEQ ID NO: 86 - protein, 15 aa, Unknown
  peptide
IDAYKTFPPTEPKKD

SEQ ID NO: 87 - protein, 9 aa, Unknown
  peptide
ILLNKHIDA

SEQ ID NO: 88 - protein, 9 aa, Unknown
  peptide
ALNTPKDHI

SEQ ID NO: 89 - protein, 9 aa, Unknown
  peptide
KTFPPTEPK

SEQ ID NO: 90 - protein, 10 aa, Unknown
  peptide
KTFPPTEPKK

SEQ ID NO: 91 - protein, 9 aa, Unknown
  peptide
LALLLLDRL

SEQ ID NO: 92 - protein, 9 aa, Unknown
  peptide
LLLDRLNQL

SEQ ID NO: 93 - protein, 10 aa, Unknown
  peptide
LLLLDRLNQL

SEQ ID NO: 94 - protein, 10 aa, Unknown
  peptide
APSASAFFGM

SEQ ID NO: 95 - protein, 9 aa, Unknown
  peptide
LQLPQGTTL

SEQ ID NO: 96 - protein, 9 aa, Unknown
  peptide
AQFAPSASA

SEQ ID NO: 97 - protein, 9 aa, Unknown
  peptide
LSPRWYFYY

SEQ ID NO: 98 - protein, 15 aa, Unknown
  peptide
MSRIGMEVTPSGTWL

SEQ ID NO: 99 - protein, 9 aa, Unknown
  peptide
ASAFFGMSR

SEQ ID NO: 100 - protein, 15 aa, Unknown
  peptide
NKHIDAYKTFPPTEP

SEQ ID NO: 101 - protein, 10 aa, Unknown
  peptide
ATEGALNTPK

SEQ ID NO: 102 - protein, 10 aa, Unknown
  peptide
QLPQGTTLPK

SEQ ID NO: 103 - protein, 9 aa, Unknown
  peptide
QQQGQTVTK

SEQ ID NO: 104 - protein, 10 aa, Unknown
  peptide
QQQQGQTVTK

SEQ ID NO: 105 - protein, 9 aa, Unknown
  peptide
ALNTLVKQL

SEQ ID NO: 106 - protein, 15 aa, Unknown
  peptide
ISGINASVVNIQKEI

SEQ ID NO: 107 - protein, 15 aa, Unknown
  peptide
LDKYFKNHTSPDVDL

SEQ ID NO: 108 - protein, 10 aa, Unknown
  peptide
APHGVVFLHV

SEQ ID NO: 109 - protein, 15 aa, Unknown
  peptide
LGDISGINASVVNIQ

SEQ ID NO: 110 - protein, 15 aa, Unknown
  peptide
LGFIAGLIAIVMVTI

SEQ ID NO: 111 - protein, 10 aa, Unknown
  peptide
LIDLQELGKY

SEQ ID NO: 112 - protein, 9 aa, Unknown
  peptide
LITGRLQSL

SEQ ID NO: 113 - protein, 9 aa, Unknown
  peptide
LLLQYGSFC

SEQ ID NO: 114 - protein, 9 aa, Unknown
  peptide
LLQYGSFCT

SEQ ID NO: 115 - protein, 15 aa, Unknown
  peptide
LNTLVKQLSSNFGAI

SEQ ID NO: 116 - protein, 15 aa, Unknown
  peptide
LQDVVNQNAQALNTL

SEQ ID NO: 117 - protein, 9 aa, Unknown
  peptide
LQIPFAMQM

SEQ ID NO: 118 - protein, 15 aa, Unknown
  peptide
LQSLQTYVTQQLIRA

SEQ ID NO: 119 - protein, 15 aa, Unknown
  peptide
LQTYVTQQLIRAAEI

SEQ ID NO: 120 - protein, 9 aa, Unknown
  peptide
AQALNTLVK

SEQ ID NO: 121 - protein, 15 aa, Unknown
  peptide
AQKFNGLTVLPPLLT

SEQ ID NO: 122 - protein, 9 aa, Unknown
  peptide
MTSCCSCLK

SEQ ID NO: 123 - protein, 9 aa, Unknown
  peptide
ASANLAATK

SEQ ID NO: 124 - protein, 9 aa, Unknown
  peptide
NLNESLIDL

SEQ ID NO: 125 - protein, 15 aa, Unknown
  peptide
PCSFGGVSVITPGTN

SEQ ID NO: 126 - protein, 9 aa, Unknown
  peptide
PYRVVVLSF

SEQ ID NO: 127 - protein, 10 aa, Unknown
  peptide
QELGKYEQYI

SEQ ID NO: 128 - protein, 15 aa, Unknown
  peptide
QIPFAMQMAYRFNGI

SEQ ID NO: 129 - protein, 10 aa, Unknown
  peptide
QPYRVVVLSF

SEQ ID NO: 130 - protein, 15 aa, Unknown
  peptide
QQLIRAAEIRASANL

SEQ ID NO: 131 - protein, 15 aa, Unknown
  peptide
QTYVTQQLIRAAEIR

SEQ ID NO: 132 - protein, 9 aa, Unknown
  peptide
RLDKVEAEV

SEQ ID NO: 133 - protein, 9 aa, Unknown
  peptide
RLNEVAKNL

SEQ ID NO: 134 - protein, 9 aa, Unknown
  peptide
RLQSLQTYV

SEQ ID NO: 135 - protein, 9 aa, Unknown
  peptide
RVDFCGKGY

SEQ ID NO: 136 - protein, 15 aa, Unknown
  peptide
AYRFNGIGVTQNVLY

SEQ ID NO: 137 - protein, 10 aa, Unknown
  peptide
SLIDLQELGK

SEQ ID NO: 138 - protein, 15 aa, Unknown
  peptide
SSNFGAISSVLNDIL

SEQ ID NO: 139 - protein, 9 aa, Unknown
  peptide
SVLNDILSR

SEQ ID NO: 140 - protein, 15 aa, Unknown
  peptide
TGRLQSLQTYVTQQL

SEQ ID NO: 141 - protein, 10 aa, Unknown
  peptide
TQNVLYENQK

SEQ ID NO: 142 - protein, 10 aa, Unknown
  peptide
CMTSCCSCLK

SEQ ID NO: 143 - protein, 9 aa, Unknown
  peptide
VLNDILSRL

SEQ ID NO: 144 - protein, 10 aa, Unknown
  peptide
VQIDRLITGR

SEQ ID NO: 145 - protein, 9 aa, Unknown
  peptide
VRFPNITNL

SEQ ID NO: 146 - protein, 9 aa, Unknown
  peptide
VVFLHVTYV

SEQ ID NO: 147 - protein, 15 aa, Unknown
  peptide
WLGFIAGLIAIVMVT

SEQ ID NO: 148 - protein, 15 aa, Unknown
  peptide
CVNFNFNGLTGTGVL

SEQ ID NO: 149 - protein, 10 aa, Unknown
  peptide
YEQYIKWPWY

SEQ ID NO: 150 - protein, 15 aa, Unknown
  peptide
DKYFKNHTSPDVDLG

SEQ ID NO: 151 - protein, 10 aa, Unknown
  peptide
AEIRASANLA

SEQ ID NO: 152 - protein, 16 aa, Unknown
  peptide
GAALQIPFAMQMAYRF

SEQ ID NO: 153 - protein, 16 aa, Unknown
  peptide
MAYRFNGIGVTQNVLY

SEQ ID NO: 154 - protein, 18 aa, Unknown
  peptide
QLIRAAEIRASANLAATK

SEQ ID NO: 155 - protein, 10 aa, Unknown
  peptide
SASAFFGMSR

SEQ ID NO: 156 - protein, 9 aa, Unknown
  peptide
SPRWYFYYL

SEQ ID NO: 157 - protein, 10 aa, Unknown
  peptide
SQASSRSSSR

SEQ ID NO: 158 - protein, 9 aa, Unknown
  peptide
TPSGTWLTY

SEQ ID NO: 159 - protein, 9 aa, Unknown
  peptide
TTLPKGFYA

SEQ ID NO: 160 - protein, 10 aa, Unknown
  peptide
VLQLPQGTTL

SEQ ID NO: 161 - protein, 15 aa, Unknown
  peptide
VLQLPQGTTLPKGFY

SEQ ID NO: 162 - protein, 10 aa, Unknown
  peptide
VTPSGTWLTY

SEQ ID NO: 163 - protein, 10 aa, Unknown
  peptide
AEGSRGGSQA

SEQ ID NO: 164 - protein, 10 aa, Unknown
  peptide
FLCLFLLPSL

SEQ ID NO: 165 - protein, 9 aa, Unknown
  peptide
FLGRYMSAL

SEQ ID NO: 166 - protein, 9 aa, Unknown
  peptide
FLLNKEMYL

SEQ ID NO: 167 - protein, 9 aa, Unknown
  peptide
FLLPSLATV

SEQ ID NO: 168 - protein, 9 aa, Unknown
  peptide
FLNGSCGSV

SEQ ID NO: 169 - protein, 9 aa, Unknown
  peptide
FLNRFTTTL

SEQ ID NO: 170 - protein, 9 aa, Unknown
  peptide
FLPRVFSAV

SEQ ID NO: 171 - protein, 9 aa, Unknown
  peptide
FRYMNSQGL

SEQ ID NO: 172 - protein, 10 aa, Unknown
  peptide
FTYASALWEI

SEQ ID NO: 173 - protein, 9 aa, Unknown
  peptide
AIILASFSA

SEQ ID NO: 174 - protein, 9 aa, Unknown
  peptide
GVYDYLVST

SEQ ID NO: 175 - protein, 9 aa, Unknown
  peptide
ILASFSAST

SEQ ID NO: 176 - protein, 9 aa, Unknown
  peptide
ILGTVSWNL

SEQ ID NO: 177 - protein, 9 aa, Unknown
  peptide
IQPGQTFSV

SEQ ID NO: 178 - protein, 9 aa, Unknown
  peptide
ALRANSAVK

SEQ ID NO: 179 - protein, 9 aa, Unknown
  peptide
ALWEIQQVV

SEQ ID NO: 180 - protein, 9 aa, Unknown
  peptide
KLWAQCVQL

SEQ ID NO: 181 - protein, 9 aa, Unknown
  peptide
LLSAGIFGA

SEQ ID NO: 182 - protein, 9 aa, Unknown
  peptide
MPASWVMRI

SEQ ID NO: 183 - protein, 9 aa, Unknown
  peptide
NVLAWLYAA

SEQ ID NO: 184 - protein, 9 aa, Unknown
  peptide
QLMCQPILL

SEQ ID NO: 185 - protein, 10 aa, Unknown
  peptide
QLMCQPILLL

SEQ ID NO: 186 - protein, 9 aa, Unknown
  peptide
AVLQSGFRK

SEQ ID NO: 187 - protein, 9 aa, Unknown
  peptide
SLLSVLLSM

SEQ ID NO: 188 - protein, 9 aa, Unknown
  peptide
TLGVYDYLV

SEQ ID NO: 189 - protein, 9 aa, Unknown
  peptide
TVLSFCAFA

SEQ ID NO: 190 - protein, 9 aa, Unknown
  peptide
VLAWLYAAV

SEQ ID NO: 191 - protein, 9 aa, Unknown
  peptide
VLSFCAFAV

SEQ ID NO: 192 - protein, 9 aa, Unknown
  peptide
YIFFASFYY

SEQ ID NO: 193 - protein, 9 aa, Unknown
  peptide
FPPTSFGPL

SEQ ID NO: 194 - protein, 9 aa, Unknown
  peptide
FVDGVPFVV

SEQ ID NO: 195 - protein, 9 aa, Unknown
  peptide
AIMTRCLAV

SEQ ID NO: 196 - protein, 9 aa, Unknown
  peptide
GVAMPNLYK

SEQ ID NO: 197 - protein, 9 aa, Unknown
  peptide
ALLADKFPV

SEQ ID NO: 198 - protein, 9 aa, Unknown
  peptide
ILGLPTQTV

SEQ ID NO: 199 - protein, 9 aa, Unknown
  peptide
ILHCANFNV

SEQ ID NO: 200 - protein, 9 aa, Unknown
  peptide
IPRRNVATL

SEQ ID NO: 201 - protein, 9 aa, Unknown
  peptide
ISDYDYYRY

SEQ ID NO: 202 - protein, 9 aa, Unknown
  peptide
IVDTVSALV

SEQ ID NO: 203 - protein, 9 aa, Unknown
  peptide
KLFAAETLK

SEQ ID NO: 204 - protein, 9 aa, Unknown
  peptide
KLNVGDYFV

SEQ ID NO: 205 - protein, 9 aa, Unknown
  peptide
KLSYGIATV

SEQ ID NO: 206 - protein, 9 aa, Unknown
  peptide
KMQRMLLEK

SEQ ID NO: 207 - protein, 9 aa, Unknown
  peptide
KQFDTYNLW

SEQ ID NO: 208 - protein, 9 aa, Unknown
  peptide
LLDDFVEII

SEQ ID NO: 209 - protein, 9 aa, Unknown
  peptide
LLLDDFVEI

SEQ ID NO: 210 - protein, 9 aa, Unknown
  peptide
LLMPILTLT

SEQ ID NO: 211 - protein, 9 aa, Unknown
  peptide
LMIERFVSL

SEQ ID NO: 212 - protein, 9 aa, Unknown
  peptide
LQLGFSTGV

SEQ ID NO: 213 - protein, 9 aa, Unknown
  peptide
LVLSVNPYV

SEQ ID NO: 214 - protein, 9 aa, Unknown
  peptide
MLWCKDGHV

SEQ ID NO: 215 - protein, 9 aa, Unknown
  peptide
MMISAGFSL

SEQ ID NO: 216 - protein, 10 aa, Unknown
  peptide
MVMCGGSLYV

SEQ ID NO: 217 - protein, 9 aa, Unknown
  peptide
NLWNTFTRL

SEQ ID NO: 218 - protein, 9 aa, Unknown
  peptide
NMLRIMASL

SEQ ID NO: 219 - protein, 9 aa, Unknown
  peptide
ATVVIGTSK

SEQ ID NO: 220 - protein, 9 aa, Unknown
  peptide
RILGAGCFV

SEQ ID NO: 221 - protein, 9 aa, Unknown
  peptide
RLYYDSMSY

SEQ ID NO: 222 - protein, 9 aa, Unknown
  peptide
RQLLFVVEV

SEQ ID NO: 223 - protein, 9 aa, Unknown
  peptide
SSNVANYQK

SEQ ID NO: 224 - protein, 9 aa, Unknown
  peptide
TLIGDCATV

SEQ ID NO: 225 - protein, 9 aa, Unknown
  peptide
TLVPQEHYV

SEQ ID NO: 226 - protein, 9 aa, Unknown
  peptide
TMADLVYAL

SEQ ID NO: 227 - protein, 9 aa, Unknown
  peptide
TTLPVNVAF

SEQ ID NO: 228 - protein, 9 aa, Unknown
  peptide
VLQAVGACV

SEQ ID NO: 229 - protein, 9 aa, Unknown
  peptide
VLWAHGFEL

SEQ ID NO: 230 - protein, 9 aa, Unknown
  peptide
VMCGGSLYV

SEQ ID NO: 231 - protein, 9 aa, Unknown
  peptide
VVDKYFDCY

SEQ ID NO: 232 - protein, 9 aa, Unknown
  peptide
VVYRGTTTY

SEQ ID NO: 233 - protein, 9 aa, Unknown
  peptide
YLDAYNMMI

SEQ ID NO: 234 - protein, 9 aa, Unknown
  peptide
YLNTLTLAV

SEQ ID NO: 235 - protein, 9 aa, Unknown
  peptide
YQKVGMQKY

SEQ ID NO: 236 - protein, 9 aa, Unknown
  peptide
YTMADLVYA

SEQ ID NO: 237 - protein, 9 aa, Unknown
  peptide
YVFCTVNAL

SEQ ID NO: 238 - protein, 9 aa, Unknown
  peptide
HLVDFQVTI

SEQ ID NO: 239 - protein, 10 aa, Unknown
  peptide
HPLADNKFAL

SEQ ID NO: 240 - protein, 9 aa, Unknown
  peptide
KLFIRQEEV

SEQ ID NO: 241 - protein, 15 aa, Unknown
  peptide
QECVRGTTVLLKEPC

SEQ ID NO: 242 - protein, 10 aa, Unknown
  peptide
CELYHYQECV

SEQ ID NO: 243 - protein, 9 aa, Unknown
  peptide
SVSPKLFIR

SEQ ID NO: 244 - protein, 10 aa, Unknown
  peptide
YEGNSPFHPL

SEQ ID NO: 245 - protein, 9 aa, Unknown
  peptide
AFLLFLVLI

SEQ ID NO: 246 - protein, 15 aa, Unknown
  peptide
AFLLFLVLIMLIIFW

SEQ ID NO: 247 - protein, 9 aa, Unknown
  peptide
FLAFLLFLV

SEQ ID NO: 248 - protein, 10 aa, Unknown
  peptide
FLAFLLFLVL

SEQ ID NO: 249 - protein, 15 aa, Unknown
  peptide
FLAFLLFLVLIMLII

SEQ ID NO: 250 - protein, 9 aa, Unknown
  peptide
FLLFLVLIM

SEQ ID NO: 251 - protein, 10 aa, Unknown
  peptide
FLLFLVLIML

SEQ ID NO: 252 - protein, 15 aa, Unknown
  peptide
FLLFLVLIMLIIFWF

SEQ ID NO: 253 - protein, 9 aa, Unknown
  peptide
FLVLIMLII

SEQ ID NO: 254 - protein, 15 aa, Unknown
  peptide
FLVLIMLIIFWFSLE

SEQ ID NO: 255 - protein, 9 aa, Unknown
  peptide
FYLCFLAFL

SEQ ID NO: 256 - protein, 10 aa, Unknown
  peptide
FYLCFLAFLL

SEQ ID NO: 257 - protein, 10 aa, Unknown
  peptide
IDFYLCFLAF

SEQ ID NO: 258 - protein, 10 aa, Unknown
  peptide
IMLIIFWFSL

SEQ ID NO: 259 - protein, 15 aa, Unknown
  peptide
LAFLLFLVLIMLIIF

SEQ ID NO: 260 - protein, 15 aa, Unknown
  peptide
LFLVLIMLIIFWFSL

SEQ ID NO: 261 - protein, 9 aa, Unknown
  peptide
LIDFYLCFL

SEQ ID NO: 262 - protein, 9 aa, Unknown
  peptide
LLFLVLIML

SEQ ID NO: 263 - protein, 10 aa, Unknown
  peptide
LLFLVLIMLI

SEQ ID NO: 264 - protein, 15 aa, Unknown
  peptide
LLFLVLIMLIIFWFS

SEQ ID NO: 265 - protein, 9 aa, Unknown
  peptide
MLIIFWFSL

SEQ ID NO: 266 - protein, 9 aa, Unknown
  peptide
YLCFLAFLL

SEQ ID NO: 267 - protein, 15 aa, Unknown
  peptide
YLCFLAFLLFLVLIM

SEQ ID NO: 268 - protein, 10 aa, Unknown
  peptide
DSFKEELDKY

SEQ ID NO: 269 - protein, 9 aa, Unknown
  peptide
AEVQIDRLI

SEQ ID NO: 270 - protein, 10 aa, Unknown
  peptide
AEVQIDRLIT

SEQ ID NO: 271 - protein, 9 aa, Unknown
  peptide
FIAGLIAIV

SEQ ID NO: 272 - protein, 10 aa, Unknown
  peptide
FPNITNLCPF

SEQ ID NO: 273 - protein, 15 aa, Unknown
  peptide
GAALQIPFAMQMAYR

SEQ ID NO: 274 - protein, 10 aa, Unknown
  peptide
GLIAIVMVTI

SEQ ID NO: 275 - protein, 9 aa, Unknown
  peptide
GRLQSLQTY

SEQ ID NO: 276 - protein, 9 aa, Unknown
  peptide
GSFCTQLNR

SEQ ID NO: 277 - protein, 9 aa, Unknown
  peptide
GVVFLHVTY

SEQ ID NO: 278 - protein, 15 aa, Unknown
  peptide
GWTFGAGAALQIPFA

SEQ ID NO: 279 - protein, 10 aa, Unknown
  peptide
GYQPYRVVVL

SEQ ID NO: 280 - protein, 15 aa, Unknown
  peptide
IDRLITGRLQSLQTY

SEQ ID NO: 281 - protein, 9 aa, Unknown
  peptide
IGAGICASY

SEQ ID NO: 282 - protein, 9 aa, Unknown
  peptide
IITTDNTFV

SEQ ID NO: 283 - protein, 17 aa, Unknown
  epitope
DVVNQNAQALNTLVKQL

SEQ ID NO: 284 - protein, 17 aa, Unknown
  epitope
FFGMSRIGMEVTPSGTW

SEQ ID NO: 285 - protein, 17 aa, Unknown
  epitope
EAEVQIDRLITGRLQSL

SEQ ID NO: 286 - protein, 18 aa, Unknown
  epitope
GLPNNTASWFTALTQHGK

SEQ ID NO: 287 - protein, 28 aa, Unknown
  epitope
EIDRLNEVAKNLNESLIDLQELGKYEQY

SEQ ID NO: 288 - protein, 6 aa, Unknown
  epitope
GTTLPK

SEQ ID NO: 289 - protein, 17 aa, Unknown
  epitope
EVAKNLNESLIDLQELG

SEQ ID NO: 290 - protein, 17 aa, Unknown
  epitope
IRQGTDYKHWPQIAQFA

SEQ ID NO: 291 - protein, 17 aa, Unknown
  epitope
GAALQIPFAMQMAYRFN

SEQ ID NO: 292 - protein, 20 aa, Unknown
  epitope
KHIDAYKTFPPTEPKKDKKK

SEQ ID NO: 293 - protein, 8 aa, Unknown
  epitope
GAGICASY

SEQ ID NO: 294 - protein, 17 aa, Unknown
  epitope
KHWPQIAQFAPSASAFF

SEQ ID NO: 295 - protein, 17 aa, Unknown
  epitope
AISSVLNDILSRLDKVE

SEQ ID NO: 296 - protein, 19 aa, Unknown
  epitope
YNVTQAFGRRGPEQTQGNF

SEQ ID NO: 297 - protein, 8 aa, Unknown
  epitope
GSFCTQLN

SEQ ID NO: 298 - protein, 15 aa, Unknown
  epitope
KTFPPTEPKKDKKKK

SEQ ID NO: 299 - protein, 17 aa, Unknown
  epitope
ILSRLDKVEAEVQIDRL

SEQ ID NO: 300 - protein, 6 aa, Unknown
  epitope
LLPAAD

SEQ ID NO: 301 - protein, 8 aa, Unknown
  epitope
KGIYQTSN

SEQ ID NO: 302 - protein, 17 aa, Unknown
  epitope
LNKHIDAYKTFPPTEPK

SEQ ID NO: 303 - protein, 8 aa, Unknown
  epitope
AMQMAYRF

SEQ ID NO: 304 - protein, 10 aa, Unknown
  epitope
LPQGTTLPKG

SEQ ID NO: 305 - protein, 17 aa, Unknown
  epitope
KNHTSPDVDLGDISGIN

SEQ ID NO: 306 - protein, 8 aa, Unknown
  epitope
LPQRQKKQ

SEQ ID NO: 307 - protein, 17 aa, Unknown
  epitope
MAYRFNGIGVTQNVLYE

SEQ ID NO: 308 - protein, 18 aa, Unknown
  epitope
PKGFYAEGSRGGSQASSR

SEQ ID NO: 309 - protein, 17 aa, Unknown
  epitope
AATKMSECVLGQSKRVD

SEQ ID NO: 310 - protein, 17 aa, Unknown
  epitope
QFAPSASAFFGMSRIGM

SEQ ID NO: 311 - protein, 17 aa, Unknown
  epitope
PFAMQMAYRFNGIGVTQ

SEQ ID NO: 312 - protein, 8 aa, Unknown
  epitope
QGTDYKHW

SEQ ID NO: 313 - protein, 17 aa, Unknown
  epitope
QALNTLVKQLSSNFGAI

SEQ ID NO: 314 - protein, 15 aa, Unknown
  epitope
QLPQGTTLPKGFYAE

SEQ ID NO: 315 - protein, 17 aa, Unknown
  epitope
QLIRAAEIRASANLAAT

SEQ ID NO: 316 - protein, 18 aa, Unknown
  epitope
QLPQGTTLPKGFYAEGSR

SEQ ID NO: 317 - protein, 6 aa, Unknown
  epitope
QQFGRD

SEQ ID NO: 318 - protein, 22 aa, Unknown
  epitope
QLPQGTTLPKGFYAEGSRGGSQ

SEQ ID NO: 319 - protein, 17 aa, Unknown
  epitope
RASANLAATKMSECVLG

SEQ ID NO: 320 - protein, 8 aa, Unknown
  epitope
TFPPTEPK

SEQ ID NO: 321 - protein, 17 aa, Unknown
  epitope
RLITGRLQSLQTYVTQQ

SEQ ID NO: 322 - protein, 15 aa, Unknown
  epitope
RRPQGLPNNTASWFT

SEQ ID NO: 323 - protein, 28 aa, Unknown
  epitope
EIDRLNEVAKNLNESLIDLQELGKYEQY

SEQ ID NO: 324 - protein, 8 aa, Unknown
  epitope
SQASSRSS

SEQ ID NO: 325 - protein, 17 aa, Unknown
  epitope
SLQTYVTQQLIRAAEIR

SEQ ID NO: 326 - protein, 16 aa, Unknown
  epitope
SRGGSQASSRSSSRSR

SEQ ID NO: 327 - protein, 17 aa, Unknown
  epitope
DLGDISGINASVVNIQK

SEQ ID NO: 328 - protein, 66 aa, Unknown
  peptide
VGDSAEVAVKMFDAYVNTFSSTFNVPMEKLKTLVATAEAELAKNVSLDNVLSTFISAARQGFVDSD

SEQ ID NO: 329 - protein, 41 aa, Unknown
  peptide
MSYEDQDALFAYTKRNVIPTITQMNLKYAISAKNRARTVAG

SEQ ID NO: 330 - protein, 87 aa, Unknown
  peptide
DIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 331 - protein, 46 aa, Unknown
  peptide
AVCRHHANEYRLYLDAYNMMISAGFSLWVYKQFDTYNLWNTFTRLQ

SEQ ID NO: 332 - protein, 98 aa, Unknown
  peptide
NFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQT

SEQ ID NO: 333 - protein, 83 aa, Unknown
  peptide
ECDIPIGAGICASYQTQTNSPRRARSVASQSIIAYTMSLGAENSVAYSNNSIAIPTNFTISVTTEILPVSMTKTSVDCTMYIC

SEQ ID NO: 334 - protein, 70 aa, Unknown
  peptide
DKSVYYTSNPTTFHLDGEVITFDNLKTLLSLREVRTIKVFTTVDNINLHTQVVDMSMTYGQQFGPTYLDG

SEQ ID NO: 335 - protein, 82 aa, Unknown
  peptide
AQKFNGLTVLPPLLTDEMIAQYTSALLAGTITSGWTFGAGAALQIPFAMQMAYRFNGIGVTQNVLYENQKLIANQFNSAIGK

SEQ ID NO: 336 - protein, 117 aa, Unknown
  peptide
DAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALK

SEQ ID NO: 337 - protein, 84 aa, Unknown
  peptide
CREHEHEIAWYTERSEKSYELQTPFEIKLAKKFDTFNGECPNFVFPLNSIIKTIQPRVEKKKLDGFMGRIRSVYPVASPNECNQ

SEQ ID NO: 338 - protein, 85 aa, Unknown
  peptide
AAYVDNSSLTIKKPNELSRVLGLKTLATHGLAAVNSVPWDTIANYAKPFLNKVVSTTTNIVTRCLNRVCTNYMPYFFTLLLQLCT

SEQ ID NO: 339 - protein, 86 aa, Unknown
  peptide
IADKYVRNLQHRLYECLYRNRDVDTDFVNEFYAYLRKHFSMMILSDDAVVCFNSTYASQGLVASIKNFKSVLYYQNNVFMSEAKCW

SEQ ID NO: 340 - protein, 80 aa, Unknown
  peptide
RQFHQKLLKSIAATRGATVVIGTSKFYGGWHNMLKTVYSDVENPHLMGWDYPKCDRAMPNMLRIMASLVLARKHTTCCSL

SEQ ID NO: 341 - protein, 80 aa, Unknown
  peptide
GHFDGQQGEVPVSIINNTVYTKVDGVDVELFENKTTLPVNVAFELWAKRNIKPVPEVKILNNLGVDIAANTVIWDYKRDA

SEQ ID NO: 342 - protein, 92 aa, Unknown
  peptide
LHPTQAPTHLSVDTKFKTEGLCVDIPGIPKDMTYRRLISMMGFKMNYQVNGYPNMFITREEAIRHVRAWIGFDVEGCHATREAVGTNLPLQL

SEQ ID NO: 343 - protein, 55 aa, Unknown
  peptide
DTNVLEGSVAYESLRPDTRYVLMDGSIIQFPNTYLEGSVRVVTTFDSEYCRHGTC

SEQ ID NO: 344 - protein, 96 aa, Unknown
  peptide
DKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDG

SEQ ID NO: 345 - protein, 107 aa, Unknown
  peptide
KVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPD

SEQ ID NO: 346 - protein, 80 aa, Unknown
  peptide
KGIYQTSNFRVQPTESIVRFPNITNLCPFGEVFNATRFASVYAWNRKRISNCVADYSVLYNSASFSTFKCYGVSPTKLND

SEQ ID NO: 347 - protein, 91 aa, Unknown
  peptide
EFVFKNIDGYFKIYSKHTPINLVRDLPQGFSALEPLVDLPIGINITRFQTLLALHRSYLTPGDSSSGWTAGAAAYYVGYLQPRTFLLKYNE

SEQ ID NO: 348 - protein, 102 aa, Unknown
  peptide
EAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPM

SEQ ID NO: 349 - protein, 97 aa, Unknown
  peptide
SQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDS

SEQ ID NO: 350 - protein, 77 aa, Unknown
  peptide
DCVVLHSYFTSDYYQLYSTQLSTDTGVEHVTFFIYNKIVDEPEEHVQIHTIDGSSGVVNPVMEPIYDEPTTTTSVPL

SEQ ID NO: 351 - protein, 37 aa, Unknown
  peptide
LCAYCCNIVNVSLVKPSFYVYSRVKNLNSSRVPDLLV

SEQ ID NO: 352 - protein, 124 aa, Unknown
  peptide
SFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 353 - protein, 85 aa, Unknown
  peptide
SGTYEGNSPFHPLADNKFALTCFSTQFAFACPDGVKHVYQLRARSVSPKLFIRQEEVQELYSPIFLIVAAIVFITLCFTLKRKTE

SEQ ID NO: 354 - protein, 107 aa, Unknown
  peptide
TKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKD

SEQ ID NO: 355 - protein, 81 aa, Unknown
  peptide
STKHFYWFFSNYLKRRVVFNGVSFSTFEEAALCTFLLNKEMYLKLRSDVLLPLTQYNRYLALYNKYKYFSGAMDTTSYREA

SEQ ID NO: 356 - protein, 84 aa, Unknown
  peptide
VPLNIIPLTTAAKLMVVIPDYNTYKNTCDGTTFTYASALWEIQQVVDADSKIVQLSEISMDNSPNLAWPLIVTALRANSAVKLQ

SEQ ID NO: 357 - protein, 80 aa, Unknown
  peptide
EGSEGLNDNLLEILQKEKVNINIVGDFKLNEEIAIILASFSASTSAFVETVKGLDYKAFKQIVESCGNFKVTKGKAKKGA

SEQ ID NO: 358 - protein, 108 aa, Unknown
  peptide
IGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDW

SEQ ID NO: 359 - protein, 77 aa, Unknown
  peptide
GSEDNQTTTIQTIVEVQPQLEMELTPVVQTIEVNSFSGYLKLTDNVYIKNADIVEEAKKVKPTVVVNAANVYLKHGG

SEQ ID NO: 360 - protein, 81 aa, Unknown
  peptide
NKGEDIQLLKSAYENFNQHEVLLAPLLSAGIFGADPIHSLRVCVDTVRTNVYLAVFDKNLYDKLVSSFLEMKSEKQVEQKI

SEQ ID NO: 361 - protein, 104 aa, Unknown
  peptide
SAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVT

SEQ ID NO: 362 - protein, 104 aa, Unknown
  peptide
IKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYK

SEQ ID NO: 363 - protein, 82 aa, Unknown
  peptide
NSHEGKTFYVLPNDDTLRVEAFEYYHTTDPSFLGRYMSALNHTKKWKYPQVNGLTSIKWADNNCYLATALLTLQQIELKFNP

SEQ ID NO: 364 - protein, 76 aa, Unknown
  peptide
TTRTQLPPAYTNSFTRGVYYPDKVFRSSVLHSTQDLFLPFFSNVTWFHAIHVSGTNGTKRFDNPVLPFNDGVYFAS

SEQ ID NO: 365 - protein, 83 aa, Unknown
  peptide
REQIDGYVMHANYIFWRNTNPIQLSSYSLFDMSKFPLKLRGTAVMSLKEGQINDMILSLLSKGRLIIRENNRVVISSDVLVNN

SEQ ID NO: 366 - protein, 61 aa, Unknown; identical to SEQ ID NO: 7
  peptide

SEQ ID NO: 367 - protein, 84 aa, Unknown
  peptide
DVVNQNAQALNTLVKQLSSNFGAISSVLNDILSRLDKVEAEVQIDRLITGRLQSLQTYVTQQLIRAAEIRASANLAATKMSECV

SEQ ID NO: 368 - protein, 63 aa, Unknown
  peptide
AIASEFSSLPSYAAFATAQEAYEQAVANGDSEVVLKKLKKSLNVAKSEFDRDAAMQRKLEKMA

SEQ ID NO: 369 - protein, 80 aa, Unknown
  peptide
TTKGGRFVLALLSDLQDLKWARFPKSDGTGTIYTELEPPCRFVTDTPKGPKVKYLYFIKGLNNLNRGMVLGSLAATVRLQ

SEQ ID NO: 370 - protein, 45 aa, Unknown
  peptide
KMFDAYVNTFSSTFNVPMEKLKTLVATAEAELAKNVSLDNVLSTF

SEQ ID NO: 371 - protein, 41 aa, Unknown; identical to SEQ ID NO: 329
  peptide

SEQ ID NO: 372 - protein, 45 aa, Unknown
  peptide
DGTLMIERFVSLAIDAYPLTKHPNQEYADVFHLYLQYIRKLHDEL

SEQ ID NO: 373 - protein, 43 aa, Unknown
  peptide
RHHANEYRLYLDAYNMMISAGFSLWVYKQFDTYNLWNTFTRLQ

SEQ ID NO: 374 - protein, 41 aa, Unknown
  peptide
NFNVLFSTVFPPTSFGPLVRKIFVDGVPFVVSTGYHFRELG

SEQ ID NO: 375 - protein, 45 aa, Unknown
  peptide
RSVASQSIIAYTMSLGAENSVAYSNNSIAIPTNFTISVTTEILPV

SEQ ID NO: 376 - protein, 47 aa, Unknown
  peptide
DKSVYYTSNPTTFHLDGEVITFDNLKTLLSLREVRTIKVFTTVDNIN

SEQ ID NO: 377 - protein, 45 aa, Unknown
  peptide
LTDEMIAQYTSALLAGTITSGWTFGAGAALQIPFAMQMAYRFNGI

SEQ ID NO: 378 - protein, 45 aa, Unknown
  peptide
AVVYRGTTTYKLNVGDYFVLTSHTVMPLSAPTLVPQEHYVRITGL

SEQ ID NO: 379 - protein, 41 aa, Unknown
  peptide
ERSEKSYELQTPFEIKLAKKFDTFNGECPNFVFPLNSIIKT

SEQ ID NO: 380 - protein, 45 aa, Unknown
  peptide
AAYVDNSSLTIKKPNELSRVLGLKTLATHGLAAVNSVPWDTIANY

SEQ ID NO: 381 - protein, 41 aa, Unknown
  peptide
VDTDFVNEFYAYLRKHFSMMILSDDAVVCFNSTYASQGLVA

SEQ ID NO: 382 - protein, 33 aa, Unknown
  peptide
FNSTYASQGLVASIKNFKSVLYYQNNVFMSEAK

SEQ ID NO: 383 - protein, 43 aa, Unknown
  peptide
VYSDVENPHLMGWDYPKCDRAMPNMLRIMASLVLARKHTTCCS

SEQ ID NO: 384 - protein, 45 aa, Unknown
  peptide
KDMTYRRLISMMGFKMNYQVNGYPNMFITREEAIRHVRAWIGFDV

SEQ ID NO: 385 - protein, 41 aa, Unknown
  peptide
YESLRPDTRYVLMDGSIIQFPNTYLEGSVRVVTTFDSEYCR

SEQ ID NO: 386 - protein, 41 aa, Unknown
  peptide
SKGFFKEGSSVELKHFFFAQDGNAAISDYDYYRYNLPTMCD

SEQ ID NO: 387 - protein, 43 aa, Unknown
  peptide
KTLQPVSELLTPLGIDLDEWSMATYYLFDESGEFKLASHMYCS

SEQ ID NO: 388 - protein, 45 aa, Unknown
  peptide
PTESIVRFPNITNLCPFGEVFNATRFASVYAWNRKRISNCVADYS

SEQ ID NO: 389 - protein, 43 aa, Unknown
  peptide
FASVYAWNRKRISNCVADYSVLYNSASFSTFKCYGVSPTKLND

SEQ ID NO: 390 - protein, 45 aa, Unknown
  peptide
TRFQTLLALHRSYLTPGDSSSGWTAGAAAYYVGYLQPRTFLLKYN

SEQ ID NO: 391 - protein, 42 aa, Unknown
  peptide
VVLHSYFTSDYYQLYSTQLSTDTGVEHVTFFIYNKIVDEPEE

SEQ ID NO: 392 - protein, 31 aa, Unknown
  peptide
NIVNVSLVKPSFYVYSRVKNLNSSRVPDLLV

SEQ ID NO: 393 - protein, 43 aa, Unknown
  peptide
PKEITVATSRTLSYYKLGASQRVAGDSGFAAYSRYRIGNYKLN

SEQ ID NO: 394 - protein, 42 aa, Unknown
  peptide
VKHVYQLRARSVSPKLFIRQEEVQELYSPIFLIVAAIVFITL

SEQ ID NO: 395 - protein, 42 aa, Unknown
  peptide
HWPQIAQFAPSASAFFGMSRIGMEVTPSGTWLTYTGAIKLDD

SEQ ID NO: 396 - protein, 46 aa, Unknown
  peptide
STKHFYWFFSNYLKRRVVFNGVSFSTFEEAALCTFLLNKEMYLKLR

SEQ ID NO: 397 - protein, 46 aa, Unknown
  peptide
LLNKEMYLKLRSDVLLPLTQYNRYLALYNKYKYFSGAMDTTSYREA

SEQ ID NO: 398 - protein, 44 aa, Unknown
  peptide
VPLNIIPLTTAAKLMVVIPDYNTYKNTCDGTTFTYASALWEIQQ

SEQ ID NO: 399 - protein, 42 aa, Unknown
  peptide
NINIVGDFKLNEEIAIILASFSASTSAFVETVKGLDYKAFKQ

SEQ ID NO: 400 - protein, 43 aa, Unknown
  peptide
SILSPLYAFASEAARVVRSIFSRTLETAQNSVRVLQKAAITIL

SEQ ID NO: 401 - protein, 43 aa, Unknown
  peptide
SEDNQTTTIQTIVEVQPQLEMELTPVVQTIEVNSFSGYLKLTD

SEQ ID NO: 402 - protein, 43 aa, Unknown
  peptide
FGADPIHSLRVCVDTVRTNVYLAVFDKNLYDKLVSSFLEMKSE

SEQ ID NO: 403 - protein, 43 aa, Unknown
  peptide
KIQEGVVDYGARFYFYTSKTTVASLINTLNDLNETLVTMPLGY

SEQ ID NO: 404 - protein, 41 aa, Unknown
  peptide
KTFYVLPNDDTLRVEAFEYYHTTDPSFLGRYMSALNHTKKW

SEQ ID NO: 405 - protein, 46 aa, Unknown
  peptide
HLVDFQVTIAEILLIIMRTFKVSIWNLDYIINLIIKNLSKSLTENK

SEQ ID NO: 406 - protein, 44 aa, Unknown
  peptide
VEAEVQIDRLITGRLQSLQTYVTQQLIRAAEIRASANLAATKMS

SEQ ID NO: 407 - protein, 36 aa, Unknown
  peptide
AIASEFSSLPSYAAFATAQEAYEQAVANGDSEVVLK

SEQ ID NO: 408 - protein, 41 aa, Unknown
  peptide
CRFVTDTPKGPKVKYLYFIKGLNNLNRGMVLGSLAATVRLQ

SEQ ID NO: 409 - protein, 6 aa, Unknown
  peptide
VKLHYT

SEQ ID NO: 410 - protein, 9744 aa, Unknown
  SARS-CoV-2 concatenated Polyprotein
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNNMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTMDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPLMYSFVSEETGTLIVNSVLLFLAFVVFLLVTLAILTALRLCAYCCNIVNVSLVKPSFYVYSRVKNLNSSRVPDLLVMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQMFHLVDFQVTIAEILLIIMRTFKVSIWNLDYIINLIIKNLSKSLTENKYSQLDEEQPMEIDMKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTEMIELSLIDFYLCFLAFLLFLVLIMLIIFWFSLELQDHNETCHAMKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFIMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAMGYINVFAFPFTIYSLLLCRMNSRNYIAQVDVVNFNLT

SEQ ID NO: 411 - protein, 242 aa, Unknown
  SARS-CoV-2 spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNF

SEQ ID NO: 412 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVFNFN

SEQ ID NO: 413 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKCVFNFN

SEQ ID NO: 414 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVFNFN

SEQ ID NO: 415 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVFNFN

SEQ ID NO: 416 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVFNFN

SEQ ID NO: 417 - protein, 242 aa, Unknown
  SARS-CoV spike potein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVFNFN

SEQ ID NO: 418 - protein, 25 aa, Unknown
  Human OSM
MGVLLTQRTLLSLVLALLFPSMASM

SEQ ID NO: 419 - protein, 16 aa, Unknown
  VSV-G
MKCLLYLAFLFIGVNC

SEQ ID NO: 420 - protein, 21 aa, Unknown
  Mouse Ig Kappa
METDTLLLWVLLLWVPGSTGD

SEQ ID NO: 421 - protein, 19 aa, Unknown
  Human IgG2 H
MGWSCIILFLVATATGVHS

SEQ ID NO: 422 - protein, 17 aa, Unknown
  BM40
MRAWIFFLLCLAGRALA

SEQ ID NO: 423 - protein, 21 aa, Unknown
  Secrecon
MWWRLWWLLLLLLLLWPMVWA

SEQ ID NO: 424 - protein, 22 aa, Unknown
  Human IgKVIII
MDMRVPAQLLGLLLLWLRGARC

SEQ ID NO: 425 - protein, 16 aa, Unknown
  CD33
MPLLLLLPLLWAGALA

SEQ ID NO: 426 - protein, 23 aa, Unknown
  tPA
MDAMKRGLCCVLLLCGAVFVSPS

SEQ ID NO: 427 - protein, 27 aa, Unknown
  tPA
MDAMKRGLCCVLLLCGAVFVSPSGTGS

SEQ ID NO: 428 - protein, 18 aa, Unknown
  Human Chymotrypsinogen
MAFLWLLSCWALLGTTFG

SEQ ID NO: 429 - protein, 15 aa, Unknown
  Human trypsinogen-2
MNLLLILTFVAAAVA

SEQ ID NO: 430 - protein, 20 aa, Unknown
  Human IL-2
MYRMQLLSCIALSLALVTNS

SEQ ID NO: 431 - protein, 17 aa, Unknown
  Gaussia luciferase
MGVKVLFALICIAVAEA

SEQ ID NO: 432 - protein, 16 aa, Unknown
  Albumin(HSA)
MKWVTFISLLFSSAYS

SEQ ID NO: 433 - protein, 16 aa, Unknown
  Influenza Haemagglutinin
MKTIIALSYIFCLVLG

SEQ ID NO: 434 - protein, 24 aa, Unknown
  Human insulin
MALWMRLLPLLALLALWGPDPAAA

SEQ ID NO: 435 - protein, 16 aa, Unknown
  Silkworm Fibroin LC
MKPIFLVLLVVTSAYA

SEQ ID NO: 436 - protein, 18 aa, Unknown
  adenovirus protein E3/gp19K
MRYMILGLLALAAVCSAA

SEQ ID NO: 437 - protein, 19 aa, Unknown
  IgG
MKHLWFFLLLVAAPRWVLS

SEQ ID NO: 438 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNFTF

SEQ ID NO: 439 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKSVNFTF

SEQ ID NO: 440 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKSVNFTF

SEQ ID NO: 441 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTF

SEQ ID NO: 442 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTF

SEQ ID NO: 443 - protein, 242 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTF

SEQ ID NO: 444 - protein, 756 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNFTFGGGGSGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTFGGGGSGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTF

SEQ ID NO: 445 - protein, 778 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNFTFGGGGSGGGGSRRKRSVGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTFGGGGSGGGGSRRKRSVGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTF

SEQ ID NO: 446 - protein, 197 aa, Unknown
  SARS-CoV-2 surface glycoprotein
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 447 - protein, 19 aa, Unknown
  SARS Co-V spike protein
SFIEDLLFNKVTLADAGFI

SEQ ID NO: 448 - protein, 10 aa, Unknown
  SARS Co-V spike protein
SRLDKVEAEV

SEQ ID NO: 449 - protein, 39 aa, Unknown
  SARS Co-V spike protein
CCMTSCCSCLKGCCSCGSCCKFDEDDSEPVLKGVKLHYT

SEQ ID NO: 450 - protein, 11 aa, Unknown
  peptide
NSPRRARSVAS

SEQ ID NO: 451 - protein, 11 aa, Unknown
  peptide
NSPQQAQSVAS

SEQ ID NO: 452 - protein, 18 aa, Unknown
  peptide
KRSFIEDLLFNKVTLADA

SEQ ID NO: 453 - protein, 8 aa, Unknown
  peptide
KRSFIADA

SEQ ID NO: 454 - protein, 10 aa, Unknown
  peptide
SRLDKVEAEV

SEQ ID NO: 455 - protein, 10 aa, Unknown
  peptide
SRLDPPEAEV

SEQ ID NO: 456 - protein, 11 aa, Unknown
  peptide
NSPSGAGSVAS

SEQ ID NO: 457 - protein, 9 aa, Unknown
  peptide
PSKPSKQSF

SEQ ID NO: 458 - protein, 9 aa, Unknown
  pepide
PSKPSKNSF

SEQ ID NO: 459 - protein, 9 aa, Unknown
  peptide
PSKPSNASF

SEQ ID NO: 460 - protein, 242 aa, Unknown; identical to SEQ ID NO: 441
  SARS-CoV spike protein

SEQ ID NO: 461 - protein, 6 aa, Unknown
  peptide
NSPVAS

SEQ ID NO: 462 - protein, 8 aa, Unknown
  peptide
LQYGSFCT

SEQ ID NO: 463 - protein, 7 aa, Unknown
  peptide
QEINSSY

SEQ ID NO: 464 - protein, 7 aa, Unknown
  peptide
SHPRLSA

SEQ ID NO: 465 - protein, 7 aa, Unknown
  peptide
SMPNPMV

SEQ ID NO: 466 - protein, 7 aa, Unknown
  peptide
GLQQVLL

SEQ ID NO: 467 - protein, 7 aa, Unknown
  peptide
HELSVLL

SEQ ID NO: 468 - protein, 7 aa, Unknown
  peptide
YAPQRLP

SEQ ID NO: 469 - protein, 7 aa, Unknown
  peptide
TPRTLPT

SEQ ID NO: 470 - protein, 7 aa, Unknown
  peptide
APVHSSI

SEQ ID NO: 471 - protein, 7 aa, Unknown
  peptide
APPHALS

SEQ ID NO: 472 - protein, 7 aa, Unknown
  peptide
TFSNRFI

SEQ ID NO: 473 - protein, 7 aa, Unknown
  peptide
VVPTPPY

SEQ ID NO: 474 - protein, 7 aa, Unknown
  peptide
ELAPDSP

SEQ ID NO: 475 - protein, 1527 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKSVNFTFGGGGSGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKSVNFTFGGGGSGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKSVNFTFGGGGSGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTFGGGGSGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTFGGGGSGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTF

SEQ ID NO: 476 - protein, 1567 aa, Unknown
  SARS-CoV spike protein
TLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKSVNFTFGSGGGGSRRKRSVGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKSVNFTFGSGGGGSRRKRSVGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKSVNFTFGSGGGGSRRKRSVGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTFGSGGGGSRRKRSVGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTFGSGGGGSRRKRSVGGGGSGGGGSTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFTF

(19) United States
(12) Patent Application Publication
(10) Pub. No.: US 2021/0260181 A1
(43) Pub. Date: Aug. 26, 2021

(54) CORONAVIRUS IMMUNOGENIC COMPOSITIONS AND USES THEREOF

(71) Applicant: Altimmune, Inc, Gaithersburg, MD (US)

(72) Inventors: Bertrand Victor Gilbert Georges, Leeds (GB); M. Scot Roberts, Gaithersburg, MD (US)

(73) Assignee: Altimmune, Inc, Gaithersburg, MD (US)

(21) Appl. No.: 17/175,144

(22) Filed: Feb. 12, 2021

Related U.S. Application Data
(60) Provisional application No. 63/148,374, filed on Feb. 11, 2021.
(60) Provisional application No. 63/142,077, filed on Jan. 27, 2021.
(60) Provisional application No. 63/140,128, filed on Jan. 21, 2021.
(60) Provisional application No. 63/088,736, filed on Oct. 7, 2020.
(60) Provisional application No. 63/069,792, filed on Aug. 25, 2020.
(60) Provisional application No. 62/050,844, filed on Sep. 16, 2014.
(60) Provisional application No. 63/016,902, filed on Apr. 28, 2020.
(60) Provisional application No. 63/005,923, filed on Apr. 6, 2020.
(60) Provisional application No. 62/992,553, filed on Mar. 20, 2020.
(60) Provisional application No. 62/977,078, filed on Feb. 14, 2020.

(51) Int. Cl. A61K 39/215; C12N 15/86; C12N 7/00; A61K 9/00; A61P 31/14

(52) U.S. Cl. CPC A61K 39/215; C12N 15/86; C12N 7/00; A61K 9/0043; A61K 2039/543; C12N 2750/14143; C12N 2750/14034; C12N 2750/14171; A61P 31/14

(57) ABSTRACT

Provided in the present disclosure are immunogenic compounds, pharmaceutical formulations thereof and their use for inducing a protective immune response against 2019 novel coronavirus (SARS-CoV-2) infection and variants in a mammal.

### RELATED APPLICATIONS

This application claims priority to provisional application Nos. U.S. Ser. No. 62/977,078 filed 14 Feb. 2020; U.S. Ser. No. 62/992,553 filed 20 Mar. 2020; U.S. Ser. No. 63/005,923 filed 6 Apr. 2020; U.S. Ser. No. 63/016,902 filed 28 Apr. 2020; U.S. Ser. No. 63/050,844 filed 12 Jul. 2020; U.S. Ser. No. 63/069,792 filed 25 Aug. 2020; U.S. Ser. No. 63/088,736 filed 7 Oct. 2020; U.S. Ser. No. 63/140,128 filed 21 Jan. 2021; U.S. Ser. No. 63/142,077 filed 27 Jan. 2021; and, U.S. Ser. No. 63/148,374 filed 11 Feb. 2021, each of which are hereby incorporated into this application in their entirety.

### SEQUENCE LISTING

The instant application contains a Sequence Listing which has been submitted electronically in ASCII format via EFS-Web and hereby incorporated by reference in its entirety. Said ASCII copy, created on 3 May 2021, is named ALT2029US1T_Corr_ST25.txt and is 479,367 bytes in size.

### FIELD OF THE DISCLOSURE

This application pertains generally to an adenoviral vectored coronavirus antigen pharmaceutical formulation for administration to a mammalian subject that induces an immune response in the subject and optionally provides protection against novel 2019 Coronavirus (SARS-CoV-2).

### BACKGROUND OF THE DISCLOSURE

The coronaviruses are a diverse group of large enveloped, positive-stranded RNA (ss RNA) viruses that cause respiratory and enteric diseases in humans and other animals. For example, Human coronaviruses 229E (HCoV-229E), OC43 (HCoV-OC43), NL63, and HKU1 are endemic in the human population and cause up to 30% of common colds. Coronaviruses of animals (e.g., porcine transmissible gastroenteritis virus (TGEV), murine hepatitis virus (MHV) and avian infectious bronchitis virus (IBV)) cause respiratory, gastrointestinal, neurological, or hepatic disease in their respective hosts.

Coronavirus has a positive-sense, non-segmented, single-stranded RNA genome, which encodes at least 18 viral proteins (such as non-structural proteins (NSP) 1-13, structural proteins E, M, N, S, and an RNA-dependent RNA polymerase). Coronavirus has three major surface glycoproteins (designated S, E, and M), and some coronaviruses have another surface glycoprotein referred to as hemagglutinin esterase (HE), in addition, the N (nucleocapsid) protein is a basic phosphoprotein, which is generally associated with the genome and has been reported to be antigenic (Holmes and Lai, Fields Virology, Chapter 34, 1996). The S (spike) protein, a major antigen of coronavirus, has two domains: Sl, which is believed to be involved in receptor binding and S2, believed to mediate membrane fusion between the virus and target cell (Holmes and Lai, 1996, supra).

The S (spike) protein may form non-covalently linked homotrimers (oligomers), which may mediate receptor binding and virus infectivity. Homotrimers of S proteins are likely necessary for presenting the correct native conformation of receptor binding domains and for eliciting a neutralizing antibody response. In addition, intracellular processing of S protein is associated with significant posttranslation oligosaccharide modification. The posttranslation oligosaccharide modification (glycosylation) expected by N-glycan motif analysis indicates that the S protein has as many as 23 sites for such modification. In addition, C-terminal cysteine residues may also participate in protein folding and preserving the native (functional) S protein conformation. The S protein of some coronaviruses can be proteolytically processed near the center of the S protein by a trypsin-like protease in the Golgi apparatus or by extracellularly localized enzymes into to a linked polypeptide, containing an N-terminal Sl and a C-terminal S2 polypeptide. Presently, the coronaviruses are subdivided into α-genus, β-genus (e.g., MERS, SARS, SARS-CoV-2), and γ-genus. See FIG. 14.

Coronavirus infection is achieved through fusion of the lipid bilayer of the viral envelope with host cell membranes. Membrane fusion is mediated by the viral spike (S) glycoprotein on the viral envelope. The S-glycoprotein is synthesized as a precursor of about 180 kDa that oligomerizes in the endoplasmic reticulum and is incorporated into budding virions in a pre-Golgi compartment. S1 contains the receptor-binding site and thus contributes to defining the host range of the virus. S2 is the transmembrane subunit which contributes to mediating fusion between viral and cellular membranes. S2 contains two 4,3-hydrophobic repeat domains (HR) that are predicted to form coiled-coil structures. These regions are denoted HR-1 and HR-2, and are separated by an intervening stretch of amino acid residues called the interhelical domain. These coiled-coil regions may play an important role in defining the oligomeric structure of the spike protein in its native state and its fusogenic ability.

The novel coronovirus SARS-CoV-2 (initally reported as 2019-nCoV and officially named SARS-CoV-2 by the Coronavirus Study Group (a working group of the International Committee on Taxonomy of Viruses) based on phylogeny, taxonomy and estabilished practice (BioRxiv; doi.org/10.1101/2020.02.07.937862)) is a new strain that has not been previously identified in humans and was first reported in Wuhan, Hubei Province, China. SARS-CoV-2 is the cause of the ongoing 2019-20 Wuhan coronavirus outbreak, a global health emergency. Genomic sequencing has shown that it is a positive-sense, single-stranded RNA coronavirus (GenBank Accession No. MN908947.3; RefSeq NC_045512; “Wuhan seafood market pneumonia virus isolate Wuhan-Hu-1”). Coronaviruses are primarily spread through close contact, in particular through respiratory droplets from coughs and sneezes within a range of about 6 feet (1.8 m). Common signs of infection include respiratory symptoms, fever, cough, shortness of breath and breathing difficulties. In more severe cases, infection can cause pneumonia, severe acute respiratory syndrome, kidney failure and even death. There is an urgent need for vectors and immunogenic compositions comprising the same that can be used to induce an immune response against SARS-CoV-2. This disclosure addresses these issues.

### SUMMARY OF THE DISCLOSURE

In some embodiments, this disclosure provides reagents, compositions, and methods for inducing and/or improving (e.g., enhancing) an immune response against coronavirus, in particular novel 2019 coronavirus SARS-CoV-2. For instance, in some embodiments, this disclosure provides replication defective adenoviral vectors encoding at least one SARS-CoV-2 antigen(s) (e.g., E1A/E3 deletion human Adenovirus type 5 (hAd5) (hAd5-SARS-CoV-2)), and/or another one or more exogenous antigens of a different type of infectious agent (e.g., a different type of virus such as influenza (e.g., Ad-HA)), or lacking a transgene (“hAdE”; e.g., not encoding at least one antigen or immunogen of an exogenous infectious agent “empty”), which can be referred to herein collectively as “SARS-CoV-2 immunization vectors”. As discussed herein, such vectors (and/or immunogenic compositions comprising the same) can be used to induce mucosal, cell-mediated and/or humoral immune responses against SARS-CoV-2 (e.g., against protective SARS-CoV-2 epitopes such as spike (S) protein receptor binding domain (RBD)). In some embodiments, this disclosure describes the administration of such vectors (e.g., hAd5-SARS-CoV-2 and/or hAd5) to animals and/or human beings to induce and/or enhance an immune response (e.g., the production of antibodies and/or CD8⁺ T cells (and/or other T cells)) having specificity for SARS-CoV-2 T epitope(s) (e.g., a dominant epitopes). In some embodiments, the immune response induced by the vector administered intranasal induce and/or enhance a T cell response with a resident memory phenotypes in the respiratory tract. In some embodiments, such immune response is protective against SARS-CoV-2 and/or effective in ameliorating the symptoms and/or infection by SARS-CoV-2, and in some embodiments can be protective against a SARS-CoV-2 challenge. Thus, in some embodiments, this disclosure describes the use of an immunogenic composition(s) comprising hAd5-SARS-CoV-2 to provide solutions to art-recognized problems regarding SARS-CoV-2 transmission and infection.

In certain embodiments provided herein are immunogenic compositions, formulations thereof and methods of use for treating and/or preventing COVID-19 related diseases caused by SARS-Cov-2 viral infection, wherein the immunogenic composition comprises a replication defective adenoviral (rdAd) vector comprising a nucleic acid sequence encoding SEQ ID NO: 446 or a variant comprising at least 90%, or at least 95% identity to SEQ ID NO: 446. In embodiments, the immunogenic composition comprises a replication defective adenoviral (rdAd) vector comprising a nucleic acid sequence encoding SEQ ID NO: 15, or a variant comprising at least 90%, or at least 95% identity to SEQ ID NO: 15. SEQ ID NO: 15 comprises the RBD sequence (SEQ ID NO: 446) with long flanking sequences of the 51 domain of the spike protein (SEQ ID NO: 3) and a leader sequence. In certain embodiments, the nucleic acid sequence encodes SEQ ID NO: 13 (51 domain). In embodiments, the nucleic acid sequence encodes a sequence comprising one or more point mutations of SEQ ID NO: 3.

As used herein “variant” refers to one or more mutations in the RBD sequence. In certain embodiments, the nucleic acid sequence encodes one or more of SEQ ID NOS: 412-417, SEQ ID NOS: 438-445, SEQ ID NOS: 475-476 and SEQ ID NO: 460 (RBD sequences comprising one or more mutations as compared to the RBD sequence of SEQ ID NO: 3, SEQ ID NO: 446, SEQ ID NO: 13 or SEQ ID NO: 15). In embodiments, the nucleic acid sequence encodes a sequence comprising one or more mutations at positions 333-388, 390-395, 397-399, 401-411, 413-415, 417-419, 424, 426-435, 437, 439-442, 444-446, 449, 450, 452, 453, 455-463, 465, 467-473, 475-479, 481-486, 490, 491, 493-495, 499-510, or 513-526 wherein amino acid numbering corresponds to SEQ ID NO: 411 or SEQ ID NO: 3 (full length spike protein). In embodiments, the nucleic acid sequence encodes a sequence comprising one or more mutations at amino acid positions 367, 403, 439, 417, 446, 447, 449, 452, 453, 455, 456, 470, 473, 475, 476, 477, 478, 484, 486, 487, 490, 493, 494, 496, 499, 500, 501, 502, 503, 504, and/or 505, wherein amino acid numbering corresponds to SEQ ID NO: 411 or SEQ ID NO: 3 (full length spike protein).

In embodiments, the one or more mutations are selected from substitution of amino acid 417 (K) by N; substitution of amino acid 446 (G) by V, S or A; substitution of amino acid 449 (Y) by N; substitution at amino acid 453 (Y) by F; substitution of amino acid 455 (L) by F; substitution of amino acid 456 (F) by L; substitution of amino acid 473 (Y) by V; substitution of amino acid 475 (A) by V; substitution of amino acid 476 (G) by S or A; substitution of amino acid 477 (S) by N, R, T, G, A or I; substitution at amino acid 484 (E) by Q, K, D, A or R; substitution of amino acid 486 (F) by L or S; substitution of amino acid 453 (Y) by F; substitution of amino acid 493 (Q) by L or R; substitution of amino acid 495 (Y) by N or F; substitution of amino acid 500 (T) by I; substitution of amino acid 501 (N) by Y, T or S; substitution of amino acid 502 (G) by R, D or C; substitution of amino acid 503 (V) by L, I or F; or, substitution of amino acid 505 (Y) by H, E, W or C, wherein amino acid numbering corresponds to SEQ ID NO: 411. In embodiments, the nucleic acid sequence encodes a sequence comprising one or more mutations selected from K417T, K417N, E484K, L452R and/or N501Y, wherein amino acid numbering corresponds to SEQ ID NO: 411 or SEQ ID NO: 3 (full length spike protein). In embodiments, the nucleic acid sequence encoding SEQ ID NO: 446 further comprises a leader sequence encoded by a nucleic acid sequence encoding a sequence selected from SEQ ID NOS: 418 to 437.

In embodiments provided herein is an immunogenic composition wherein the coding sequence of the transgene is codon optimized for a mammalian subject. In embodiments, the replication defective adenoviral vector is a bovine adenovirus, a canine adenovirus, a non-human primate adenovirus, a chicken adenovirus, a porcine or swine adenovirus, or a human adenovirus. In embodiments, the non-human primate adenovirus is a chimpanzee or gorilla adenovirus. In embodiments, the replication defective adenoviral vector is a human adenovirus. In embodiments, the human adenovirus is Ad5 or Ad26.

In embodiments provided herein is a pharmaceutical formulation, comprising an effective amount of the immunogenic composition (e.g., comprising a replication defective adenoviral (rdAd) vector comprising a nucleic acid sequence encoding SEQ ID NO: 446 or a variant comprising at least 90%, or at least 95% identity to SEQ ID NO: 446), the composition comprising at least one pharmaceutically acceptable diluent or carrier, optionally wherein the diluent is phosphate-buffered saline. In embodiments, the formulation is configured for non-invasive or intranasal administration, optionally wherein the pharmaceutically acceptable carrier is in a spray or aerosol form.

In embodiments provided herein is a method for inducing an immune response against SARS-CoV-2, the method comprising administering an effective amount of a present immunogenic composition ((e.g., comprising a replication defective adenoviral (rdAd) vector comprising a nucleic acid sequence encoding SEQ ID NO: 446 or a variant comprising at least 90%, or at least 95% identity to SEQ ID NO: 446) to a human being. Ion embodiments, the effective amount is at least 10⁸ viral particles (vp), at least 10⁹ viral particles (vp), or at least 10¹⁰ viral particles (vp). In embodiments, the immunogenic composition is administered intranasally.

In some embodiments, compositions of the disclosure can be used in the treatment or prevention of SARS-CoV-2, and compositions of the disclosure can be used in the manufacture of a medicament to provide treatment or prevention of SARS-Cov-2. Accordingly, while the invention comprehends methods of treating and prevention of SARS-CoV-2, the invention also comprehends use of the compositions of the invention and such uses can parallel any of the inventive methods and involve any or all of the inventive compositions.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1A-J Exemplary SARS-CoV-2 complete genome (Wuhan seafood market pneumonia virus isolate Wuhan-Hu-1, complete genome; GenBank: MN908947.3; SEQ ID NO: 1).

FIG. 2A-E. SARS-CoV-2 ORF lab Polyprotein (GenBank: QHD43415.1; SEQ ID NO: 2).

FIG. 3A. SARS-CoV-2 surface glycoprotein (e.g. Spike protein) including the native leader sequence (GenBank: QHD43416.1; SEQ ID NO: 3).

FIG. 3B. RBD region of SARS-CoV-2 surface glycoprotein spike protein (SED ID NO: 446).

FIG. 4. SARS-CoV-2 ORF3A protein (GenBank: QHD43417.1; SEQ ID NO: 4).

FIG. 5. SARS-CoV-2 envelope protein (GenBank: QHD43418.1; SEQ ID NO: 5).

FIG. 6. SARS-CoV-2 membrane glycoprotein (GenBank: QHD43419.1; SEQ ID NO: 6).

FIG. 7. SARS-CoV-2 ORF6 protein (GenBank: QHD43420.1; SEQ ID NO: 7).

FIG. 8. SARS-CoV-2 ORF7a protein (GenBank: QHD43421.1; SEQ ID NO: 8).

FIG. 9. SARS-CoV-2 ORF8 protein (GenBank: QHD43422.1; SEQ ID NO: 9).

FIG. 10. SARS-CoV-2 nucleocapsid phosphoprotein (GenBank: QHD43423.2; SEQ ID NO: 10).

FIG. 11. SARS-CoV-2 ORF10 protein (GenBank: QHI42199.1; SEQ ID NO: 11).

FIG. 12 shows the schematic diagram of the Wuhan-Hu-1 SARS-CoV-2 complete genome (GenBank MN908497; NCBI Reference Sequence: NC_045512.2; SEQ ID NO: 1).

FIG. 13 shows the schematic diagram of the Spike protein polypeptide and its domains (e.g. SEQ ID NO: 3)

FIG. 14 shows the taxonomy of coronavirus via the 51 and S2 domains of coronavirus spike protein.

FIG. 15 shows a table of conservative amino acid substitutions at positions 455, 486, 493, 494 and 501 in the Receptor Binding Domain (RBD) of the 51 portion of Spike protein.

FIG. 16. SARS-CoV-2 surface glycoprotein (e.g. Spike protein) (GenBank: QHD43416.1) with a pTA signal sequence underlined. (SEQ ID NO: 12).

FIG. 17A. SARS-CoV-2 spike protein 51 domain with pTA signal sequence underlined (SEQ ID NO: 13). When SEQ ID NO: 13 is inserted into an adenoviral vector it is herein referred to as “51 vector”.

FIG. 17B. SARS-CoV-2 variant mutations in the S1 domain (“Sequence” is SEQ ID NO: 411).

FIG. 18. SARS-CoV-2 spike protein Receptor Binding Domain (RBD) of the S1 domain with pTA signal sequence and short flanking sequence underlined. (SEQ ID NO: 14).

FIG. 19. SARS-CoV-2 spike protein Receptor Binding Domain (RBD) of the S1 domain with pTA signal sequence and long flanking sequence underlined (SEQ ID NO: 15). When SEQ ID NO: 15 is inserted into an adenoviral vector it is herein referred to as “RBD vector”.

FIG. 20. SARS-CoV-2 spike protein Receptor Binding Domain (RBD) of the S1 domain with conservative substitutions at positions 455, 486, 493, 494 and 501 (SEQ ID NO: 16).

FIG. 21. SARS-CoV-2 spike protein, portion of the Receptor Binding Domain (RBD) of the S1 domain with conservative substitutions at positions 455, 486, 493, 494 and 501 (SEQ ID NO: 17).

FIG. 22. SARS-CoV-2 surface glycoprotein (e.g. Spike protein) with a pTA signal sequence underlined and substitutions at the S1/S2, S2′ and HR1 sites (SEQ ID NO: 18).

FIG. 23. SARS-CoV-2 surface glycoprotein (e.g. Spike protein) with a pTA signal sequence underlined and substitutions at the S1/S2, S2′ and HR1 sites (SEQ ID NO: 19).

FIG. 24. SARS-CoV-2 surface glycoprotein (e.g. Spike protein) with a pTA signal sequence underlined and substitutions at the S1/S2, S2′, HR1, fusion peptide and ER retention motif sites (SEQ ID NO: 20).

FIG. 25 shows a dose-dependent vector shedding that is absent at 2 weeks post-administration (of the present monovalent influenza vaccine composition (e.g., NasoVAX)) with no replication competent virus found (as determined via polymerase chain reaction (“PCR”) assay) and anti-vector antibody presented as GMR at Day 29 vs baseline wherein only a 2.3-fold induction after 1 month at highest dose was demonstrated. The present monovalent influenza vaccine composition demonstrates a transient shedding (Advector) with limited anti-vector (Ad-vector) immune response

FIG. 26 shows the effect of NasoVAX administration (high dose; 11×10¹¹ vp) of pre-existing (baseline Ad5 serostatus) anti-vector (Ad5) immunity as measured for humoral (“HAI” or microneutralization “MN” at day 29), mucosal (“IgA” at day 29) and cellular (“ELISpot” at day 8) wherein no difference in an immune response between Ad5 seronegative or Ad5 seropositive subjects was observed. Median titer of Ad5+ subjects was 22-fold above the lower limit of quantification (LLOQ).

FIG. 27. Profiles of high affinity HLA class I and HLA class II binding motifs across the entire SARS-CoV-2 proteome.

FIG. 28. Profiles of high and moderate affinity HLA class I and HLA class II binding motifs across the entire SARS-CoV-2 proteome.

FIG. 29. Map of HLA class I and HLA class II binding motifs for SEQ ID NO: 328.

FIG. 30. Map of HLA class I and HLA class II binding motifs for SEQ ID NO: 329.

FIG. 31. Map of HLA class I and HLA class II binding motifs for SEQ ID NO: 330.

FIG. 32. Map of HLA class I and HLA class II binding motifs for SEQ ID NO: 331.

FIG. 33. Map of HLA class I and HLA class II binding motifs for SEQ ID NO: 332.

FIG. 34. Map of HLA class I and HLA class II binding motifs for SEQ ID NO: 333.

FIG. 35. Map of HLA class I and HLA class II binding motifs for SEQ ID NO: 334.

FIG. 36. Map of HLA class I and HLA class II binding motifs for SEQ ID NO: 335.

FIG. 37. Map of HLA class I and HLA class II binding motifs for SEQ ID NO: 336.

FIG. 38. Map of HLA class I and HLA class II binding motifs for SEQ ID NO: 337.

FIG. 39. Map of HLA class I and HLA class II binding motifs for SEQ ID NO: 338.

FIG. 40. Map of HLA class I and HLA class II binding motifs for SEQ ID NO: 339.

FIG. 41. Map of HLA class I and HLA class II binding motifs for SEQ ID NO: 340.

FIG. 42. Map of HLA class I and HLA class II binding motifs for SEQ ID NO: 341.

FIG. 43. Map of HLA class I and HLA class II binding motifs for SEQ ID NO: 342.

FIG. 44. Map of HLA class I and HLA class II binding motifs for SEQ ID NO: 343.

FIG. 45. Map of HLA class I and HLA class II binding motifs for SEQ ID NO: 344.

FIG. 46. Map of HLA class I and HLA class II binding motifs for SEQ ID NO: 345.

FIG. 47. Map of HLA class I and HLA class II binding motifs for SEQ ID NO: 346.

FIG. 48. Map of HLA class I and HLA class II binding motifs for SEQ ID NO: 347.

FIG. 49. Map of HLA class I and HLA class II binding motifs for SEQ ID NO: 348.

FIG. 50. Map of HLA class I and HLA class II binding motifs for SEQ ID NO: 349.

FIG. 51. Map of HLA class I and HLA class II binding motifs for SEQ ID NO: 350.

FIG. 52. Map of HLA class I and HLA class II binding motifs for SEQ ID NO: 351.

FIG. 53. Map of HLA class I and HLA class II binding motifs for SEQ ID NO: 352.

FIG. 54. Map of HLA class I and HLA class II binding motifs for SEQ ID NO: 353.

FIG. 55. Map of HLA class I and HLA class II binding motifs for SEQ ID NO: 354.

FIG. 56. Map of HLA class I and HLA class II binding motifs for SEQ ID NO: 355.

FIG. 57. Map of HLA class I and HLA class II binding motifs for SEQ ID NO: 356.

FIG. 58. Map of HLA class I and HLA class II binding motifs for SEQ ID NO: 357.

FIG. 59. Map of HLA class I and HLA class II binding motifs for SEQ ID NO: 358.

FIG. 60. Map of HLA class I and HLA class II binding motifs for SEQ ID NO: 359.

FIG. 61. Map of HLA class I and HLA class II binding motifs for SEQ ID NO: 360.

FIG. 62. Map of HLA class I and HLA class II binding motifs for SEQ ID NO: 361.

FIG. 63. Map of HLA class I and HLA class II binding motifs for SEQ ID NO: 362.

FIG. 64. Map of HLA class I and HLA class II binding motifs for SEQ ID NO: 363.

FIG. 65. Map of HLA class I and HLA class II binding motifs for SEQ ID NO: 364.

FIG. 66. Map of HLA class I and HLA class II binding motifs for SEQ ID NO: 365.

FIG. 67. Map of HLA class I and HLA class II binding motifs for SEQ ID NO: 366.

FIG. 68. Map of HLA class I and HLA class II binding motifs for SEQ ID NO: 367.

FIG. 69. Map of HLA class I and HLA class II binding motifs for SEQ ID NO: 368.

FIG. 70. Map of HLA class I and HLA class II binding motifs for SEQ ID NO: 369.

FIG. 71. Measurements of anti-SAR-CoV-2 IgG (μg/ml) in the serum (A), BAL anti-SARS-Cov-2 IgG (ng/ml) (B) and BAL anti-SARS-CoV-2 IgA (ng/ml) (C) from bronchoalveolar lavages obtained at different time points from individual C57BL/6 mice that have received a single intranasal dose of the replication-deficient Ad5 vector expressing the RBD domain (as shown in SEQ ID NO: 15). Results as expressed as the geometric mean response +/−95% confidence interval. Day 0 reports control group values for all groups. Day 7 and day 14 for group receiving 2 intranasal doses report values from group receiving a single administration at the same dose.

FIG. 72. Serum neutralizing antibodies against SARS-CoV-2 measured by focus reduction neutralization test (PRNT) in ten C57BL/6 mice that have received a single intranasal high dose of the replication-deficient Ad5 vector expressing the RBD domain (SEQ ID NO: 15). Each graph (FIG. 72A through 72J) corresponds to the result obtained from one immunized mouse. Lines in black correspond to the negative control serum and lines in red correspond to the tested serum samples.

FIG. 73. Serum neutralizing antibodies against SARS-CoV-2 measured by focus reduction neutralization test (PRNT) in five C57BL/6 mice that have received a single intranasal mid-dose of replication-deficient Ad5 vector expressing the RBD domain (SEQ ID NO: 15). Each graph (FIG. 73A through 73E) corresponds to the result obtained from one immunized mouse. Lines in black correspond to the negative control serum and lines in red correspond to the tested serum samples.

FIG. 74. low cytometry analysis of immune cells in lungs from individual C57BL/6 mice that have received a single intranasal high dose of the replication-deficient Ad5 vector expressing the RBD domain (SEQ ID NO: 15). Results for the groups are expressed as the geometric mean response +/−95% confidence interval. Each graph (FIG. 74A through FIG. 74L) corresponds to an individual immune cell type.

FIG. 75. Flow cytometry analysis of immune cells in bronchoalveolar lavages from individual C57BL/6 mice that have received a single intranasal mid dose of the replication-deficient Ad5 vector expressing the RBD domain (SEQ ID NO: 15). Results for the groups are expressed as the geometric mean response +/−95% confidence interval. Each graph (FIG. 75A through FIG. 75E) corresponds to an individual immune cell type at three time points.

FIG. 76. Flow cytometry analysis of immune cells in mediastinal lymph nodes from individual C57BL/6 mice that have received a single intranasal high dose of the replication-deficient Ad5 vector expressing the RBD domain (SEQ ID NO: 15). Results for the groups are expressed as the geometric mean response +/−95% confidence interval. Each graph (FIG. 76A through FIG. 76J) corresponds to an individual immune cell type at three time points.

FIG. 77. Flow cytometry analysis of immune cells (FIGS. 77A through 77L) in spleens from individual C57BL/6 mice that have received a single intranasal high dose of the replication-deficient Ad5 vector expressing the RBD domain (SEQ ID NO: 15). Results for the groups are expressed as the geometric mean response +/−95% confidence interval. Each graph (FIG. 77A through FIG. 77L) corresponds to an individual immune cell type at three time points.

FIG. 78. Measurements of anti-SAR-CoV-2 IgG (μg/ml) in the serum (A), BAL anti-SARS2 S IgG (ng/ml) (B) and, BAL anti-SARS-2 S IgA (ng/ml) (C) from bronchoalveolar lavages (BAL) obtained at different time points from individual C57BL/6 mice that have received a single intranasal dose of the replication-deficient Ad5 vector expressing the S1 domain (SEQ ID NO: 13). Results for the groups as expressed as the geometric mean response +/−95% confidence interval. Day 0 reports control group values for all groups. Day 7 and day 14 for group receiving 2 intranasal doses report values from group receiving a single administration at the same dose.

FIG. 79. Serum neutralizing antibodies against SARS-CoV-2 measured by focus reduction neutralization test (PRNT) in five C57BL/6 mice that have received a single intranasal high dose of the replication-deficient Ad5 vector expressing the S1 domain (SEQ ID NO: 13). Each graph corresponds to the result obtained from one immunized mouse (FIG. 79A through 79E). Lines in black correspond to the negative control serum, lines in blue correspond to the positive control and lines in red correspond to the tested serum samples.

FIG. 80. Serum neutralizing antibodies against SARS-CoV-2 measured by focus reduction neutralization test (PRNT) in five C57BL/6 mice that have received a single intranasal mid-dose of replication-deficient Ad5 vector expressing the S1 domain (SEQ ID NO: 13). Each graph corresponds to the result obtained from one immunized mouse (FIGS. 80A through 80E). Lines in back correspond to the negative control serum, lines un blue correspond to the positive control and lines in red correspond to the tested serum samples.

FIG. 81. Flow cytometry analysis of immune cells in lungs from individual C57BL/6 mice that have received a single intranasal high dose of the replication-deficient Ad5 vector expressing the S1 domain (SEQ ID NO: 13). Results for the groups are expressed as the geometric mean response +/−95% confidence interval. Each graph (FIG. 81A through FIG. 81L) corresponds to an individual immune cell type at three time points.

FIG. 82. Flow cytometry analysis of immune cell in bronchoalveolar lavages from individual C57BL/6 mice that have received a single intranasal mid dose of the replication-deficient Ad5 vector expressing the S1 domain (SEQ ID NO: 13). Results for the groups are expressed as the geometric mean response +/−95% confidence interval. Each graph (FIG. 82A through FIG. 82E) corresponds to an individual immune cell type at three time points.

FIG. 83. Flow cytometry analysis of immune cells in mediastinal lymph nodes from individual C57BL/6 mice that have received a single intranasal high dose of the replication-deficient Ad5 vector expressing the S1 domain (SEQ ID NO: 13). Results for the groups are expressed as the geometric mean response +/−95% confidence interval. Each graph (FIG. 83A through FIG. 83J) corresponds to an individual immune cell type at three time points.

FIG. 84. Flow cytometry analysis of immune cells in spleens from individual C57BL/6 mice that have received a single intranasal high dose of the replication-deficient Ad5 vector expressing the S1 domain (SEQ ID NO: 13). Results for the groups are expressed as the geometric mean response +/−95% confidence interval. Each graph (FIG. 84A through FIG. 84L) corresponds to an individual immune cell type at three time points.

FIG. 85. Measurements of anti-SAR-CoV-2 IgG (μg/ml) in the serum (A), BAL anti-SARS-CoV-2 IgG (ng/ml) (B) and BAL anti-SARS-COV-2 IgA (ng/ml) (C) in bronchoalveolar lavages (BAL) obtained at different time points from individual CD-1 mice that have received one or two intranasal doses of the replication-deficient Ad5 vector expressing the RBD domain (SEQ ID NO: 15). Results for the groups are expressed as the geometric mean response +/−95% confidence interval. Day 0 reports control group values for all groups. Day 7 and day 14 for group receiving 2 intranasal doses report values from group receiving a single administration at the same dose.

FIG. 86. Measurements of anti-SAR-CoV-2 IgG (μg/ml) in the serum (A), BAL anti-SARS-CoV-2 IgG (ng/ml) (B) and BAL anti-SARS-CoV-2 IgA (ng/ml) (C) from bronchoalveolar lavages obtained at different time points from individual CD-1 mice that have received a single intranasal dose of the replication-deficient Ad5 vector expressing the S1 domain (SEQ ID NO: 13). Results for the groups are expressed as the geometric mean response +/−95% confidence interval. Day 0 reports control group values for all groups.

FIGS. 87A through 87E. SARS-CoV-2 concatenated Polyprotein; Derived from SEQ ID NO: 1 (GenBank: MN908947.3).

FIG. 88. Measurement of lung T cells at day 10 (A) and day 14 (B) and spleen T cells at day 10 (C) and day 14 (D) from individual CD-1 mice that received a single intranasal dose of the replication-deficient Ad5 vector expressing the RBD domain (SEQ ID NO: 15) (“RBD vector”) or control groups that received the A195 buffer only. Results for the groups are expressed as the geometric mean response +/−95% confidence interval.

FIG. 89. Measurements of lung CD4+ IFN-g+ CD11a+ T cells (A), Lung CD4+ TNF-a+ CD11a+ T cells (B), Lung CD4+ IFN-g+ CD11a+ T cells (C) and Lung CD4+ TNF-a+ CD11a+ T cells (D) at day 14 from individual CD-1 mice that received a single intranasal dose of the replication-deficient Ad5 vector expressing the RBD domain (SEQ ID NO: 15) (“RBD vector”) or that received the control A195 buffer only. Prior to flow cytometry analysis, cells were re-stimulated with a RBD peptide pool at 2 mcg/peptide/mL. Lines correspond to geometric mean response +/−95% confidence interval.

FIG. 90. Measurements of Spleen CD4+ IFN-g+ CD11a+ T cells (A), Spleen CD4+ TNF-a+ CD11a+ T cells (B), Spleen CD4+ IFN-g+CD11a+ T cells (C) and Spleen CD4+ TNF-a+CD11a+ T cells (D) at day 14 from individual CD-1 mice that received a single intranasal dose of the replication-deficient Ad5 vector expressing the RBD domain (SEQ ID NO: 15) (“RBD vector”) or that received the control A195 buffer only. Lines correspond to geometric mean response +/−95% confidence interval.

FIG. 91. Measurements of lung resident memory CD4+ (A) and CD8+ (B) T cells expressing the tissue-resident memory T cell (TRM) markers CD103 and CD69 at day 14 from individual CD-1 mice that have received a single intranasal dose of the replication-deficient Ad5 vector expressing the RBD domain (SEQ ID NO: 15) (“RBD vector”) or that received the control A195 buffer only. Lines correspond to geometric mean response +/−95% confidence interval.

FIG. 92. Serum neutralizing antibodies against SARS-CoV-2 measured by focus reduction neutralization test (FRNT) in ten CD-1 mice that have received a single intranasal high dose of the replication-deficient Ad5 vector expressing the RBD domain (SEQ ID NO: 15). Each graph corresponds to the result obtained from one immunized mouse (FIGS. 92A through 92J). Lines in black correspond to the negative control serum and lines in red correspond to the tested serum samples.

FIG. 93. Expression and detection of spike transgene in S1 or RBD vector infected cells. Per.C6 cells were infected at MOI:5 with S1 (SEQ ID NO: 13) or RBD (SEQ ID NO: 15) vector (“S1 vector” or “RBD vector”) respectively. 24 hrs post-infection, cells were harvested, fixed and permeabilized and then stained with mouse neutralizing spike-specific monoclonal antibodies MM-43 (upper panel) or MM-57 (bottom panel) filled histograms. Non-infected cells were used as negative control (open histogram).

FIG. 94. SARS-CoV-2 neutralizing antibody responses in serum following single intranasal administration of the S1 vector and RBD vector. A: Neutralizing antibody response by C57BL/6 or CD-1 mice vaccinated 28 days earlier with the mid or high dose of the S1 or RBD vector as indicated. Results are expressed as the reciprocal of the dilution of serum samples required to achieve 50% neutralization (FRNT₅₀) of wild-type SARS-CoV-2 infection of permissive Vero E6 cells. Line represents the group median value. B: Correlation between neutralizing antibody response and Spike-specific IgG response in serum of vaccinated animals. Correlation analysis was performed with a two-tailed Spearman test. High-dose intranasal vaccination with the RBD vector induced neutralizing antibody responses above background in 10/10 C57BL/6 and 8/10 CD-1 mice.

FIG. 95. Intracellular cytokine production by lung resident memory T cells at 14 days after single intranasal administration with the RBD vector. CD-1 mice were given a single intranasal administration of vehicle (Ctrl) or high dose RBD vector as described in Example 17. Lung cells (n=10 mice/vaccine, 3 mice/control) were isolated at day 14, re-stimulated with the RBD peptide pool for 5 hrs and analyzed by flow cytometry to identify CD69⁺CD103⁺ resident memory T cells (Trm). Results are expressed as the % of CD4+ or CD8+ T cells for individual mice. Lines presented as the mean response +/−SD for the groups. Statistical analysis was performed with a Mann-Whitney test: *, P<0.05; **, P<0.01; ***, P<0.001; ****, P<0.0001.

FIG. 96. Secreted cytokine production by splenic T cells 10 days after single intranasal administration with RBD vector. CD-1 mice were given a single intranasal administration of vehicle (Ctrl) or high dose RBD vector as described in Example 18. Spleen cells (n=10 mice/vaccine, 3 mice/control) were isolated at day 10 and re-stimulated with the RBD peptide pool for 48 hrs. Secreted cytokines were detected in the supernatant using a cytokine multiplex assay. Results are expressed in pg/ml. Lines represent mean response +/−SD. Statistical analysis was performed with a Mann-Whitney test: *, P<0.05; **, P<0.01; ***, P<0.001; ****, P<0.0001.

FIG. 97. Longitudinal spike-specific serum IgG responses following single intranasal administration of the RBD vector. C57BL/6J mice (n=20) received a single intranasal administration of the RBD vector. Sera were collected at days 0 pre-vaccination), and at days 15, 30, 63 and 120 post-vaccination and analyzed individually for quantification of spike-specific IgG. Results are expressed in μg/ml. See Example 18.

FIG. 98. Quantification of RBD-specific antibody secreting cells (“ASCs”) produced IgG and IgA antibodies in the bone marrow and lungs following single intranasal administration of the RBD vector. CD-1 mice (n=5) received a single intranasal administration of the RBD vector. Lung cells (LG) and bone marrow cells (BM) were collected 69 days post-immunization (“69 dpi”) and analyzed individually for the quantification of ASCs by ELISpot. (A) shows an example of a B-cell ELIspot plate wells. (B) Shows measurement of RBD-specific ASCs producing IgG and IgA shown as either the frequency or number per 10⁶ total cells in bone marrow. (C) Shows measurement of RBD-specific ASCs producing IgG and IgA shown as either the frequency or number per 10⁶ total cells in lung. See Example 19.

FIG. 99. Quantification of long-lived RBD-specific memory B cells at day 168 after single intranasal administration of the RBD vector. C57BL/6J (n-4) mice received a single intranasal administration of the RBD vector. Mediastinal lymph nodes were collected 168 days (24 weeks) post-immunization and analyzed individually for the quantification of RBD-specific memory B cells by flow cytometry. Naïve C57BL/6J (n=5) were used as negative control. (A) shows the gating strategy for the flow cytometry analysis. (B) Shows the absolute number of RBD specific memory B cells measured in vaccinated animals compared to naïve animals. See Example 20.

FIG. 100. Quantification of long lived RBD-specific ASCs produced IgG and IgA antibodies in the bone marrow following single intranasal administration of the RBD vector. C57BL/6J (n-4) mice received a single intranasal administration of the RBD vector. Bone marrow cells (BM) were collected 168 days (24 weeks) post-immunization and analyzed individually for the quantification of ASCs by ELISpot. Naïve C57BL/6J (n=5) were used as negative control. (A) Shows an example of B-cell ELIspot plate wells. (B) Shows the measurement of RBD-specific ASCs producing IgG and IgA shown number per 106 total cells in bone marrow. See Example 20.

### DETAILED DESCRIPTION

The present disclosure relates to an immunogenic composition (e.g., vaccine) comprising an adenoviral vector encoding at least one 2019 novel coronavirus SARS-CoV-2 antigen(s) (“a SARS-Cov-2 transgene”), compositions comprising the same, and the use thereof for inducing a protective immune response against SARS-CoV-2. Adenovirus is a naturally occurring respiratory virus that has been used frequently as a vector to introduce genetic material into cells, wherein the adenoviral vector can transduce the SARS-CoV-2 antigen genes into cells of the nasal mucosa (via intranasal administration), leading to transient expression of the encoded SARS-CoV-2 antigen proteins or peptides thereof in such cells. Subsequent production of the SARS-CoV-2 antigen in normal human epithelial cells allows for an immune response against the SARS-CoV-2 antigen as it occurs in naturally circulating coronavirus (e.g., SARS-CoV-2). SARS-CoV-2, initially reported as 2019-nCoV is a new and highly pathogenic virus, only emerging in December 2019. In humans, SARS-CoV-2 is responsible for an illness referred to as the coronavirus disease 2019 (COVID-19) as officially defined by the World Health Organization (WHO). The compositions disclosed herein are one of the first to target SARS-CoV-2 and provide protection against COVID-19 and related disease. Accordingly, the immunogenic compositions disclosed herein provide for prevention and treatments for this new and pathogenic SARS-CoV-2 virus, for which prior treatment did not exist and potential for a pandemic remains. In some embodiments, the immunogenic composition prevents and/or reduces severity of COVID-19 as defined by FDA Guidance for Industry “Development and Licensure of Vaccines to Prevent COVID-19” June 2020 and FDA Guidance for Industry “Emergency Use Authorization for Vaccines to Prevent COVID-19” October 2020, each incorporated herein by reference. In some preferred embodiments, the immunogenic composition reduces the incidence of infection or virologically confirmed asymptomatic or symptomatic cases of COVID-19. In some preferred embodiments, the immunogenic composition reduces the incidence of severe and/or non-severe (mild or moderate) COVID-19 or the incidence of COVID-19 related hospital admissions. In some preferred embodiments, the immunogenic composition reduces the incidence of mild or moderate COVID-19. In some preferred embodiments, the immunogenic composition reduces the incidence of severe COVID-19. In some preferred embodiments, the immunogenic composition reduces the incidence of COVID-19-related Emergency Department visits, COVID-19 hospitalization and/or COVID-19-related death. In some preferred embodiments, the immunogenic composition reduces the severity of COVID-19-related diseases. In some preferred embodiments, the immunogenic composition reduces the transmission of SARS-CoV-2. In some preferred embodiments, the immunogenic composition reduces the transmission of SARS-CoV-2.

In embodiments, this disclosure provides replication defective adenoviral vectors encoding at least one SARS-CoV-2 antigen (e.g., E1A/E3 deletion human Adenovirus type 5 (hAd5) (hAd5-SARS-CoV-2)), and/or another one or more exogenous antigens of a different type of infectious agent (e.g., a different type of virus such as influenza (e.g., Ad-HA)), or lacking a transgene (“hAdE”; e.g., not encoding at least one antigen or immunogen of an exogenous infectious agent “empty”), as well as expression cassettes, e.g., for containing and/or inserting coding sequence(s) into vector(s), comprising a SARS-CoV-2 antigen coding sequence encoding at least one SARS-CoV-2 antigen (and/or another or one or more exogenous antigens of a different type of infectious agent). Such vectors are referred to herein collectively as “SARS-CoV-2 immunization vectors”. As discussed herein, such SARS-CoV-2 immunization vectors (and/or immunogenic compositions comprising the same) are preferably used to induce mucosal, cell-mediated and/or humoral immune responses against SARS-CoV-2 (e.g., against protective SARS-CoV-2 epitopes such as spike (S) protein receptor binding domain (RBD)). In some embodiments, such SARS-CoV-2 immunization vectors (and/or immunogenic compositions comprising the same) stimulate an innate immune response supporting the adaptive immunity of the vector if used prophylactically or interfering directly with SARS-CoV-2 infection if administered during the pre-exposure period (few days before infection) or during the post-exposure period. In some embodiments, this disclosure describes the administration of such vectors (e.g., hAd5-SARS-CoV-2 and/or hAd5) to animals and/or human beings to induce and/or enhance an immune response (e.g., the production of antibodies and/or CD8⁺ T cells (and/or other T cells)) having specificity for SARS-CoV-2 T cell epitope(s) (e.g., a dominant epitopes). In some embodiments, such immune response is protective against SARS-CoV-2 and/or effective in ameliorating the symptoms and/or infection by SARS-CoV-2 and/or reducing transmission of SARS-CoV-2, and in some embodiments can be protective against a SARS-CoV-2 challenge. Thus, in some embodiments, this disclosure describes the use of an immunogenic composition(s) comprising hAd5-SARS-CoV-2 to provide solutions to art-recognized problems regarding SARS-CoV-2 transmission and infection.

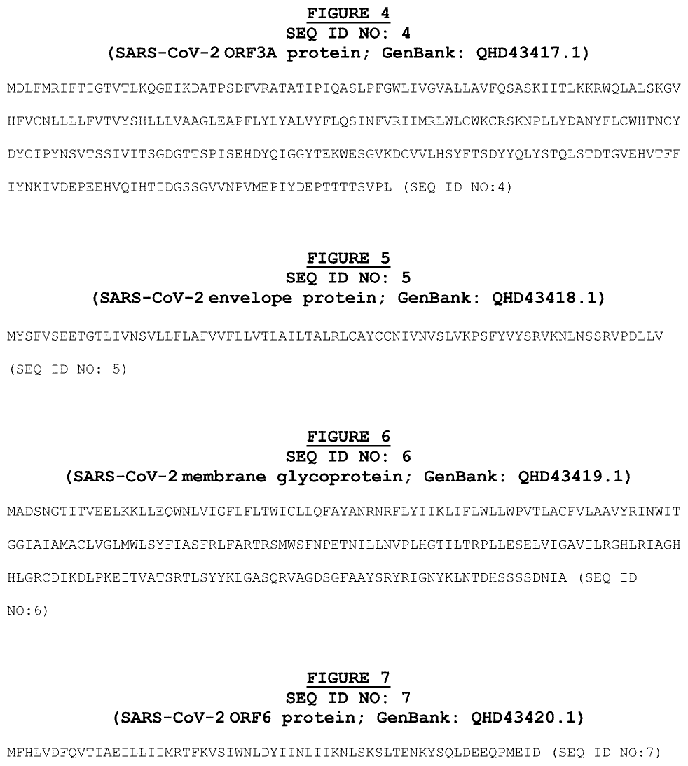

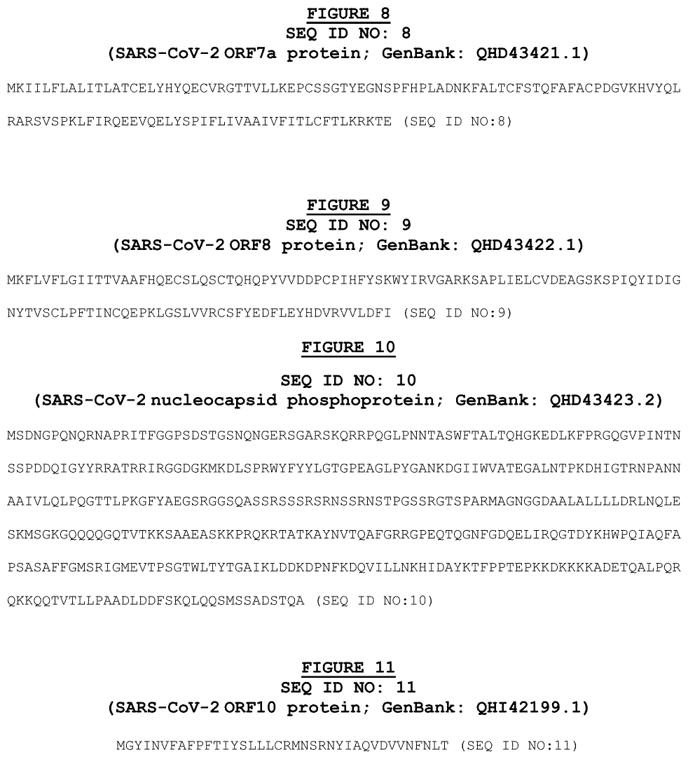

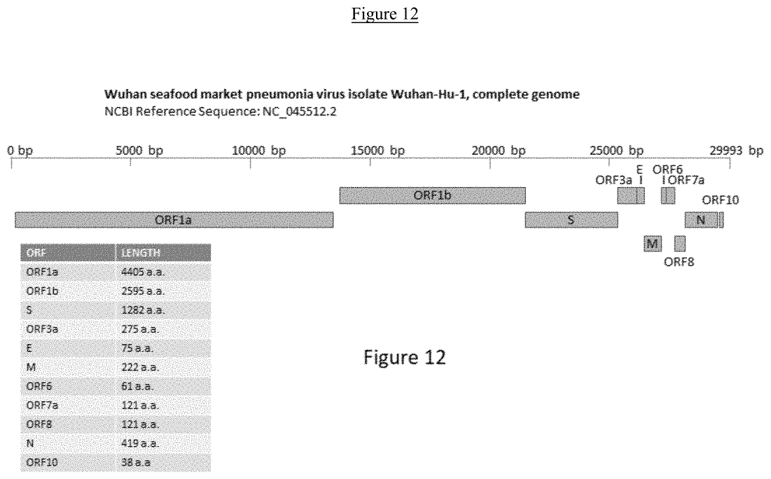

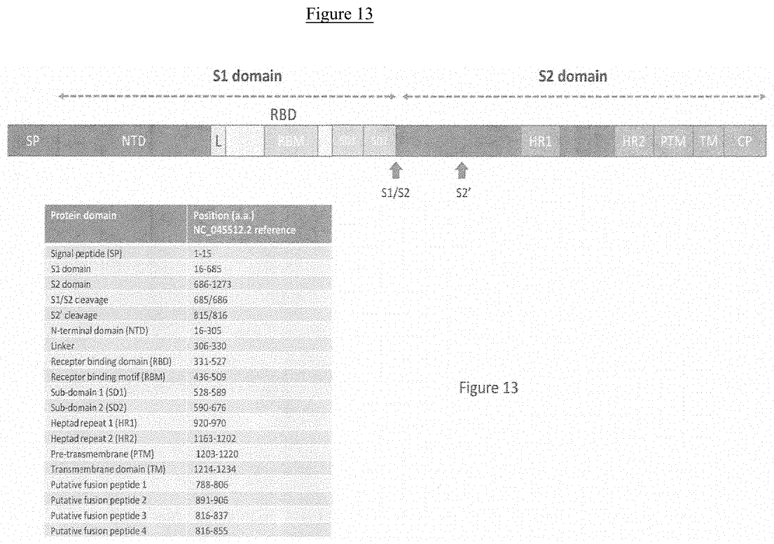

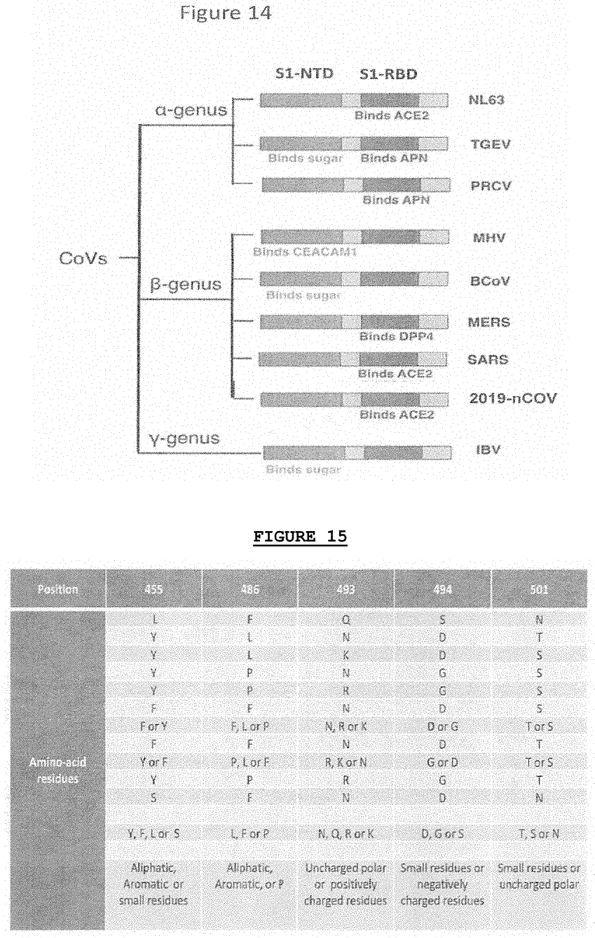

In embodiments, the SARS-CoV-2 antigen can be a spike (S) antigen or other SARS-CoV-2 antigen as disclosed herein, or as may be otherwise available to those of ordinary skill in the art. In certain embodiments, the SARS-CoV-2 antigen can be a full length spike (S) protein, an immunogenic fragment thereof, or a consensus spike (S) antigen derived from the sequences of spike antigens from multiple strains of SARS-CoV-2 (or closely related SARS isolates) identified during the 2019/2020 outbreak and initially sequenced and provided in GenBank MN908497; NCBI Reference Sequence: NC_045512.2 “Wuhan seafood market pneumonia virus isolate Wuhan-Hu-1” (incorporated herein by reference); see also Tegally et al., “Sixteen novel lineages of SARS-CoV-2 in South Africa”, Nat Med (2021), available via internet at doi.org/10.1038/s41591-021-01255-3 (incorporated herein by reference along with all data and code (including extended data) cited in Tegally, and all references cited in Tegally et al. are also hereby incorporated herein by reference); Conti et al., “The British variant of the new coronavirus-19 (Sars-Cov-2) should not create a vaccine problem”, J Biol Regul Homeost Agents“, December 30; 35(1) (2020) available via internet at: doi: 10.23812/21-3-E (incorporated herein by reference); Fiorentini et al., “First detection of SARS-Cov-2 spike protein N501 mutation in Italy in August, 2020”, Lancet Infect Dis (2021) available online at: doi.org/10.1016/ S1473-3099(21)00007-4 (incorporated herein by reference, along with the references cited in Fiorentini also hereby incorporated herein by reference). In some embodiments, the expression cassette comprising a coding sequence encoding at least one coronavirus antigen comprises at least the S1 and/or S2 domains of spike protein, or immunogenic fragments thereof (e.g., RBD sequence of the S1 domain of the spike protein). See FIGS. 13, 14 and 15. In some embodiments, a pre-fusion stabilized spike protein can be used (e.g., by introducing proline residues in S2 (HR1 domains) as shown improved expression in MERS and SARS (see U.S. Pat. Pub. No. 2020/0061185 A1 (Graham, et al.; see, e.g., FIG. 4 thereof), substituting the wild-type amino acid sequence NSPRRARSVAS (SEQ ID NO: 450) with NSPQQAQSVAS (SEQ ID NO: 451) at the S1/S2 cleavage site (QQAQ mutation), KRSFIEDLLFNKVTLADA (SEQ ID NO: 452) with KRSFIADA (SEQ ID NO: 453) at the S2′ cleavage site (fusion peptide truncation, 819-828 deletion), and/or SRLDKVEAEV (SEQ ID NO: 454) with SRLDPPEAEV (SEQ ID NO: 455) (2P mutation, K986P/V987P)). The use of other types of S proteins/antigens is also contemplated herein as would be understood by those of ordinary skill in the art. See Stabilized Spike Protein Antigen Design section below.

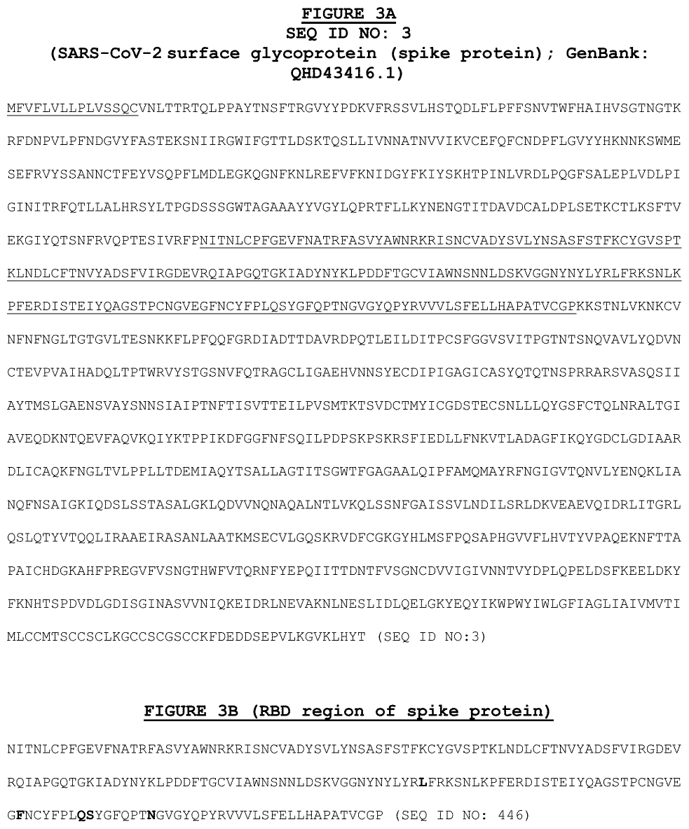

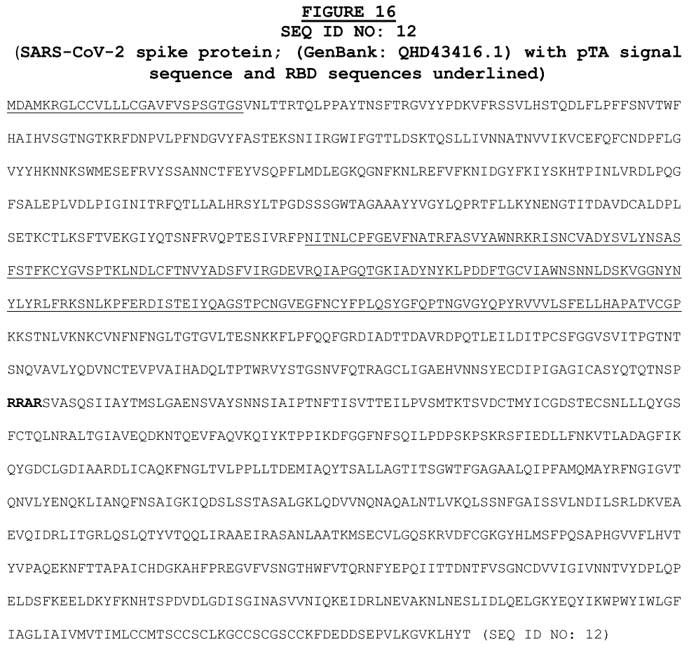

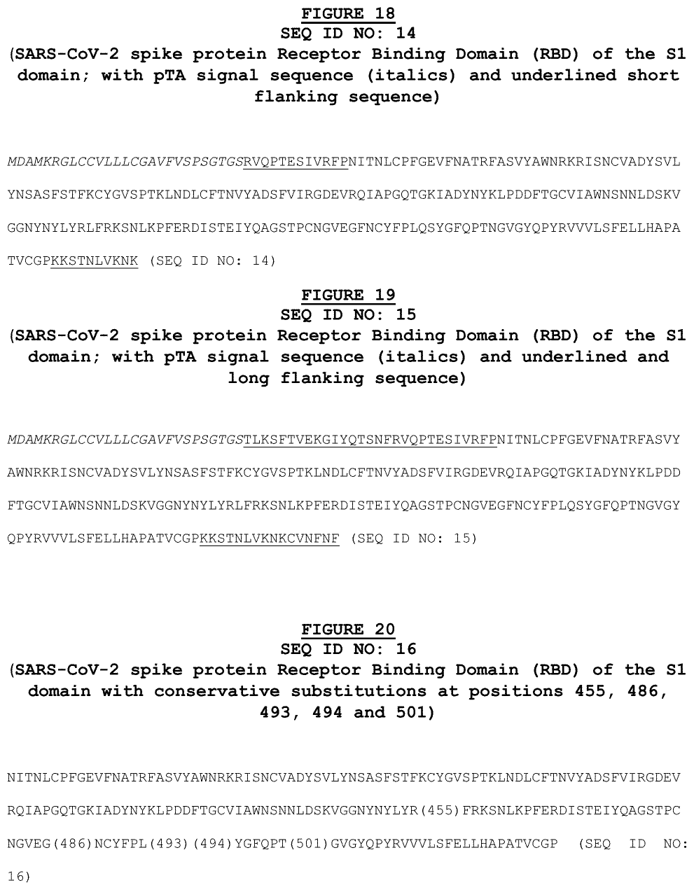

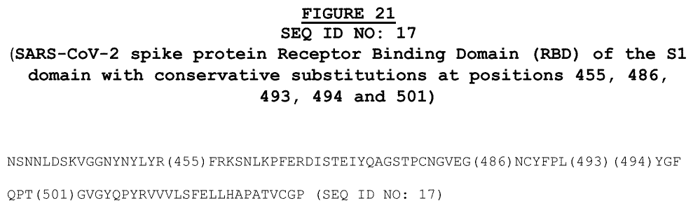

In some embodiments, the expression cassette comprising a coding sequence encoding at least one coronavirus antigen comprises the receptor binding domain (RBD) and/or N-terminal domain (NTD) of S1. See, e.g., FIGS. 13, 16-21. In certain embodiments, the expression cassette comprising a coding sequence encoding SEQ ID NO: 3 (FIG. 3A), or at least one domain sequence (e.g., B cell epitope or T cell epitope) of SEQ ID NO: 3, or at least the RBD domain coding sequence encoding SEQ ID NO: 446 (FIG. 3B). In embodiments, the adenoviral vector encoding a SARS-CoV-2 antigen may be monovalent or multivalent (i.e. one, or more than one antigen epitope). In embodiments, the expression cassette comprises a leader sequence (e.g., tPA) for better expression and secretion of the encoded sequence, wherein the coding sequence is optionally codon optimized for the mammalian subject (e.g., human). In embodiments, the expression cassette comprises the RBD sequence (e.g. SEQ ID NO: 446) optionally further comprising long or short flanking sequences native to the SARS-CoV-2 virus (e.g. SEQ ID NOS: 14 and 15), which may enhance expression of the RBD in its native globular conformation. In embodiments, the immunogenic composition comprises one or more recombinant adenovirus, each encoding one or more SARS-CoV-2 antigen(s).

In some embodiments, this disclosure provides compositions and methods for inducing an immune response against coronavirus in a mammalian subject, including human subjects. In certain embodiments provided herein is an immunogenic composition comprising a replication defective adenoviral vector comprising an expression cassette comprising a coding sequence encoding at least one coronavirus antigen or at least one immunogenic fragment thereof. An immunogenic composition as used herein refers to any one or more Ad-vectored compounds or agents or expressed immunogens and/or antigens capable of priming, potentiating, activating, eliciting, stimulating, augmenting, boosting, amplifying, or enhancing an adaptive (specific) immune response, which may be cellular (T cell), humoral (B cell) and/or mucosal, or a combination thereof. The cellular response may be a peripheral T cell response or a resident T cell response in the nasal mucosa or respiratory tract. Also, cellular responses may preferably be driven by CD8+ T cells and/or CD4+ T cells with an antiviral phenotype (e.g. production interferon-gamma). Preferably, the adaptive immune response is protective, which may include neutralization of a virus (decreasing or eliminating virus infectivity) and/or reduction in symptoms or viral shedding (i.e., transmission).

In some embodiments, this disclosure provides immunological compositions comprising an empty (i.e., without an exogenous non-Ad pathogen antigen encoded in the Ad5 genome) adenovirus vector (AdE) as a therapeutic against coronavirus via activation of an innate immune response including a mucosal innate immune response. See Example 2. In some embodiments, a single intranasal administration can provide protection when the AdE is administered about two to about 20 days before exposure to SARS-Cov-2. In some embodiments, administration of the immunogenic composition can induce increased levels of MCP-1 and IFN-γ both post-vaccination and post-challenge, leading to the recruitment of monocytes, neutrophils, and/or lymphocytes, which can then stimulate production of IFN-γ. In some embodiments, administration of such immunogenic compositions can induce significant decreases (e.g., as compared to placebo controls) in IL-1α, IL-6, and/or IL-12p70, cytokines demonstrated to mediate pulmonary interstitial inflammation in COVID-19. See Example 7.

In some embodiments, this disclosure provides reagents (e.g., immunogenic compositions) and methods for intranasal (i.n.) administration of AdE vectors (i.e., replication deficient ΔE1E3 adenovirus type 5 (Ad5)) viral particles without an exogenous non-Ad pathogen antigen encoded in the Ad5 genome) to confer prophylactic therapy against SARS-CoV-2 in mammals, preferably a human being. In preferred embodiments, such AdE immunogenic compositions can be used to induce an anti-SARS-CoV-2 immune response in human beings (e.g., it is an immunogenic composition) and demonstrate an acceptable safety profile. In preferred embodiments, the resultant immune response is statistically significant, and even more preferably, protective (i.e., it is a SARS-CoV-2 vaccine). In preferred embodiments, such AdE immunogenic compositions can be used to treat a human being infected by SARS-CoV-2 (e.g., hospitalized patients). See Example 3.

In some embodiments, this disclosure provides an E1/E3 deleted, replication defective hAd5 comprises an expression cassette comprising a leader sequence (e.g., tissue plasminogen activator (tPA)) and a codon-optimized nucleotide sequence encoding at least one SARS-CoV-2 protein(s) (e.g., any one or more of SEQ ID NOS: 2-11, and/or one or more fragment(s) and/or derivative(s) thereof), operably linked to a promoter (e.g., cytomegalovirus (CMV)), immunogenic compositions and methods for using the same to induce an immune response against SARS-CoV-2. See Example 4. In some embodiments, the SARS-CoV-2 coding sequence are inserted into the E1 region of the hAd5 (“hAd5-SARS-CoV-2”). In some embodiments, the hAd5-SARS-CoV-2 can be based on a replication-deficient, E1- and E3-deleted adenovirus type 5 vector platform (Tang et al 2009) to express the human codon-optimized gene for the S1 domain (residues 16 to 685 (see, e.g., Examples 14, 16)) or RBD domain (residues 331-527 of the S1 domain (see, e.g., Examples 15, 17)) of SARS-CoV-2 spike protein (accession number QHD43416.1 (SEQ ID NO: 3)). In preferred embodiments, such Ad5-vectored S1 and RBD transgenes included a human tissue plasminogen activator leader sequence and can be expressed under the control of the cytomegalovirus immediate early promoter/enhancer (see, e.g., the preferred embodiments of SEQ ID NO: 13 and SEQ ID NO: 15, respectively). In some embodiments, a human being can be intranasally (i.n.) immunized with a sufficient number of hAd5-SARS-CoV-2 viral particles (vp) or infectious units (ifu) (e.g., at least 1×10⁷, or at least 1×10⁸, or at least 1×10⁹, or at least 1×10¹⁰, or at least 1×10¹¹ vp or ifu), such that neutralizing antibodies are induced. In some embodiments, the hAd5-SARS-CoV-2 vaccine can induce a protective response leading to reduce disease severity. In some embodiments, the hAdv5-SARS-CoV-2 composition can be used to induce an anti-SARS-CoV-2 immune response in human beings (i.e., it is an immunogenic composition), and exhibits an acceptable safety profile. It is preferred that that immune response be statistically significant, and even more preferably, a protective immune response (i.e., it is a SARS-CoV-2 vaccine). In some embodiments, such immunogenic compositions can improve the time to clinical improvement and/or recovery in patients (e.g., hospitalized patients) infected with SARS-CoV-2. See Example 5. In some embodiments, a single (or as part of a prime-boost schedule, same or different immunogenic composition) intranasal administration of a replication-deficient Ad5 vector expressing the RBD domain of the Spike protein (see, e.g., preferred embodiment SEQ ID NO: 15) stimulates the production of IgG antibodies in the serum indicating the induction of systemic responses as well as the production of IgG and IgA antibodies in a mammal, preferably a human being. In preferred embodiments, animals that receive a single administration of the high dose vaccine show the presence of neutralizing antibodies (preferably persistent (see, e.g., Example 18) against SARS-CoV-2 as measured by focus reduction neutralization test (FRNT) and/or induces the recruitment and/or proliferation of innate and adaptive immune cells in different immune compartments (see Example 13B). In preferred embodiments, administration of such immunogenic compositions can induce systemic and mucosal T cell immunity (e.g., in preferred embodiments a Th-1-biased response) against SARS-CoV-2 in the mammal, preferably a human being, as can be determined using flow cytometry (see, e.g., Example 17). In preferred embodiments, an immunogenic composition comprising hAd5-SARS-CoV-2, when administered as a single intranasal dose to a mammal can induce an antibody response against the spike protein that is durable for at least 4 months, for at least about 5 months, at least about 6 months, at least about 7 months, at least about 8 months, at least about 9 months, at least about 10 months, at least about 11 months, or at least about 12 months (one year). See Example 18. In some embodiments, administration of the immunogenic compositions (e.g., hAd5-SARS-CoV-2 such as the RBD vector) can induce bone marrow and lung resident memory antibody secreting cells in a mammal, preferably a human being (e.g., in preferred embodiments bone marrow and lung resident memory antibody secreting cells that secrete both anti-spike IgG and IgA). See Example 19. In preferred embodiments, intranasal administration of the replication incompetent Ad5 vector expressing SARS-CoV-2 spike RBD sequence (see, e.g., preferred embodiment SEQ ID NO: 15) can generate humoral and cellular immune responses in both systemic and mucosal sites, particularly within the lung, which represents a major site for infection and clinical disease (see, e.g., Example 20).

In some embodiments, this disclosure provides methods for intranasal (i.n.) administration of a combination of rdAd anti-SARS-CoV-2 vectors (e.g., a “combined SARS-CoV-2 composition”) to confer prophylactic therapy against SARS-CoV-2. See Example 10. The components of the combined SARS-CoV-2 composition (e.g., AdE, AdD, and/or hAd5-SARS-CoV-2; “AdD” referring to replication-defective adenoviral vector for use in treating and/or preventing coronavirus infection can be one that does not express one or more coronavirus antigens, but expresses one or more antigens of a different type of infectious agent (e.g., influenza virus); “AdE” referring to an rdAd vector for use in preventing and/or treating coronavirus infection can be one that does not express an exogenous antigen (exogenous as to the adenovirus from the adenoviral vector is derived); and “rdAD” or “rdAd” referring to replication-defective adenoviral) can be contained within a single composition or can be contained in different compositions that can be administered simultaneously or at different times (e.g., as part of a prime-boost protocol) and at the same or different sites on a subject (e.g., a mammal, preferably a human being). Preferred prime boost protocols include the administration of first composition to a mammal, preferably a human being, followed by administration of a second composition an appropriate time later (in some preferred embodiments, seven to 21 days later, preferably 7 days later) (i.e., separate administration of the first and second compositions), wherein the first and second compositions comprise the same or different rdAd anti-SARS-CoV-2 vectors. In preferred embodiments, the combined SARS-CoV-2 compositions are configured to induce neutralizing antibody, IgA and/or cellular immune response(s) and/or other response(s) disclosed herein (e.g., avoiding or shortening the time of hospitalization for Covid-19 a patient) against SARS-CoV-2 in a mammalian subject, preferably a human being, to which said immunogenic composition(s) is/are administered. In preferred embodiments, the combined SARS-CoV-2 composition can be used to induce an anti-SARS-CoV-2 immune response in human beings (e.g., it is an immunogenic composition), and with an acceptable safety profile. It is preferred that that immune response be statistically significant, and even more preferably, a protective immune response (i.e., it is a SARS-CoV-2 vaccine). In preferred embodiments, the data shows the combined SARS-CoV-2 composition can be used to treat subjects infected by SARS-CoV-2 (e.g., hospitalized patients).

In some embodiments, the combined SARS-CoV-2 composition can comprise: a) an rdAd vector lacking a coding sequence encoding an exogenous, non-adenoviral, antigen; b) an rdAd vector comprising an expression cassette comprising a SARS-CoV-2 antigen coding sequence encoding at least one SARS-CoV-2 antigen (i.e., hAd5-SARS-CoV-2), optionally wherein said antigen comprises a SARS-CoV-2 spike (S) protein receptor binding domain (RBD); c) an rdAd vector comprising an expression cassette comprising a coding sequence encoding at least one exogenous antigen of an infectious agent other than SARS-CoV-2; d) a combination of the vectors of a) and b); e) a combination of the vectors of b) and c); f) a combination of any of the rdAd vectors of any of a), b), or c); and/or, g) a combination of two different types of rdAd vectors of b) (i.e., hAd5-SARS-CoV-2), wherein each type comprises an expression cassette encoding at least one SARS-CoV-2 antigen different from that encoded by at least one other type of hAd5-SARS-CoV-2 vector in the combination (“multivalent COVID-19 vaccine”). In some embodiments, the components of the combined SARS-CoV-2 composition can comprise one or both of AdE and/or AdD and/or one or more type of hAd5-SARS-CoV-2. In some embodiments, the combined SARS-CoV-2 composition can comprise a first composition comprising AdE and/or AdD that is administered to a human being, followed by administration of a second composition comprising at least one type of hAd5-SARS-CoV-2. In some embodiments, the combined SARS-CoV-2 composition can comprise at least two types of hAd5-SARS-CoV-2, wherein each type of hAd5-SARS-CoV-2 comprises an expression cassette encoding at least one SARS-CoV-2 antigen different from that encoded by the other type(s) of hAd5-SARS-CoV-2 vectors in the combination. Thus, in some embodiments, the combined SARS-CoV-2 composition can comprise a first type of hAd5-SARS-CoV-2 expressing a first SARS-CoV-2 antigen and a second type of hAd5-SARS-CoV-2 encoding a second SARS-CoV-2 antigen, the second SARS-CoV-2 antigen being different from the first SARS-CoV-2 antigen. In some embodiments, the combined SARS-CoV-2 composition can comprise a first composition comprising AdE, AdD, and/or a first type of hAd5-SARS-CoV-2 that is administered to a human being, which is followed by administration of a second composition comprising at least one second type of hAd5-SARS-CoV-2, different from the first type of hAd5-SARS-CoV-2 (or first where the first composition is AdE or AdD) an appropriate time later (in some preferred embodiments, seven to 21 days later, preferably 7 days later) (i.e., separate administration of the first and second compositions).

In some embodiments, this disclosure provides immunogenic compositions comprising and methods for intranasal (i.n.) administration of AdD vectors (i.e., replication deficient ΔE1E3 adenovirus type 5 (Ad5) viral particles encoding a pathogen antigen derived from an infectious agent other than SARS-CoV-2, e.g., influenza such as NasoVAX which is an AdVector (Ad5) expressing influenza hemagglutinin (HA) antigen, described in, e.g., U.S. application Ser. No. 16/840,723, filed Apr. 6, 2020, claiming priority from U.S. application Ser. No. 62/830,444 filed 6 Apr. 2019, and published as US 2020/0316188, each of which, together with all references cited in each of these applications and publications, is incorporated herein by reference and discloses preparation of NasoVAX; see also U.S. Pat. Nos. 6,706,693; 6,716,823; 6,348,450; and US Patent Publications Nos. 2003/0045492; 2004/0009936; 2005/0271689; 2007/0178115; and 2012/0276138, which may pertain to adenoviral vector(s) prepared for administration to a mammal, which may comprise and express an influenza antigen, each of which, with all references cited in each, being hereby incorporated by reference) to confer prophylactic therapy against SARS-CoV-2 (with it mentioned that while NasoVAX is a particular product, in instances where the term “NasoVAX” is used in this disclosure, the skilled person can read both the particular product and also can broadly read an AdVector (Ad5), advantageously an E1 and/or E3 Ad5 vector, expressing an influenza hemagglutinin (HA) antigen). In such embodiments, the AdD vector can induce an innate immune response, preferably a protective immune response, against both SARS-CoV-2 and the pathogen associated with the expressed exogenous antigen of the AdD vector. For example, NasoVAX (or more broadly an immunogenic composition comprising an AdD vector expressing influenza antigen(s)) can be used to induce an immune response against both influenza and coronavirus including SARS-CoV-2. In this way, AdD is a dual vaccine inducing an innate immune response against two respiratory infectious agents, and a protective adaptive immune response against the expressed antigen. In some embodiments, such immunogenic compositions can be administered to patients already infected by SARS-CoV-2 and can improve time to clinical improvement and/or recovery. In some embodiments, then, the AdD composition can be used to induce an anti-SARS-CoV-2 immune response in human beings (e.g., it is an immunogenic composition) with an acceptable safety profile. It is preferred that that immune response be statistically significant, and even more preferably, a protective immune response (i.e., it is a SARS-CoV-2 vaccine). In preferred embodiments, the data shows the AdD composition can be used to treat subjects infected by SARS-CoV-2 (e.g., hospitalized patients). In some embodiments, NasoVAX can be used as therapy for the early phases of infection or as a concomitant therapy for COVID-19, in some embodiments in combination with direct antiviral agents (e.g., chloroquine, azithromycin). See Example 7. At some juncture, the drug substance could transition into a product in which the vector alone (e.g., sans a transgene as in AdE) is administered. In some embodiments, NasoVAX can be effective in reducing rates of ICU admission and mechanical ventilation in patients with early onset COVID-19, and/or reduce the severity of COVID-19 in patients with early onset COVID-19 who require hospitalization. In some embodiments, a decrease in expression of inflammatory cytokines such as IL-1α, IL-5, IL-6, IL-12, IL-17, MCP-1, tumor necrosis factor alpha (TNF-α), granulocyte macrophage colony stimulating factor (GM-CSF), and/or RANTES (CCL5) (see, e.g., Example 2) following administration of NasoVAX to subjects can occur, and can in some embodiments be used to diagnose COVID-19, and/or predict recovery therefrom and used to adjust treatment protocols (e.g., non-NasoVAX treatments) accordingly. In some embodiments, an increase in MCP1 and/or RANTES shortly after administration of NasoVAX, can be used to predict (e.g., as a marker) recovery from COVID-19 and amelioration of symptoms. It is preferred that that immune response be statistically significant, and even more preferably, a protective immune response (i.e., it is a SARS-CoV-2 vaccine). In preferred embodiments, the data shows that NasoVAX can be used to treat subjects infected by SARS-CoV-2 (e.g., hospitalized patients). In preferred embodiments, such an immunogenic composition can be administered repeatedly (e.g., as a seasonal vaccine administered about once every 11-14 months) without inducing a significant immune response against the adenoviral vector itself. See Example 9.

In some embodiments, this disclosure provides adenoviral vectored vaccine compositions (e.g., AdD, NasoVAX) that is stable for about 3 months at an ambient temperature, such as room temperature (e.g., 15 to 30° C., preferably 20-25° C.). In some embodiments, such adenoviral vectored vaccine compositions can be stored, or shipped, without the need for refrigeration or specific storage conditions. In certain embodiments, such adenoviral vectored vaccine compositions can be configured to induce an immune response against SARS-CoV-2 virus (a pandemic coronavirus strain) infection and/or to ameliorate COVID-19 disease symptoms, and may be shipped directly to the user for administration to patients (preferably intranasal administration). See Example 8.

In some embodiments, as shown in Example 2 herein, administration of AdE to mice decreased the expression of certain cytokines known to be involved in the progression and symptoms of infectious diseases caused by viruses such as influenza. For instance, it was shown that non-infected mice (by influenza), 25 days after administration of AdE, exhibited an increase in expression of monocyte chemoattractant protein (MCP-1 (CCL2)), interferon gamma (IFN-γ), and RANTES (CCL5). At 28 days post-administration of AdE, such non-infected mice exhibited increased expression of MCP-1 and IFN-γ but also a decrease in IL-12 expression. Mice challenged with influenza at day 3 post-administration of AdE, mice were found to exhibit decreased expression of IL-1α, IL-6, IL-12, MCP-1, tumor necrosis factor alpha (TNF-α), granulocyte macrophage colony stimulating factor (GM-CSF), and RANTES. At day six (6) post-administration of AdE, the infected mice exhibited decreased expression of IL-5, IL-6, IL-12, IL-17, MCP-1 and GM-CSF, and increased expression of macrophage inflammatory protein 1 alpha (MIP-la (CCL3)) and RANTES (CCL5). These results are consistent with the development of a “cytokine storm” during infection by SARS-CoV-2. In some embodiments, then, to prevent and/or treat SARS-CoV-2 infection by, for instance, inhibiting the development of or suppressing a cytokine storm, a SARS-CoV-2 immunogenic composition is administered to a human being with one or more anti-cytokine reagent(s) (e.g., one or more anti-IL-1α reagent(s), one or more anti-IL5 reagent(s), one or more anti-IL-6 reagent(s), one or more anti-IL-12 reagent(s), one or more anti-IL-17 reagent(s), one or more anti-MCP-1 reagent(s), one or more anti-TNF-α reagent(s), one or more anti-GM-CSF reagent(s), and/or one or more anti-RANTES reagent(s). See Example 11. In some embodiments, the one or more anti-cytokine reagents would not include one or more anti-MIPα reagent(s) and/or one or more anti-RANTES reagent(s). Exemplary anti-cytokine reagents that can be used as described herein can include, for example, any of those shown in Table 1 herein. In some embodiments, such anti-cytokine reagents can be administered with the SARS-CoV-2 immunogenic composition at the same time (i.e., simultaneously), or essentially the same time, by a suitable route appropriate for each reagent (e.g., intranasal administration of the SARS-CoV-2 immunogenic composition and subcutaneous injection for the anti-cytokine reagent(s)) in effective amounts. In some embodiments, the one or more anti-cytokine reagent(s) can be co-administered with the SARS-CoV-2 composition and, in some embodiments, the one or more anti-cytokine reagents are subsequently administered as the sole active agents. In preferred embodiments, the combination of SARS-CoV-2 composition(s) and one or more anti-cytokine reagent(s) can be useful for inducing an anti-SARS-CoV-2 immune response in human beings (e.g., it is an immunogenic composition), with an acceptable safety profile, and with alleviation of symptoms related to the deleterious effects of cytokines experienced by some patients (e.g., the aforementioned cytokine storm). In preferred embodiments, the immune response be statistically significant, and even more preferably, that it is a protective and/or curative immune response (i.e., it is a SARS-CoV-2 vaccine). In preferred embodiments, the combination of SARS-CoV-2 composition(s) and one or more anti-cytokine reagent(s) can be used to treat subjects infected by SARS-CoV-2 (e.g., hospitalized patients).

As described herein, the S protein (spike protein) immunogen, fragments, and variants thereof described herein contain one or more epitopes that elicit or induce an immune response, preferably a protective immune response, which may be a humoral response, a mucosal IgA response and/or a cell-mediated immune response. A protective immune response may be manifested by at least one of the following: preventing infection of a host by a coronavirus; modifying or limiting the infection; aiding, improving, enhancing, or stimulating recovery of the host from infection; and generating immunological memory that will prevent or limit a subsequent infection by a coronavirus. A humoral response may include production of antibodies that neutralize infectivity, lyse the virus and/or infected cell, facilitate removal of the virus by host cells (for example, facilitate phagocytosis), and/or bind to and facilitate removal of viral antigenic material. An antibody response may also include a mucosal response, which comprises eliciting or inducing a specific mucosal IgA response. In certain embodiments is provided a method for inducing a combined mucosal, humoral and/or cell-mediated protective immune response in a human subject against SARS-CoV-2 infection.

Provided herein are pharmaceutically acceptable compositions (which may also be referred to as formulations) suitable and/or configured for intranasal administration to a mammalian subject and that are configured to induce an immune response against an antigen (e.g., an immunogen), and optionally induce a protective immune response (i.e., as a vaccine). In some embodiments, the pharmaceutical formulation is an immunogenic composition that upon administration induces an immune response against an antigen in a mammalian subject. In some embodiments, the pharmaceutical formulation is a vaccine or therapeutic composition configured to induce a protective immune response in a mammalian subject, which is protective against foreign infectious agents, and in preferred embodiments induce or stimulate a protective response against SARS-CoV-2 infection.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

The compositions, formulations and methods of the present invention may comprise, consist essentially of, or consist of the components and ingredients of the present invention as well as other ingredients described herein. As used herein, “consisting essentially of” and “consists essentially of” means that the compositions, formulations and methods may include additional steps, components or ingredients, but only if the additional steps, components or ingredients do not materially alter the basic and novel characteristics of the claimed compositions, formulations and methods. Terms such as “comprises”, “comprised”, “comprising” and the like are synonymous with terms such as “including,” “containing,” or “characterized by,” and are inclusive or open-ended terms that not exclude additional, unrecited elements, components or ingredients or steps. In this regard, it is an object of the invention not to encompass within the invention any previously known product, process of making the product, or method of using the product such that Applicant(s) reserve the right and hereby disclose a disclaimer of any previously known product, process, or method. It is further noted that the invention does not intend to encompass within the scope of the invention any product, process, or making of the product or method of using the product, which does not meet the written description, enablement and/or clarity or definiteness requirements of US Law/the USPTO (e.g. 35 USC § 112(a), (b)) or the sufficiency requirements of EPC/the EPO (e.g. Article 83 of the EPC), such that Applicant(s) reserve the right and hereby disclose a disclaimer of any subject matter not meeting written description, enablement and/or clarity/definiteness and/or sufficiency requirements and/or that which is a previously described or known product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. All rights to explicitly disclaim that which is in any prior-filed but not prior published patent application or patent is explicitly reserved (with “prior-filed but not prior published” being relative to the filing date accorded this disclosure). Nothing herein is to be construed as a promise. Nor is any citation or identification of any document in this application an admission that such document is available as prior art.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

As used herein, an “adjuvant” refers to a substance that enhances the body's immune response to an antigen. In embodiments, the present monovalent influenza pharmaceutical formulation is a non-adjuvanted vaccine composition.

By “administration” is meant introducing an immunogenic or vaccine composition of the present disclosure into a subject; it may also refer to the act of providing a composition of the present disclosure to a subject (e.g., by prescribing or administering).

As used herein, the term “ambient temperature” is the air temperature for storing the present monovalent influenza pharmaceutical formulation. In embodiments, the ambient temperature is a room temperature, such as selected from any temperature within the range from about 15 to 30° C., preferably from about 20 to 25° C.

The term “therapeutically effective amount” as used herein refers to that amount of the compound being administered which will induce a combined, mucosal, humoral and cell mediated immune response. The term also refers to an amount of the present compositions that will relieve or prevent to some extent one or more of the symptoms of the condition to be treated. In reference to conditions/diseases that can be directly treated with a composition of the disclosure, a therapeutically effective amount refers to that amount which has the effect of preventing the condition/disease from occurring in a mammal that may be predisposed to the disease but does not yet experience or exhibit symptoms of the condition/disease (prophylactic treatment), alleviation of symptoms of the condition/disease, diminishment of extent of the condition/disease, stabilization (e.g., not worsening) of the condition/disease, preventing the spread of condition/disease, delaying or slowing of the condition/disease progression, amelioration or palliation of the condition/disease state, and combinations thereof. The term “effective amount” refers to that amount of the compound being administered which will produce a reaction (e.g., a protective immune response and/or as provided by a vaccine) that is distinct from a reaction that would occur in the absence of the compound.

As used herein, the term “percent (%) homology” or “percent (%) identity” and grammatical variations thereof in the context of two sequences (e.g., protein sequences), refers to two or more sequences or subsequences (i.e., fragment thereof) that have at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity (homology), when compared and aligned for maximum correspondence, as measured using one of the well-known sequence comparison algorithms or by visual inspection. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877. Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448. Advantageous for use is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein). In addition, from this definition, when this disclosure speaks about percent (%) homology, the reader can also understand percent (%) identity. In addition, it should be understood that proteins within this invention may differ from the exact sequences illustrated and described in this disclosure. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention. Nucleic acid sequences within the invention, as to such proteins, will similarly vary from this explicitly disclosed herein. The invention thus encompasses nucleotide sequences encoding functionally and/or antigenically equivalent variants and derivatives of the antigens or proteins herein disclosed and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and, phenylalanine/tyrosine/tryptophan.

As used herein, the term “human adenovirus” is intended to encompass all human adenoviruses of the Adenoviridae family, which include members of the Mastadenovirus genera. To date, over fifty-one human serotypes of adenoviruses have been identified (see, e.g., Fields et al., Virology 2, Ch. 67 (3d ed., Lippincott-Raven Publishers)). The adenovirus may be of serogroup A, B, C, D, E, or F. The human adenovirus may be a serotype 1 (Ad 1), serotype 2 (Ad2), serotype 3 (Ad3), serotype 4 (Ad4), serotype 5 (Ad5), serotype 6 (Ad6), serotype 7 (Ad7), serotype 8 (Ad8), serotype 9 (Ad9), serotype 10 (Ad10), serotype 11 (Ad11), serotype 12 (Ad12), serotype 13 (Ad13), serotype 14 (Ad14), serotype 15 (Ad15), serotype 16 (Ad16), serotype 17 (Ad17), serotype 18 (Ad18), serotype 19 (Ad19), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or combinations thereof, but are not limited to these examples. In certain embodiments, the adenovirus is serotype 5 (Ad5).

As used herein, an “immunogenic composition” refers to a composition, typically comprising at least one type of replication defective adenoviral vector as disclosed herein and at least one pharmaceutically acceptable carrier, that when administered to a host induces and/or enhances an immune response against an antigen and/or infectious agent against which such immune response is directed (e.g., an antigen encoded by a replication defective adenoviral vector, and/or as may be induced/enhanced by an “empty” hAd5 vector). A “vaccine” refers to such an immunogenic composition that when administered induces a protective immune response against an infectious agent (e.g., protects the host against challenge with the infectious agent). In certain embodiments, an immunogenic composition (e.g., vaccine) can comprise one or more viral vector(s) containing and/or expressing an antigen, along with other components of an immunogenic composition (e.g., vaccine) suitable for administration to a mammalian host, including for example one or more adjuvants, slow release compounds, solvents, buffers, etc. In certain embodiments, an immunogenic composition and/or vaccine can comprise a protein and/or carbohydrate and/or lipid and/or other antigen, including but not limited to one or more killed antigen(s) (e.g., a killed or completely inactive virus) or a live attenuated antigen (e.g., an attenuated virus). In some embodiments, the immunogenic composition(s) and/or vaccine(s) improve immune responses to any antigen regardless of the antigen source or its function.

As used herein, a “pharmaceutically acceptable carrier” refers to a carrier or diluent that does not cause significant irritation to the human subject and does not abrogate the biological activity and properties of the administered immunogenic or vaccine compositions.

As used here, the term “seroconversion” is defined as a 4-fold or greater increase in serum neutralization antibody titers (e.g., anti-S1/S2 antibody or anti-RBD of 51 antibody) after vaccination (e.g., administration of a present immunogenic or vaccine composition).

As used herein, the term “seropositive” means a measurable (e.g., detectable in an in vitro assay) in serum neutralization antibody after vaccination (e.g., administration of a present immunogenic composition).

As used herein, the term “protection” indicates that a protective immune response has been elicited, and a protective immune response may be manifested by at least one of the following: preventing infection of a host by a coronavirus; modifying or limiting the infection; aiding, improving, enhancing, or stimulating recovery of the host from infection; and generating immunological memory that will prevent or limit a subsequent infection by a coronavirus. A humoral response may include production of antibodies that neutralize infectivity, lyse the virus and/or infected cell, facilitate removal of the virus by host cells (for example, facilitate phagocytosis), and/or bind to and facilitate removal of viral antigenic material. An antibody response may also include a mucosal response, which comprises eliciting or inducing a specific mucosal IgA response. As used herein, the term “seroprotected” means a subject post vaccination that is protected from infection via generation of serum neutralization antibodies. In a population, this is referred to as a percentage (%) of seroprotected individuals (e.g., 50%). In embodiments, the present immunogenic compositions and methods of use provide seroprotection to the mammalian subject, such as a human subject, against SARS-CoV-2 infection. The duration of protection can be at least about one month to at least about 14 months. Seroprotection can last at least about 1 month, 2 months, 4 months, 6 months, 8 months, 10 months, 12 month or at least about 13 months.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also mean prolonging survival as compared to expected survival if not receiving treatment and/or can be therapeutic in terms of a partial or complete cure for a disease and/or adverse effect attributable to the disease. As used herein, the terms “prophylactically treat” or “prophylactically treating” refers completely, substantially, or partially preventing a disease/condition or one or more symptoms thereof in a host. Similarly, “delaying the onset of a condition” can also be included in “prophylactically treating” and refers to the act of increasing the time before the actual onset of a condition in a patient that is predisposed to the condition.

In this disclosure, a “vaccine” advantageously refers to a composition comprising a replication defective adenoviral vector containing and expressing a coronavirus antigen or other infectious agent, and/or lacking a coding sequence for an exogenous antigen (e.g., empty Advector), along with other components of a vaccine formulation, including for example adjuvants, slow release compounds, solvents, etc., for inducing a protective immune response. Such compositions within this disclosure that comprise a replication defective adenoviral vector containing and expressing a coronavirus antigen or other infectious agent, and/or lacking a coding sequence for an exogenous antigen (e.g., empty Advector), along with other components of a vaccine formulation, including for example adjuvants, slow release compounds, solvents, etc. can also be for inducing an immune response, and are within “immunogenic compositions” herein-discussed. In embodiments of the invention, vaccines or immunogenic compositions can improve immune responses to any antigen regardless of the antigen source or its function.

As referred to herein, a “vector” carries a genetic code, or a portion thereof, for an antigen, however it is not the antigen itself. In an exemplary aspect, a vector can include a viral vector, such as an adenoviral vector. As referred to herein an “antigen” means a substance that induces and/or enhances a specific immune response against the antigen, and/or an infectious agent expressing such antigen, in a subject, including humans and/or animals. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a polypeptide, an epitope, a hapten, or any combination thereof. In various aspects, the antigen is a virus, bacterium, a subunit of an organism, an auto-antigen, or a cancer antigen.

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

Immunogenic Compositions and Vaccines

Provided herein are replication-defective adenoviral (“rdAd”) vector and immunogenic compositions comprising the same, (in some embodiments vaccine formulations) suitable and/or configured for administration to a mammalian subject for the prevention and/or treatment of coronavirus infection (“coronavirus pharmaceutical formulations”), preferably wherein the coronavirus is SARS-CoV-2. In some embodiments, any adenoviral vector (Ad-vector) known to one of skill in art, and prepared for administration to a mammal, which may comprise and express a coronavirus antigen, preferably a SARS-CoV-2 antigen, but also may not express an exogenous (i.e., non-Ad) antigen, or may be an empty AdVector (e.g., no exogenous antigen transgene) may be used in the compositions and with the methods of this application. Such Ad-vectors include any of those known to those of ordinary skill in the art including but not limited to those described in U.S. Pat. Nos. 6,706,693; 6,716,823; 6,348,450; and/or US Patent Publication Nos. 2003/0045492; 2004/0009936; 2005/0271689; 2007/0178115; and/or, 2012/0276138; all of which being incorporated herein incorporated by reference in their entireties. In certain embodiments, the non-replicating adenoviral viral vector (rdAd) is a human adenovirus. In alternative embodiments, the adenovirus is a bovine adenovirus, a canine adenovirus, a non-human primate adenovirus (e.g., chimp), a chicken adenovirus, or a porcine or swine adenovirus. In exemplary embodiments, the non-replicating viral vector is a human adenovirus. In some embodiments, the non-replicating adenoviral vectors are particularly useful for gene transfer into eukaryotic cells and immunogenic composition (e.g., vaccine) development, and in animal models.

In certain embodiments the recombinant adenovirus vector may be non-replicating or replication-deficient (RD) requiring complementing E1 activity for replication. In embodiments the recombinant adenovirus vector may include E1-defective, E3-defective, and/or E4-defective adenovirus vectors, or the “gutless” adenovirus vector in which viral genes are deleted. The E1 mutation raises the safety margin of the vector because E1-defective adenovirus mutants are replication incompetent in non-permissive cells. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. In exemplary embodiments, the recombinant adenovirus vector is an E1 and E3 defective vector. The “gutless” adenovirus vector replication requires a helper virus and a special human 293 cell line expressing both Ela and Cre, a condition that does not exist in natural environment; the vector is deprived of viral genes, thus the vector as an immunogenic composition (e.g., vaccine) carrier is non-immunogenic and may be inoculated for multiple times for re-vaccination. The “gutless” adenovirus vector also contains 36 kb space for accommodating transgenes, thus allowing co-delivery of a large number of antigen genes into cells. Specific sequence motifs such as the RGD motif may be inserted into the H-I loop of an adenovirus vector to enhance its infectivity. An adenovirus recombinant may be constructed by cloning specific transgenes or fragments of transgenes into any of the adenovirus vectors such as those described below. The adenovirus recombinant vector is used to transduce epidermal cells of a vertebrate in a non-invasive mode for use as an immunizing agent. The adenovirus vector may also be used for invasive administration methods, such as intravenous, intramuscular, or subcutaneous injection.

In some embodiments, such an rdAd vector for use in preventing and/or treating coronavirus infection can be one that does not express an exogenous antigen (exogenous as to the adenovirus from the adenoviral vector is derived), such vectors being referred to herein as “AdE” vectors. In some embodiments, the replication-defective adenoviral vector for use in treating and/or preventing coronavirus infection can be one that does not express one or more coronavirus antigens, but expresses one or more antigens of a different type of infectious agent (e.g., influenza virus) (referred to herein as “AdD”). In certain embodiments is provided an immunogenic composition comprising a rdAd vector comprising an expression cassette comprising a coding sequence encoding at least one SARS-CoV-2 antigen, referred to herein as hAd5-SARS-CoV-2 vectors. In some embodiments, the immunogenic compositions of this disclosure can comprise a different type of such vectors (e.g., AdE, or AdD), alone or in combination with hAd5-SARS-CoV-2 vectors. In some embodiments, these types of vectors can be collectively, or a subset of at least two such vectors, referred to as “rdAd anti-SARS-CoV-2 vectors”. A SARS-CoV-2 immunogenic composition (e.g., vaccine) is a pharmaceutical formulation comprising one or more such rdAd anti-SARS-CoV-2 vectors.

An AdE vector is a rdAd vector that does not encode an exogenous antigen (i.e., an antigen exogenous as to the adenovirus from the adenoviral vector is derived, e.g., an antigen of a different type of infectious agent such as influenza). Such hAd5 vectors can also be referred to as “empty”, lacking an exogenous transgene, and/or being “transgene-free”. In some embodiments, an AdE vector can be a ΔE1E3 Ad5 vector (e.g., lacking the E1 region of the viral genome (nucleotides 343 to 3511) and nucleotides 28132 to 30813 in the E3 region). This disclosure provides some embodiments, comprising immunogenic compositions comprising AdE vectors (including AdE viral particles) and the use of such immunogenic compositions to prevent and/or treat coronavirus infection, preferably wherein the coronavirus is SARS-CoV-2, and methods for doing so. In some embodiments, such AdE vectors can be co-administered with one or more other rdAd anti-SARS-CoV-2 vectors. In some embodiments, such co-administration can refer to administration of a single immunogenic composition comprising AdE vectors and one or more rdAd anti-SARS-CoV-2 vectors, and/or essentially simultaneous and/or sequential administration of multiple immunogenic compositions comprising AdE vectors and another immunogenic composition comprising one or more other rdAd anti-SARS-CoV-2 vectors. Such AdE vectors can also be administered as part of a prime-boost protocol, in which an immunogenic composition comprising AdE is administered before or after (e.g., 7-28 days before and/or after) administration of an immunogenic composition comprising one or more other types of rdAd anti-SARS-CoV-2 vectors. In some embodiments, this disclosure provides methods for inducing (and/or enhancing) an immune response against SARS-CoV-2 in a mammalian subject in need thereof by administering (e.g., intranasally) an effective amount of such composition(s) (e.g., at least about 10⁷ infectious units (ifu) or virus particles (vp) (e.g., at least 1×10⁷, or at least 1×10⁸, or at least 1×10⁹, or at least 1×10¹⁰, or at least 1×10¹¹ vp or ifu of AdE and, where present, at least one other rdAd anti-SARS-CoV-2 vectors). In some embodiments, the immune response against SARS-CoV-2 induced or enhanced by administration of such immunogenic compositions preferably begins within about twenty-four hours of administration and preferably lasts for at least about 21 days. In preferred embodiments, such methods comprise intranasal administration of such immunogenic compositions in an effective amount of (e.g., at least about 10⁷ ifu of the AdE and, where present, at least one other rdAd anti-SARS-CoV-2 vectors). In preferred embodiments, the second rdAd anti-SARS-CoV-2 vector encodes at least one heterologous antigen of SARS-CoV-2 and/or at least one other infectious agent (e.g., AdD), thereby providing a drug-vaccine duo regimen. In some embodiments, the administering of multiple doses of AdE vectors (and/or other rdAd anti-SARS-CoV-2 vectors) can be about any of 7, 10, 14, 21, 28, 35, 42, 49, or 56 days apart. Preferably, such immunogenic compositions can be administered intranasally. In some embodiments, the host is an animal, such as an adult or child human being, optionally wherein the host is immunocompromised. In preferred embodiments, the immune response against the coronavirus lasts for at least about 40-50 days, and can be re-initiated by re-administration of AdE with or without the one or more SARS-CoV-2 vectors. Other embodiments of such AdE vectors, immunogenic compositions, and/or methods are also contemplated herein as would be understood by those of ordinary skill in the art.

An AdD vector is a rdAd vector that encodes an exogenous antigen of an infectious agent other than coronavirus and/or SARS-CoV-2 (e.g., an antigen of a different type of infectious agent such as influenza (e.g., swine influenza, seasonal influenza, avian influenza, H1N1 influenza, or H5N1 influenza). In some embodiments, an AdD vector can be a ΔE1E3 Ad5 vector encoding at least one heterologous (e.g., non-Ad) antigen, preferably optimized for expression in a host (e.g., a mammal). Representative examples of antigens which can be used to produce an immune response against SARS-CoV-2 using the methods described herein can include influenza hemagglutinin, influenza nuclear protein, influenza M2, tetanus toxin C-fragment, anthrax protective antigen, anthrax lethal factor, rabies glycoprotein, HBV surface antigen, HIV gp 120, HIV gp 160, human carcinoembryonic antigen, malaria CSP, malaria SSP, malaria MSP, malaria pfg, and Mycobacterium tuberculosis HSP, etc. In one embodiment, an AdD vector can be the AdNC.H1.1 vector encoding the A/New Caledonia/20/99 H1N1 IFV (NC20) HAI domain (see, e.g., Tang et al. Expert Rev Vaccines 8: 469-481 (2009)). In one embodiment, the AdD vector can contain a genetic insert encoding the hemaglutinnin (HA) surface protein antigen from an A/California/04/2009(H1N1)-like strain of influenza (AdcoCA09.HA “NasoVAX”), preferably manufactured by propagation in replication-permissive PER.C6 cells, followed by purification of the virus from the infected cell harvest, and prepared as a final product including the following excipients: Tris HCl (pH 7.4), histidine, sucrose, sodium chloride, magnesium chloride, polysorbate 80, ethylenediaminetetraacetic acid, and ethanol. This disclosure provides some embodiments, that comprise immunogenic compositions comprising AdD vectors (including AdD viral particles) and the use of such immunogenic compositions to prevent and/or treat coronavirus infection, preferably wherein the coronavirus is SARS-CoV-2, and methods for doing so. In some embodiments, such AdD vectors can be co-administered with one or more other rdAd anti-SARS-CoV-2 vectors. In some embodiments, such co-administration can refer to administration of a single immunogenic composition comprising AdD vectors and one or more rdAd anti-SARS-CoV-2 vectors, and/or essentially simultaneous and/or sequential administration of multiple immunogenic compositions comprising AdD vectors and another immunogenic composition comprising one or more other rdAd anti-SARS-CoV-2 vectors. Such AdD vectors can also be administered as part of a prime-boost protocol, in which an immunogenic composition comprising AdD vectors (e.g., as viral particles) is administered before or after (e.g., 7-21 days before and/or after) administration of an immunogenic composition comprising one or more other types of rdAd anti-SARS-CoV-2 vectors. In some embodiments, this disclosure provides methods for inducing (and/or enhancing) an immune response against SARS-CoV-2 in a mammalian subject in need thereof by administering (e.g., intranasally) an effective amount of such composition(s) (e.g., at least about 10⁷ viral particles (vp) or infectious units (ifu) (e.g., at least 1×10⁷, or at least 1×10⁸, or at least 1×10⁹, or at least 1×10¹⁰, or at least 1×10¹¹ vp or ifu) of each of the AdD vector and, where present, at least one other rdAd anti-SARS-CoV-2 vectors). In some embodiments, the immune response against SARS-CoV-2 induced or enhanced by administration of such immunogenic compositions preferably begins within about twenty-four hours of administration and preferably lasts for at least about 21 days. In preferred embodiments, such methods comprise intranasal administration of such immunogenic compositions in an effective amount of (e.g., at least about 10⁷ vp or ifu (e.g., at least 1×10⁷, or at least 1×10⁸, or at least 1×10⁹, or at least 1×10¹⁰, or at least 1×10¹¹ vp or ifu) of each of the AdD vector and, where present, at least one other rdAd anti-SARS-CoV-2 vectors). In preferred embodiments, the second rdAd anti-SARS-CoV-2 vector is AdE and/or encodes at least one heterologous antigen of SARS-CoV-2 and/or at least one other infectious agent, thereby providing a drug-vaccine duo regimen. In some embodiments, the administering of multiple doses of AdD vectors (and/or other rdAd anti-SARS-CoV-2 vectors) can be about any of 7, 10, 14, 21, 28, 35, 42, 49, or 56 days apart. Preferably, such immunogenic compositions can be administered intranasally. In some embodiments, the host is an animal, such as an adult or child human being, optionally wherein the host is immunocompromised. In preferred embodiments, the immune response against the coronavirus lasts for at least about 40-50 days, and can be re-initiated by re-administration of AdD with or without the one or more SARS-CoV-2 vectors. Other embodiments of such AdD vectors, immunogenic compositions comprising the same, and/or methods for using the same are also contemplated herein as would be understood by those of ordinary skill in the art.

In some embodiments, this disclosure provides an immunogenic composition comprising one or more SARS-CoV-2 vectors comprising a rdAd vector comprising an expression cassette comprising a coding sequence encoding at least one SARS-CoV-spike (S) protein receptor binding domain (RBD), or at least one immunogenic fragment thereof, wherein the composition is configured to induce neutralizing antibody to the spike protein RBD, in a mammalian subject. Putative studies indicate the spike protein via its receptor binding domain of S1 binds to the angiotensin-converting enzyme 2 (ACE2) receptor (Y. Wan et al.; receptor recognition by novel coronavirus from Wuhan: An analysis based on decade-long structural studies of SARS; J. Virol. doi:10.1128/JVI.00127-20; posted online 29 Jan. 2020). Generating an immune response against at least the RBD of spike protein is an attractive target for inducing neutralization antibodies, wherein spike protein mediates coronavirus entry into host cells by first binding to a host receptor (e.g., ACE2) and then fusing viral and host membranes. The spike protein for SARS-CoV-2 is provided herein as SEQ ID NO: 3 (GenBank: QHD43416.1). See FIG. 3.

In certain embodiments, the immunogenic composition (e.g. vaccine) comprises one or more coronavirus antigens. In certain embodiments, the coronavirus is SARS-CoV-2, wherein the coding sequence for the Wuhan 2019 isolate (SARS-CoV-2) is provided herein as SEQ ID NO: 1. In embodiments, the replication deficient adenoviral vector comprises one or more coding sequences of SEQ ID NO: 1. Those sequences comprise one or more immunogenic domains such as a B cell and/or T cell epitope. In certain embodiments, the replication deficient adenoviral vector comprises and/or expresses (e.g., comprises an expression cassette encoding) one or more immunogenic domains provided in any of encoded SEQ ID NO: 2 to 20. In preferred embodiments, the replication deficient adenoviral vector comprises and/or expresses (e.g., comprises an expression cassette encoding) SEQ ID NO: 15, or a variant thereof (i.e., in either the RBD sequence (amino acids 57-253 of SEQ ID NO: 15 (comprising SEQ ID NO: 446)), and/or the signal and/or leader and/or flanking sequences). In embodiments, the coding sequence of the replication deficient adenovirus vector encodes at least one or more B cell epitopes, one or more CD8⁺ T cell epitopes, and/or one or more CD4⁺ T cell epitopes. One of skill in the art understands how to identify those epitopes within a larger sequence using bioinformatic methodologies such as publicly available tools accessible at the immune epitope database (IEDB), in vitro assay based on PBMCs from infection-positive subjects combined with short linear peptides scanning the antigen sequence or in vitro assay based on serum using short linear or conformational peptides scanning the antigen sequence.

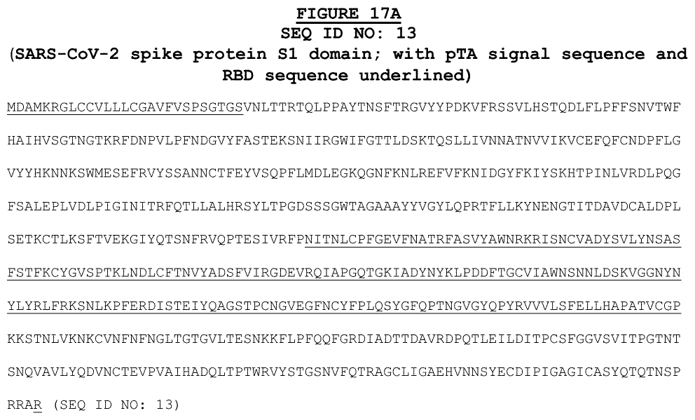

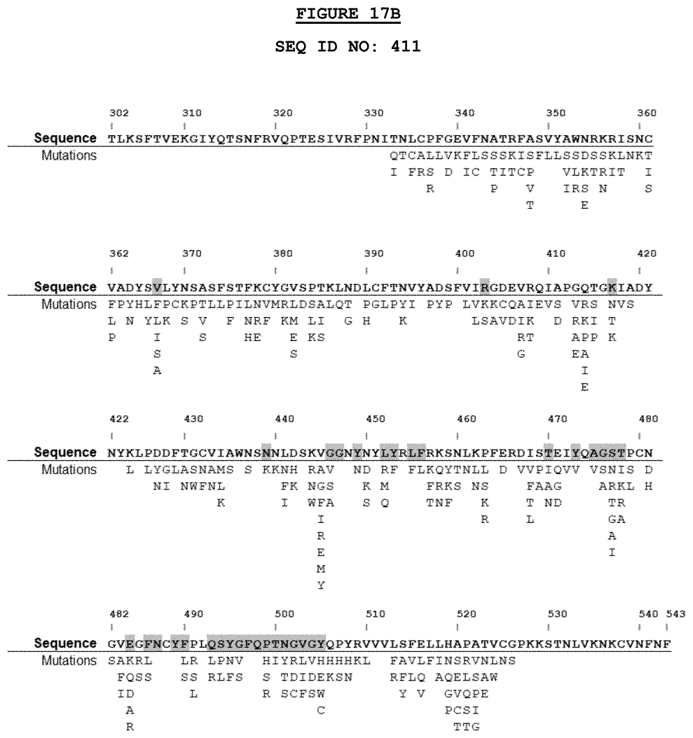

In embodiments, the replication defective adenoviral vector comprises an expression cassette comprising a coding sequence encoding an antigen of SEQ ID NO: 3, SEQ ID NO: 12, SEQ ID NO: 15, the RBD amino acid sequence (amino acids 57-253 of SEQ ID NO: 15 (SEQ ID NO: 446)), or an immunogenic fragment thereof. In embodiments, the replication defective adenoviral vector comprises an expression cassette comprising a coding sequence encoding SARS-CoV-2 spike protein, S1 domain, S2 domain, or an immunogenic fragment thereof. In embodiments, the expression cassette of the immunogenic composition comprises a coding sequence for spike (S) protein (e.g., as in preferred embodiments SEQ ID NO: 3 and SEQ ID NO: 12), 51 domain (e.g., as in preferred embodiment SEQ ID NO: 13) of the spike protein, or an immunogenic fragment thereof (e.g., as in preferred embodiments SEQ ID NOS: 14-17 (FIGS. 17B-21)). In a preferred embodiment, the coding sequence encodes the RBD sequence comprising amino acids 57-253 of SEQ ID NO: 15 (SEQ ID NO: 446). In certain embodiments, those encoded sequences comprise a leader signal sequence, either native or as a recombinant sequence comprising a pTA signal sequence (e.g., preferred embodiment MDAMKRGLCCVLLLCGAVFVSPSGTGS (SEQ ID NO: 427)).

In certain embodiments, the encoded sequence of the immunogenic composition is a sequence, or immunogenic fragment thereof, presented in SEQ ID NO: 3, or a sequence having at least 80% homology to SEQ ID NO: 3. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 3. In certain embodiments, the encoded sequence of the immunogenic composition is a sequence, or immunogenic fragment thereof, presented in SEQ ID NO: 12, or a sequence having at least 80% homology and/or identity to SEQ ID NO: 12. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 12. In certain preferred embodiments, the encoded sequence of the immunogenic composition is a sequence, or immunogenic fragment thereof, presented in SEQ ID NO: 13, or a sequence having at least 80% homology and/or identity to SEQ ID NO: 13. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 13. In certain embodiments, the encoded sequence of the immunogenic composition is a sequence, or immunogenic fragment thereof, presented in SEQ ID NO: 14, or a sequence having at least 80% homology and/or identity to SEQ ID NO: 14. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 14. In certain preferred embodiments, the encoded sequence of the immunogenic composition is a sequence, or immunogenic fragment thereof, presented in SEQ ID NO: 15, or a sequence having at least 80% homology and/or identity to SEQ ID NO: 15. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 15. In certain preferred embodiments, the encoded sequence of the immunogenic composition is a sequence, or immunogenic fragment thereof, presented in SEQ ID NO: 16, or a sequence having at least 80% homology and/or identity to SEQ ID NO: 16. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 16. In certain preferred embodiments, the encoded sequence of the immunogenic composition is a sequence, or immunogenic fragment thereof, presented in SEQ ID NO: 17, or a sequence having at least 80% homology and/or identity to SEQ ID NO: 17. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 17. In certain preferred embodiments, the encoded sequence of the immunogenic composition is a sequence, or immunogenic fragment thereof, presented in SEQ ID NO: 446, or a sequence having at least 80% homology and/or identity to SEQ ID NO: 446. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 446. In certain preferred embodiments, the encoded sequence of the immunogenic composition is a sequence, or immunogenic fragment thereof, presented in any of SEQ ID NOS: 412-417, SEQ ID NOS: 438-445, SEQ ID NO: 460 and SEQ ID NOS: 475-476, or a sequence having at least 80% homology and/or identity to SEQ ID NOS: 412-417, SEQ ID NOS: 438-445, SEQ ID NO: 460 and SEQ ID NOS: 475-476. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to any of SEQ ID NOS: 412-420.

In certain other embodiments, the expression cassette of the immunogenic composition comprises a coding sequence for the S1 domain of the spike protein (e.g. SEQ ID NO: 13), or an immunogenic fragment thereof. In certain embodiments, the coding sequence encodes at least amino acid resides 331 to 527 of SEQ ID NO: 3, SEQ ID NO: 12, or SEQ ID NO: 13, wherein the amino acid position numbering is based on the full-length spike protein sequence. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to: amino acid resides 331 to 527 of SEQ ID NO: 3; SEQ ID NO: 12; or SEQ ID NO: 13; wherein the amino acid position numbering is based on the full length spike protein sequence (FIG. 3).

In certain embodiments, the expression cassette of the immunogenic composition comprises a coding sequence for the S1 domain of the spike protein (e.g., SEQ ID NO: 13; FIG. 17A), or a fragment (preferably immunogenic fragment) thereof (e.g., SEQ ID NO: 14 or 15), wherein the sequence comprises one or more of the following residues: L455, F486, Q493, S494 and/or N501 (amino acid position numbering based on full-length spike protein). See., e.g., FIGS. 3, 15, and 17B-21. In embodiments, the encoded sequence comprises Q493 and N501. In certain embodiments, the encoded sequence comprises Q493. Those particular amino acid residues of SARS-CoV-2 at positions 455, 486, 493, 494 and/or 501 are believed to directly interact with their receptor. Independently those amino acid residues may be modified via conservative substitutions based on physico-chemical properties of the amino acids of SARS-CoV-2 at positions 455, 486, 493, 494 and 501 of the spike protein. In this way alternative sequences may be generated that would potentially capture escapees (e.g., viral mutations that evade acquired neutralizing antibodies but still bind to a host receptor) wherein the immunogenic compositions would induce neutralizing antibodies based on those alternative sequence(s). In embodiments, the expression cassette of the immunogenic composition comprises a coding sequence for the S1 domain of the spike protein, RBD of S1 domain, or an immunogenic fragment thereof, wherein the sequence comprises SEQ ID NO: 16 wherein 455 (L) is selected from Y, F, L or S; 486 (F) is selected from L, F, S or P; 493 (Q) is selected from L, N, Q, R or K; 494 (S) is selected from D, G, P, L or S; and, 501 (N) is selected from T, S, N or Y. In embodiments, the expression cassette of the immunogenic composition comprises a coding sequence for the S1 domain of the spike protein, RBD of S1, or an immunogenic fragment thereof, wherein the sequence comprises SEQ ID NO: 17 wherein 455 (L) is selected from Y, F, L or S; 486 (F) is selected from L, F, S or P; 493 (Q) is selected from L, N, Q, R or K; 494 (S) is selected from D, G, P, L or S; and, 501 (N) is selected from T, S, N, or Y.

In some embodiments, the expression cassette of the immunogenic composition comprises a coding sequence for modifications of a subsection of the spike protein sequence of SEQ ID NO. 13, the amino acid sequence of that subsection being shown in FIG. 17B (“Sequence”) and below:

(SEQ ID NO: 411)
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPTN
GVGYQPYRVVVLSFELLHAPATVCGPKKSTNLVKNKCVNFNF

In some embodiments, the coding sequence can encode at least one substitution of any of the amino acids of SEQ ID NO: 411. In some embodiments, the coding sequence can encode at least one substitution (“Mutations”) to SEQ ID NO: 411. Preferably, the one or more substitutions to SEQ ID NO: 411 is one of those shown in FIG. 17B. In some embodiments, the one or more substitutions can be to any one or more of amino acids 333-388, 390-395, 397-399, 401-411, 413-415, 417-419, 424, 426-435, 437, 439-442, 444-446, 449, 450, 452, 453, 455-463, 465, 467-473, 475-479, 481-486, 490, 491, 493-495, 499-510, or 513-526, the numbering corresponding to that shown in FIG. 17B. In preferred embodiments, the substitution can be at one or more of the amino acids that serve as main contact residues with the human ACE2 receptor, preferably amino acids 417, 446, 447, 449, 453, 455, 456, 473, 475-477, 484, 486, 487, 493, 495-498, 500-503, and/or 505 (the numbering corresponding to that shown in FIG. 17B). In some embodiments, the substitution to SEQ ID NO: 411 is at least one of: the substitution of amino acid 417 (K) by N; the substitution of amino acid 446 (G) is V, S or A; the substitution of amino acid 449 (Y) is N; the substitution at amino acid 453 (Y) by F; the substitution of amino acid 455 (L) by F; the substitution of amino acid 456 (F) by L; the substitution of amino acid 473 (Y) by V; the substitution of amino acid 475 (A) by V; the substitution of amino acid 476 (G) by S or A; the substitution of amino acid 477 (S) by N, R, T, G, A or I; the substitution at amino acid 484 (E) is Q, K, D, A or R; the substitution of amino acid 486 (F) by L or S; the substitution of amino acid 453 (Y) by F; the substitution of amino acid 493 (Q) by L or R; the substitution of amino acid 495 (Y) by N or F; the substitution of amino acid 500 (T) by I; the substitution of amino acid 501 (N) by Y, T or S; the substitution of amino acid 502 (G) by R, D or C; the substitution of amino acid 503 (V) by L, I or F; and/or, the substitution of amino acid 505 (Y) by H, E, W or C; the numbering corresponding to that shown in FIG. 17B.

In embodiments, the encoded spike protein RBD sequence comprises a residue selected from Y455, F455 or S455. In other embodiments, the encoded spike protein RBD sequence comprises a residue selected from L486 or P486. In certain embodiments, the encoded spike protein RBD sequence comprises a residue selected from N493, R493 or K493. In embodiments, the encoded spike protein RBD sequence comprises a residue selected from D494 or G494. In embodiments, the encoded spike protein RBD sequence comprises a residue selected from T501 or 5501.

Stabilized Spike Protein Antigen Design

The development of a viral vector immunogenic composition or vaccine against Covid-19 (e.g. SARS-CoV-2) faces some key challenges related to: (1) the ability of the viral vector to express the protein with a quaternary structure representative of the native pre-fusion protein expressed by the coronavirus and compatible with the induction of neutralizing antibodies against the SARS-CoV-2 virus; and, (2) the ability to propagate the viral vector during its manufacture in a cell-based expression system with limited interference that may be associated with the concomitant expression of the transgenic spike protein.

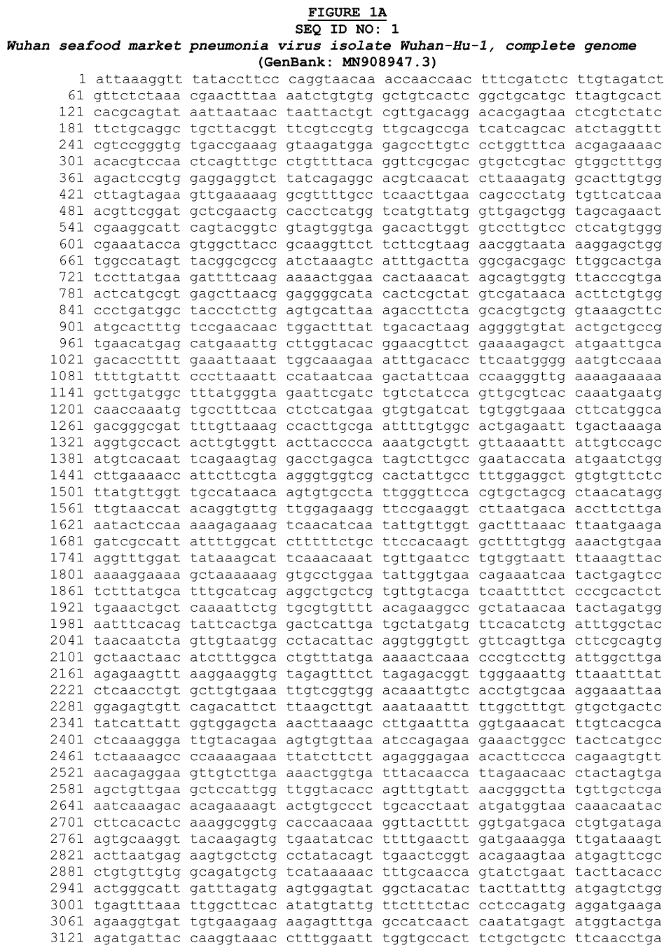

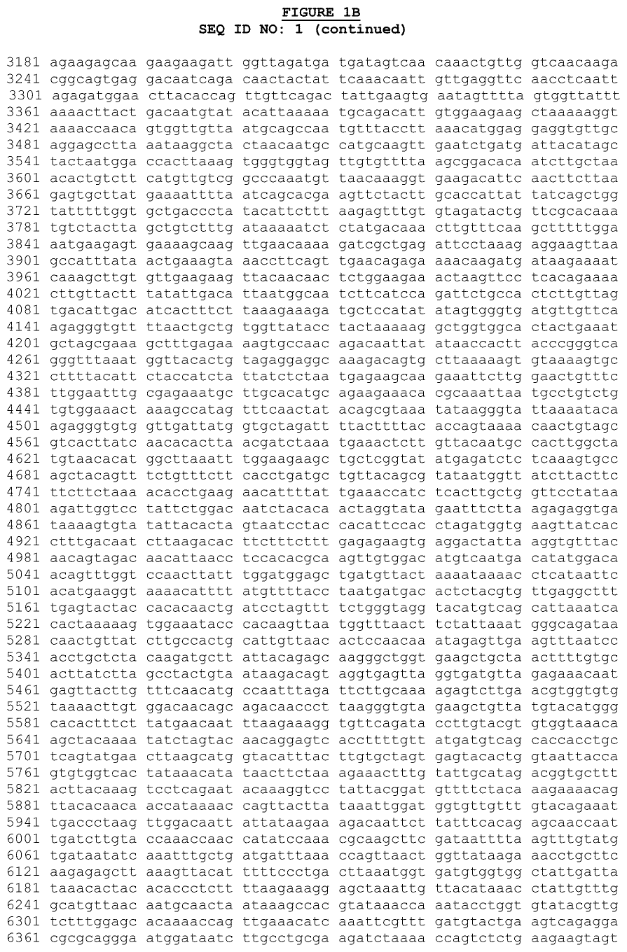

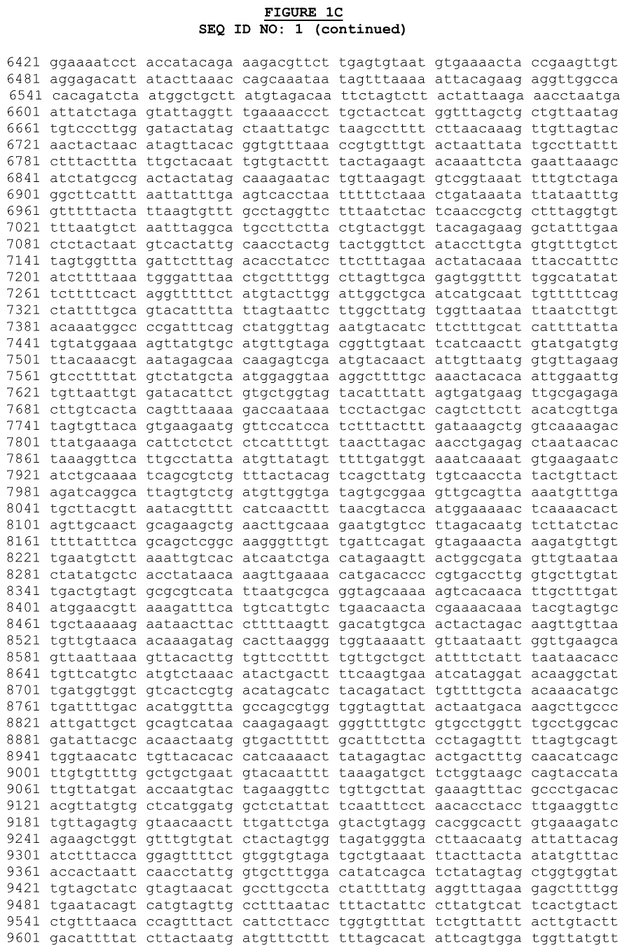

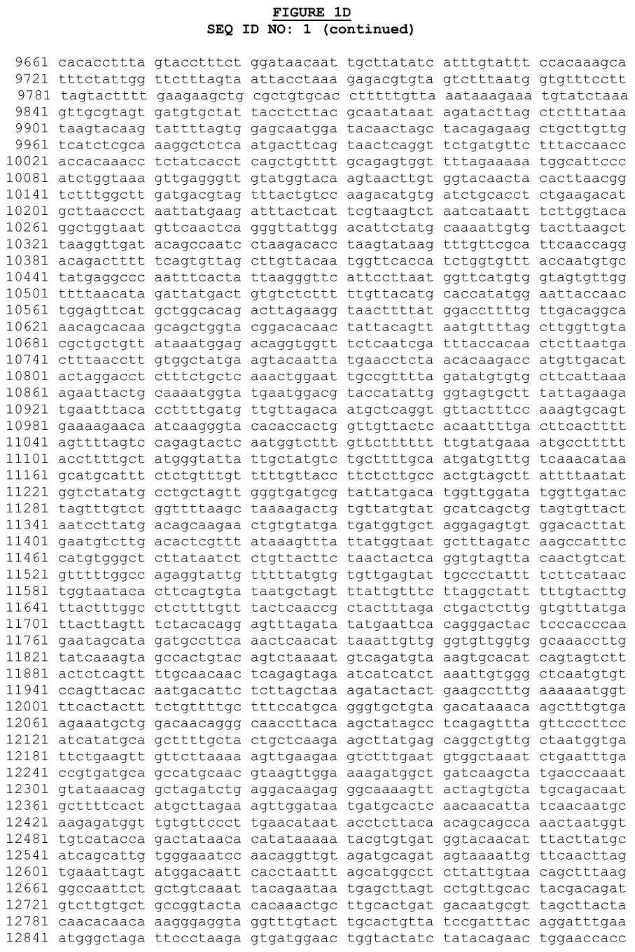

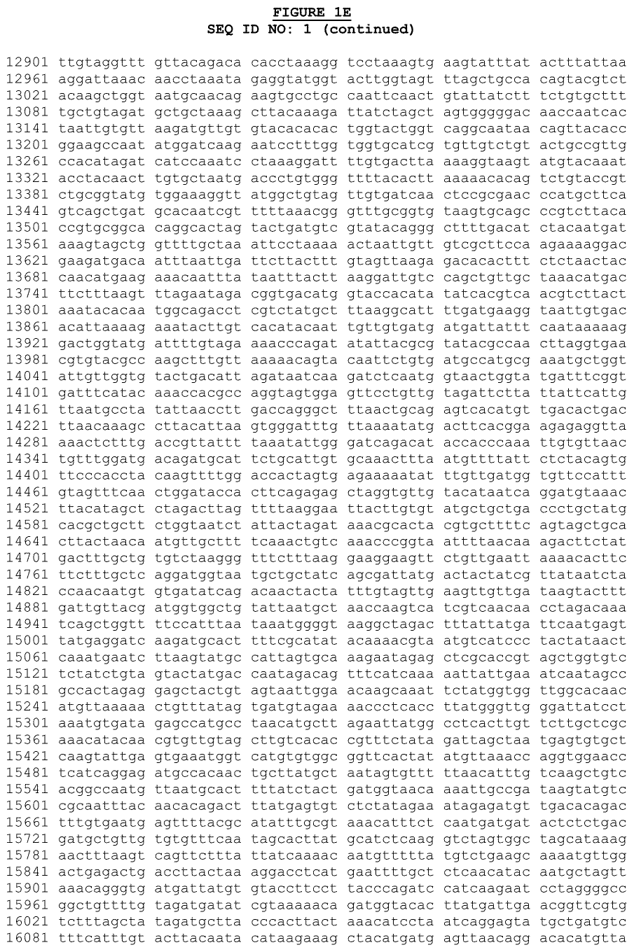

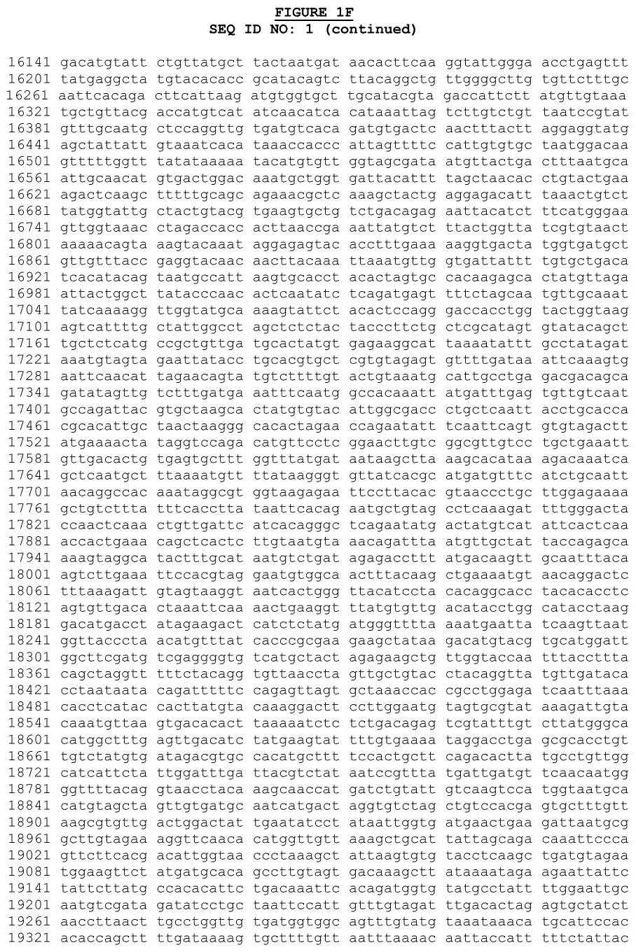

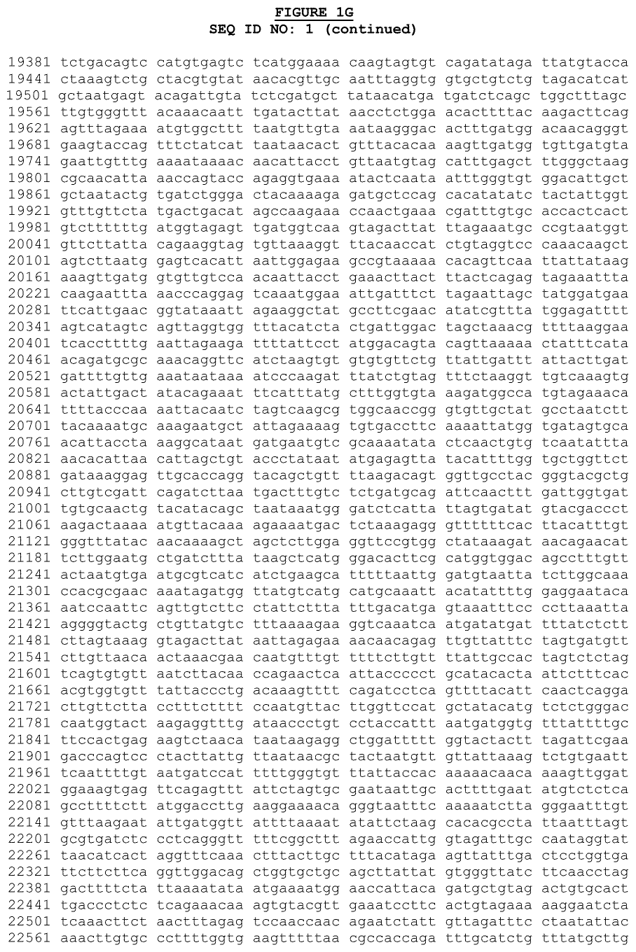

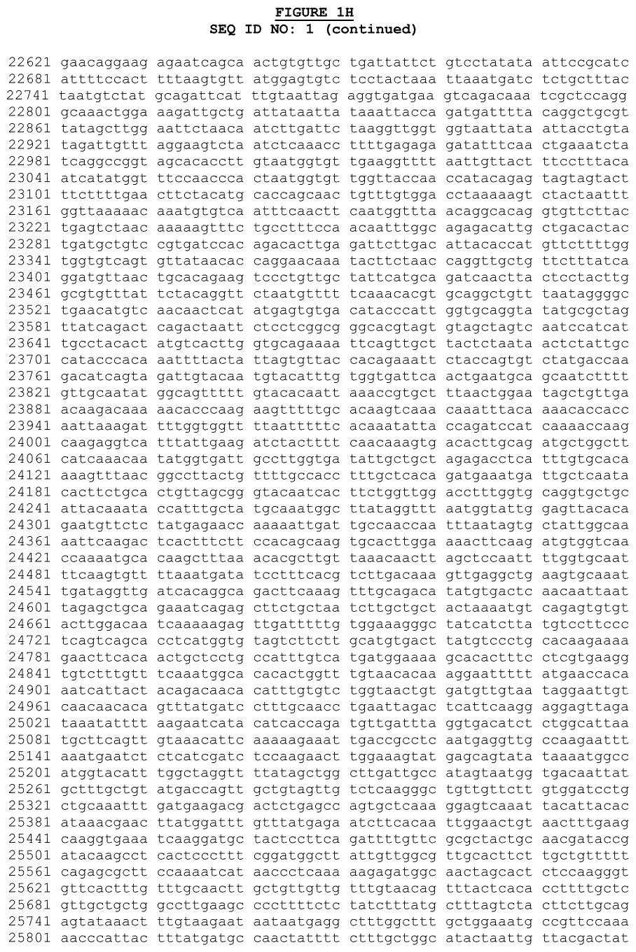

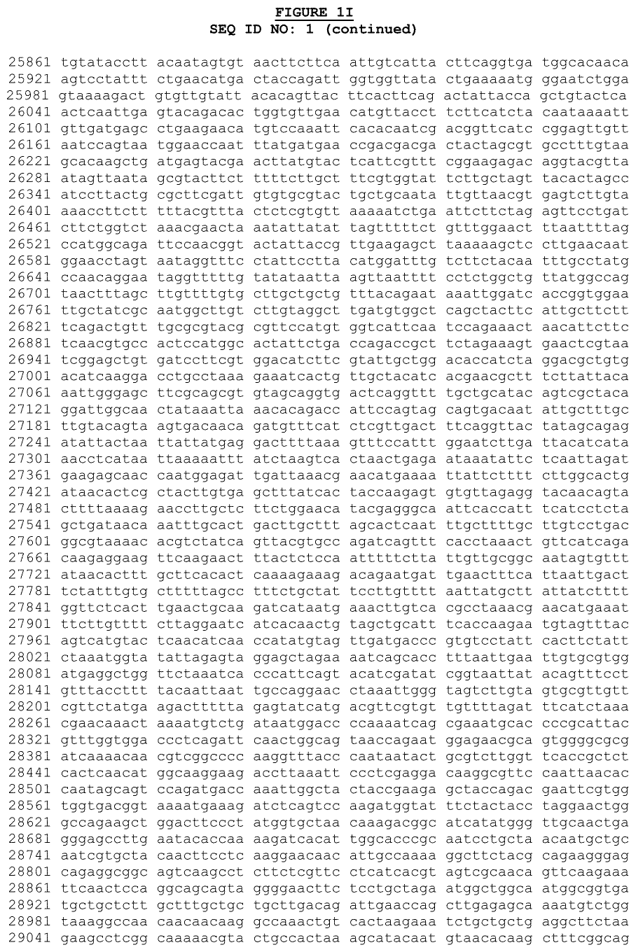

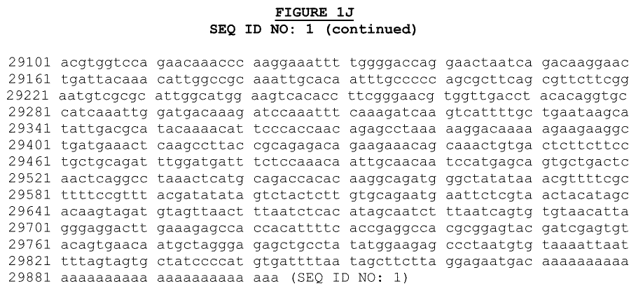

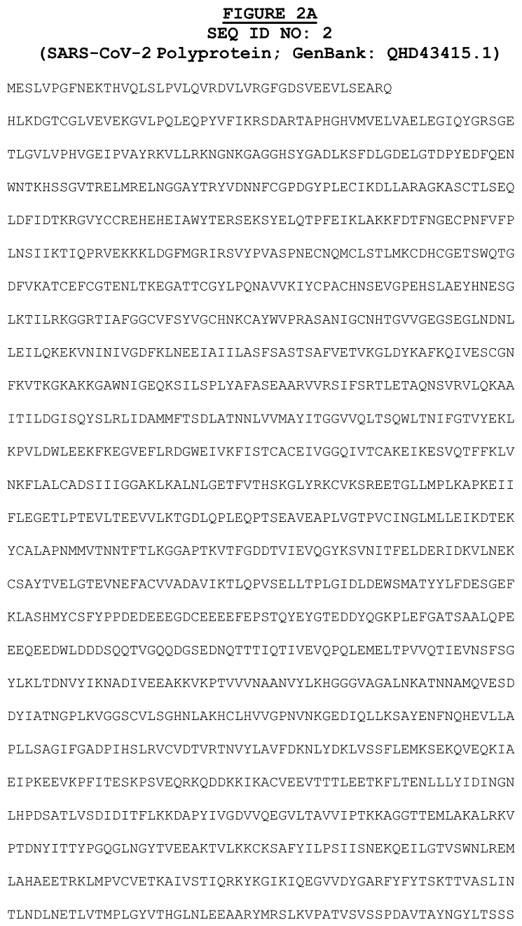

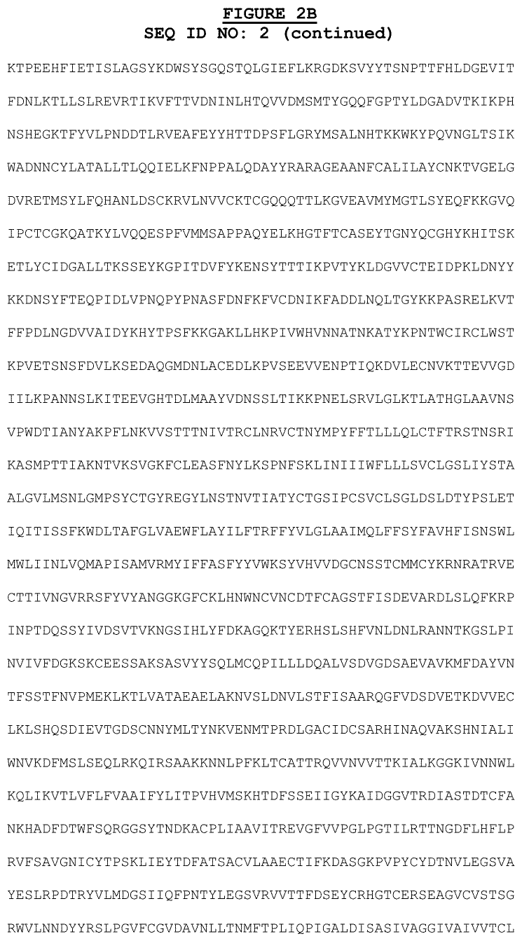

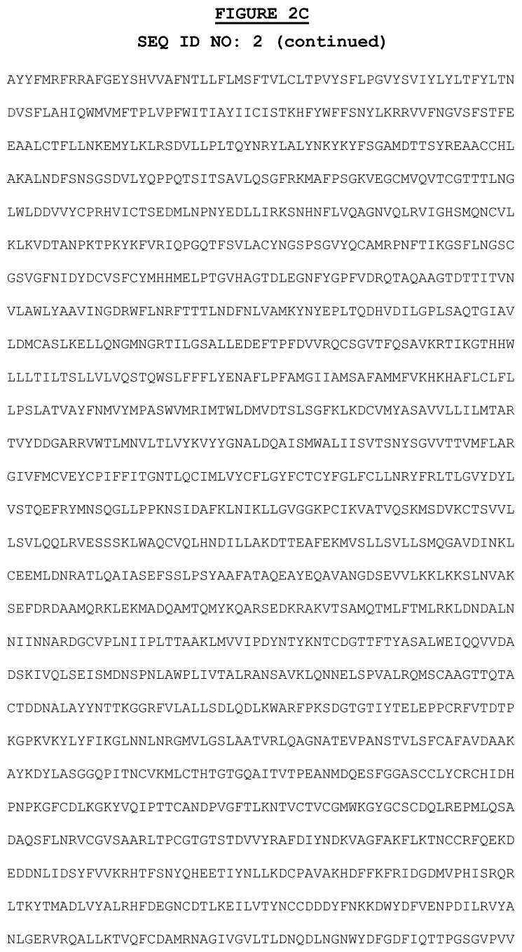

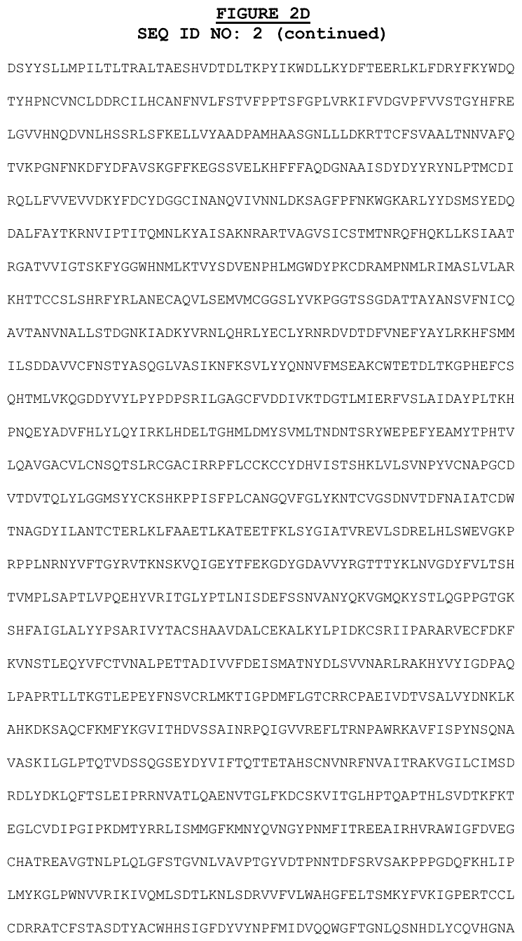

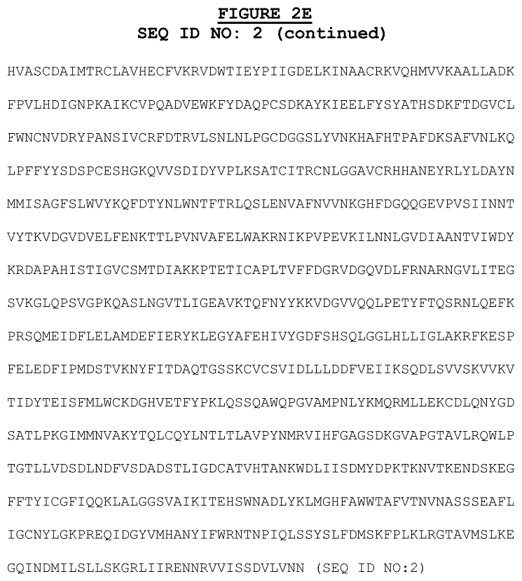

In embodiments, designing a viral vector expressing a stabilized spike antigen in a pre-fusion trimeric conformation expressed on the surface of infected cells may be desirable for the development of a Covid-19 immunogenic composition or vaccine to address both the antigenicity and manufacturing of the vaccine. In other embodiments, immunogenic fragments thereof (spike protein), such as the S1 or receptor binding domain (RBD), may be desirable for the development of a Covid-19 immunogenic composition or vaccine, which also address both the antigenicity and manufacturing of the immunogenic composition or vaccine. Those challenges are due, in part, to the “fusogenic” properties of the SARS-Cov-2 spike protein, which as used herein refers to the fusion properties of the spike protein to gain entry in a host cell (e.g., as a coat protein on the surface of viral particles) using ACE2 as the entry receptor or when expressed on the host cell to fuse with neighbor cells to form larger syncytia or syncytium (multi-nucleated host cells) as a mean to propagate very rapidly by using this cell-cell fusion independently of the entry receptor. Priming of coronavirus S proteins by host cell proteases is essential for viral entry into cells expressing ACE2 and encompasses S protein cleavage at the S1/S2 and the S2′ sites. The S1/S2 cleavage site of SARS-CoV-2 S harbors several arginine residues (multibasic), which indicates high cleavability (Hoffmann et al. SARS-CoV-2 Cell Entry Depends on ACE2 and TMPRSS2 and Is Blocked by a Clinically Proven Protease Inhibitor; Cell; 2020 Apr. 16; 181(2): 271-280.e8). Successful conformational changes of S proteins, leading to membrane fusion, not only require receptor binding, but also appropriate protease activation. There is a furin site between S1 and S2 (amino acids 682-685, RRAR) subunits in SARS-CoV-2 S protein, similar to those found in highly pathogenic influenza viruses. That furin site (RRAR) is not present in other coronavirus S proteins (e.g., See FIG. 1C of Walls et al. Structure, Function, and Antigenicity of the SARS-CoV-2 Spike Glycoprotein; Cell; 2020 Mar. 9, incorporated herein by reference), and may contribute to instability, possibly confounding viral-vectored vaccine development. Therefore, in certain embodiments, those amino acid residues in the SARS-Cov-2 spike protein may be removed and/or modified to ameliorate or remove the furin enzymatic cleave site between 51 and S2 domain present in SARS-Cov-2 spike protein.

Hence, one approach to stabilize the spike protein is to render the protein more resistant to proteolytic degradation (1) during expression of the protein in the cell-based manufacturing such as in E1 complementing cell lines during production of the (adenovirus) viral vector and/or (2) during expression of the protein in a mammal following administration of the viral vector. Proteolytic cleavage sites can be modified by amino-acid substitutions, insertion or deletions in order to prevent or reduce enzymatic degradation. Proteolytic cleavage sites of interest for this type of approach are preferably found in solvent-accessible regions of the protein that form the solvent-facing surface of the three-dimensional structure of the protein trimer complex. These solvent-accessible sites include the S1/S2 junction QTQTNSPRRARSVASQ (SEQ ID NO: 25) and S2′ junction PDPSKPSKRSF (SEQ ID NO: 26). Other solvent accessible areas can be identified by known methods that calculate the relative solvent accessible score area (SASA). Potential enzymes involved in proteolytic cleavage of spike protein include Furin, Trypsin, Elastase, Plasmin, TMPRSS2, Chymotrypsin, Cathepsin-L, Cathepsin-B, TMPRSS11D, Dipeptidyl Peptidase IV, MMP-13, MMP-12, MMP-2, MMP-9, MMP-3, Caspase-3, Caspase-8, Caspase-9. Protease cleavage sites can be predicted using known algorithms such as Proper, Properous, PeptideCutter, Pripper, CasCleave, CasCleave 2.0, CASVM and iProt-Sub.

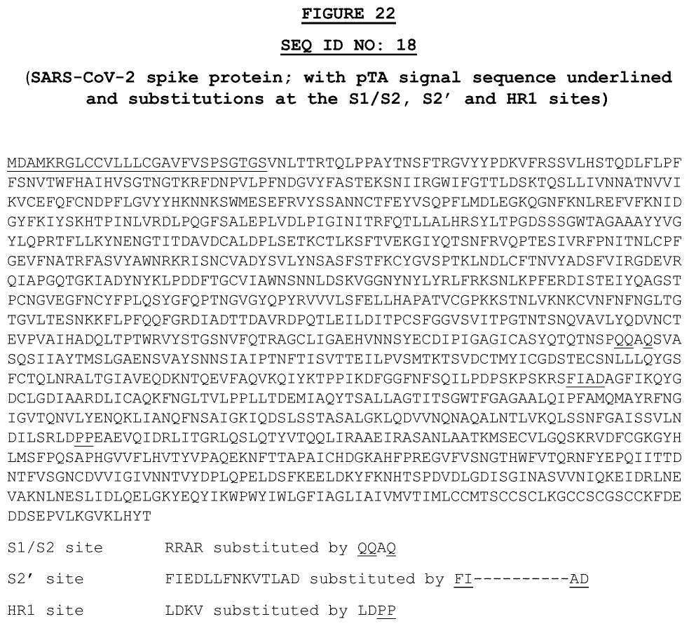

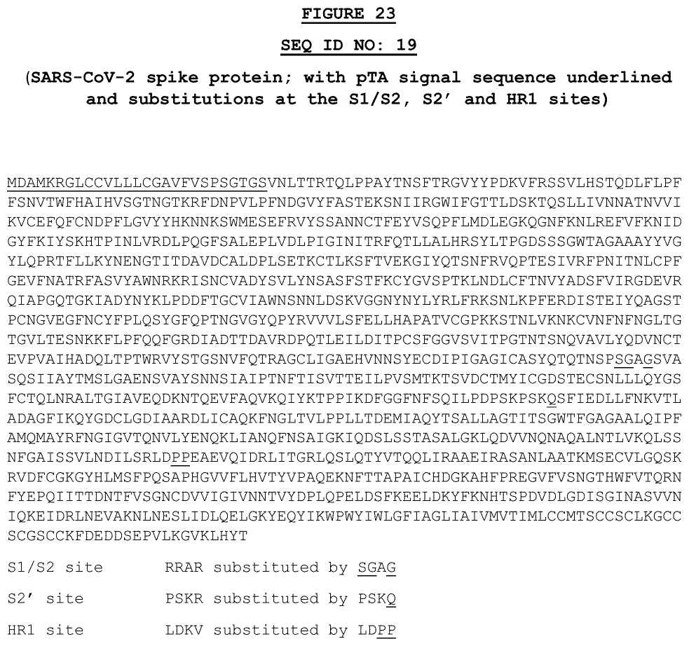

Cleavage of the S1/S2 junction QTQTNSPRRARSVASQ (SEQ ID NO: 25) from SARS-CoV-2 and/or corresponding sequences in other coronaviruses, has been demonstrated to be primarily dependent upon arginine residues such as arginine at positions 685, 682 and/or 683 from SEQ ID NO: 3 and SEQ ID NO: 12. To interfere with proteolytic cleavage, hydrophilic, non-positively charged amino-acids or small amino-acids such as N, Q, D, E, T, S, G or A can be considered for substitution of arginine residues. In addition, the S1/S2 junction can be rendered resistant to proteolytic cleavage by deletion or insertion of amino acids, provided that those sequence modifications do not alter the three-dimensional conformation of the spike antigen so to preserve its antigenicity and ability to stimulate the production of neutralizing antibodies. A similar approach can also be applied to the S2′ junction PDPSKPSKRSF (SEQ ID NO: 26) for the Lysine residue at position 814 and Arginine residue at position 815 in sequence SEQ ID NO: 3 and SEQ ID NO: 12. In embodiments, the expression cassette of the immunogenic composition comprises a coding sequence of SEQ ID NO: 18 (a preferred embodiment); SEQ ID NO: 19 (a preferred embodiment); or SEQ ID NO: 20. See FIGS. 22 to 24. Thus, in certain preferred embodiments, the encoded sequence of the immunogenic composition is a sequence, or immunogenic fragment thereof, presented in SEQ ID NO: 18 or SEQ ID NO: 19, or a sequence having at least 80% homology and/or identity to SEQ ID NO: 18 or SEQ ID NO: 19. In certain embodiments, the encoded sequence of the immunogenic composition is a sequence with at least about 80%, 81%, 82%, 83%, 84%, 85%, 86%, 87%, 88%, 89%, 90%, 91%, 92%, 93%, 94%, 95%, 96%, 97%, 98%, 99%, homology and/or identity to SEQ ID NO: 18 or SEQ ID NO: 19.

In other embodiments, another approach for stabilizing the spike protein includes maintaining the metastable spike protein in its prefusion conformation. Modifications of the sequence to stabilize coronavirus spike protein in a prefusion conformation have been previously disclosed in Pallesen et al. 2017 (Immunogenicity and structures of a rationally designed prefusion MERS-CoV spike antigen. Proc Natl Acad Sci USA. 2017 Aug. 29; 114(35):E7348-E7357) and Wrapp et al. 2020 (Cryo-EM structure of the 2019-nCoV spike in the prefusion conformation. Science. 2020 Mar. 13; 367(6483): 1260-1263), showing that proline substitutions in the loop between the first heptad repeat (HR1) and the central helix (CH) restrict premature triggering of the fusion protein and often increase expression yields of prefusion ectodomains. See FIGS. 22 to 24 and SEQ ID NOS: 18, 19 and 20.

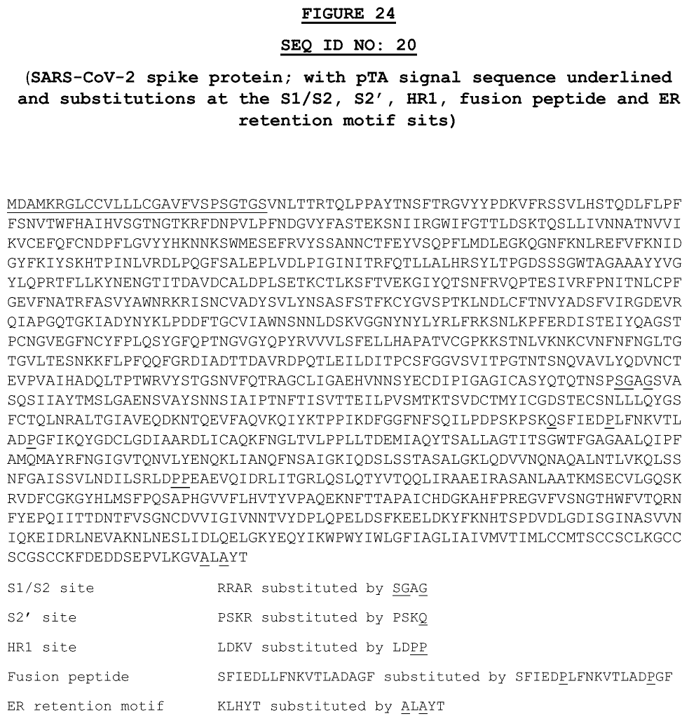

In embodiments, other approaches consist of facilitating the expression of the protein at the surface of infected cells. The spike protein may be retained in endoplasmic reticulum and/or the pre-golgi/golgi, a phenomenon associated with the nature of the transmembrane and/or intracellular domain. Preventing intracellular retention can be achieved by modifying the intracellular domain (IC) by substitution of cysteine in cysteine-rich domains or through the modification of the C-terminal endoplasmic reticulum retention motif (VKLHYT (SEQ ID NO: 409)). See FIG. 24 and SEQ ID NO: 20.

In certain embodiments, limiting the toxicity of the transgenic spike protein during manufacturing of the viral vector can be achieved by modifying the fusion peptide contained within the spike antigen sequence. Modification by deletion, insertion of amino-acids and/or substitution of amino-acids can be introduced in the spike sequence to disrupt the alpha-helix conformation of the fusion peptide. Disruption of the alpha helix conformation of the fusion peptide can be achieve by the substitution of specific amino acids by proline residues.

Accordingly, in embodiments the design of a viral vector immunogenic composition or vaccine against Covid-19 expressing a stabilized full-length trimeric spike antigen may comprise one or more of the modifications described above. Example of those modifications are presented in Table 1 (S1/S2, S2′ and fusion domains) and Table 2 (HR1/CH and IC domains) (FIGS. 22-24, SEQ ID NOS: 18-20).

TABLE 1
Exemplary modifications for increase the stability or
surface expression of full-length spike

| Region | S1/S2 | S2′ | Fusion |
|---|---|---|---|
| Sequence | QTQTNSPRRARSVASQ | PSKPSKRSF | SFIEDLLFNKVTLADAGFI |
| from SEQ ID | (SEQ ID NO: 25) | (SEQ ID | (SEQ ID NO: 447) |
| NO: 3 or SEQ | | NO: 26) | |
| ID NO: 12 | | | |
| Modifications | ......XX.X..... | ......X.. | .....P............. |
| | ......QQ.Q..... | ......Q.. | ...............P... |
| | ......SG.G..... | ......N.. | .....P.........P... |
| | ......-----.... | .....NA.. | ....---------...... |
| | | .....--.. | |

“.” = conserved amino-acid; X = N, Q, D, E, T, S, G or A; and, “-” = deletion.

TABLE 2
Exemplary modifications to increase the stability
or surface expression of full-length spike protein

| Region | HR1/CH | IC |
|---|---|---|
| Sequence | SRLDKVEAEV | CCMTSCCSCLKGCCSCGSCCKFDEDDSEPVLKGVKLHYT |
| from SEQ ID | (SEQ ID | (SEQ ID NO: 449) |
| NO: 3 or SEQ | NO: 448) | |
| ID NO: 12 | | |
| Modifications | ....PP.... | .................................------ |
| | | ......................----------------- |
| | | ...........---------------------------- |
| | | -----------.......................Z.#.. |
| | | ...........Z........................... |
| | | ............Z.......................... |
| | | ..............Z........................ |
| | | .................Z..................... |
| | | ..................Z.................... |

“.” = conserved amino-acid; Z = N, Q, D, E, T, S, G or A; and, “-” = deletion

In embodiments, the present immunogenic composition is a multivalent composition. In certain embodiments, the expression cassette of the replication defective adenoviral vector further comprises a coding sequence encoding one or more of SARS-CoV-2 structural proteins envelope (E), membrane (M) or nucleocapsid (N). Each of those structural proteins is presented herein as SEQ ID NO: 5; SEQ ID NO: 6 and SEQ ID NO: 10, respectively. See also FIGS. 5, 6 and 10. In alternative embodiments, those structural proteins may be encoded from a separate replication defective adenoviral vector and provided as a multivalent formulation with a replication defective adenoviral vector comprising an expression cassette comprising a coding sequence encoding at least SARS-CoV-2 (2019 novel coronavirus; 2019-nCoV) spike (S) protein receptor binding domain (RBD), or at least one immunogenic fragment thereof.

Neutralizing the Post-Fusion Spike Antigen

As discussed above, unlike other beta-coronaviruses of subgroup B, the S protein of SARS-CoV-2 harbors a unique S1/S2 furin-recognizable site, indicating that its S protein may possess unique infectious properties. Indeed, in active SARS-CoV-2 infection, syncytium phenomenon were shown to be naturally formed by infected cells, which is rarely reported in SARS-CoV infection, demonstrating a high capacity to mediate membrane fusion (Xu Z. et al. Pathological findings of COVID-19 associated with acute respiratory distress syndrome. Lancet Respir Med. 2020 April; 8(4):420-422.). These cytopathic syncytia are presumably responsible for, at least in part, the pathology observed in COVID-19 patients. Also, the formation of syncytia helps the virus to propagate very rapidly by using this cell-cell fusion mechanism independently of the receptor (receptor independent spread). Therefore, induction of neutralizing antibody against the S antigen in its post-fusion form represents a means for limiting the formation of syncytia and/or preventing the rapid spread of the virus in an entry receptor-independent manner. The post-fusion spike antigen is achieved after cleavage of spike protein by proteases. Cleavage induces a tridimensional rearrangement of the S2 domain as presented in Wall et al. 2017 (Walls, et al. Tectonic conformational changes of a coronavirus spike glycoprotein promote membrane fusion. Proc Natl Acad Sci USA. 2017; 114(42):11157-11162.).

In some embodiments, SARS-CoV-2 immunization vector can also or alternatively comprise a polynucleotide causing expression in a cell of spike antigen without the S1 domain such as the S2 domain of spike (approximately from amino-acid residue 686 of SEQ ID NO: 3) or the S2′ domain of spike (approximately from amino-acid residue 816 of SEQ ID NO: 3) or a sequence starting beyond residue 816 of SEQ ID NO: 3 and at minimum comprising the HR1, HR2 and a transmembrane domain. In some preferred embodiments, these SARS-CoV-2 immunization vectors induce an immune response that neutralizes the post-fusion spike protein (e.g. antibodies against the S2 domain) and provide protection by preventing the formation of syncytia and/or by preventing the propagation of the virus in an entry-receptor independent manner. In some embodiments, the expression cassette of the immunogenic composition comprises a coding sequence for the S2 portion of the spike (S) protein (e.g., SEQ ID NO: 21, SEQ ID NO: 22, SEQ ID NO: 23). In some embodiments, the expression cassette of the immunogenic composition comprises a coding sequence for the S2 portion of the spike (S) protein containing an additional transmembrane domain to replace the fusion peptide (e.g., SEQ ID NO: 24). The polypeptides of SEQ ID NOS: 21, 22, 23, and 24 are shown below:

MDAMKRGLCCVLLLCGAVFVSPSGTGSSVASQSIIAYTMSLGAENSVAYS
NNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPD
PSKPSKRSFIEDLLFNKVTLADAGFIKQYGDCLGDIAARDLICAQKFNGL
TVLPPLLTDEMIAQYTSALLAGTITSGWTFGAGAALQIPFAMQMAYRFNG
IGVTQNVLYENQKLIANQFNSAIGKIQDSLSSTASALGKLQDVVNQNAQA
LNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPH
GVVFLHVTYVPAQEKNFTTAPAICHDGKAHFPREGVFVSNGTHWFVTQRN
FYEPQITTTDNTFVSGNCDVVIGIVNNTVYDPLQPELDSFKEELDKYFKN
HTSPDVDLGDISGINASVVNIQKEIDRLNEVAKNLNESLIDLQELGKYEQ
YIKWPWYIWLGFIAGLIAIVMVTIMLCCMTSCCSCLKGCCSCGSCCKFDE
DDSEPVLKGVKLHYT
(SEQ ID NO: 21; SARS-CoV-2 spike protein - S2
domain; with pTA signal);
MDAMKRGLCCVLLLCGAVFVSPSGTGSSFIEDLLFNKVTLADAGFIKQYG
DCLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDSL
SSTASALGKLQDVVNQNAQALNTLVKQLSSNFGAISSVLNDILSRLDKVE
AEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNE
VAKNLNESLIDLQELGKYEQYIKWPWYIWLGFIAGLIAIVMVTIMLCCMT
SCCSCLKGCCSCGSCCKFDEDDSEPVLKGVKLHYT
(SEQ ID NO: 22; SARS-CoV-2 spike protein - S2′
domain; with pTA signal);
MDAMKRGLCCVLLLCGAVFVSPSGTGSGIGVTQNVLYENQKLIANQFNSA
IGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMV
TIMLCCMTSCCSCLKGCCSCGSCCKFDEDDSEPVLKGVKLHYT
(SEQ ID NO: 23; SARS-CoV-2 spike protein - HR1 +
HR2 domain; with pTA signal);
and,
GPPLSSSLGLALLLLLLALLFWLYIVMGLTVLPPLLTDEMIAQYTSALLA
GTITSGWTFGAGAALQIPFAMQMAYRFNGIGVTQNVLYENQKLIANQFNS
AIGKIQDSLSSTASALGKLQDVVNQNAQALNTLVKQLSSNFGAISSVLND
ILSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVV
IGIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIVM
VTIMLCCMTSCCSCLKGCCSCGSCCKFDEDDSEPVLKGVKLHYT
(SEQ ID NO: 24; SARS-CoV-2 spike protein - S2′
domain + additional transmembrane domain).

T Cell Epitope Peptides

In embodiments, the SARS-CoV-2 immunization vectors comprise one or more peptide-encoding sequences from 8 to 60 amino acids in length (and concatenated if more than one), wherein each peptide comprise at least one CD8+ T cell epitope and/or at least one CD4+ T cell epitope. In this case, the concatenated peptide-encoding polynucleotide express a polypeptide where T cell epitopes or T-cell epitope-containing sequences are separated by a spacer sequence containing amino-acids such as glycine, serine or alanine residues. The polynucleotide may further comprise polynucleotide sequences coding for ubiquitin or a signal peptide. In some embodiments, the SARS-CoV-2 immunization vectors comprise one or more polynucleotides encoding one or more S protein antigens. (Ahmed, et al. Preliminary Identification of Potential Vaccine Targets for the COVID-19 Coronavirus (SARS-CoV-2) Based on SARS-CoV Immunological Studies. Viruses, 12: 254 (2020)). The one or more S protein peptide antigens can comprise any T cell epitope, for instance, any of the peptides shown in Table 3A:

TABLE 3A

| SEQ ID | |
|---|---|
| NO: | Peptide |
| 27/78 | ILLNKHID |
| 28 | AFFGMSRIGMEVTPSGTW |
| 29/80 | MEVTPSGTWL |
| 30/83 | GMSRIGMEV |
| 31/87 | ILLNKHIDA |
| 32/88 | ALNTPKDHI |
| 33 | IRQGTDYKHWPQIAQFA |
| 34 | KHWPQIAQFAPSASAFF |
| 35/91 | LALLLLDRL |
| 36/92 | LLLDRLNQL |
| 37 | LLNKHIDAYKTFPPTEPK |
| 38/95 | LQLPQGTTL |
| 39 | AQFAPSASAFFGMSR |
| 40 | AQFAPSASAFFGMSRIGM |
| 41 | RRPQGLPNNTASWFT |
| 42 | YKTFPPTEPKKDKKKK |
| 43/152 | GAALQIPFAMQMAYRF |
| 44/153 | MAYRFNGIGVTQNVLY |
| 45/154 | QLIRAAEIRASANLAATK |
| 46/271 | FIAGLIAIV |
| 47/105 | ALNTLVKQL |
| 48/112 | LITGRLQSL |
| 49/124 | NLNESLIDL |
| 50/313 | QALNTLVKQLSSNFGAI |
| 51/133 | RLNEVAKNL |
| 52/143 | VLNDILSRL |
| 53/146 | VVFLHVTYV |
| 54/63 | SEETGTLIV |
| 55 | FLWLLWPVT |
| 56 | FLWLLWPVTL |
| 57 | FLWLLWPVTLACFVL |
| 58 | IKDLPKEITVATSRT |
| 59 | LEQWNLVIGF |
| 60 | LFARTRSMW |
| 61 | LWLLWPVTL |
| 62 | LWPVTLACF |
| 63/54 | SEETGTLIV |
| 64 | MWSFNPETNI |
| 65 | NLVIGFLFL |
| 66 | PKEITVATSRTLSYY |
| 67 | ATSRTLSYY |
| 68 | ATSRTLSYYK |
| 69 | QWNLVIGFLF |
| 70 | RYRIGNYKL |
| 71 | SELVIGAVI |
| 72 | SFNPETNIL |
| 73 | SMWSFNPET |
| 74 | TSRTLSYYK |
| 75 | TVATSRTLSY |
| 76 | WLLWPVTLA |
| 77 | WPVTLACFVL |
| 78/27 | ILLNKHID |
| 79 | FPRGQGVPI |
| 80/29 | MEVTPSGTWL |
| 81 | GMEVTPSGTWL |
| 82 | LLLLDRLNQ |
| 83/30 | GMSRIGMEV |
| 84 | GTTLPKGFY |
| 85 | ALALLLLDR |
| 86 | IDAYKTFPPTEPKKD |
| 87/31 | ILLNKHIDA |
| 88/32 | ALNTPKDHI |
| 89 | KTFPPTEPK |
| 90 | KTFPPTEPKK |
| 91/35 | LALLLLDRL |
| 92/36 | LLLDRLNQL |
| 93 | LLLLDRLNQL |
| 94 | APSASAFFGM |
| 95/38 | LQLPQGTTL |
| 96 | AQFAPSASA |
| 97 | LSPRWYFYY |
| 98 | MSRIGMEVTPSGTWL |
| 99 | ASAFFGMSR |
| 100 | NKHIDAYKTFPPTEP |
| 101 | ATEGALNTPK |
| 102 | QLPQGTTLPK |
| 103 | QQQGQTVTK |
| 104 | QQQQGQTVTK |
| 105/47 | ALNTLVKQL |
| 106 | ISGINASVVNIQKEI |
| 107 | LDKYFKNHTSPDVDL |
| 108 | APHGVVFLHV |
| 109 | LGDISGINASVVNIQ |
| 110 | LGFIAGLIAIVMVTI |
| 111 | LIDLQELGKY |
| 112/48 | LITGRLQSL |
| 113 | LLLQYGSFC |
| 114 | LLQYGSFCT |
| 115 | LNTLVKQLSSNFGAI |
| 116 | LQDVVNQNAQALNTL |
| 117 | LQIPFAMQM |
| 118 | LQSLQTYVTQQLIRA |
| 119 | LQTYVTQQLIRAAEI |
| 120 | AQALNTLVK |
| 121 | AQKFNGLTVLPPLLT |
| 122 | MTSCCSCLK |
| 123 | ASANLAATK |
| 124/49 | NLNESLIDL |
| 125 | PCSFGGVSVITPGTN |
| 126 | PYRVVVLSF |
| 127 | QELGKYEQYI |
| 128 | QIPFAMQMAYRFNGI |
| 129 | QPYRVVVLSF |
| 130 | QQLIRAAEIRASANL |
| 131 | QTYVTQQLIRAAEIR |
| 132 | RLDKVEAEV |
| 133/51 | RLNEVAKNL |
| 134 | RLQSLQTYV |
| 135 | RVDFCGKGY |
| 136 | AYRFNGIGVTQNVLY |
| 137 | SLIDLQELGK |
| 138 | SSNFGAISSVLNDIL |
| 139 | SVLNDILSR |
| 140 | TGRLQSLQTYVTQQL |
| 141 | TQNVLYENQK |
| 142 | CMTSCCSCLK |
| 143/52 | VLNDILSRL |
| 144 | VQIDRLITGR |
| 145 | VRFPNITNL |
| 146/53 | VVFLHVTYV |
| 147 | WLGFIAGLIAIVMVT |
| 148 | CVNFNFNGLTGTGVL |
| 149 | YEQYIKWPWY |
| 150 | DKYFKNHTSPDVDLG |
| 151 | AEIRASANLA |
| 152/43 | GAALQIPFAMQMAYRF |
| 153/44 | MAYRFNGIGVTQNVLY |
| 154/45 | QLIRAAEIRASANLAATK |
| 155 | SASAFFGMSR |
| 156 | SPRWYFYYL |
| 157 | SQASSRSSSR |
| 158 | TPSGTWLTY |
| 159 | TTLPKGFYA |
| 160 | VLQLPQGTTL |
| 161 | VLQLPQGTTLPKGFY |
| 162 | VTPSGTWLTY |
| 163 | AEGSRGGSQA |
| 164 | FLCLFLLPSL |
| 165 | FLGRYMSAL |
| 166 | FLLNKEMYL |
| 167 | FLLPSLATV |
| 168 | FLNGSCGSV |
| 169 | FLNRFTTTL |
| 170 | FLPRVFSAV |
| 171 | FRYMNSQGL |
| 172 | FTYASALWEI |
| 173 | AIILASFSA |
| 174 | GVYDYLVST |
| 175 | ILASFSAST |
| 176 | ILGTVSWNL |
| 177 | IQPGQTFSV |
| 178 | ALRANSAVK |
| 179 | ALWEIQQVV |
| 180 | KLWAQCVQL |
| 181 | LLSAGIFGA |
| 182 | MPASWVMRI |
| 183 | NVLAWLYAA |
| 184 | QLMCQPILL |
| 185 | QLMCQPILLL |
| 186 | AVLQSGFRK |
| 187 | SLLSVLLSM |
| 188 | TLGVYDYLV |
| 189 | TVLSFCAFA |
| 190 | VLAWLYAAV |
| 191 | VLSFCAFAV |
| 192 | YIFFASFYY |
| 193 | FPPTSFGPL |
| 194 | FVDGVPFVV |
| 195 | AIMTRCLAV |
| 196 | GVAMPNLYK |
| 197 | ALLADKFPV |
| 198 | ILGLPTQTV |
| 199 | ILHCANFNV |
| 200 | IPRRNVATL |
| 201 | ISDYDYYRY |
| 202 | IVDTVSALV |
| 203 | KLFAAETLK |
| 204 | KLNVGDYFV |
| 205 | KLSYGIATV |
| 206 | KMQRMLLEK |
| 207 | KQFDTYNLW |
| 208 | LLDDFVEII |
| 209 | LLLDDFVEI |
| 210 | LLMPILTLT |
| 211 | LMIERFVSL |
| 212 | LQLGFSTGV |
| 213 | LVLSVNPYV |
| 214 | MLWCKDGHV |
| 215 | MMISAGFSL |
| 216 | MVMCGGSLYV |
| 217 | NLWNTFTRL |
| 218 | NMLRIMASL |
| 219 | ATVVIGTSK |
| 220 | RILGAGCFV |
| 221 | RLYYDSMSY |
| 222 | RQLLFVVEV |
| 223 | SSNVANYQK |
| 224 | TLIGDCATV |
| 225 | TLVPQEHYV |
| 226 | TMADLVYAL |
| 227 | TTLPVNVAF |
| 228 | VLQAVGACV |
| 229 | VLWAHGFEL |
| 230 | VMCGGSLYV |
| 231 | VVDKYFDCY |
| 232 | VVYRGTTTY |
| 233 | YLDAYNMMI |
| 234 | YLNTLTLAV |
| 235 | YQKVGMQKY |
| 236 | YTMADLVYA |
| 237 | YVFCTVNAL |
| 238 | HLVDFQVTI |
| 239 | HPLADNKFAL |
| 240 | KLFIRQEEV |
| 241 | QECVRGTTVLLKEPC |
| 242 | CELYHYQECV |
| 243 | SVSPKLFIR |
| 244 | YEGNSPFHPL |
| 245 | AFLLFLVLI |
| 246 | AFLLFLVLIMLIIFW |
| 247 | FLAFLLFLV |
| 248 | FLAFLLFLVL |
| 249 | FLAFLLFLVLIMLII |
| 250 | FLLFLVLIM |
| 251 | FLLFLVLIML |
| 252 | FLLFLVLIMLIIFWF |
| 253 | FLVLIMLII |
| 254 | FLVLIMLIIFWFSLE |
| 255 | FYLCFLAFL |
| 256 | FYLCFLAFLL |
| 257 | IDFYLCFLAF |
| 258 | IMLIIFWFSL |
| 259 | LAFLLFLVLIMLIIF |
| 260 | LFLVLIMLIIFWFSL |
| 261 | LIDFYLCFL |
| 262 | LLFLVLIML |
| 263 | LLFLVLIMLI |
| 264 | LLFLVLIMLIIFWFS |
| 265 | MLIIFWFSL |
| 266 | YLCFLAFLL |
| 267 | YLCFLAFLLFLVLIM |
| 268 | DSFKEELDKY |
| 269 | AEVQIDRLI |
| 270 | AEVQIDRLIT |
| 271/46 | FIAGLIAIV |
| 272 | FPNITNLCPF |
| 273 | GAALQIPFAMQMAYR |
| 274 | GLIAIVMVTI |
| 275 | GRLQSLQTY |
| 276 | GSFCTQLNR |
| 277 | GVVFLHVTY |
| 278 | GWTFGAGAALQIPFA |
| 279 | GYQPYRVVVL |
| 280 | IDRLITGRLQSLQTY |
| 281 | IGAGICASY |
| 282 | IITTDNTFV |

In some preferred embodiments, the vectors can encode multiple epitopes, separately or as part of a single polypeptide (e.g., concatenated, optionally separated by a linker amino acid sequence of two to ten amino acids). In some embodiments, the vectors can encode multiple epitopes as in the exemplary groups shown in Table 3B:

TABLE 3B

| Group | Peptides |
|---|---|
| 1 | FIAGLIAIV (SEQ ID NO: 46), GLIAIVMVTI (SEQ ID NO: 274), IITTDNTFV |
| | (SEQ ID NO: 282), ALNTLVKQL (SEQ ID NO: 105), LITGRLQSL (SEQ ID |
| | NO: 48), LLLQYGSFC (SEQ ID NO: 113), LQYGSFCT (SEQ ID NO: 465), |
| | NLNESLIDL (SEQ ID NO: 49), RLDKVEAEV (SEQ ID NO: 132), |
| | RLNEVAKNL (SEQ ID NO: 51), RLQSLQTYV (SEQ ID NO: 134), |
| | VLNDILSRL (SEQ ID NO: 52), VVFLHVTYV (SEQ ID NO: 53), ILLNKHID |
| | (SEQ ID NO: 27), FPRGQGVPI (SEQ ID NO: 79), LLLLDRLNQ (SEQ ID NO: |
| | 82), GMSRIGMEV (SEQ ID NO: 30), ILLNKHIDA (SEQ ID NO: 31), |
| | ALNTPKDHI (SEQ ID NO: 32), LALLLLDRL (SEQ ID NO: 35), |
| | LLLDRLNQL (SEQ ID NO: 36), LLLLDRLNQL (SEQ ID NO: 93), |
| | LQLPQGTTL (SEQ ID NO: 38), AQFAPSASA (SEQ ID NO: 96), |
| | TTLPKGFYA (SEQ ID NO: 159), VLQLPQGTTL (SEQ ID NO: 160), |
| | VRFPNITNL (SEQ ID NO: 145) and YEQYIKWPWY (SEQ ID NO: 149) |
| 2 | GYQPYRVVVL (SEQ ID NO: 279), PYRVVVLSF (SEQ ID NO: 126) and, |
| | LSPRWYFYY (SEQ ID NO: 97) |
| 3 | DSFKEELDKY (SEQ ID NO: 268), LIDLQELGKY (SEQ ID NO: 111), |
| | PYRVVVLSF (SEQ ID NO: 126), GTTLPKGFY (SEQ ID NO: 84) and |
| | VTPSGTWLTY (SEQ ID NO: 162) |
| 4 | GSFCTQLNR (SEQ ID NO: 276), GVVFLHVTY (SEQ ID NO: 277), |
| | AQALNTLVK (SEQ ID NO: 120), MTSCCSCLK (SEQ ID NO: 122), |
| | ASANLAATK (SEQ ID NO: 123), SLIDLQELGK (SEQ ID NO: 137), |
| | SVLNDILSR (SEQ ID NO: 139), TQNVLYENQK (SEQ ID NO: 141), |
| | CMTSCCSCLK (SEQ ID NO: 142), VQIDRLITGR (SEQ ID NO: 144), |
| | KTFPPTEPK (SEQ ID NO: 89), and KTFPPTEPKK (SEQ ID NO: 90) |
| 5 | LSPRWYFYY (SEQ ID NO: 97), ASAFFGMSR (SEQ ID NO: 99), |
| | ATEGALNTPK (SEQ ID NO: 101), QLPQGTTLPK (SEQ ID NO: 102), |
| | QQQGQTVTK (SEQ ID NO: 103), QQQQGQTVTK (SEQ ID NO: 104), |
| | SASAFFGMSR (SEQ ID NO: 155), SQASSRSSSR (SEQ ID NO: 157) and |
| | TPSGTWLTY (SEQ ID NO: 158) |
| 6 | GSFCTQLNR (SEQ ID NO: 276), GVVFLHVTY (SEQ ID NO: 277), |
| | AQALNTLVK (SEQ ID NO: 120), MTSCCSCLK (SEQ ID NO: 122), |
| | ASANLAATK (SEQ ID NO: 123), SLIDLQELGK (SEQ ID NO: 137), |
| | SVLNDILSR (SEQ ID NO: 139), TQNVLYENQK (SEQ ID NO: 141), |
| | CMTSCCSCLK (SEQ ID NO: 142), VQIDRLITGR (SEQ ID NO: 144), |
| | KTFPPTEPK (SEQ ID NO: 89), KTFPPTEPKK (SEQ ID NO: 90), |
| | LSPRWYFYY (SEQ ID NO: 97), ASAFFGMSR (SEQ ID NO: 99), |
| | ATEGALNTPK (SEQ ID NO: 101), QLPQGTTLPK (SEQ ID NO: 102), |
| | QQQGQTVTK (SEQ ID NO: 103), QQQQGQTVTK (SEQ ID NO: 104), |
| | SASAFFGMSR (SEQ ID NO: 155), SQASSRSSSR (SEQ ID NO: 157) and |
| | TPSGTWLTY (SEQ ID NO: 158) |
| 7 | GYQPYRVVVL (SEQ ID NO: 279), PYRVVVLSF (SEQ ID NO: 126) and |
| | LSPRWYFYY (SEQ ID NO: 97) |
| 8 | GSFCTQLNR (SEQ ID NO: 276), GVVFLHVTY (SEQ ID NO: 277), |
| | AQALNTLVK (SEQ ID NO: 120), MTSCCSCLK (SEQ ID NO: 122), |
| | ASANLAATK (SEQ ID NO: 123), SLIDLQELGK (SEQ ID NO: 137), |
| | SVLNDILSR (SEQ ID NO: 139), TQNVLYENQK (SEQ ID NO: 141), |
| | CMTSCCSCLK (SEQ ID NO: 142), VQIDRLITGR (SEQ ID NO: 144), |
| | KTFPPTEPK (SEQ ID NO: 89), KTFPPTEPKK (SEQ ID NO: 90), |
| | LSPRWYFYY (SEQ ID NO: 97), ASAFFGMSR (SEQ ID NO: 99), |
| | ATEGALNTPK (SEQ ID NO: 101), QLPQGTTLPK (SEQ ID NO: 102), |
| | QQQGQTVTK (SEQ ID NO: 103), QQQQGQTVTK (SEQ ID NO: 104), |
| | SASAFFGMSR (SEQ ID NO: 155), SQASSRSSSR (SEQ ID NO: 157) and |
| | TPSGTWLTY (SEQ ID NO: 158) |
| 9 | FPNITNLCPF (SEQ ID NO: 272), APHGVVFLHV (SEQ ID NO: 108), |
| | FPRGQGVPI (SEQ ID NO: 79) and APSASAFFGM (SEQ ID NO: 94) |
| 10 | GAALQIPFAMQMAYR (SEQ ID NO: 273), GWTFGAGAALQIPFA (SEQ ID |
| | NO: 278), IDRLITGRLQSLQTY (SEQ ID NO: 280), ISGINASVVNIQKEI |
| | (SEQ ID NO: 106), LDKYFKNHTSPDVDL (SEQ ID NO: 107), |
| | LGDISGINASVVNIQ (SEQ ID NO: 109), LGFIAGLIAIVMVTI (SEQ ID NO: |
| | 110), LNTLVKQLSSNFGAI (SEQ ID NO: 115), LQDVVNQNAQALNTL |
| | (SEQ ID NO: 116), LQSLQTYVTQQLIRA (SEQ ID NO: 118), |
| | LQTYVTQQLIRAAEI (SEQ ID NO: 119), AQKFNGLTVLPPLLT (SEQ ID |
| | NO: 121), PCSFGGVSVITPGTN (SEQ ID NO: 125), QIPFAMQMAYRFNGI |
| | (SEQ ID NO: 128), QQLIRAAEIRASANL (SEQ ID NO: 130), |
| | QTYVTQQLIRAAEIR (SEQ ID NO: 131), AYRFNGIGVTQNVLY (SEQ ID |
| | NO: 136), SSNFGAISSVLNDIL (SEQ ID NO: 138), TGRLQSLQTYVTQQL |
| | (SEQ ID NO: 140), WLGFIAGLIAIVMVT (SEQ ID NO: 147), |
| | CVNFNFNGLTGTGVL (SEQ ID NO: 148), DKYFKNHTSPDVDLG (SEQ ID |
| | NO: 150), IDAYKTFPPTEPKKD (SEQ ID NO: 86), MSRIGMEVTPSGTWL |
| | (SEQ ID NO: 98), NKHIDAYKTFPPTEP (SEQ ID NO: 100) and |
| | VLQLPQGTTLPKGFY (SEQ ID NO: 161) |
| 11 | FPNITNLCPF (SEQ ID NO: 272), APHGVVFLHV (SEQ ID NO: 108), |
| | FPRGQGVPI (SEQ ID NO: 79) and APSASAFFGM (SEQ ID NO: 94) |
| 12 | LQIPFAMQM (SEQ ID NO: 117) and RVDFCGKGY (SEQ ID NO: 135) |
| 13 | GRLQSLQTY (SEQ ID NO: 275), RVDFCGKGY (SEQ ID NO: 135) and |
| | VRFPNITNL (SEQ ID NO: 145) |
| 14 | MTSCCSCLK (SEQ ID NO: 122), SLIDLQELGK (SEQ ID NO: 137), |
| | CMTSCCSCLK (SEQ ID NO: 142), VQIDRLITGR (SEQ ID NO: 144), |
| | SASAFFGMSR (SEQ ID NO: 155), and SQASSRSSSR (SEQ ID NO: 157) |
| 15 | LQIPFAMQM (SEQ ID NO: 117) and RVDFCGKGY (SEQ ID NO: 135) |

Other combinations of epitopes are also contemplated herein as would be understood by those of ordinary skill in the art.

In some embodiments, the vector(s) can encode one or more of the following epitopes that can be B cell epitopes: DVVNQNAQALNTLVKQL (SEQ ID NO: 283), FFGMSRIGMEVTPSGTW (SEQ ID NO: 284), EAEVQIDRLITGRLQSL (SEQ ID NO: 285), GLPNNTASWFTALTQHGK (SEQ ID NO: 286), EIDRLNEVAKNLNESLIDLQELGKYEQY (SEQ ID NO: 287), GTTLPK (SEQ ID NO: 288), EVAKNLNESLIDLQELG (SEQ ID NO: 289), IRQGTDYKHWPQIAQFA (SEQ ID NO: 290), GAALQIPFAMQMAYRFN (SEQ ID NO: 291), KHIDAYKTFPPTEPKKDKKK (SEQ ID NO: 292), GAGICASY (SEQ ID NO: 293), KHWPQIAQFAPSASAFF (SEQ ID NO: 294), AISSVLNDILSRLDKVE (SEQ ID NO: 295), YNVTQAFGRRGPEQTQGNF (SEQ ID NO: 296), GSFCTQLN (SEQ ID NO: 297), KTFPPTEPKKDKKKK (SEQ ID NO: 298), ILSRLDKVEAEVQIDRL (SEQ ID NO: 299), LLPAAD (SEQ ID NO: 300), KGIYQTSN (SEQ ID NO: 301), LNKHIDAYKTFPPTEPK (SEQ ID NO: 302), AMQMAYRF (SEQ ID NO: 303), LPQGTTLPKG (SEQ ID NO: 304), KNHTSPDVDLGDISGIN (SEQ ID NO: 305), LPQRQKKQ (SEQ ID NO: 306), MAYRFNGIGVTQNVLYE (SEQ ID NO: 307), PKGFYAEGSRGGSQASSR (SEQ ID NO: 308), AATKMSECVLGQSKRVD (SEQ ID NO: 309), QFAPSASAFFGMSRIGM (SEQ ID NO: 310), PFAMQMAYRFNGIGVTQ (SEQ ID NO: 311), QGTDYKHW (SEQ ID NO: 312), QALNTLVKQLSSNFGAI (SEQ ID NOS: 313, 50), QLPQGTTLPKGFYAE (SEQ ID NO: 314), QLIRAAEIRASANLAAT (SEQ ID NO: 315), QLPQGTTLPKGFYAEGSR (SEQ ID NO: 316), QQFGRD (SEQ ID NO: 317), QLPQGTTLPKGFYAEGSRGGSQ (SEQ ID NO: 318), RASANLAATKMSECVLG (SEQ ID NO: 319), TFPPTEPK (SEQ ID NO: 320), RLITGRLQSLQTYVTQQ (SEQ ID NO: 321), RRPQGLPNNTASWFT (SEQ ID NO: 322), EIDRLNEVAKNLNESLIDLQELGKYEQY (SEQ ID NO: 323), SQASSRSS (SEQ ID NO: 324), SLQTYVTQQLIRAAEIR (SEQ ID NO: 325), SRGGSQASSRSSSRSR (SEQ ID NO: 326), and DLGDISGINASVVNIQK (SEQ ID NO: 327); and/or combinations of the same. In some preferred embodiments, the vectors can encode multiple of such epitopes, separately or as part of a single polypeptide (e.g., in some embodiments concatenated, optionally separated by a linker amino acid sequence of two to ten amino acids).

In some embodiments, SARS-CoV-2 peptide sequences encoded by the SARS-CoV-2 immunization vectors can be selected based on the ability to stimulate CD4⁺ and CD8⁺ T cell responses, and can in some embodiments be selected based on the prediction of proteome regions containing the highest number of HLA class I and HLA class II binding motifs across a range of selected HLA alleles. In some embodiments, analysis of HLA class II binding motifs across the SARS-CoV-2 sequences can be performed using NetMHCpan EL 4.0 available at IEDB (http://tools.iedb.org/mhci/; Jurtz, et al. NetMHCpan-4.0: Improved Peptide-MHC Class I Interaction Predictions Integrating Eluted Ligand and Peptide Binding Affinity Data. J Immunol. 2017; 199(9):3360-3368). In some embodiments, the NetMHCpan EL 4.0 can be used to identify binding motifs having a length varying from 9 to 11 amino acids to HLA class I molecules and assigned a percentage rank (% Rank). In some embodiments, high affinity binding peptides can be identified as those exhibiting a %-Rank ≤0.1 while moderate affinity binding peptides can be considered to have a %-rank comprised between >0.1 and ≤0.5. In preferred embodiments, the NetMHCpan EL 4.0 prediction can be performed with a set of 18 HLA-A alleles, 32 HLA-B alleles and 20 HLA-C alleles shown here: HLA-A*01:01, HLA-A*02:01, HLA-A*02:06, HLA-A*03:01, HLA-A*11:01, HLA-A*23:0, HLA-A*24:02, HLA-A*25:01, HLA-A*26:01, HLA-A*29:02, HLA-A*30:01, HLA-A*30:02, HLA-A*31:01, HLA-A*32:01, HLA-A*33:03, HLA-A*68:01, HLA-A*68:02, HLA-A*74:01, HLA-B*07:02, HLA-B*08:01, HLA-B*13:01, HLA-B*13:02, HLA-B*14:02, HLA-B*15:01, HLA-B*15:02, HLA-B*15:25, HLA-B*18:01, HLA-B*27:02, HLA-B*27:05, HLA-B*35:01, HLA-B*35:03, HLA-B*37:01, HLA-B*38:01, HLA-B*39:01, HLA-B*40:01, HLA-B*40:02, HLA-B*44:02, HLA-B*44:03, HLA-B*46:01, HLA-B*48:01, HLA-B*49:01, HLA-B*50:01, HLA-B*51:01, HLA-B*52:01, HLA-B*53:01, HLA-B*55:01, HLA-B*56:01, HLA-B*57:01, HLA-B*58:01, HLA-B*58:02, HLA-C*01:02, HLA-C*02:02, HLA-C*02:09, HLA-C*03:02, HLA-C*03:03, HLA-C*03:04, HLA-C*04:01, HLA-C*05:01, HLA-C*06:02, HLA-C*07:01, HLA-C*07:02, HLA-C*07:04, HLA-C*08:01, HLA-C*08:02, HLA-C*12:02, HLA-C*12:03, HLA-C*14:02, HLA-C*15:02, HLA-C*16:01 and HLA-C*17:01. Other HLA class I alleles may also be suitable as would be understood by those of ordinary skill in the art.

In some embodiments, HLA class II binding motifs within the SARS-CoV-2 polypeptide sequences can be performed using NetMHCII 2.3 (http://www.cbs.dtu.dk/services/NetMHCII/; Jensen et al. Improved methods for predicting peptide binding affinity to MHC class II molecules. Immunology. 2018 July; 154(3):394-406.) which is based on ensembles of artificial neural networks trained on quantitative peptide binding affinity data from the Immune Epitope Database (IEDB). In some embodiments, NetMHCII 2.3 can be used to identify peptides that can presented by HLA class II molecules by determining, e.g., the percentage rank (%-Rank) (related to the affinity of the peptides for the HLA molecules) and the core nine amino acid binding motif. In some embodiments, high affinity HLA class II binding peptides can be identified as those exhibiting a %-Rank ≤2 while moderate affinity binding peptides can be considered to have a %-Rank >2 and 10. In preferred embodiments, the NetMHCII 2.3 system can be based on a set of 20 HLA-DR alleles, 20 HLA-DQ alleles and 9 HLA-DP alleles shown here: DRα*10101-DRβ1*0101, DRα1*0101-DRβ1*0301, DRα1*0101-DRβ1*0401, DRα1*0101-DRβ1*0701, DRα1*0101-DRβ1*0801, DRα1*0101-DRβ1*0802, DRα1*0101-DRβ1*0901, DRα*10101-DRβ1*1001, DRα*10101-DRβ1*1101, DRα*10101-DRβ1*1201, DRα*10101-DRβ1*1301, DRα1*0101-DRβ1*1302, DRα1*0101-DRβ1*150, DRα1*0101-DRβ1*1602, DRα1*0101-DRβ3*0101, DRα1*0101-DRβ3*0202, DRα1*0101-DRβ3*0301, DRα1*0101-DRβ4*0101, DRα1*0101-DRβ4*0103, DRα1*0101-DRβ5*0101, DPα1*0103-DPβ1*0301, DPα1*0103-DPβ1*0401, DPα1*0103-DPβ1*0402, DPα1*0103-DPβ1*0601, DPα1*0201-DPβ1*0101, DPα1*0201-DPβ1*0501, DPα1*0201-DPβ1*1401, DPα1*0301-DPβ1*0402, DPα1*0103-DPβ1*0201, DQα1*0101-DQβ1*0501, DQα1*0102-DQβ1*0501, DQα1*0102-DQβ1*0502, DQα1*0102-DQβ1*0602, DQα1*0103-DQβ1*0603, DQα1*0104-DQβ1*0503, DQα1*0201-DQβ1*0202, DQα1*0201-DQβ1*0301, DQα1*0201-DQβ1*0303, DQα1*0201-DQβ1*0402, DQα1*0301-DQβ1*0301, DQα1*0301-DQβ1*0302, DQα1*0303-DQβ1*0402, DQα1*0401-DQβ1*0402, DQα1*0501-DQβ1*0201, DQα1*0501-DQβ1*0301, DQα1*0501-DQβ1*0302, DQα1*0501-DQβ1*0303, DQα1*0501-DQβ1*0402 and DQα1*0601-DQβ1*0402. Other HLA class II alleles may also be suitable as would be understood by those of ordinary skill in the art.

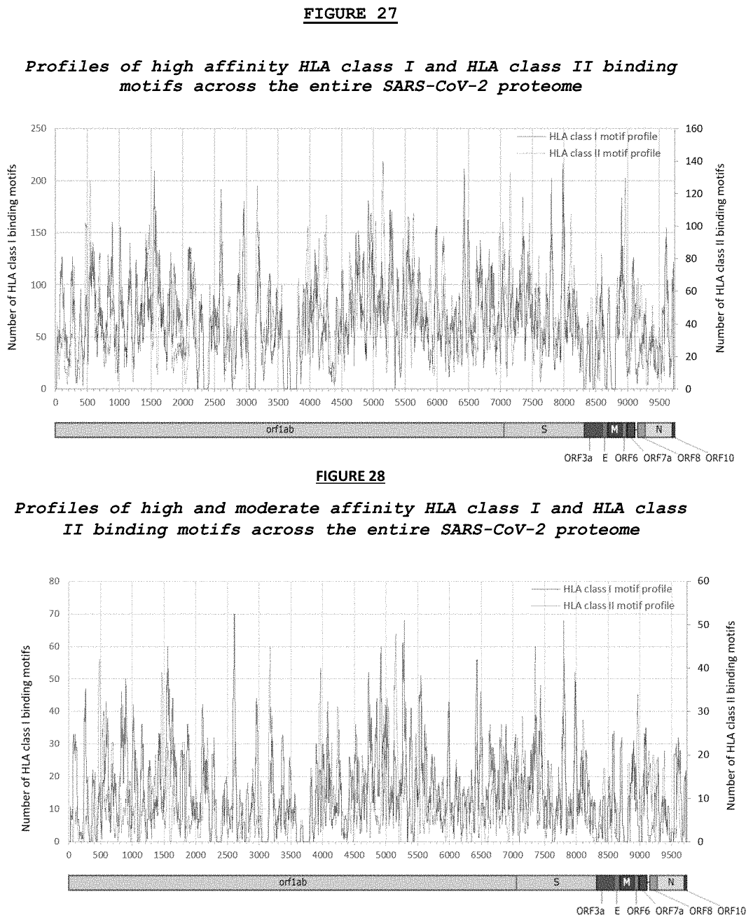

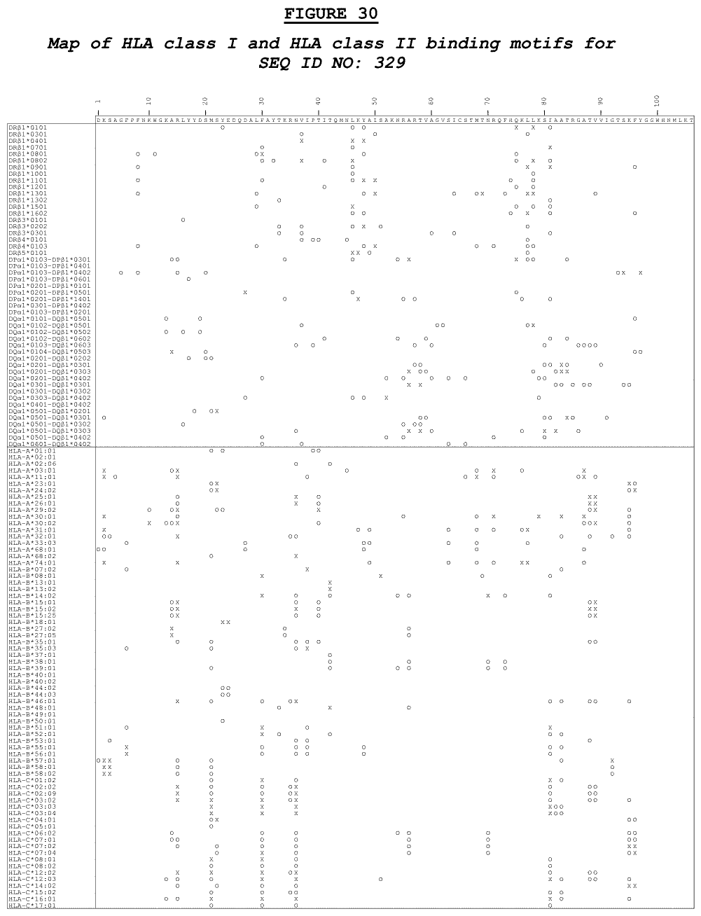

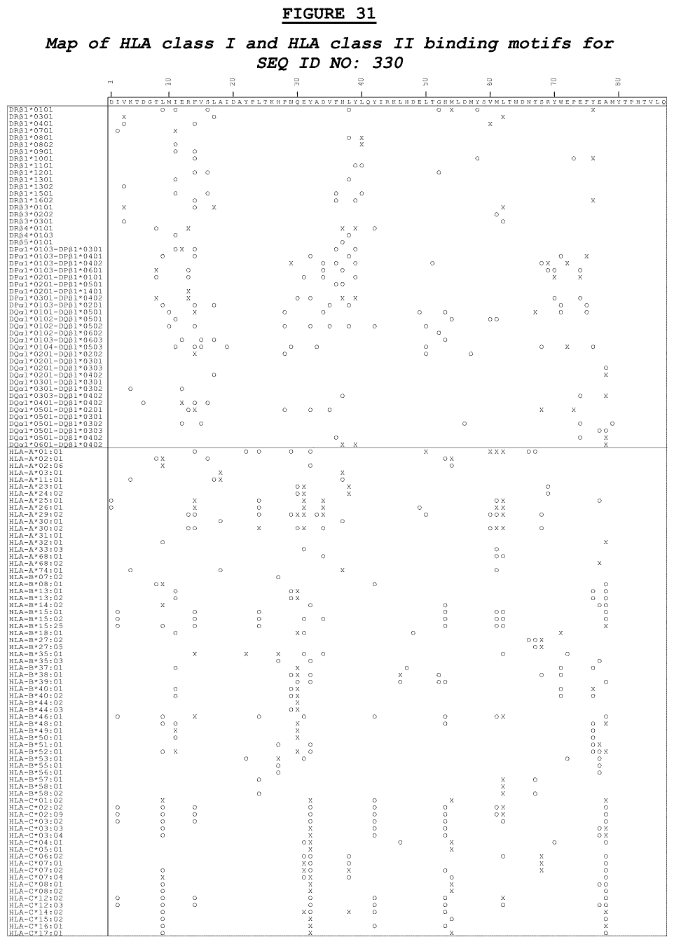

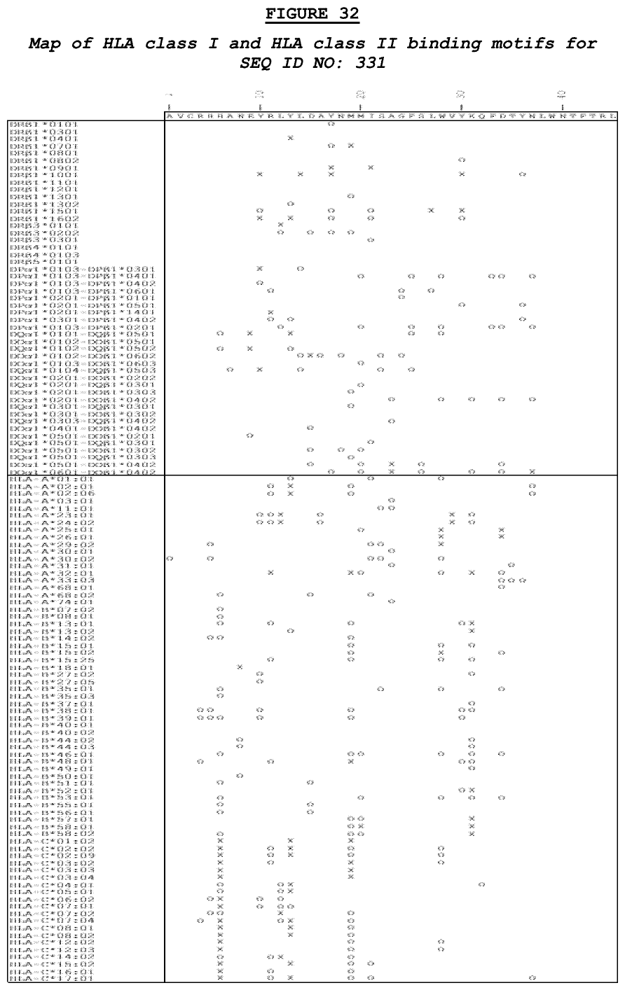

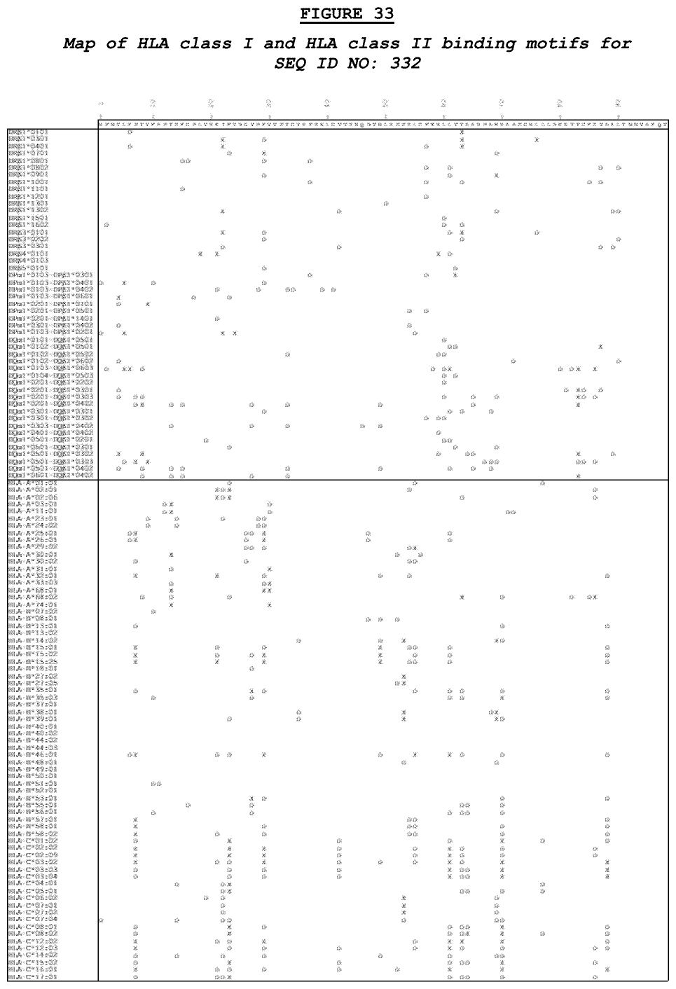

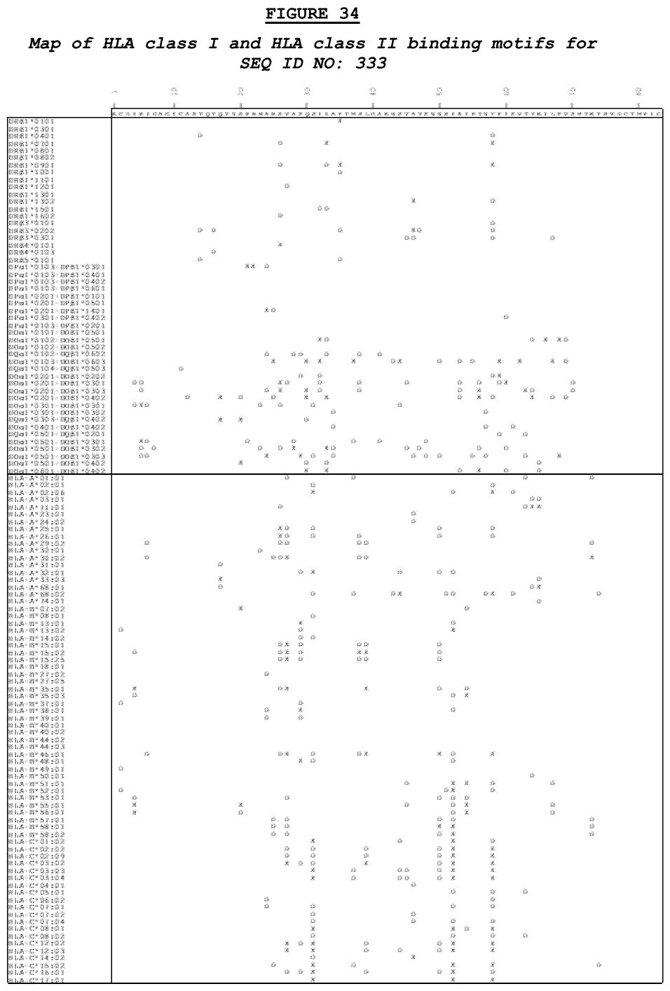

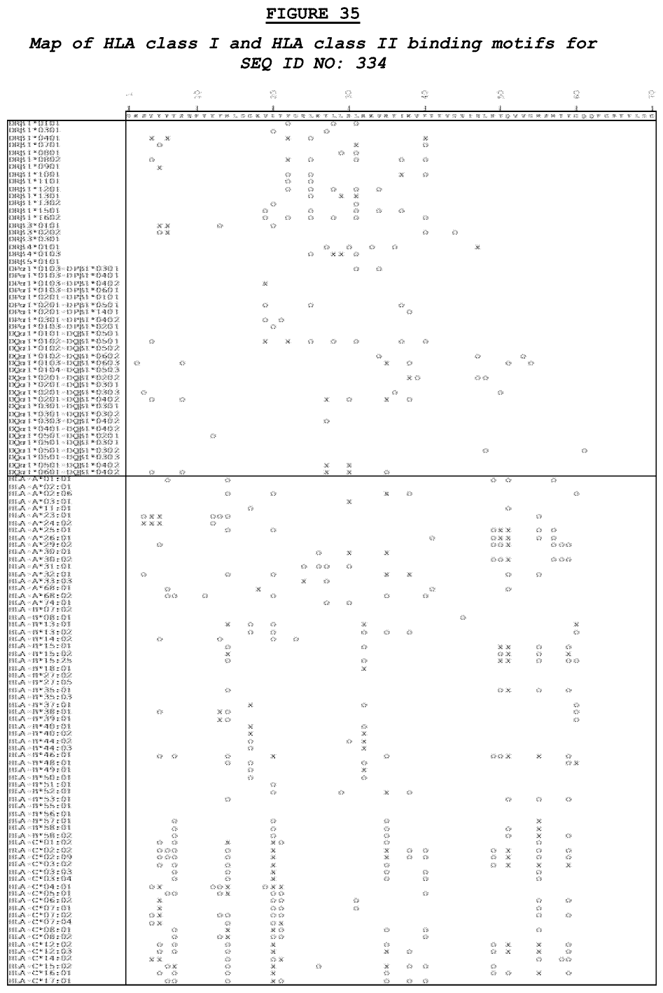

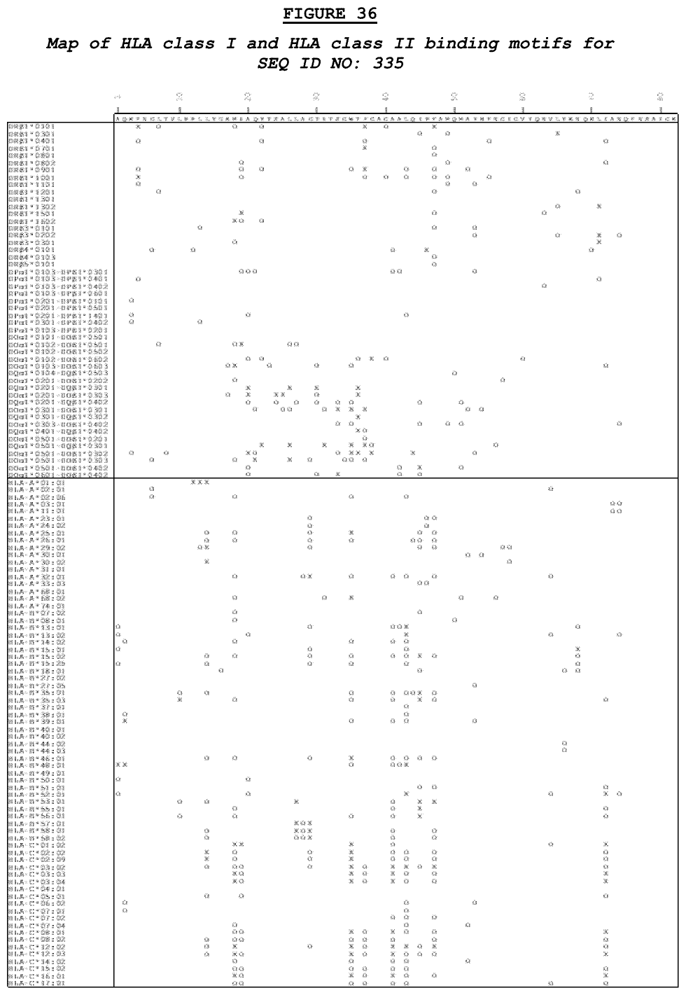

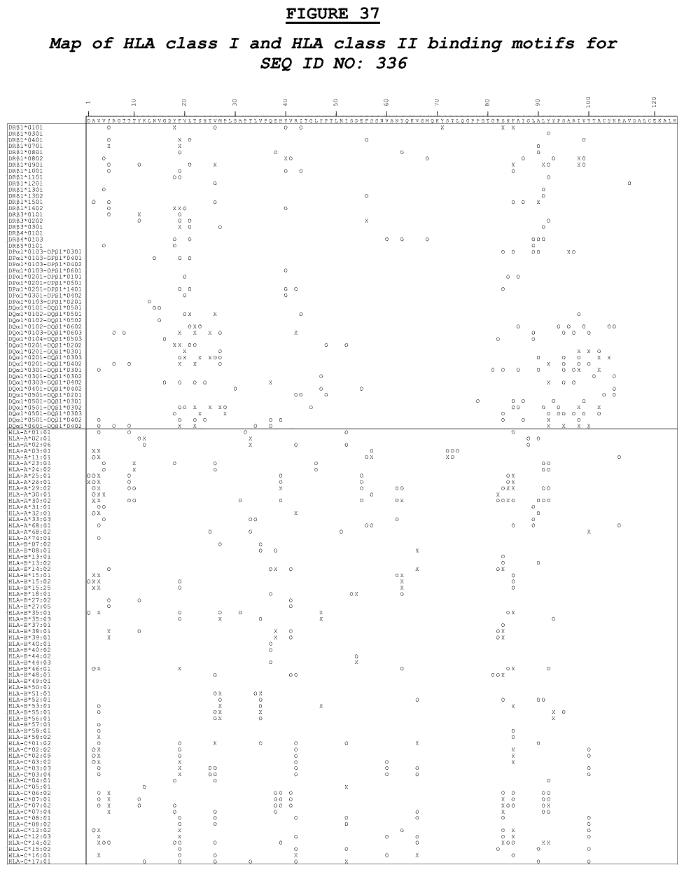

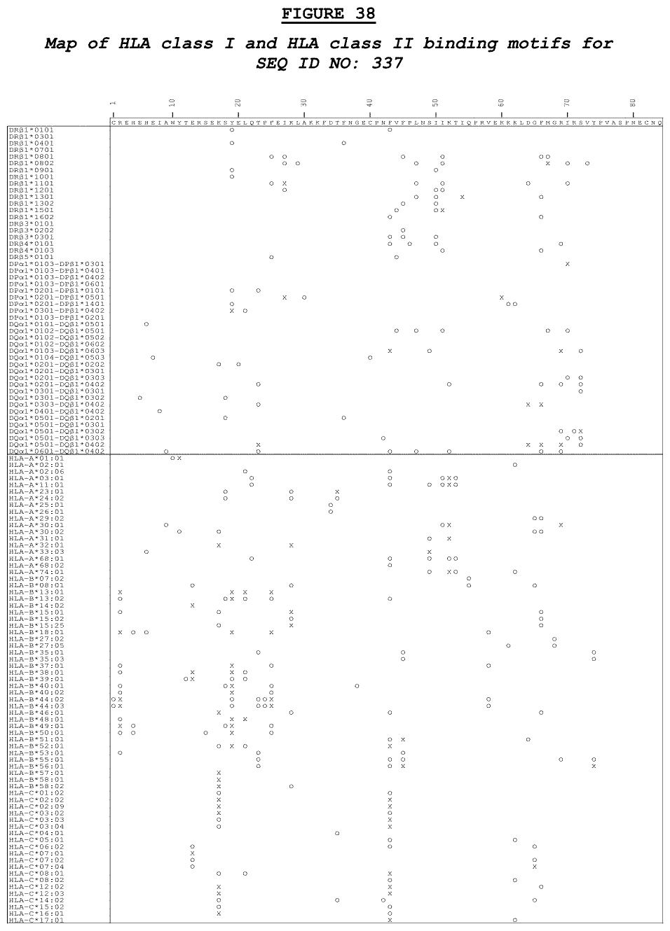

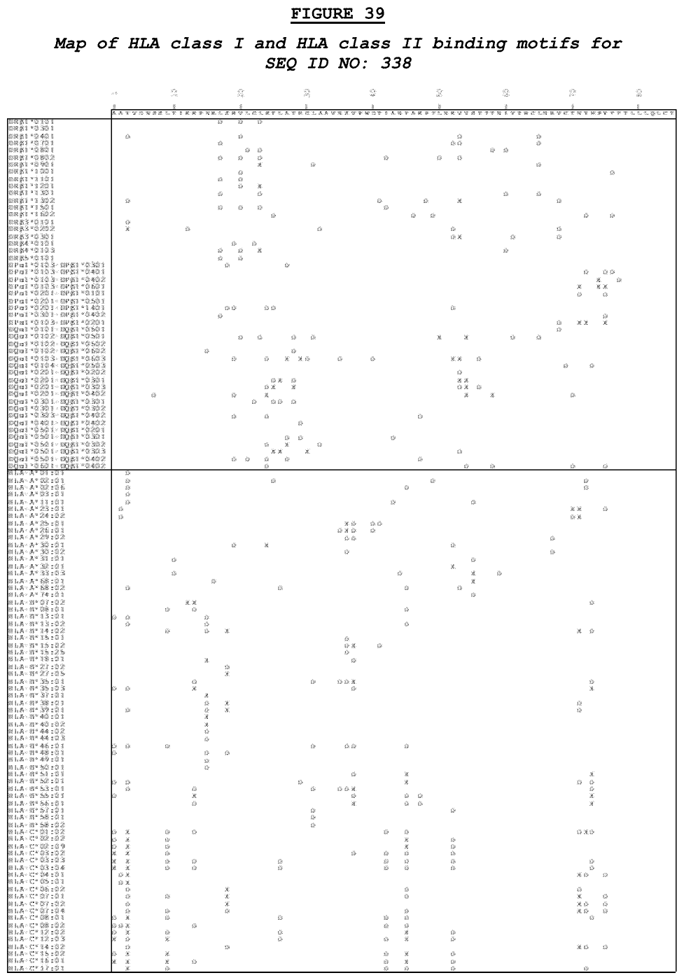

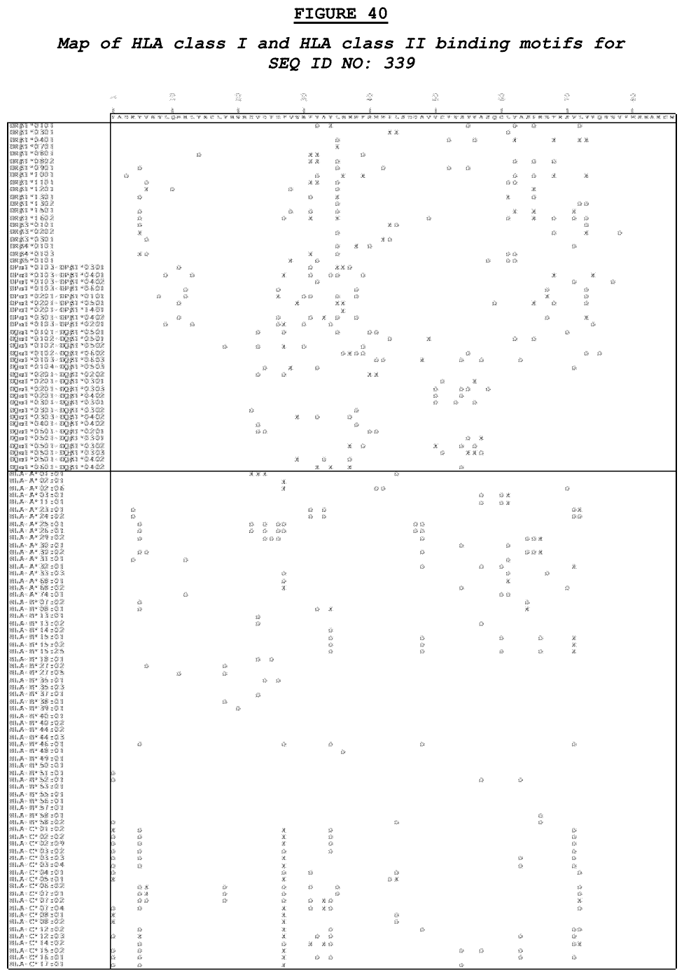

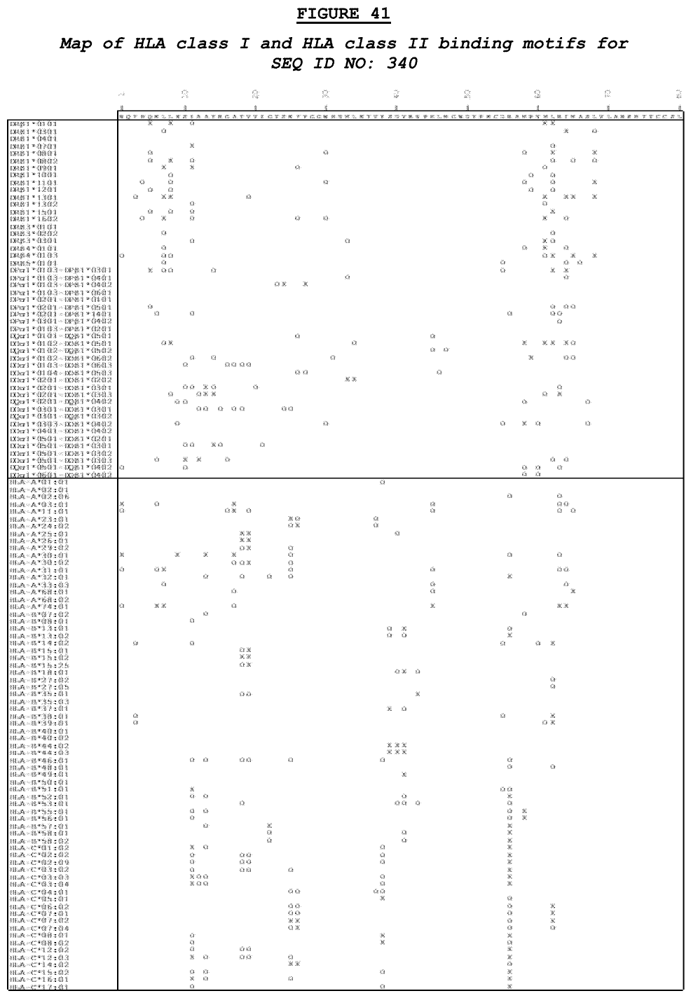

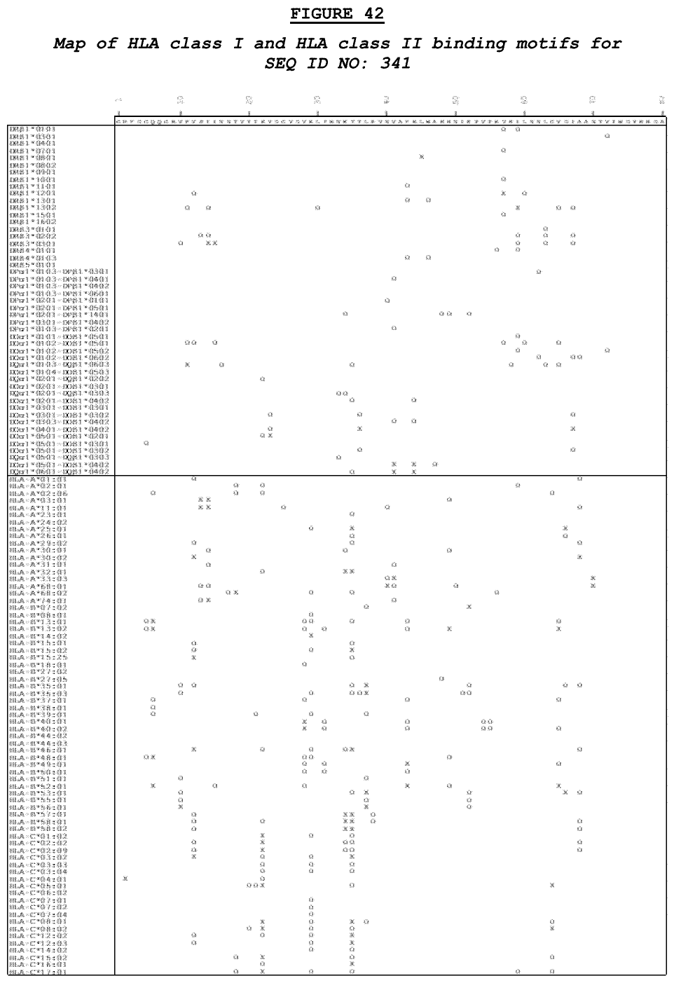

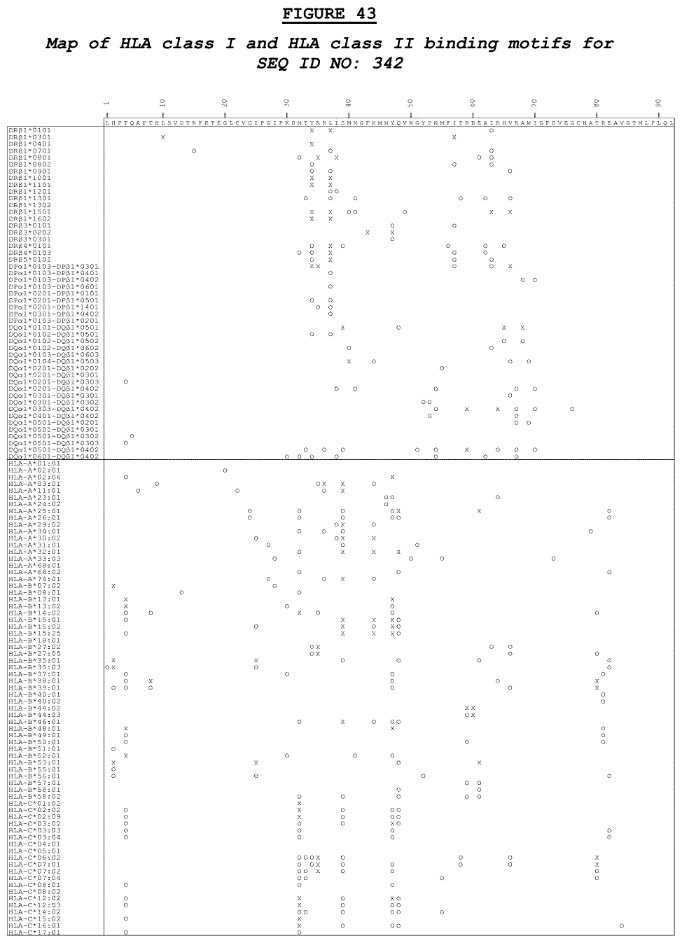

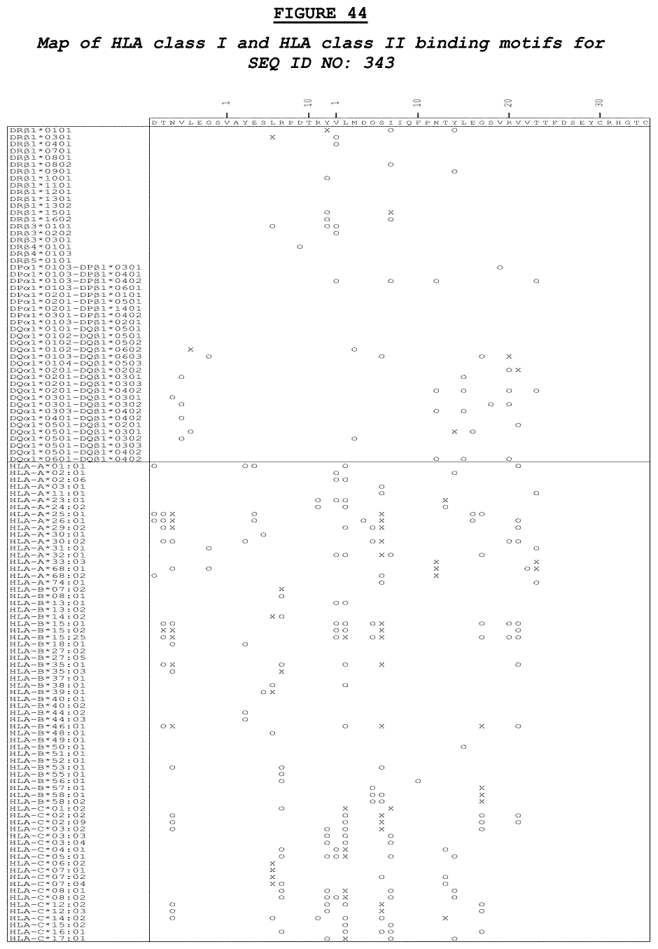

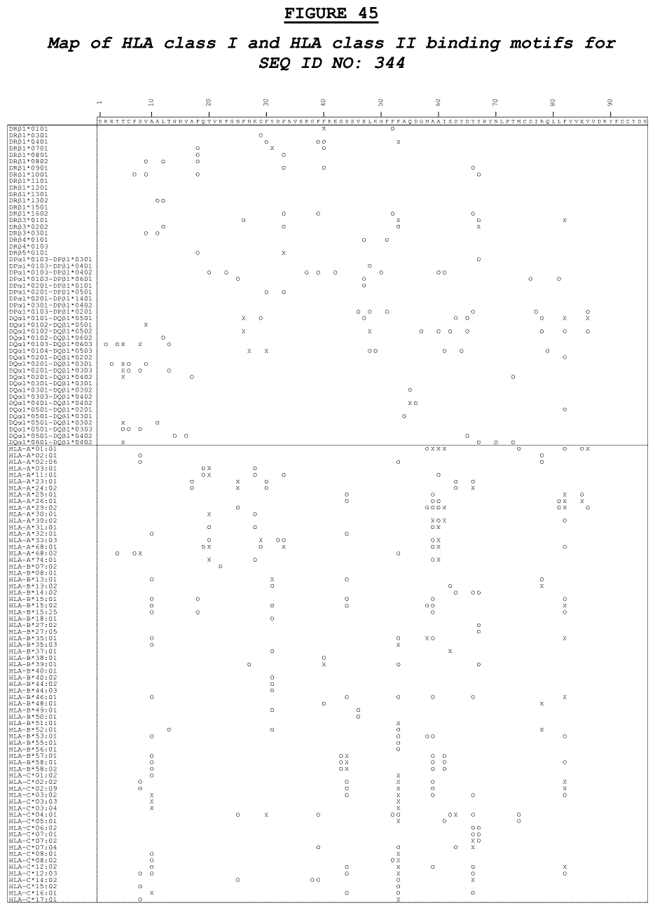

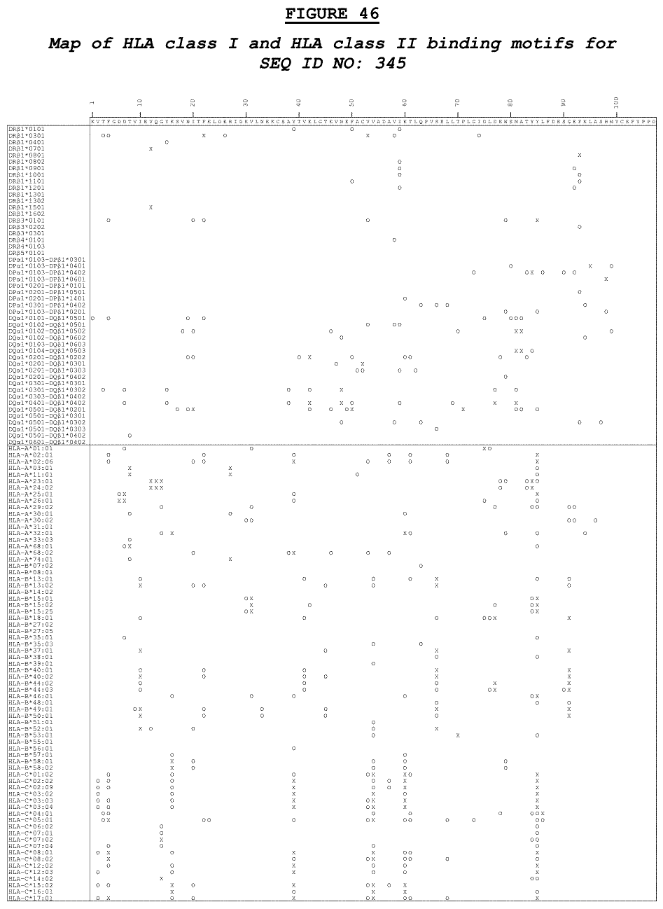

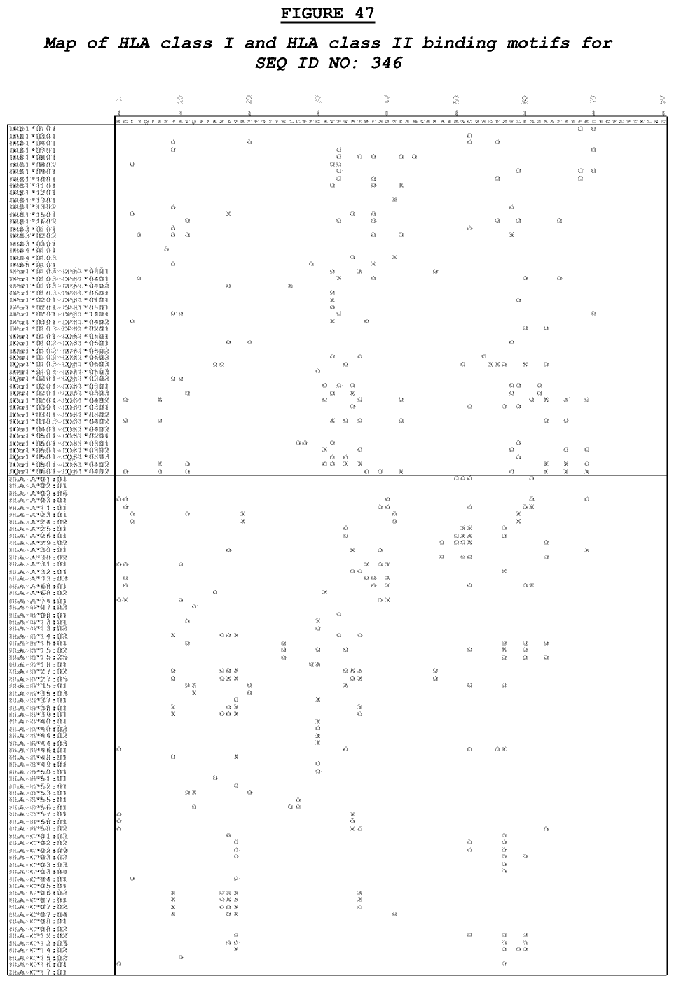

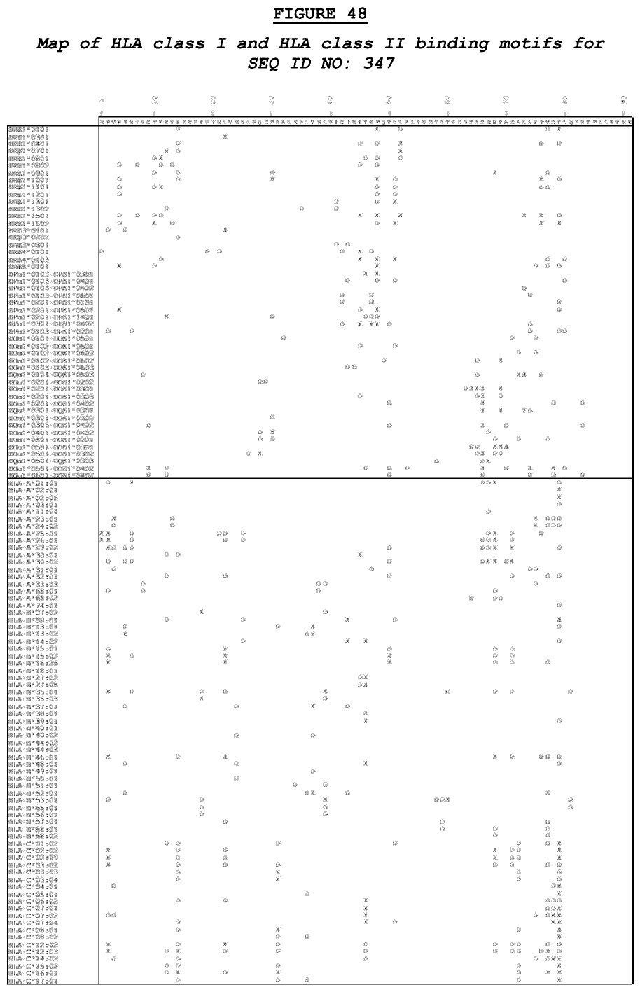

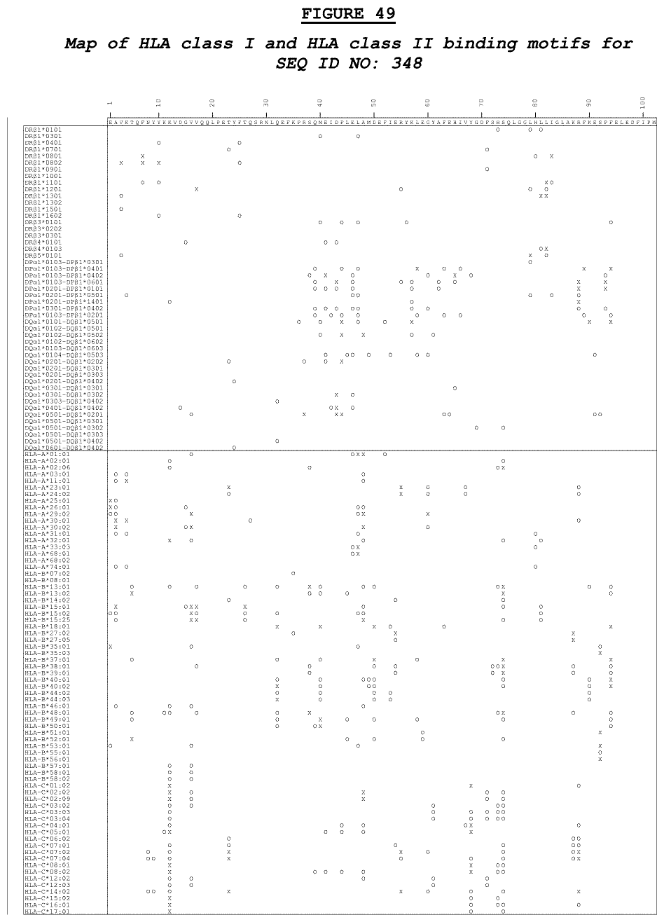

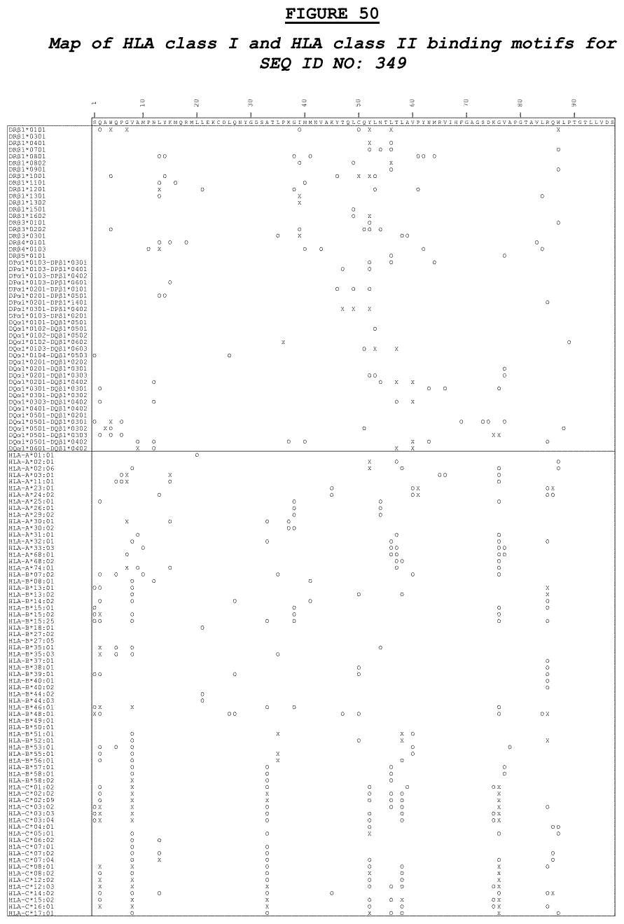

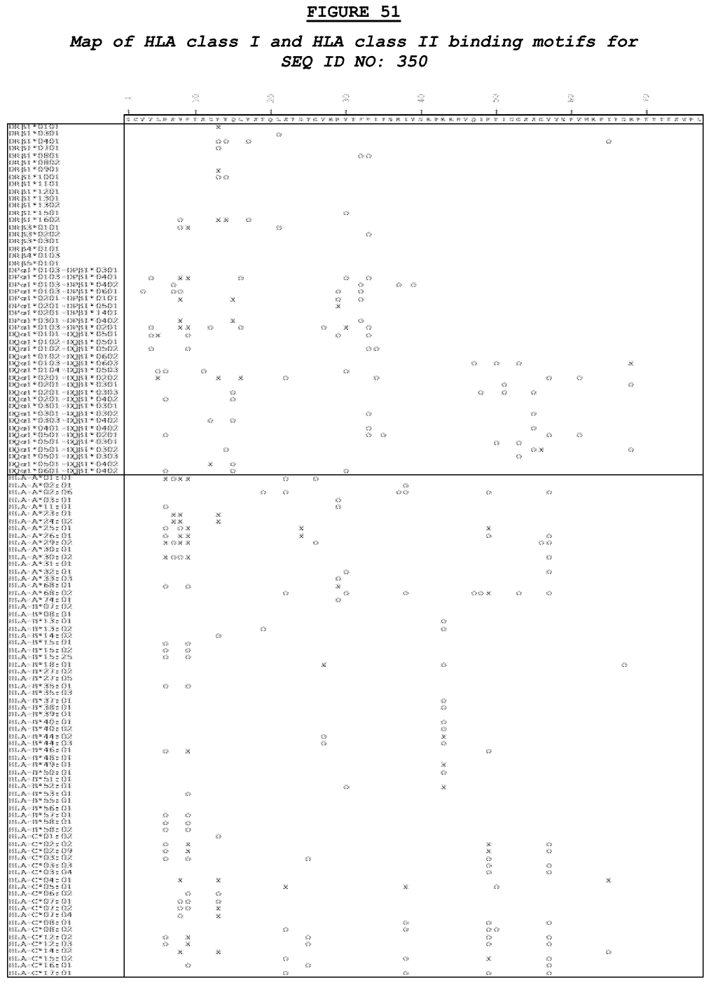

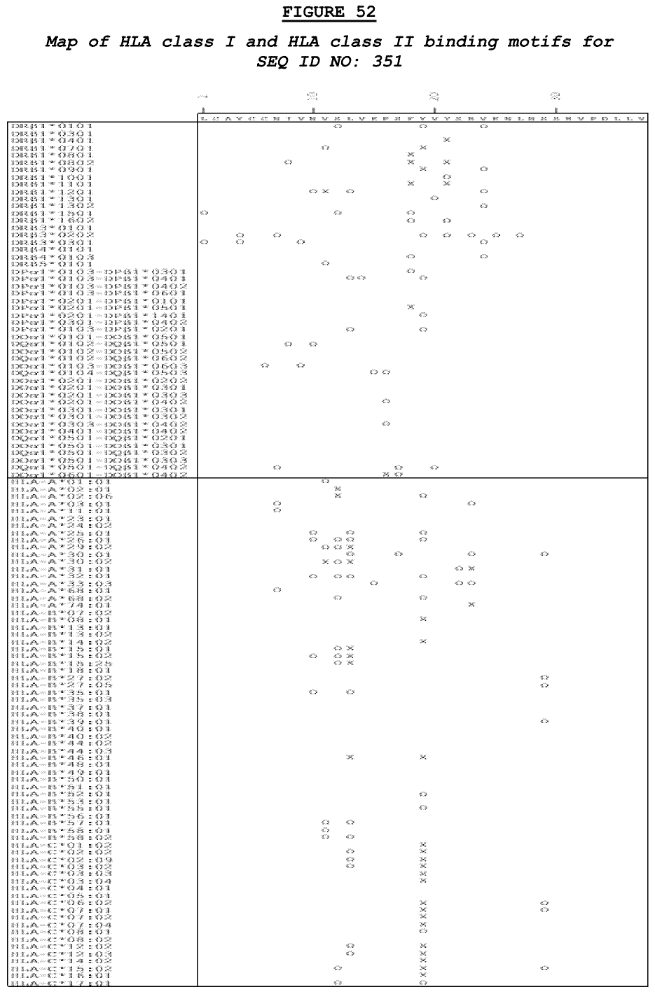

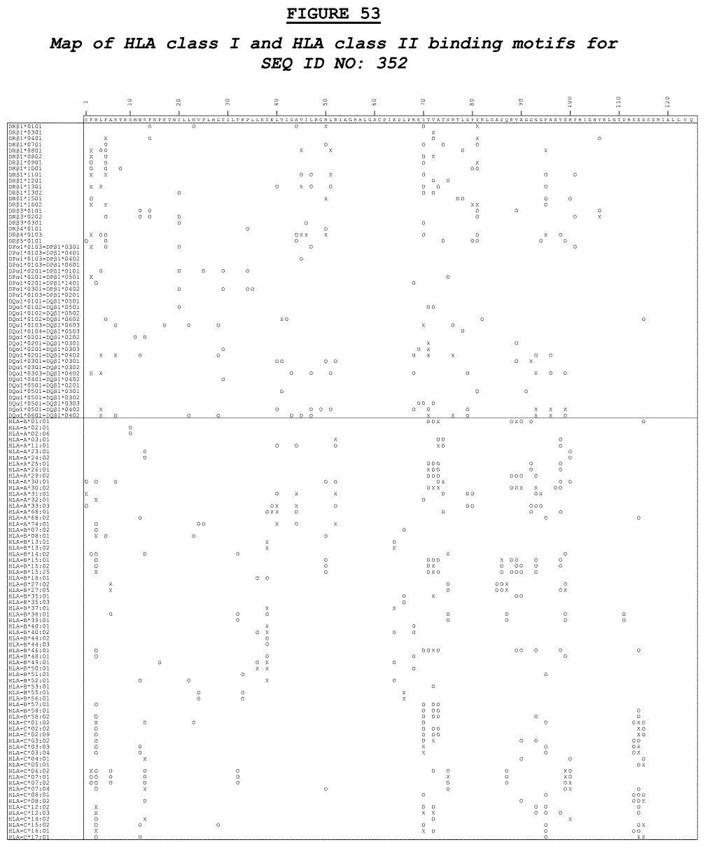

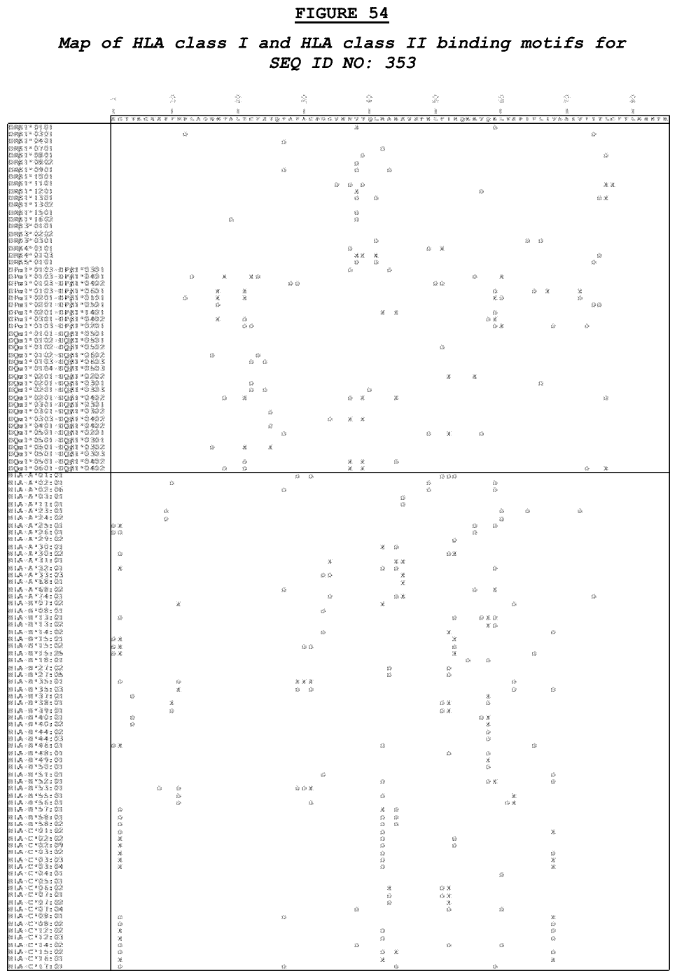

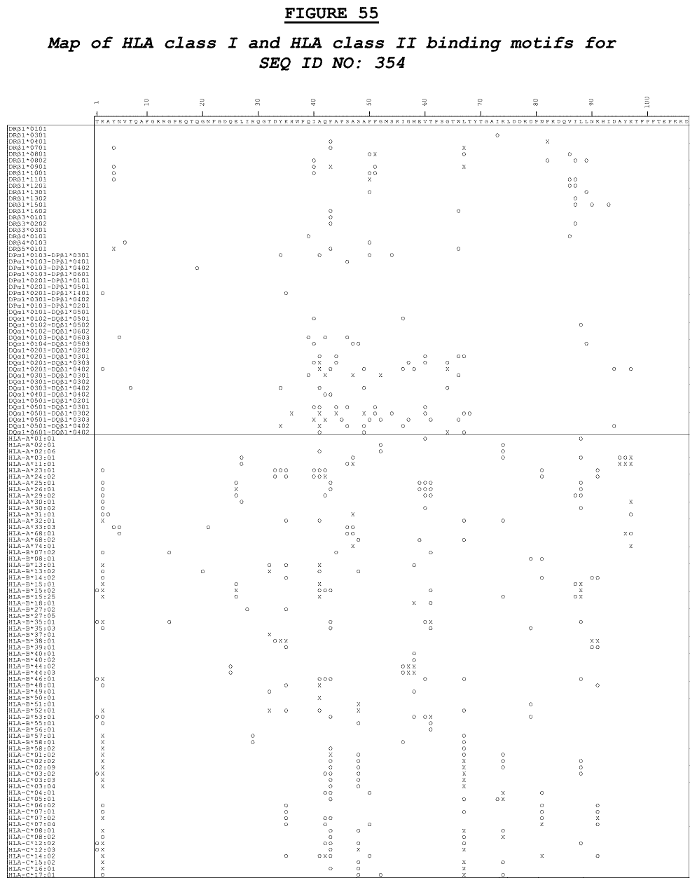

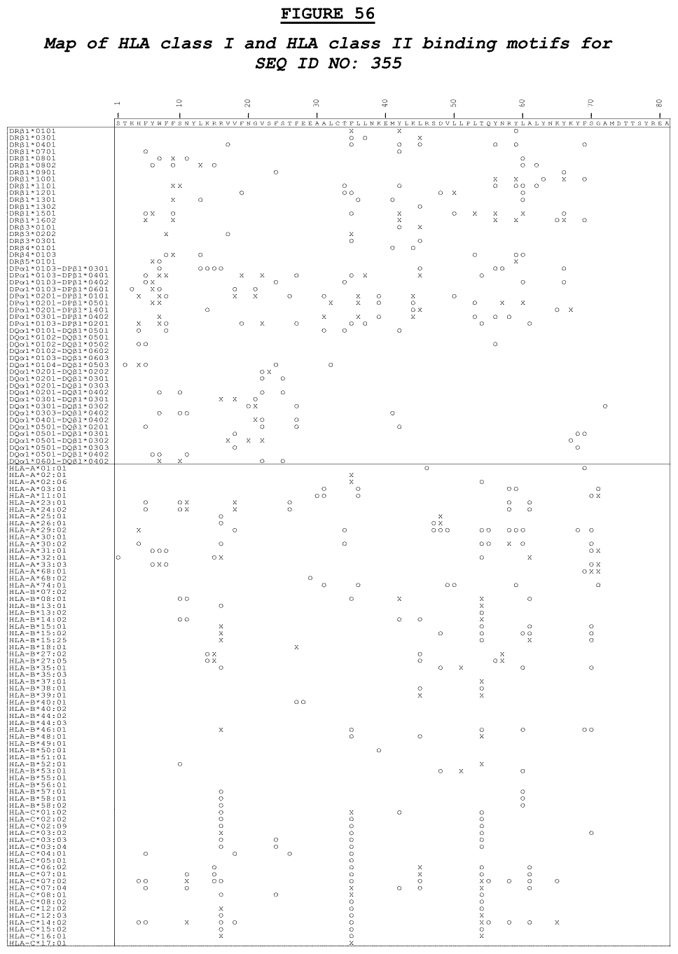

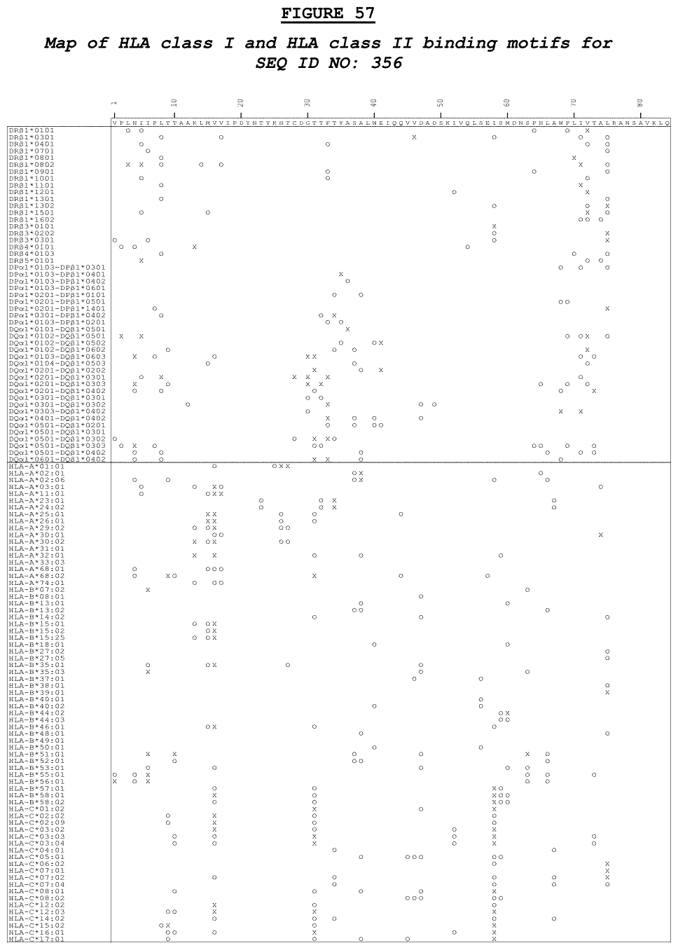

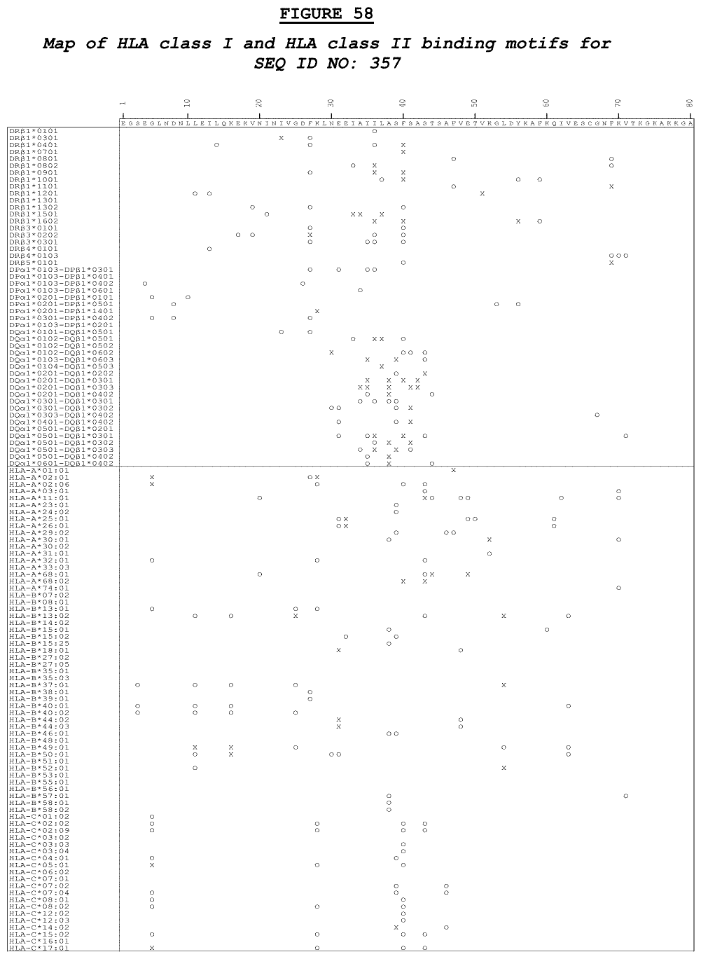

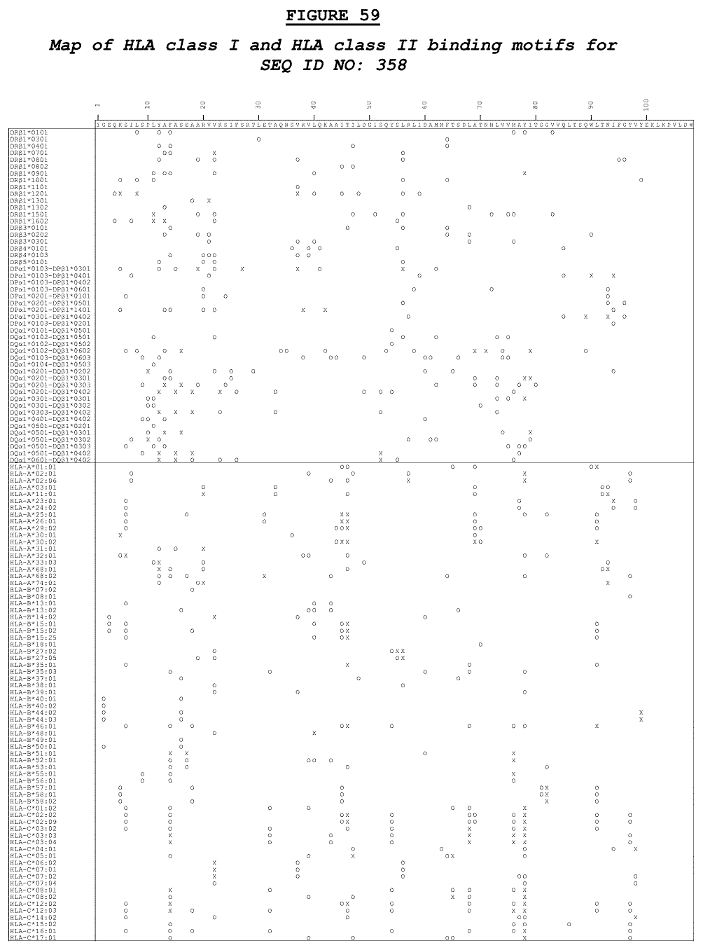

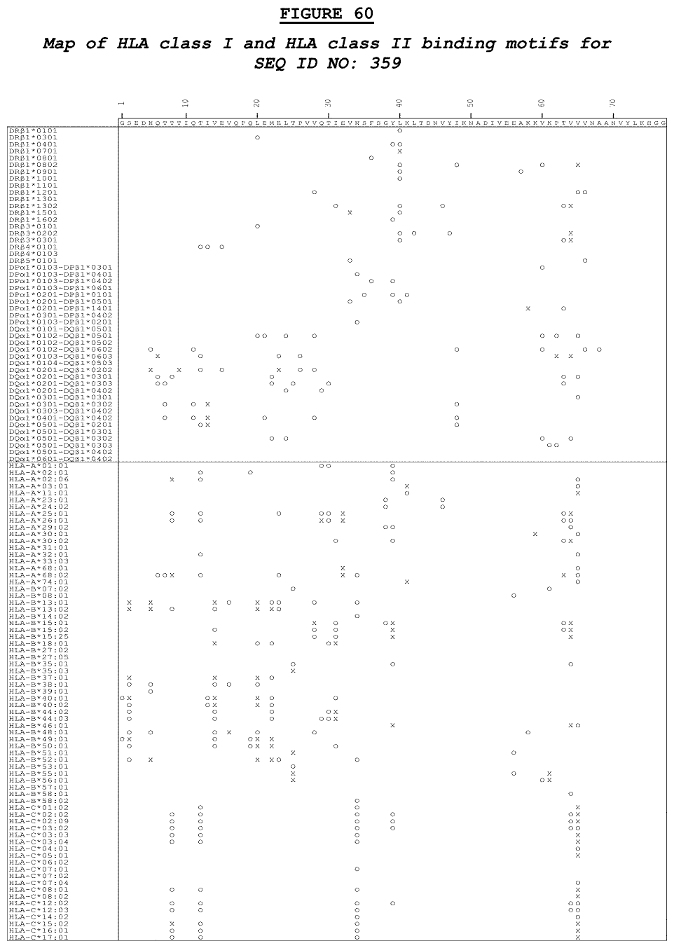

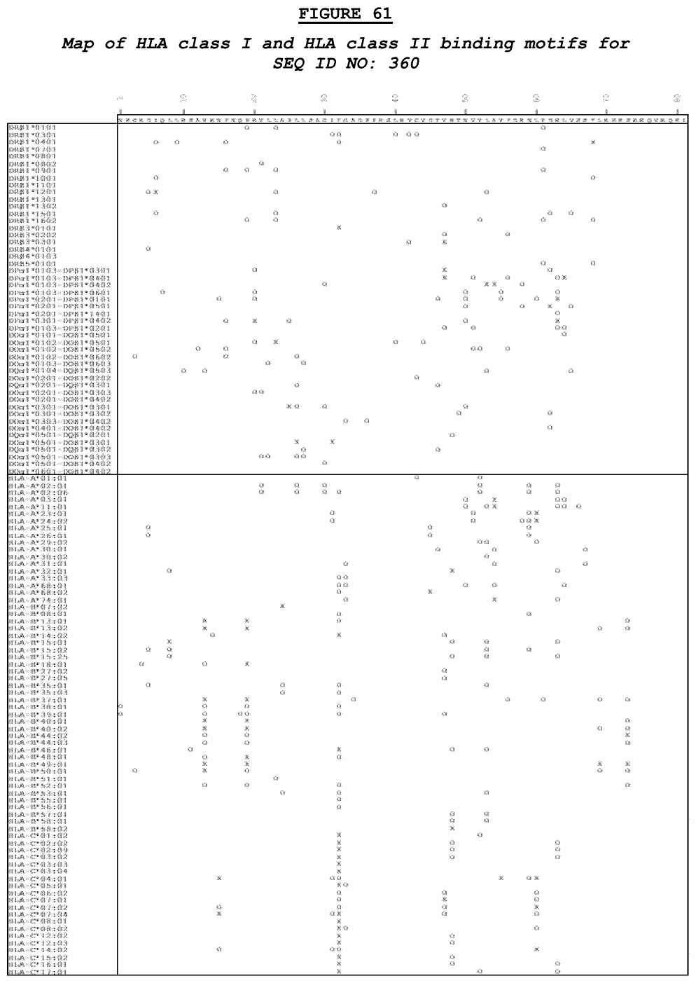

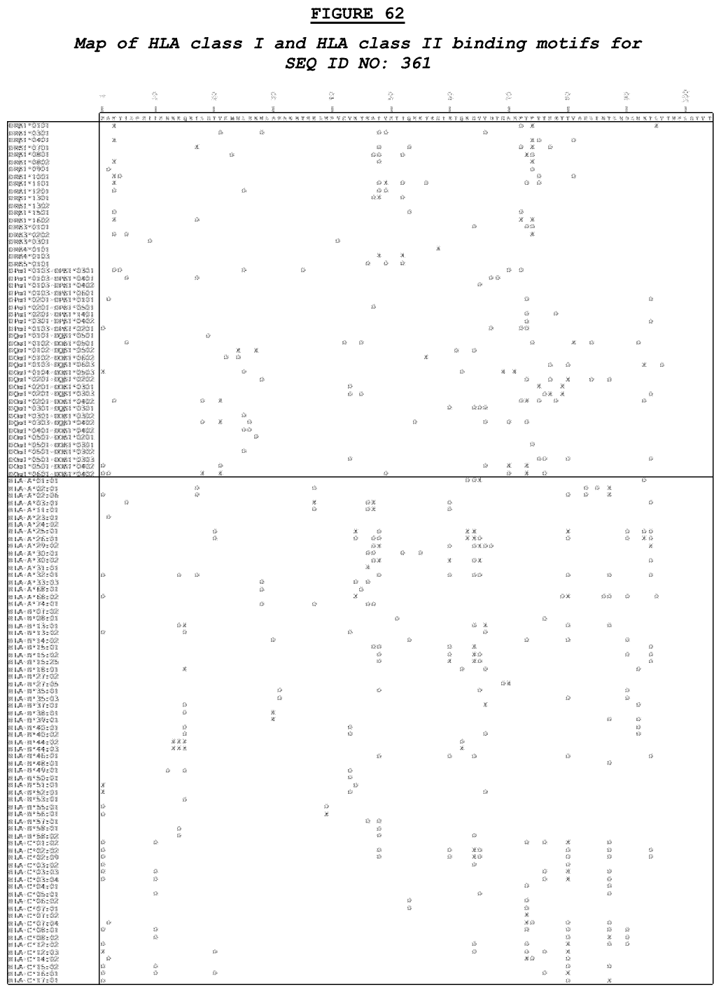

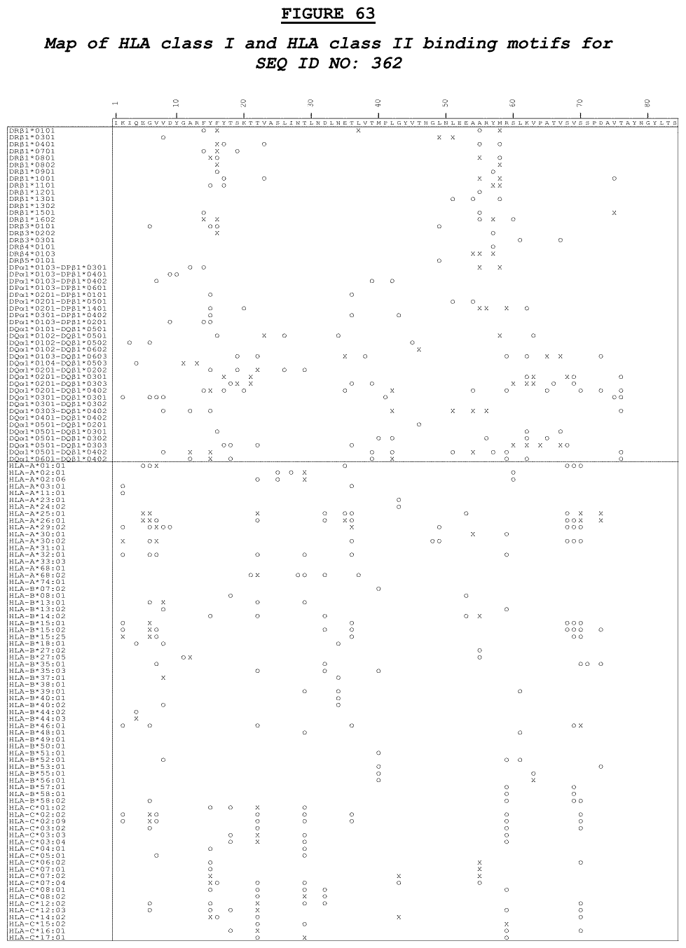

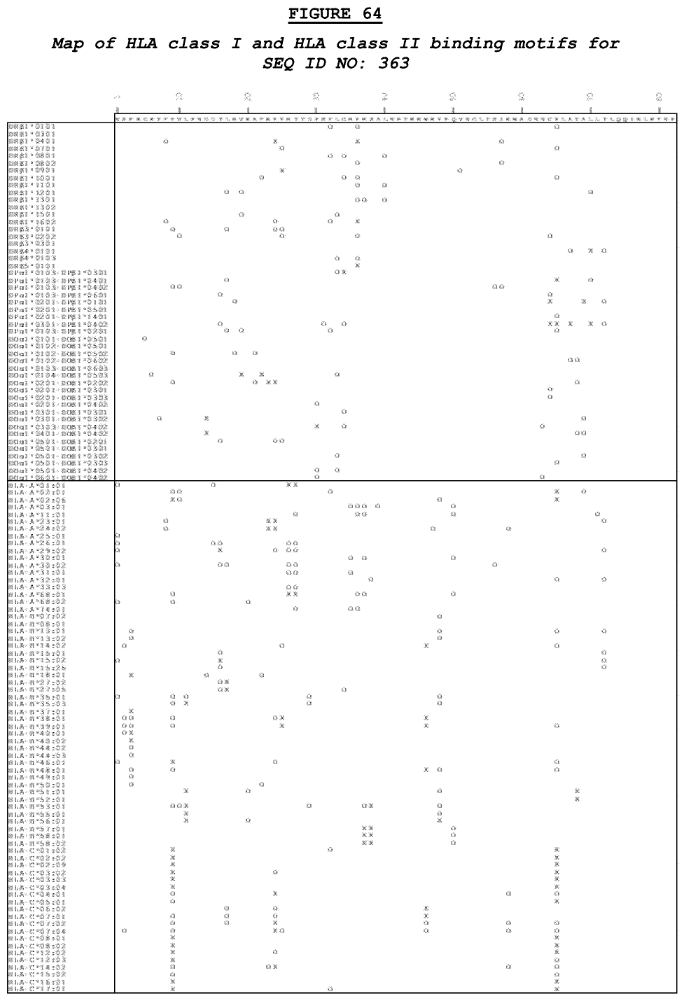

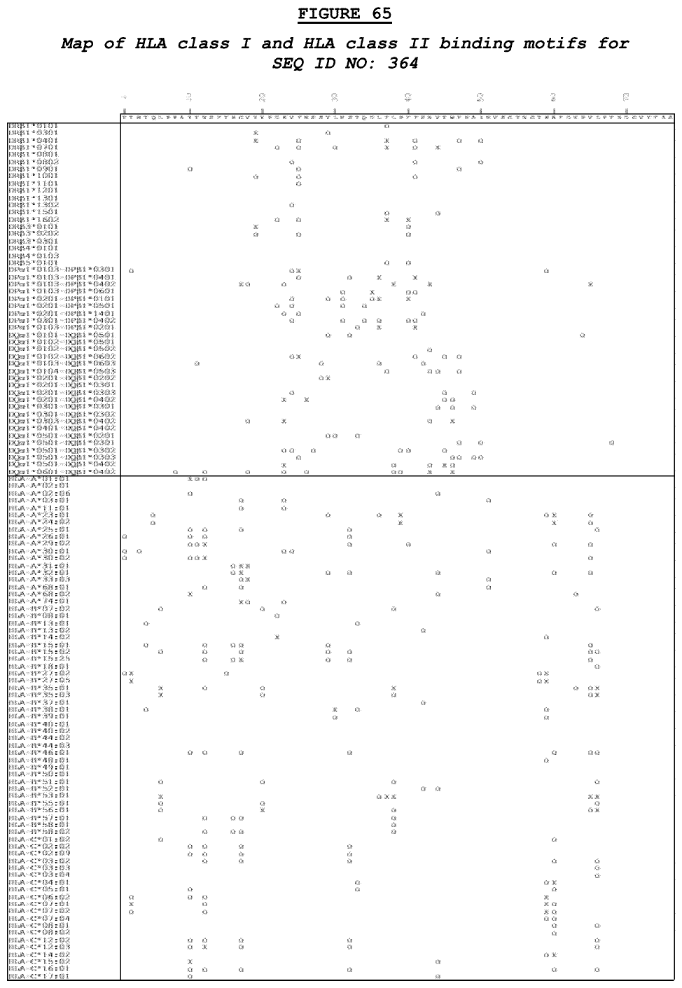

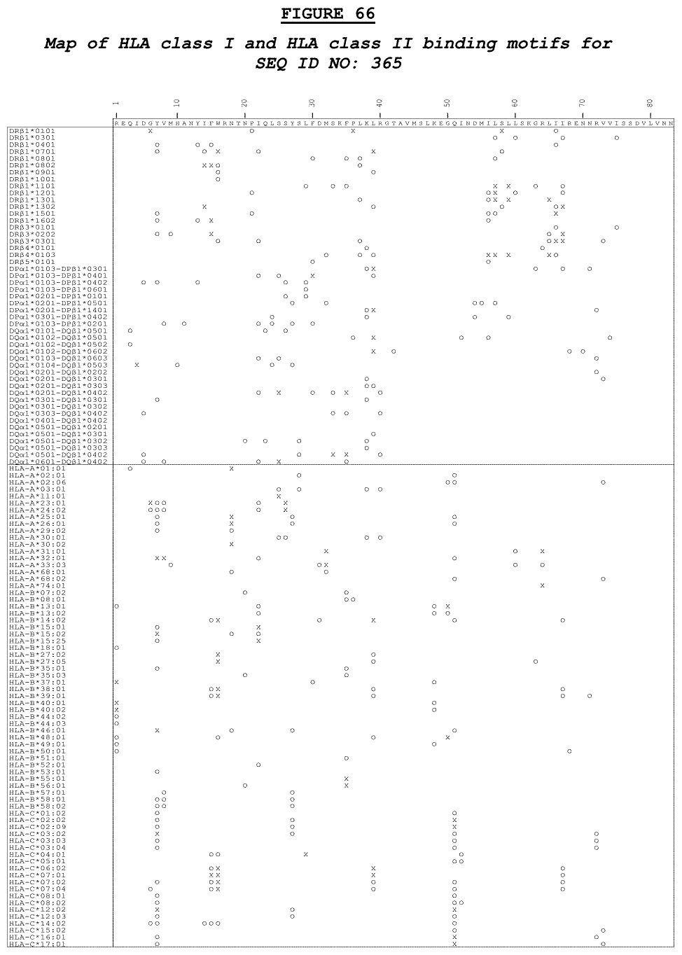

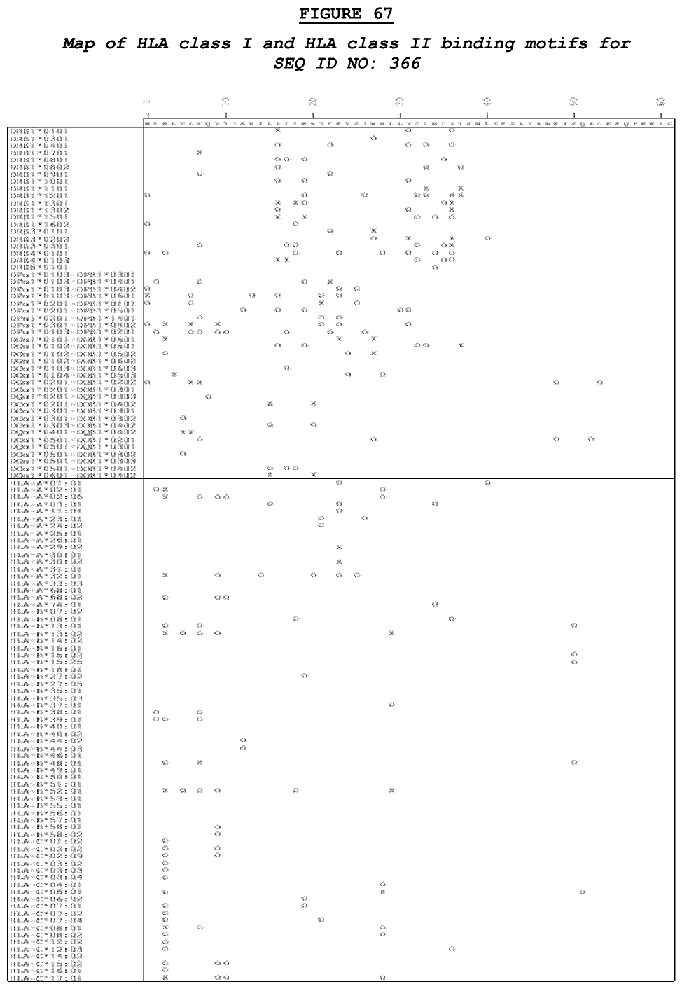

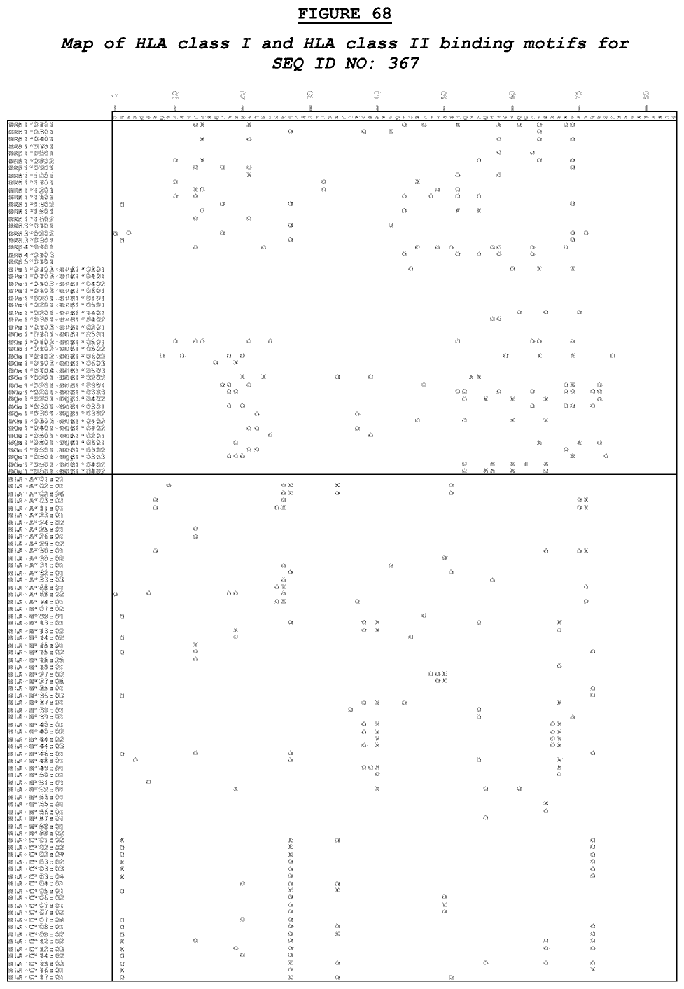

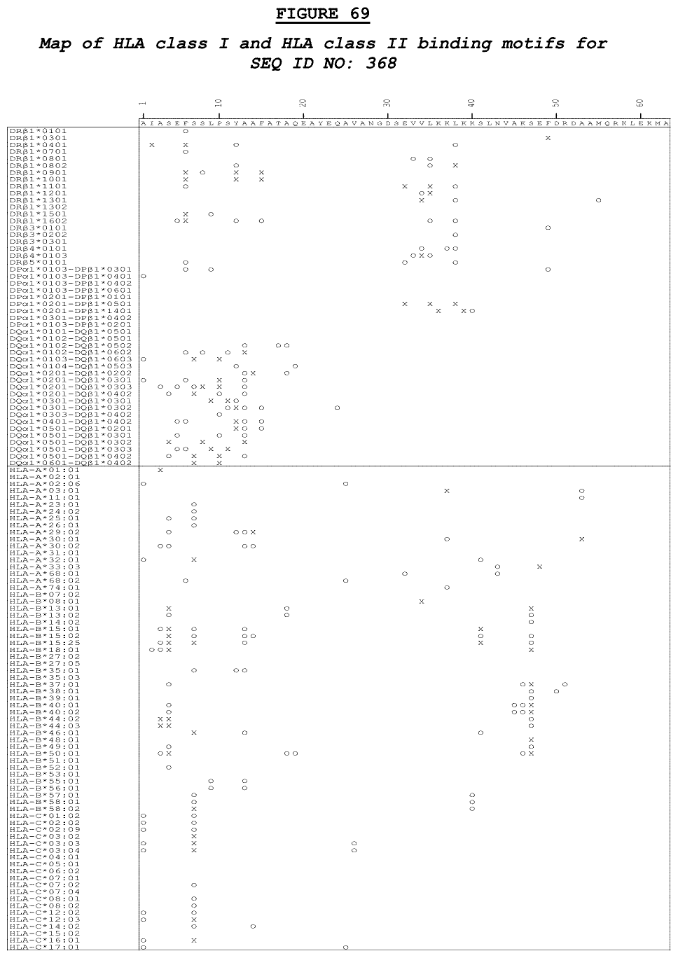

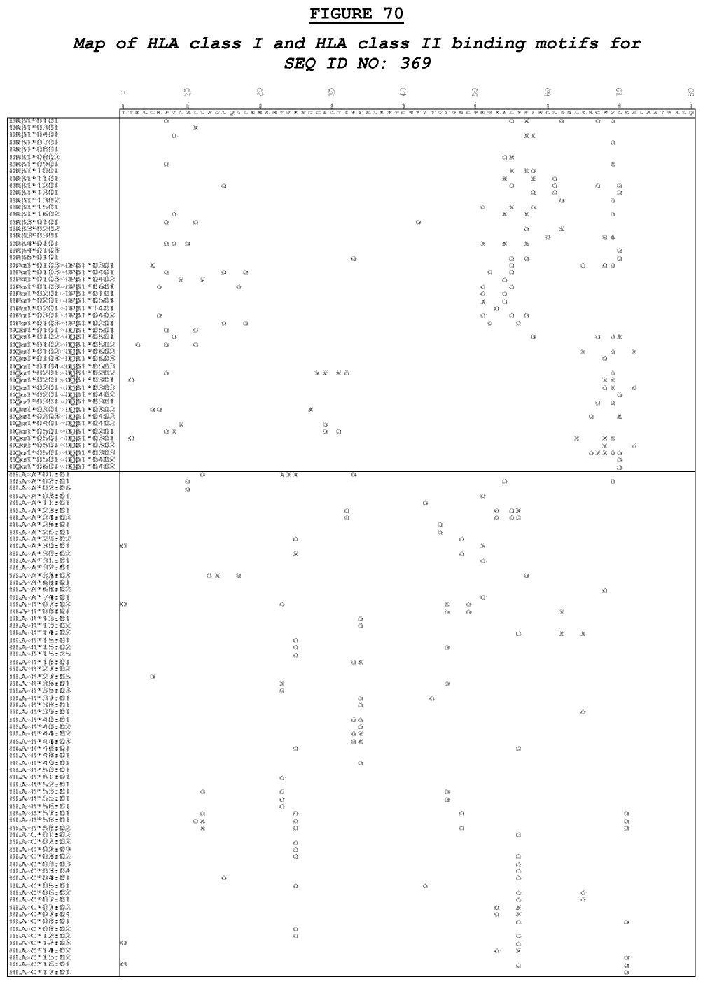

The number of HLA class I binding motifs across the selected 70 HLA class I alleles and the number of HLA class II binding motifs across the selected 49 HLA class II alleles having a high or high plus (+) moderate affinity were respectively calculated for each 41 amino-acid long window scanning the SARS-CoV-2 sequences and presented in FIGS. 27-70. For this analysis, predicted transmembrane domains were deselected due to their high hydrophobicity. Based on this analysis, forty-two (42) long peptide sequences with a length varying from 34 to 124 amino-acids as presented in Table 4 were selected based on the highest content in HLA class I and/or HLA class II motifs across the SRAS-CoV-2 proteome (SEQ ID NO. 410).

TABLE 4
Selected SARS-CoV-2 long peptide sequences containing
high density HLA class I and/or HLA class II
binding motifs (SEQ ID NO: 328 to 369)

| | | N- | C- | |
|---|---|---|---|---|
| | | terminal | terminal | |
| | | position | position | |
| | | in SEQ | in SEQ | |
| SEQ | | ID NO: | ID NO: | |
| ID No. | Length | 410 | 410 | Sequence |
| 328 | 66 | 2580 | 2645 | VGDSAEVAVKMFDAYVNTFSSTFNVPMEK |
| | | | | LKTLVATAEAELAKNVSLDNVLSTFISAAR |
| | | | | QGFVDSD |
| 329 | 106 | 4891 | 4996 | DKSAGFPFNKWGKARLYYDSMSYEDQDA |
| | | | | LFAYTKRNVIPTITQMNLKYAISAKNRART |
| | | | | VAGVSICSTMTNRQFHQKLLKSIAATRGAT |
| | | | | VVIGTSKFYGGWHNMLKT |
| 330 | 87 | 5238 | 5324 | DIVKTDGTLMIERFVSLAIDAYPLTKHPNQ |
| | | | | EYADVFHLYLQYIRKLHDELTGHMLDMYS |
| | | | | VMLTNDNTSRYWEPEFYEAMYTPHTVLQ |
| 331 | 45 | 6407 | 6452 | AVCRHHANEYRLYLDAYNMMISAGFSLW |
| | | | | VYKQFDTYNLWNTFTRLQ |
| 332 | 98 | 4704 | 4801 | NFNVLFSTVFPPTSFGPLVRKIFVDGVPFVV |
| | | | | STGYHFRELGVVHNQDVNLHSSRLSFKELL |
| | | | | VYAADPAMHAASGNLLLDKRTTCFSVAAL |
| | | | | TNNVAFQT |
| 333 | 83 | 7757 | 7839 | ECDIPIGAGICASYQTQTNSPRRARSVASQS |
| | | | | IIAYTMSLGAENSVAYSNNSIAIPTNFTISV |
| | | | | TTEILPVSMTKTSVDCTMYIC |
| 334 | 70 | 1532 | 1601 | DKSVYYTSNPTTFHLDGEVITFDNLKTLLS |
| | | | | LREVRTIKVFTTVDNINLHTQVVDMSMTY |
| | | | | GQQFGPTYLDG |
| 335 | 82 | 7948 | 8029 | AQKFNGLTVLPPLLTDEMIAQYTSALLAGT |
| | | | | ITSGWTFGAGAALQIPFAMQMAYRFNGIG |
| | | | | VTQNVLYENQKLIANQFNSAIGK |
| 336 | 117 | 5531 | 5647 | DAVVYRGTTTYKLNVGDYFVLTSHTVMPL |
| | | | | SAPTLVPQEHYVRITGLYPTLNISDEFSSNV |
| | | | | ANYQKVGMQKYSTLQGPPGTGKSHFAIGL |
| | | | | ALYYPSARIVYTACSHAAVDALCEKALK |
| 337 | 84 | 231 | 314 | CREHEHEIAWYTERSEKSYELQTPFEIKLA |
| | | | | KKFDTFNGECPNFVFPLNSIIKTIQPRVEKK |
| | | | | KLDGFMGRIRSVYPVASPNECNQ |
| 338 | 85 | 2097 | 2181 | AAYVDNSSLTIKKPNELSRVLGLKTLATHG |
| | | | | LAAVNSVPWDTIANYAKPFLNKVVSTTTNI |
| | | | | VTRCLNRVCTNYMPYFFTLLLQLCT |
| 339 | 86 | 5107 | 5192 | IADKYVRNLQHRLYECLYRNRDVDTDFVN |
| | | | | EFYAYLRKHFSMMILSDDAVVCFNSTYAS |
| | | | | QGLVASIKNFKSVLYYQNNVFMSEAKCW |
| 340 | 80 | 4961 | 5040 | RQFHQKLLKSIAATRGATVVIGTSKFYGG |
| | | | | WHNMLKTVYSDVENPHLMGWDYPKCDR |
| | | | | AMPNMLRIMASLVLARKHTTCCSL |
| 341 | 80 | 6465 | 6544 | GHFDGQQGEVPVSIINNTVYTKVDGVDVE |
| | | | | LFENKTTLPVNVAFELWAKRNIKPVPEVKI |
| | | | | LNNLGVDIAANTVIWDYKRDA |
| 342 | 92 | 5943 | 6034 | LHPTQAPTHLSVDTKFKTEGLCVDIPGIPKD |
| | | | | MTYRRLISMMGFKMNYQVNGYPNMFITRE |
| | | | | EAIRHVRAWIGFDVEGCHATREAVGTNLP |
| | | | | LQL |
| 343 | 55 | 2935 | 2989 | DTNVLEGSVAYESLRPDTRYVLMDGSIIQF |
| | | | | PNTYLEGSVRVVTTFDSEYCRHGTC |
| 344 | 96 | 4782 | 4877 | DKRTTCFSVAALTNNVAFQTVKPGNFNKD |
| | | | | FYDFAVSKGFFKEGSSVELKHFFFAQDGNA |
| | | | | AISDYDYYRYNLPTMCDIRQLLFVVEVVD |
| | | | | KYFDCYDG |
| 345 | 107 | 822 | 928 | KVTFGDDTVIEVQGYKSVNITFELDERIDK |
| | | | | VLNEKCSAYTVELGTEVNEFACVVADAVI |
| | | | | KTLQPVSELLTPLGIDLDEWSMATYYLFDE |
| | | | | SGEFKLASHMYCSFYPPD |
| 346 | 80 | 7406 | 7485 | KGIYQTSNFRVQPTESIVRFPNITNLCPFGE |
| | | | | VFNATRFASVYAWNRKRISNCVADYSVLY |
| | | | | NSASFSTFKCYGVSPTKLND |
| 347 | 91 | 7287 | 7377 | EFVFKNIDGYFKIYSKHTPINLVRDLPQGFS |
| | | | | ALEPLVDLPIGINITRFQTLLALHRSYLTPG |
| | | | | DSSSGWTAGAAAYYVGYLQPRTFLLKYNE |
| 348 | 102 | 6622 | 6723 | EAVKTQFNYYKKVDGVVQQLPETYFTQSR |
| | | | | NLQEFKPRSQMEIDFLELAMDEFIERYKLE |
| | | | | GYAFEHIVYGDFSHSQLGGLHLLIGLAKRF |
| | | | | KESPFELEDFIPM |
| 349 | 97 | 6800 | 6896 | SQAWQPGVAMPNLYKMQRMLLEKCDLQ |
| | | | | NYGDSATLPKGIIVIMNVAKYTQLCQYLNTL |
| | | | | TLAVPYNMRVIHFGAGSDKGVAPGTAVLR |
| | | | | QWLPTGTLLVDS |
| 350 | 77 | 8568 | 8644 | DCVVLHSYFTSDYYQLYSTQLSTDTGVEH |
| | | | | VTFFIYNKIVDEPEEHVQIHTIDGSSGVVNP |
| | | | | VMEPIYDEPTTTTSVPL |
| 351 | 37 | 8683 | 8719 | LCAYCCNIVNVSLVKPSFYVYSRVKNLNSS |
| | | | | RVPDLLV |
| 352 | 124 | 8818 | 8941 | SFRLFARTRSMWSFNPETNILLNVPLHGTIL |
| | | | | TRPLLESELVIGAVILRGHLRIAGHHLGRCD |
| | | | | IKDLPKEITVATSRTLSYYKLGASQRVAGD |
| | | | | SGFAAYSRYRIGNYKLNTDHSSSSDNIALL |
| | | | | VQ |
| 353 | 85 | 9039 | 9123 | SGTYEGNSPFHPLADNKFALTCFSTQFAFA |
| | | | | CPDGVKHVYQLRARSVSPKLFIRQEEVQEL |
| | | | | YSPIFLIVAAIVFITLCFTLKRKTE |
| 354 | 107 | 9552 | 9658 | TKAYNVTQAFGRRGPEQTQGNFGDQELIR |
| | | | | QGTDYKHWPQIAQFAPSASAFFGMSRIGM |
| | | | | EVTPSGTWLTYTGAIKLDDKDPNFKDQVIL |
| | | | | LNKHIDAYKTFPPTEPKKD |
| 355 | 81 | 3149 | 3229 | STKHFYWFFSNYLKRRVVFNGVSFSTFEEA |
| | | | | ALCTFLLNKEMYLKLRSDVLLPLTQYNRY |
| | | | | LALYNKYKYFSGAMDTTSYREA |
| 356 | 84 | 4057 | 4140 | VPLNIIPLTTAAKLMVVIPDYNTYKNTCDG |
| | | | | TTFTYASALWEIQQVVDADSKIVQLSEISM |
| | | | | DNSPNLAWPLIVTALRANSAVKLQ |
| 357 | 80 | 441 | 520 | EGSEGLNDNLLEILQKEKVNINIVGDFKLN |
| | | | | EEIAIILASFSASTSAFVETVKGLDYKAFKQI |
| | | | | VESCGNFKVTKGKAKKGA |
| 358 | 108 | 523 | 630 | IGEQKSILSPLYAFASEAARVVRSIFSRTLE |
| | | | | TAQNSVRVLQKAAITILDGISQYSLRLIDAM |
| | | | | MFTSDLATNNLVVMAYITGGVVQLTSQWL |
| | | | | TNIFGTVYEKLKPVLDW |
| 359 | 77 | 993 | 1069 | GSEDNQTTTIQTIVEVQPQLEMELTPVVQTI |
| | | | | EVNSFSGYLKLTDNVYIKNADIVEEAKKVK |
| | | | | PTVVVNAANVYLKHGG |
| 360 | 81 | 1123 | 1203 | NKGEDIQLLKSAYENFNQHEVLLAPLLSAG |
| | | | | IFGADPIHSLRVCVDTVRTNVYLAVFDKNL |
| | | | | YDKLVSSFLEMKSEKQVEQKI |
| 361 | 104 | 1351 | 1454 | SAFYILPSIISNEKQEILGTVSWNLREMLAH |
| | | | | AEETRKLMPVCVETKAIVSTIQRKYKGIKIQ |
| | | | | EGVVDYGARFYFYTSKTTVASLINTLNDLN |
| | | | | ETLVTMPLGYVT |
| 362 | 104 | 1409 | 1512 | IKIQEGVVDYGARFYFYTSKTTVASLINTLN |
| | | | | DLNETLVTMPLGYVTHGLNLEEAARYMRS |
| | | | | LKVPATVSVSSPDAVTAYNGYLTSSSKTPE |
| | | | | EHFIETISLAGSYK |
| 363 | 82 | 1611 | 1692 | NSHEGKTFYVLPNDDTLRVEAFEYYHTTD |
| | | | | PSFLGRYMSALNHTKKWKYPQVNGLTSIK |
| | | | | WADNNCYLATALLTLQQIELKFNP |
| 364 | 76 | 7115 | 7190 | TTRTQLPPAYTNSFTRGVYYPDKVFRSSVL |
| | | | | HSTQDLFLPFFSNVTWFHAIHVSGTNGTKR |
| | | | | FDNPVLPFNDGVYFAS |
| 365 | 83 | 7014 | 7096 | REQIDGYVMHANYIFWRNTNPIQLSSYSLF |
| | | | | DMSKFPLKLRGTAVMSLKEGQINDMILSLL |
| | | | | SKGRLIIRENNRVVISSDVLVNN |
| 366 | 61 | 8942 | 9002 | MFHLVDFQVTIAEILLIIMRTFKVSIWNLDY |
| | | | | IINLIIKNLSKSLTENKYSQLDEEQPMEID |
| 367 | 84 | 8046 | 8129 | DVVNQNAQALNTLVKQLSSNFGAISSVLN |
| | | | | DILSRLDKVEAEVQIDRLITGRLQSLQTYVT |
| | | | | QQLIRAAEIRASANLAATKMSECV |
| 368 | 63 | 3943 | 4005 | AIASEFSSLPSYAAFATAQEAYEQAVANGD |
| | | | | SEVVLKKLKKSLNVAKSEFDRDAAMQRKL |
| | | | | EKMA |
| 369 | 80 | 4174 | 4253 | TTKGGRFVLALLSDLQDLKWARFPKSDGT |
| | | | | GTIYTELEPPCRFVTDTPKGPKVKYLYFIKG |
| | | | | LNNLNRGMVLGSLAATVRLQ |

For each selected long sequences SEQ ID NO: 328 to SEQ ID NO: 369, the number of HLA class I and HLA class II alleles for which high or moderate binding was predicted are presented in Table 5.

TABLE 5
Number of HLA class I and HLA class II alleles for which high or moderate binding
was predicted within the selected SARS-CoV-2 long peptide sequences containing high
density HLA class I and/or HLA class II binding motifs (SEQ ID NO: 328 to 369)

| | | N-terminal | C-terminal | | | | | | |
|---|---|---|---|---|---|---|---|---|---|
| | | position | position | Number of HLA class II alleles | | | Number of HLA class I alleles | | |
| | | in SEQ | in SEQ | Moderate + | | Moderate + | | | Moderate + |
| SEQ ID | | ID NO: | ID NO: | High | Moderate | High | Moderate | High | High |
| NO. | Length | 410 | 410 | affinity | affinity | affinity | affinity | affinity | affinity |
| 328 | 66 | 2580 | 2645 | 40 | 21 | 43 | 64 | 50 | 68 |
| 329 | 106 | 4891 | 4996 | 42 | 26 | 43 | 63 | 48 | 66 |
| 330 | 87 | 5238 | 5324 | 44 | 26 | 46 | 64 | 57 | 69 |
| 331 | 45 | 6407 | 6452 | 38 | 49 | 49 | 66 | 70 | 70 |
| 332 | 98 | 4704 | 4801 | 48 | 24 | 48 | 58 | 49 | 61 |
| 333 | 83 | 7757 | 7839 | 34 | 21 | 36 | 63 | 39 | 64 |
| 334 | 70 | 1532 | 1601 | 32 | 17 | 35 | 60 | 52 | 63 |
| 335 | 82 | 7948 | 8029 | 41 | 24 | 43 | 61 | 32 | 62 |
| 336 | 117 | 5531 | 5647 | 46 | 24 | 46 | 68 | 47 | 68 |
| 337 | 84 | 231 | 314 | 37 | 11 | 38 | 61 | 44 | 70 |
| 338 | 85 | 2097 | 2181 | 43 | 16 | 44 | 65 | 45 | 70 |
| 339 | 86 | 5107 | 5192 | 47 | 38 | 49 | 58 | 33 | 59 |
| 340 | 80 | 4961 | 5040 | 39 | 23 | 40 | 59 | 53 | 64 |
| 341 | 80 | 6465 | 6544 | 36 | 9 | 37 | 63 | 41 | 65 |
| 342 | 92 | 5943 | 6034 | 37 | 17 | 43 | 61 | 38 | 64 |
| 343 | 55 | 2935 | 2989 | 29 | 8 | 29 | 59 | 39 | 63 |
| 344 | 96 | 4782 | 4877 | 39 | 17 | 40 | 65 | 46 | 68 |
| 345 | 107 | 822 | 928 | 33 | 15 | 36 | 64 | 48 | 64 |
| 346 | 80 | 7406 | 7485 | 41 | 18 | 42 | 61 | 37 | 64 |
| 347 | 91 | 7287 | 7377 | 47 | 31 | 49 | 63 | 49 | 66 |
| 348 | 102 | 6622 | 6723 | 38 | 18 | 38 | 66 | 49 | 68 |
| 349 | 97 | 6800 | 6896 | 38 | 20 | 40 | 66 | 36 | 66 |
| 350 | 77 | 8568 | 8644 | 32 | 14 | 35 | 54 | 24 | 58 |
| 351 | 37 | 8683 | 8719 | 25 | 9 | 29 | 36 | 27 | 48 |
| 352 | 124 | 8818 | 8941 | 40 | 23 | 41 | 68 | 49 | 70 |
| 353 | 85 | 9039 | 9123 | 38 | 19 | 39 | 65 | 44 | 69 |
| 354 | 107 | 9552 | 9658 | 37 | 13 | 37 | 66 | 47 | 69 |
| 355 | 81 | 3149 | 3229 | 45 | 29 | 45 | 55 | 41 | 60 |
| 356 | 84 | 4057 | 4140 | 44 | 31 | 47 | 65 | 44 | 67 |
| 357 | 80 | 441 | 520 | 39 | 28 | 44 | 50 | 20 | 52 |
| 358 | 108 | 523 | 630 | 48 | 21 | 48 | 70 | 51 | 70 |
| 359 | 77 | 993 | 1069 | 36 | 12 | 37 | 59 | 41 | 62 |
| 360 | 81 | 1123 | 1203 | 41 | 13 | 43 | 64 | 41 | 70 |
| 361 | 104 | 1351 | 1454 | 45 | 24 | 47 | 64 | 38 | 67 |
| 362 | 104 | 1409 | 1512 | 41 | 26 | 44 | 61 | 35 | 63 |
| 363 | 82 | 1611 | 1692 | 41 | 14 | 42 | 59 | 47 | 69 |
| 364 | 76 | 7115 | 7190 | 39 | 17 | 39 | 63 | 26 | 63 |
| 365 | 83 | 7014 | 7096 | 46 | 21 | 46 | 63 | 38 | 67 |
| 366 | 61 | 8942 | 9002 | 37 | 23 | 43 | 44 | 11 | 46 |
| 367 | 84 | 8046 | 8129 | 38 | 19 | 38 | 60 | 35 | 62 |
| 368 | 63 | 3943 | 4005 | 35 | 27 | 40 | 52 | 26 | 56 |
| 369 | 80 | 4174 | 4253 | 46 | 27 | 47 | 64 | 17 | 64 |

In addition to the long peptide sequences SEQ ID NO: 328 to SEQ ID NO: 369, thirty-nine (39) shorter sequences with a length varying from 31 to 47 amino acid residues were also identified. The thirty-nine shorter peptide sequence correspond to portions of sequence within SEQ ID NO: 328 to SEQ ID NO: 369 with the highest number of HLA class I and class II binding motifs are shown in Table 6 (SEQ ID NOS: 370 to 408). In embodiments, these shorter peptides may be concatenated for insertion and expression from the adenoviral vector.

TABLE 6
Selected SARS-CoV-2 shorter peptide sequences containing
high density HLA class I and/or HLA class II binding
motifs (SEQ ID NO: 370 to 408)

| | Contained | | | | |
|---|---|---|---|---|---|
| SEQ | within | | | | |
| ID | SEQ ID | | N-term | C-Term | |
| No | NO: | Length | position | position | Sequence |
| 370 | 328 | 45 | 2589 | 2633 | KMFDAYVNTFSSTFNVPMEKLKTLVATAEAELAKNVSLDNVLSTF |
| 371 | 329 | 41 | 4911 | 4951 | MSYEDQDALFAYTKRNVIPTITQMNLKYAISAKNRARTVAG |
| 372 | 330 | 45 | 5243 | 5287 | DGTLMIERFVSLAIDAYPLIKHPNQEYADVEHLYLQYIRKLHDEL |
| 373 | 331 | 43 | 6410 | 6452 | RHHANEYRLYLDAYNMMISAGESLWVYKQFDTYNLWNTFTRLQ |
| 374 | 332 | 41 | 4704 | 4744 | NENVLFSTVFPPTSFGPLVRKIFVDGVPFVVSTGYHFRELG |
| 375 | 333 | 45 | 7781 | 7825 | RSVASQSIIAYTMSLGAENSVAYSNNSIAIPTNFTISVTTEILPV |
| 376 | 334 | 47 | 1532 | 1578 | DKSVYYTSNPTTFHLDGEVITFDNLKTLLSLREVRTIKVFTTVDNIN |
| 377 | 335 | 45 | 7961 | 8005 | LTDEMIAQYTSALLAGTITSGWTFGAGAALQIPFAMQMAYRFNGI |
| 378 | 336 | 45 | 5532 | 5576 | AVVYRGTTTYKLNVGDYFVLTSHTVMPLSAPTLVPQEHYVRITGL |
| 379 | 337 | 41 | 243 | 283 | ERSEKSYELQTPFEIKLAKKFDTFNGECPNFVFPLNSIIKT |
| 380 | 338 | 45 | 2097 | 2141 | AAYVDNSSLTIKKPNELSRVLGLKTLATHGLAAVNSVPWDTIANY |
| 381 | 339 | 41 | 5129 | 5169 | VDTDFVNEFYAYLRKHFSMMILSDDAVVCFNSTYASQGLVA |
| 382 | 339 | 33 | 5158 | 5190 | FNSTYASQGLVASIKNFKSVLYYQNNVFMSEAK |
| 383 | 340 | 43 | 4997 | 5039 | VYSDVENPHLMGWDYPKCDRAMPNMLRIMASLVLARKHTTCCS |
| 384 | 342 | 45 | 5972 | 6016 | KDMTYRRLISMMGFKMNYQVNGYPNMFITREEAIRHVRAWIGFDV |
| 385 | 343 | 41 | 2945 | 2985 | YESLRPDTRYVLMDGSIIQFPNTYLEGSVRVVTTFDSEYCR |
| 386 | 344 | 41 | 4817 | 4857 | SKGFFKEGSSVELKHFFFAQDGNAAISDYDYYRYNLPTMCD |
| 387 | 345 | 43 | 881 | 923 | KTLQPVSELLTPLGIDLDEWSMATYYLFDESGEFKLASHMYCS |
| 388 | 346 | 45 | 7418 | 7462 | PTESIVRFPNITNLCPFGEVFNATRFASVYAWNRKRISNCVADYS |
| 389 | 346 | 43 | 7443 | 7485 | FASVYAWNRKRISNCVADYSVLYNSASFSTFKCYGVSPTKLND |
| 390 | 347 | 45 | 7332 | 7376 | TRFQTLLALHRSYLTPGDSSSGWTAGAAAYYVGYLQPRTFLLKYN |
| 391 | 350 | 42 | 8570 | 8611 | VVLHSYFTSDYYQLYSTQLSTDTGVEHVTFFIYNKIVDEPEE |
| 392 | 351 | 31 | 8689 | 8719 | NIVNVSLVKPSFYVYSRVKNLNSSRVPDLLV |
| 393 | 352 | 43 | 8884 | 8926 | PKEITVATSRTLSYYKLGASQRVAGDSGFAAYSRYRIGNYKLN |
| 394 | 353 | 42 | 9073 | 9114 | VKHVYQLRARSVSPKLFIRQEEVQELYSPIFLIVAAIVFITL |
| 395 | 354 | 42 | 9587 | 9628 | HWPQIAQFAPSASAFFGMSRIGMEVTPSGTWLTYTGAIKLDD |
| 396 | 355 | 46 | 3149 | 3194 | STKHFYWFFSNYLKRRVVFNGVSFSTFEEAALCTFLLNKEMYLKLR |
| 397 | 355 | 46 | 3184 | 3229 | LLNKEMYLKLRSDVLLPLTQYNRYLALYNKYKYFSGAMDTTSYREA |
| 398 | 356 | 44 | 4057 | 4100 | VPLNIIPLTTAAKLMVVIPDYNTYKNTCDGTTFTYASALWEIQQ |
| 399 | 357 | 42 | 460 | 501 | NINIVGDFKLNEEIAIILASFSASTSAFVETVKGLDYKAFKQ |
| 400 | 358 | 43 | 528 | 570 | SILSPLYAFASEAARVVRSIFSRTLETAQNSVRVLQKAAITIL |
| 401 | 359 | 43 | 994 | 1036 | SEDNQTTTIQTIVEVQPQLEMELTPVVQTIEVNSFSGYLKLTD |
| 402 | 360 | 43 | 1154 | 1196 | FGADPIHSLRVCVDTVRTNVYLAVFDKNLYDKLVSSFLEMKSE |
| 403 | 361 | 43 | 1410 | 1452 | KIQEGVVDYGARFYFYTSKTTVASLINTLNDLNETLVTMPLGY |
| 404 | 363 | 41 | 1616 | 1656 | KTFYVLPNDDTLRVEAFEYYHTTDPSFLGRYMSALNHTKKW |
| 405 | 366 | 46 | 8944 | 8989 | HLVDFQVTIAEILLIIMRTFKVSIWNLDYIINLIIKNLSKSLTENK |
| 406 | 367 | 44 | 8083 | 8126 | VEAEVQIDRLITGRLQSLQTYVTQQLIRAAEIRASANLAATKMS |
| 407 | 368 | 36 | 3943 | 3978 | AIASEFSSLPSYAAFATAQEAYEQAVANGDSEVVLK |
| 408 | 369 | 41 | 4213 | 4253 | CRFVTDTPKGPKVKYLYFIKGLNNLNRGMVLGSLAATVRLQ |

For each of SEQ ID NOS: 370-408 (Table 3), the number of HLA class I and HLA class II alleles for which high or moderate binding was predicted are presented in Table 7.

TABLE 7
Number of HLA class I and HLA class II alleles for which high or moderate binding
was predicted within the selected SARS-CoV-2 shorter peptide sequences containing
high density HLA class I and/or HLA class II binding motifs (SEQ ID NOS: 370-408)

| | | N-terminal | C-terminal | | | | | | |
|---|---|---|---|---|---|---|---|---|---|
| | | position | position | Number of HLA class II alleles | | | Number of HLA class I alleles | | |
| | | in SEQ | in SEQ | | | Moderate + | | | Moderate + |
| SEQ ID | | ID NO: | ID NO: | Moderate | High | High | Moderate | High | High |
| NO: | Length | 410 | 410 | affinity | affinity | affinity | affinity | affinity | affinity |
| 370 | 45 | 2589 | 2633 | 27 | 14 | 32 | 59 | 50 | 67 |
| 371 | 41 | 4911 | 4951 | 31 | 13 | 32 | 57 | 30 | 65 |
| 372 | 45 | 5243 | 5287 | 37 | 15 | 40 | 61 | 43 | 65 |
| 373 | 43 | 6410 | 6452 | 35 | 15 | 39 | 63 | 37 | 68 |
| 374 | 41 | 4704 | 4744 | 32 | 14 | 35 | 48 | 34 | 55 |
| 375 | 45 | 7781 | 7825 | 31 | 15 | 33 | 54 | 35 | 55 |
| 376 | 47 | 1532 | 1578 | 31 | 16 | 34 | 56 | 41 | 60 |
| 377 | 45 | 7961 | 8005 | 35 | 19 | 37 | 53 | 30 | 55 |
| 378 | 45 | 5532 | 5576 | 38 | 18 | 39 | 59 | 40 | 64 |
| 379 | 41 | 243 | 283 | 28 | 5 | 30 | 47 | 36 | 60 |
| 380 | 45 | 2097 | 2141 | 34 | 10 | 35 | 63 | 36 | 68 |
| 381 | 41 | 5129 | 5169 | 45 | 32 | 49 | 44 | 21 | 47 |
| 382 | 33 | 5158 | 5190 | 33 | 14 | 37 | 37 | 13 | 41 |
| 383 | 43 | 4997 | 5039 | 32 | 15 | 35 | 45 | 37 | 57 |
| 384 | 45 | 5972 | 6016 | 32 | 17 | 38 | 50 | 31 | 56 |
| 385 | 41 | 2945 | 2985 | 24 | 6 | 24 | 56 | 37 | 61 |
| 386 | 41 | 4817 | 4857 | 23 | 6 | 23 | 52 | 32 | 60 |
| 387 | 43 | 881 | 923 | 25 | 7 | 26 | 47 | 36 | 58 |
| 388 | 45 | 7418 | 7462 | 33 | 15 | 35 | 51 | 28 | 58 |
| 389 | 43 | 7443 | 7485 | 31 | 9 | 34 | 33 | 14 | 34 |
| 390 | 45 | 7332 | 7376 | 39 | 24 | 41 | 48 | 37 | 56 |
| 391 | 42 | 8570 | 8611 | 28 | 12 | 31 | 41 | 19 | 47 |
| 392 | 31 | 8689 | 8719 | 25 | 9 | 29 | 36 | 27 | 48 |
| 393 | 43 | 8884 | 8926 | 34 | 16 | 35 | 53 | 24 | 53 |
| 394 | 42 | 9073 | 9114 | 27 | 16 | 31 | 58 | 32 | 67 |
| 395 | 42 | 9587 | 9628 | 29 | 11 | 30 | 54 | 28 | 61 |
| 396 | 46 | 3149 | 3194 | 36 | 24 | 41 | 45 | 23 | 52 |
| 397 | 46 | 3184 | 3229 | 34 | 13 | 35 | 46 | 27 | 53 |
| 398 | 44 | 4057 | 4100 | 34 | 16 | 40 | 43 | 32 | 47 |
| 399 | 42 | 460 | 501 | 33 | 26 | 40 | 46 | 13 | 48 |
| 400 | 43 | 528 | 570 | 42 | 16 | 42 | 62 | 18 | 63 |
| 401 | 43 | 994 | 1036 | 20 | 6 | 23 | 41 | 23 | 47 |
| 402 | 43 | 1154 | 1196 | 33 | 8 | 34 | 56 | 29 | 60 |
| 403 | 43 | 1410 | 1452 | 30 | 16 | 34 | 50 | 28 | 52 |
| 404 | 41 | 1616 | 1656 | 32 | 10 | 35 | 45 | 36 | 58 |
| 405 | 46 | 8944 | 8989 | 36 | 22 | 43 | 42 | 11 | 44 |
| 406 | 44 | 8083 | 8126 | 32 | 14 | 34 | 49 | 18 | 52 |
| 407 | 36 | 3943 | 3978 | 27 | 19 | 30 | 40 | 18 | 45 |
| 408 | 41 | 4213 | 4253 | 36 | 20 | 41 | 43 | 8 | 44 |

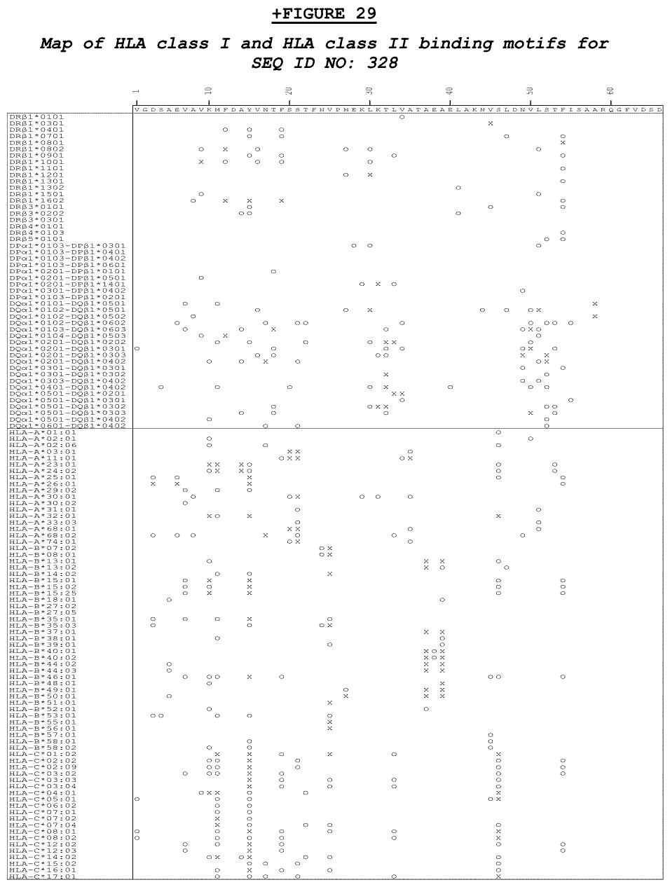

For each selected sequence from SEQ ID NO: 328 to SEQ ID NO: 369, a map of HLA class I and HLA class II binding motif are presented in FIGS. 29-70. In FIGS. 29-70, the N-terminal amino acid (i.e., amino acid #1) for each HLA class I and II motif therein is identified by an X or an O, wherein X further indicates a high affinity motif and O indicates moderate affinity for the HLA binding motif. Exemplary HLA binding motifs can be deduced from FIGS. 29 to 70 by including the X or O amino acid residue and including the subsequent eight to ten amino acids in the motif such that each motif includes nine to eleven amino acid residues (i.e., each motif is a 9-11 amino acid peptide), with reference to the SEQ ID number indicated in each figure. Any such binding motifs can be used as immunogens, alone and/or in combination, in the vectors disclosed herein. Other binding motifs of any of SEQ ID NOS. 328-369 may also be suitable for inclusion in the SARS-CoV-2 immunization vectors herein as would be understood by those of ordinary skill in the art.

RBD Sequences Matching Circulating SARS-CoV-2 Variants

As described above, rdAd vectors may be designed to encode sequences homologous to SEQ ID NO: 13, SEQ ID NO: 14, SEQ ID NO: 15 or any sequences comprising SEQ ID NO: 446 with or without additional flanking residues and incorporating single or multiple RBD mutation(s) as those described in FIG. 17B. Preferably, the RBD variant sequences may include single or combined mutations at amino acid positions 367, 403, 439, 417, 446, 447, 449, 452, 453, 455, 456, 470, 473, 475, 476, 477, 478, 484, 486, 487, 490, 493, 494, 496, 499, 500, 501, 502, 503, 504, and/or 505, wherein the amino acid position numbering corresponds to SEQ ID NO: 3 (full length spike protein) (FIG. 17B). More preferably, the RBD sequences include single or combined mutations at position K417T, K417N, E484K, L452R and/or N501Y (FIG. 17B). In preferred embodiments, the RBD variant protein sequences can be selected from the group consisting of SEQ ID NOS: 412-417 wherein the RBD sequence is underlined:

Spike RBD from SARS-CoV California CAL.20C B.1.429
lineage:
(SEQ ID NO: 412)
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNY
RYRLFRKSNLKPFERDISTEIYQAGSTPCNGVEGFNCYFPLQSYGFQPTN
GVGYQPYRVVVLSFELLHAPATVCGPKKSTNLVKNKCVFNFN
Spike RBD from SARS-CoV Brazil P.2 lineage
B.1.1.28.2 lineage:
(SEQ ID NO: 413)
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADFVI
RGDEVRQIAPGQTGKIADYNYKLPDDFTGCVIAWNSNNLDSKVGGNYNYL
YRLFRKSNLKPFERDISTEIYQAGSTPCNGVKGFNCYFPLQSYGFQPTNG
VGYQPYRVVVLSFELLHAPATVCGPKKSTNLVKNKCVFNFN
Spike RBD from SARS-CoV UK VOC 202012/01; B.1.1.7
lineage (a.k.a. 20I/501Y.V1):
(SEQ ID NO: 414)
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPTY
GVGYQPYRVVVLSFELLHAPATVCGPKKSTNLVKNKCVFNFN
Spike RBD from SARS-CoV UK B.1.1.7 lineage (E484K):
(SEQ ID NO: 415)
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNY
LYRLFRKSNLKPFERDISTEIYQAGSTPCNGVKGFNCYFPLQSYGFQPTY
GVGYQPYRVVVLSFELLHAPATVCGPKKSTNLVKNKCVFNFN
Spike RBD from SARS-CoV Brazil P.1 lineage
B.1.1.28.1 lineage (a.k.a. 20J/501Y.V3):
(SEQ ID NO: 416)
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGTIADYNYKLPDDFTGCVIAWNSNNLDSKVGGNYNY
LYRLFRKSNLKPFERDISTEIYQAGSTPCNGVKGFNCYFPLQSYGFQPTY
GVGYQPYRVVVLSFELLHAPATVCGPKKSTNLVKNKCVFNFN
South Africa 501Y.V2 B.1.351 lineage (a.k.a.
20H/501Y.V2):
(SEQ ID NO: 417)
TLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFV
IRGDEVRQIAPGQTGNIADYNYKLPDDFTGCVIAWNSNNLDSKVGGNYNY
LYRLFRKSNLKPFERDISTEIYQAGSTPCNGVKGFNCYFPLQSYGFQPTY
GVGYQPYRVVVLSFELLHAPATVCGPKKSTNLVKNKCVFNFN

In preferred embodiments, the RBD sequences to be expressed by the rdAd (a monovalent RBD vector) will be preceded by a leader/signal peptide sequence to address the expression of the polypeptide the cellular secretory pathway. Commonly used leader peptide sequences for efficient targeting of a recombinant protein expressed in mammalian cells are described in Table 9.

TABLE 9

| Leader sequence name | Sequence |
|---|---|
| Human OSM | MGVLLTQRTLLSLVLALLFPSMASM (SEQ ID NO: |
| | 418) |
| VSV-G | MKCLLYLAFLFIGVNC (SEQ ID NO: 419) |
| Mouse Ig Kappa | METDTLLLWVLLLWVPGSTGD (SEQ ID NO: 420) |
| Human IgG2 H | MGWSCIILFLVATATGVHS (SEQ ID NO: 421) |
| BM40 | MRAWIFFLLCLAGRALA (SEQ ID NO: 422) |
| Secrecon | MWWRLWWLLLLLLLLWPMVWA (SEQ ID NO: 423) |
| Human IgK VIII | MDMRVPAQLLGLLLLWLRGARC (SEQ ID NO: |
| | 424) |
| CD33 | MPLLLLLPLLWAGALA (SEQ ID NO: 425) |
| tPA | MDAMKRGLCCVLLLCGAVFVSPS (SEQ ID NO: |
| | 426) |
| | MDAMKRGLCCVLLLCGAVFVSPSGTGS (SEQ ID |
| | NO: 427) |
| Human Chymotrypsinogen | MAFLWLLSCWALLGTTFG (SEQ ID NO: 428) |
| Human trypsinogen-2 | MNLLLILTFVAAAVA (SEQ ID NO: 429) |
| Human IL-2 | MYRMQLLSCIALSLALVTNS (SEQ ID NO: 430) |
| Gaussia luciferase | MGVKVLFALICIAVAEA (SEQ ID NO: 431) |
| Albumin (HSA) | MKWVTFISLLFSSAYS (SEQ ID NO: 432) |
| Influenza Haemagglutinin | MKTIIALSYIFCLVLG (SEQ ID NO: 433) |
| Human insulin | MALWMRLLPLLALLALWGPDPAAA (SEQ ID NO: |
| | 434) |
| Silkworm Fibroin LC | MKPIFLVLLVVTSAYA (SEQ ID NO: 435) |
| adenovirus protein | MRYMILGLLALAAVCSAA (SEQ ID NO: 436) |
| E3/gp19K | |
| IgG | MKHLWFFLLLVAAPRWVLS (SEQ ID NO: 437) |

Variant RBD Sequences and Multivalent Vaccine Compositions

It may be also advantageous to develop multivalent immunogenic compositions or vaccines providing immunity against co-circulating SARS-CoV-2 variants and potentially future variants. In some embodiments, multivalent genetic immunogenic compositions or vaccine compositions can be achieved by combining several monovalent genetic immunogenic compositions or vaccine compositions, each expressing an antigen sequence variant in the same preparation. In some embodiments, a single genetic construct can also be constructed to co-express multiple antigen sequence variants to provide a multivalent RBD SARS-CoV-2 immunogenic compositions or vaccine composition. In some embodiments, for instance, an rdAd vector of this disclosure can comprise one or more expression cassettes comprising a SARS-CoV-2 antigen coding sequence that incorporates one or more mutations in the spike protein RBD region, and that would be able to protect against circulating SARS-CoV-2 variants and potentially future variants. In some embodiments, a single genetic construct may comprise multiple RBD variant units arranged co-linearly within a single expression cassette. RBD variant units may be selected from SEQ ID NO: 13, SEQ ID NO: 14, SEQ ID NO: 15 or any sequences comprising SEQ ID NO: 446 with or without additional flanking residues and incorporating single or multiple RBD mutation(s) as those described in FIG. 17B. Each RBD variant unit can be spaced from other RBD variant in the co-linear arrangement by a flexible spacer sequence comprising, for instance, glycine and serine amino acid residues. In preferred embodiments, the spacer peptide can comprise the amino acid sequence (GGGGS)n, (GGGGA)n, (GGGGQ)n, (GGGPS)n, (GPGPG)n and combinations thereof, where n is comprised between 1 and 10. Other spacers, preferably hydrophilic in nature and comprising polar amino acids may also be suitable as would be understood by those ordinary skill in the art. In some embodiments, spacers can include one or more proteolytic cleavage motifs for proprotein convertases (PCs), also known as eukaryotic subtilases, represented by a group of serine proteases comprising furin (PACE), PC1 (PC3), PC2, PC4, PACE4, PC5 (PC6), and PC7 (LPC, PC8) that generate bioactive proteins and peptides, such as hormones, receptors, and growth factors by cleaving precursor proteins at multi-basic motifs. In preferred embodiments, such multi-basic motifs include the one described in table below:

TABLE 8

| Proprotein convertase names | Cleavage site specificity |
|---|---|
| PC1/3 | KR↓ or RR↓ |
| PC2 | KR↓ or RR↓ |
| Furin; PACE | RXK/RR↓ |
| | RXXR↓ |
| | RXRXXXR/KR↓ |
| PC4 | KXXR↓ |
| | RXK/RR↓ |
| PC5/6A; PC5/6B | RXK/RR↓ |
| PACE4 | RXK/RR↓ |
| PC7; PC8; LPC; SPC7 | RXK/RR↓ |

X = any natural amino-acid;
↓ = Cleavage position

Spacer sequences are preferably optimized to avoid the introduction of neo-epitopes within the pseudo-protein sequence. Moreover, different spacers can be combined in the same sequence. In some embodiments, the spacers may include a flexible portion and a cleavable portion. In some embodiments, different spacer sequences may be used between RBD variant units within the same multivalent construct. In a preferred multivalent RBD vector, the number of RBD variant units arranged colinearly in the same genetic sequence may vary from 2 to 10 units, preferably 2 to 6 units. The respective order of the RBD variant sequence may also vary; the use of spacer being introduced so that each RBD variant unit are immunogenically-independent. Each RBD variant unit can include any single mutation or combination of mutations, as those described in FIG. 17B.

Preferably, the RBD variant sequences may include single or combined mutations at positions 367, 403, 439, 417, 446, 447, 449, 452, 453, 455, 456, 470, 473, 475, 476, 477, 478, 484, 486, 487, 490, 493, 494, 496, 499, 500, 501, 502, 503, 504, and/or 505 (FIG. 17B). More preferably, the RBD sequences include single or combined mutations at position K417T, K417N, E484K, L452R and/or N501Y, wherein the amino acid position numbering corresponds to SEQ ID NO: 3 (full length spike protein) (FIG. 17B). SEQ ID NOS: 438 to 443 and 460 are provided as preferred embodiments of RBD variant sequence that can be used for the design of the multivalent RBD vector. Preferred examples of multivalent RBD sequences comprising multiple RBD variant units are presented as SEQ ID NO: 444, SEQ ID NO: 445, SEQ ID NO: 475 and SEQ ID NO: 476. In preferred embodiments, the multivalent RBD sequences to be expressed by the rdAd (a “multivalent RBD vector”, a type of multivalent hAd5-SARS-CoV-2) will be preceded by a leader/signal peptide sequence to address the expression of the polypeptide the cellular secretory pathway. Commonly used leader peptide sequences for efficient targeting of a recombinant protein expressed in mammalian cells are described in Table 9.

In preferred embodiments, the RBD variant protein sequences can be selected from the group consisting of SEQ ID NOS: 438-443 and 460, and exemplary multivalent RBD constructs are shown in SEQ ID NOS: 444, 445, 475 and 476 below, wherein the RBD sequence is underlined:

Spike RBD from SARS-CoV (GenBank: MN908947.3):
(SEQ ID NO: 438)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVCGPKKS
TNLVKNKSVNFTF (CVFNFN substituted by SVNFT)
Spike RBD from SARS-CoV California CAL.20C B.1.429 lineage:
(SEQ ID NO: 439)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYRYRLFRKSNLKPFERDISTEIYQ
AGSTPCNGVEGFNCYFPLQSYGFQPTNGVGYQPYRVVVLSFELLHAPATVCGPKKS
TNLVKNKSVNFTF (CVFNFN substituted by SVNFT)
Spike RBD from SARS-CoV Brazil P.2 lineage B.1.1.28.2 lineage:
(SEQ ID NO: 440)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFERDISTEIYQ
AGSTPCNGVKGFNCYFPLQSYGFQPTNGVGYQPYRVVVLSFELLHAPATVCGPKKS
TNLVKNKSVNFTF (CVFNFN substituted by SVNFT)
Spike RBD from SARS-CoV UK VOC 202012/01; B.1.1.7 lineage
(a.k.a. 20I/501Y.V1):
(SEQ ID NO: 460)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFERDISTEIYQ
AGSTPCNGVEGFNCYFPLQSYGFQPTYGVGYQPYRVVVLSFELLHAPATVCGPKKS
TNLVKNKSVNFTF (CVFNFN substituted by SVNFT)
Spike RBD from SARS-CoV UK B.1.1.7 lineage (E484K):
(SEQ ID NO: 441)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFERDISTEIYQ
AGSTPCNGVKGFNCYFPLQSYGFQPTYGVGYQPYRVVVLSFELLHAPATVCGPKKS
TNLVKNKSVNFTF (CVFNFN substituted by SVNFT)
Spike RBD from SARS-CoV Brazil P.1 lineage B.1.1.28.1
lineage (a.k.a. 20J/501Y.V3):
(SEQ ID NO: 442)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGTI
ADYNYKLPDDFTGCVIAWNSNNLDSKVGGNYNYLYRLFRKSNLKPFERDISTEIYQ
AGSTPCNGVKGFNCYFPLQSYGFQPTYGVGYQPYRVVVLSFELLHAPATVCGPKKS
TNLVKNKSVNFTF (CVFNFN substituted by SVNFT)
South Africa 501Y.V2 B.1.351 lineage (a.k.a. 20H/501Y.V2):
(SEQ ID NO: 443)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGN
IADYNYKLPDDFTGCVIAWNSNNLDSKVGGNYNYLYRLFRKSNLKPFERDISTEIYQ
AGSTPCNGVKGFNCYFPLQSYGFQPTYGVGYQPYRVVVLSFELLHAPATVCGPKKS
TNLVKNKSVNFTF (CVFNFN substituted by SVNFT)
Multivalent RBD construct combination of comprising
SEQ ID NO: 438, SEQ ID NO: 443 and SEQ ID NO: 441:
(SEQ ID NO: 444)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVCGPKKS
TNLVKNKSVNFTFGGGGSGGGGSGGGGSTLKSFTVEKGIYQTSNFRVQPTESIVRFP
NITNLCPFGEVFNATRFASVYAWNRKRISNCVADYSVLYNSASFSTFKCYGVSPTKL
NDLCFTNVYADSFVIRGDEVRQIAPGQTGNIADYNYKLPDDFTGCVIAWNSNNLDS
KVGGNYNYLYRLFRKSNLKPFERDISTEIYQAGSTPCNGVKGFNCYFPLQSYGFQPT
YGVGYQPYRVVVLSFELLHAPATVCGPKKSTNLVKNKSVNFTFGGGGSGGGGSGG
GGSTLKSFTVEKGIYQTSNFRVQPTESIVRFPNITNLCPFGEVFNATRFASVYAWNRK
RISNCVADYSVLYNSASFSTFKCYGVSPTKLNDLCFTNVYADSFVIRGDEVRQIAPGQ
TGKIADYNYKLPDDFTGCVIAWNSNNLDSKVGGNYNYLYRLFRKSNLKPFERDISTE
IYQAGSTPCNGVKGFNCYFPLQSYGFQPTYGVGYQPYRVVVLSFELLHAPATVCGP
KKSTNLVKNKSVNFTF (Spacer (GGGGS)₃)
Multivalent RBD construct combination of comprising
SEQ ID NO: 438, SEQ ID NO: 443 and SEQ ID NO: 441:
(SEQ ID NO: 445)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFERDISTEIYQ
AGSTPCNGVEGFNCYFPLQSYGFQPTNGVGYQPYRVVVLSFELLHAPATVCGPKKS
TNLVKNKSVNFTFGGGGSGGGGSRRKRSVGGGGSGGGGSTLKSFTVEKGIYQTSNF
RVQPTESIVRFPNITNLCPFGEVFNATRFASVYAWNRKRISNCVADYSVLYNSASFST
FKCYGVSPTKLNDLCFTNVYADSFVIRGDEVRQIAPGQTGNIADYNYKLPDDFTGCV
IAWNSNNLDSKVGGNYNYLYRLFRKSNLKPFERDISTEIYQAGSTPCNGVKGFNCYF
PLQSYGFQPTYGVGYQPYRVVVLSFELLHAPATVCGPKKSTNLVKNKSVNFTFGGG
GSGGGGSRRKRSVGGGGSGGGGSTLKSFTVEKGIYQTSNFRVQPTESIVRFPNITNLC
PFGEVFNATRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLDSKVGGNY
NYLYRLFRKSNLKPFERDISTEIYQAGSTPCNGVKGFNCYFPLQSYGFQPTYGVGYQ
PYRVVVLSFELLHAPATVCGPKKSTNLVKNKSVNFTF (Spacer
(GGGGS)2RRKRSV(GGGGS)₂)
Multivalent RBD construct combination of comprising
SEQ ID NO: 438, SEQ ID NO: 439, SEQ ID NO: 440,
SEQ ID NO: 441, SEQ ID NO: 442 and SEQ ID NO: 443
(SEQ ID NO: 475)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYRYRLFRKSNLKPFERDISTEIYQ
AGSTPCNGVEGFNCYFPLQSYGFQPTNGVGYQPYRVVVLSFELLHAPATVCGPKKS
TNLVKNKSVNFTFGGGGSGGGGSGGGGSTLKSFTVEKGIYQTSNFRVQPTESIVRFP
NITNLCPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDS
KVGGNYNYRYRLFRKSNLKPFERDISTEIYQAGSTPCNGVEGFNCYFPLQSYGFQPT
NGVGYQPYRVVVLSFELLHAPATVCGPKKSTNLVKNKSVNFTFGGGGSGGGGSGG
GGSTLKSFTVEKGIYQTSNFRVQPTESIVRFPNITNLCPFGEVFNATRFASVYAWNRK
RISNCVADYSVLYNSASFSTFKCYGVSPTKLNDLCFTNVYADSFVIRGDEVRQIAPGQ
TGKIADYNYKLPDDFTGCVIAWNSNNLDSKVGGNYNYLYRLFRKSNLKPFERDISTE
IYQAGSTPCNGVKGFNCYFPLQSYGFQPTNGVGYQPYRVVVLSFELLHAPATVCGP
KKSTNLVKNKSVNFTFGGGGSGGGGSGGGGSTLKSFTVEKGIYQTSNFRVQPTESIV
RFPNITNLCPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNNL
DSKVGGNYNYLYRLFRKSNLKPFERDISTEIYQAGSTPCNGVKGFNCYFPLQSYGFQ
PTYGVGYQPYRVVVLSFELLHAPATVCGPKKSTNLVKNKSVNFTFGGGGSGGGGSG
GGGSTLKSFTVEKGIYQTSNFRVQPTESIVRFPNITNLCPFGEVFNATRFASVYAWNR
KRISNCVADYSVLYNSASFSTFKCYGVSPTKLNDLCFTNVYADSFVIRGDEVRQIAPG
QTGTIADYNYKLPDDFTGCVIAWNSNNLDSKVGGNYNYLYRLFRKSNLKPFERDIST
EIYQAGSTPCNGVKGFNCYFPLQSYGFQPTYGVGYQPYRVVVLSFELLHAPATVCGP
KKSTNLVKNKSVNFTFGGGGSGGGGSGGGGSTLKSFTVEKGIYQTSNFRVQPTESIV
RFPNITNLCPFGEVFNATRFASVYAWNRKRISNCVADYSVLYNSASFSTFKCYGVSP
TKLNDLCFTNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYFPLQSYGFQ
PTYGVGYQPYRVVVLSFELLHAPATVCGPKKSTNLVKNKSVNFTF (Spacer
(GGGGS)₂RRKRSV(GGGGS)₂)
Multivalent RBD construct combination of comprising
SEQ ID NO: 438, SEQ ID NO: 439, SEQ ID NO: 440,
SEQ ID NO: 441, SEQ ID NO: 442 and SEQ ID NO: 443
(SEQ ID NO: 476)
TLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGK
IADYNYKLPDDFTGCVIAWNSNNLDSKVGGNYNYRYRLFRKSNLKPFERDISTEIYQ
AGSTPCNGVEGFNCYFPLQSYGFQPTNGVGYQPYRVVVLSFELLHAPATVCGPKKS
TNLVKNKSVNFTFGSGGGGSRRKRSVGGGGSGGGGSTLKSFTVEKGIYQTSNFRVQ
PTESIVRFPNITNLCPFGEVFNATRFASVYAWNRKRISNCVADYSVLYNSASFSTFKC
YGVSPTKLNDLCFTNVYADSFVIRGDEVRQIAPGQTGKIADYNYKLPDDFTGCVIAW
NSNNLDSKVGGNYNYRYRLFRKSNLKPFERDISTEIYQAGSTPCNGVEGFNCYFPLQ
SYGFQPTNGVGYQPYRVVVLSFELLHAPATVCGPKKSTNLVKNKSVNFTFGSGGGG
SRRKRSVGGGGSGGGGSTLKSFTVEKGIYQTSNFRVQPTESIVRFPNITNLCPFGEVF
NATRFASVYAWNRKRISNCVADYSVLYNSASFSTFKCYGVSPTKLNDLCFTNVYAD
SFVIRGDEVRQIAPGQTGKIADYNYKLPDDFTGCVIAWNSNNLDSKVGGNYNYLYR
LFRKSNLKPFERDISTEIYQAGSTPCNGVKGFNCYFPLQSYGFQPTNGVGYQPYRVV
VLSFELLHAPATVCGPKKSTNLVKNKSVNFTFGSGGGGSRRKRSVGGGGSGGGGST
LKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKI
ADYNYKLPDDFTGCVIAWNSNNLDSKVGGNYNYLYRLFRKSNLKPFERDISTEIYQ
AGSTPCNGVKGFNCYFPLQSYGFQPTYGVGYQPYRVVVLSFELLHAPATVCGPKKS
TNLVKNKSVNFTFGSGGGGSRRKRSVGGGGSGGGGSTLKSFTVEKGIYQTSNFRVQ
PTESIVRFPNITNLCPFGEVFNATRFASVYAWNRKRISNCVADYSVLYNSASFSTFKC
YGVSPTKLNDLCFTNVYADSFVIRGDEVRQIAPGQTGTIADYNYKLPDDFTGCVIAW
NSNNLDSKVGGNYNYLYRLFRKSNLKPFERDISTEIYQAGSTPCNGVKGFNCYFPLQ
SYGFQPTYGVGYQPYRVVVLSFELLHAPATVCGPKKSTNLVKNKSVNFTFGSGGGG
SRRKRSVGGGGSGGGGSTLKSFTVEKGIYQTSNFRVQPTESIVRFPNITNLCPFGEVF
NATRFASVYAWNRKRISNCVADYSVLYNSASFSTFKCYGVSPTKLNDLCFTNVYAD
SFVIRGDEVRQIAPGQTGNIADYNYKLPDDFTGCVIAWNSNNLDSKVGGNYNYLYR
LFRKSNLKPFERDISTEIYQAGSTPCNGVKGFNCYFPLQSYGFQPTYGVGYQPYRVV
VLSFELLHAPATVCGPKKSTNLVKNKSVNFTF (Spacer
(GGGGS)₂RRKRSV(GGGGS)₂)

Other peptides, polypeptides, constructs, and combinations thereof are also contemplated herein as would be understood by those of ordinary skill in the art.

Receptor Binding Antagonists

In some embodiments, the SARS-CoV-2 immunization vectors (i.e., those expressing one or more exogenous antigens) can comprise polynucleotides encoding one or SARS-CoV-2 blocking proteins (e.g., receptor binding antagonists). For instance, coronaviruses such as SARS-CoV-2 are known to use homotrimers of the spike (S) protein for host cell attachment, fusion and entry into the host cell, and can involve sialic acids and/or ACE2 (a cell membrane C-terminal anchored protein that catalyzes the cleavage of angiotensin 1 into angiotensin 1-9, and of angiotensin II into the vasodilator angiotensin 1-7, thus playing a key role in systemic blood pressure regulation (Alifano, et al. Renin-angiotensin system at the heart of COVID-19 pandemic. Biochimie, 174: 30-33 (2020)). Host cell proteases are known to process coronavirus S protein to generate two subunits (S1 and S2), which remain non-covalently bound in the pre-fusion conformation of the virus (see, e.g., Tortorici, et al. Structural basis for human coronavirus attachment to sialic acid receptors. Nat. Struc. Mol. Biol. 26: 481-489 (2019)). The S1 subunit comprises four domains NTD, RBD, SD1 and SD1 with NTD and RBD separated by a linker sequence as presented in FIG. 13. In some embodiments, SARS-CoV-2 immunization vector can also or alternatively comprise a polynucleotide causing expression in a cell of at least one or both of the S1 domains (NTD and/or RBD) or as part of the full-length S protein (with or without transmembrane domain), and/or an immunogenic fragment of the same that, in some preferred embodiments, induces an immune response that interferes with the binding of the S protein (e.g., an antibody against the S1 RDB and/or NTD domain(s)) to the above-mentioned sialic acids and/or ACE2, thereby interfering with entry of SARS-CoV-2 into a host cell and/or its effect on ACE2. In some embodiments, the SARS-CoV-2 immunization vector can also or alternatively comprise a polynucleotide causing expression in a cell of a protein (e.g., an antibody or fragment thereof, or a peptide (e.g., an ACE2 mimic, RBD binding peptide)) that can bind to S protein (e.g., the S1 RDB and/or NTD domain(s) thereof, and/or S2), thereby interfering with entry of SARS-CoV-2 into a host cell (e.g., blocking the binding of the S protein to its host cells receptors such as ACE2) and/or its effect on ACE2 (collectively referred to as “RBD binding agents”). In some embodiments, the RBD binding agent can be a peptide with at least one domain corresponding to a virus-binding domain of ACE2, e.g., a peptide the conformationally matches the RBD. In some embodiments, the SARS-CoV-2 immunization vector can also or alternatively comprise a polynucleotide causing expression in a cell of a protein that can bind to ACE2 (e.g., an ACE2 binding agent) and/or otherwise prevent attachment of the S protein (e.g., the S1 RDB and/or NTD domain(s) thereof, and/or S2, can be expressed as free proteins) thereto, thereby interfering with entry of SARS-CoV-2 into a host cell and/or its effect on ACE2, preferably without interfering with the normal physiological function of ACE2 (i.e., other than its ability to serve as a receptor for SARS-CoV-2). In some embodiments, such an ACE2 binding agent can be a peptide, such as a peptide that interferes with the binding of SARS-CoV-2 to ACE2. In some embodiments, the SARS-CoV-2 immunization vector can comprise one or more polynucleotides encoding both a RBD binding agent and an ACE2 binding agent, and/or a dual RBD binding and ACE2 binding agent. In preferred embodiments, the RBD binding agent(s) and/or ACE2 binding agent(s) interfere with the interaction of ACE2 and RBD at one or more of the 15 residues from ACE2 (24(Q), 27(T), 30(D), 31(K), 34(H), 35(E), 37(E), 38(D), 41(Y), and 42(Q) are in α1, one residue (residue 82 M) comes from α2, residues 353(K), 354(G), 355(D), and 357(R) come from the linker between (33 and (34) that are currently understood to interact with RBD (Han, et al. Computational Design of ACE2-Based Peptide Inhibitors of SARS-CoV-2. ACS Nano 2020, Publication Date: Apr. 14, 2020 (https://doi.org/10.1021/acsnano.0c02857); Yan, et al. Structural Basis for the Recognition of the SARS-CoV-2 by Full-Length Human ACE2. Science 367: 1444-1448 (2020)); preferably without affecting the normal physiological function (i.e., other than its ability to serve as a receptor for SARS-CoV-2) of the ACE2 protein. Thus, in some embodiments, the SARS-CoV-2 immunization vectors of this disclosure can comprise one or more polynucleotides that encode secreted antigens (e.g., S1, NTD, RBD, ACE2 binding agent) that can bind to and block the ACE2 receptor adding additional activity in addition to immunity.

Viral Vectored Adjuvants

In some embodiments, the SARS-CoV-2 immunization vector(s) can also or alternatively comprise at least one polynucleotide encoding a polypeptide and/or peptide that improves or enhances the immunogenicity of the vector(s) (e.g., acts as an adjuvant (e.g., a molecular adjuvant)) that is expressed by a host cells and assists in the induction of an anti-SARS-CoV-2 immune response, and/or enhances an ongoing anti-SARS-CoV-2 immune response, resulting from administration of the vector(s) to a host (e.g., in preferred embodiments without inducing a systemic inflammatory response that could interfere with the recovery of a patient from SARS-CoV-2 infection). For instance, in some embodiments, the SARS-CoV-2 immunization vector(s) can encode one or more: 1) polypeptides or peptides that function as “co-stimulatory” component(s) such as, for instance, polypeptides or peptides that bind members of the CD28 family (i.e., CD28, ICOS; Hutloff, et al. Nature 1999, 397: 263-265; Peach, et al. J Exp Med 1994, 180: 2049-2058) such as the CD28 binding polypeptides B7.1 (CD80; Schwartz, 1992; Chen et al, 1992; Ellis, et al. J. Immunol., 156(8): 2700-9) and B7.2 (CD86; Ellis, et al. J. Immunol., 156(8): 2700-9); members of the integrin family (i.e., LFA-1 (CD11a/CD18); Sedwick, et al. J Immunol 1999, 162: 1367-1375; Wüllfing, et al. Science 1998, 282: 2266-2269; Lub, et al. Immunol Today 1995, 16: 479-483) including members of the ICAM family (i.e., ICAM-1, -2 or -3); CD2 family members (i.e., CD2, signalling lymphocyte activation molecule (CDw150 or “SLAM”; Aversa, et al. J Immunol 1997, 158: 4036-4044) such as CD58 (LFA-3; CD2 ligand; Davis, et al. Immunol Today 1996, 17: 177-187) or SLAM ligands (Sayos, et al. Nature 1998, 395: 462-469); heat stable antigen (HSA or CD24; Zhou, et al. Eur J Immunol 1997, 27: 2524-2528); members of the TNF receptor (TNFR) family (i.e., 4-1BB (CD137; Vinay, et al. Semin Immunol 1998, 10: 481-489)), OX40 (CD134; Weinberg, et al. Semin Immunol 1998, 10: 471-480; Higgins, et al. J Immunol 1999, 162: 486-493), and CD27 (Lens, et al. Semin Immunol 1998, 10: 491-499)) such as 4-1BBL (4-1BB ligand; Vinay, et al. Semin Immunol 1998, 10: 481-48; DeBenedette, et al. J Immunol 1997, 158: 551-559), TNFR associated factor-1 (TRAF-1; 4-1BB ligand; Saoulli, et al. J Exp Med 1998, 187: 1849-1862, Arch, et al. Mol Cell Biol 1998, 18: 558-565), TRAF-2 (4-1BB and OX40 ligand; Saoulli, et al. J Exp Med 1998, 187: 1849-1862; Oshima, et al. Int Immunol 1998, 10: 517-526, Kawamata, et al. J Biol Chem 1998, 273: 5808-5814), TRAF-3 (4-1BB and OX40 ligand; Arch, et al. Mol Cell Biol 1998, 18: 558-565; Jang, et al. Biochem Biophys Res Commun 1998, 242: 613-620; Kawamata S, et al. J Biol Chem 1998, 273: 5808-5814), OX40L (OX40 ligand; Gramaglia, et al. J Immunol 1998, 161: 6510-6517), TRAF-5 (OX40 ligand; Arch, et al. Mol Cell Biol 1998, 18: 558-565; Kawamata, et al. J Biol Chem 1998, 273: 5808-5814), and CD70 (CD27 ligand; Couderc, et al. Cancer Gene Ther., 5(3): 163-75). CD154 (CD40 ligand or “CD40L”; Gurunathan, et al. J. Immunol., 1998, 161: 4563-4571; Sine, et al. Hum. Gene Ther., 2001, 12: 1091-1102); 2) one or more cytokines (e.g., as described for retroviruses in Ohs, et al. Interleukin-Encoding Adenoviral Vectors as Genetic Adjuvant for Vaccination against Retroviral Infection. PLos One, 8(12): e82528 (December 2013)), such as interleukin-2 (IL-2) (Rosenberg, et al. Nature Med. 4: 321-327 (1998)), IL-4, IL-5, IL-6 IL-7, IL-12 (reviewed by Pardoll, 1992; Harries, et al. J. Gene Med. 2000 July-August; 2(4):243-9; Rao, et al. J. Immunol. 156: 3357-3365 (1996)), IL-15 (Xin, et al. Vaccine, 17:858-866, 1999), IL-16 (Cruikshank, et al. J. Leuk Biol. 67(6): 757-66, 2000), IL-18 (J. Cancer Res. Clin. Oncol. 2001. 127(12): 718-726), GM-CSF (CSF (Disis, et al. Blood, 88: 202-210 (1996)), IL-23, tumor necrosis factor-alpha (TNF-α), or an interferon (e.g., interferon-gamma (INF-γ)); 3) chemokines such as fusion proteins comprising CXCL10 (IP-10) and CCL7 (MCP-3) fused to a tumor self-antigen have been shown to induce anti-tumor immunity (Biragyn, et al. Nature Biotech. 1999, 17: 253-258), CCL3 (MIP-1α), and/or CCL5 (RANTES) (Boyer, et al. Vaccine, 1999, 17 (Supp. 2): S53-S64); 4) immune inhibitory proteins and/or peptides such as anti-CTLA-4 agent(s) (Shrikant, et al. Immunity, 1996, 14: 145-155; Sutmuller, et al. J. Exp. Med., 2001, 194: 823-832), anti-CD25 agent(s) (Sutmuller, supra), anti-CD4 agent(s) (Matsui, et al. J. Immunol., 1999, 163: 184-193), the fusion protein IL13Ra2-Fc (Terabe, et al. Nature Immunol., 2000, 1: 515-520), anti-cytokine agents (e.g., antibodies or fragments thereof such as anti-IL6, anti-IL6 receptor, anti-IL17 (e.g., BMS-945429 (ALD518), clazakizumab, dupilumab, elsilimomab, olokizumab (CDP6038), siltuximab (Sylvant), sirukumab (CNTO 136), toclizumab (Actemra); see also the agents listed in Table 4 herein); 5) one or more TLR agonist (LPS mimic 7-mer peptide (TLR4 agonist; e.g., Gln Glu Ile Asn Ser Ser Tyr (SEQ ID NO: 463) (RS01); Ser His Pro Arg Leu Ser Ala (SEQ ID NO: 464) (RS02); Ser Met Pro Asn Pro Met Val (SEQ ID NO: 465) (RS03); Gly Leu Gln Gln Val Leu Leu (SEQ ID NO: 466) (RS04); His Glu Leu Ser Val Leu Leu (SEQ ID NO: 467) (RS05); Tyr Ala Pro Gln Arg Leu Pro (SEQ ID NO: 468) (RS06); Thr Pro Arg Thr Leu Pro Thr (SEQ ID NO: 469) (RS07); Ala Pro Val His Ser Ser Ile (SEQ ID NO: 470) (RS08); Ala Pro Pro His Ala Leu Ser (SEQ ID NO: 471) (RS09); Thr Phe Ser Asn Arg Phe Ile (SEQ ID NO: 472) (RS10); Val Val Pro Thr Pro Pro Tyr (SEQ ID NO: 473) (RS11); and, Glu Leu Ala Pro Asp Ser Pro (SEQ ID NO: 474) (RS12)); or, Flagellin (TLR5 agonist (e.g., FliC; Skountzou, et al. Salmonella flagellins are potent adjuvants for intranasally administered whole inactivated influenza vaccine, Vaccine. May 28; 28(24): 4103-4112 (2010)); e.g., 51 subunits with integrated TLR agonist sequences (Kim et al. Microneedle array delivered recombinant coronavirus vaccines: Immunogenicity and rapid translational development. EBioMedicine (2020)); and combinations thereof. The use of other types of molecular adjuvants are also contemplated herein as would be understood by those of ordinary skill in the art.

Formulations

In some embodiments, the present replication deficient adenovirus vector that contains and expresses SARS-CoV-2 spike (S) antigen, or immunogenic fragment thereof, that can be codon-optimized for the human subject, may be combined with other coronavirus antigens (e.g. viral vector expressed antigens) to form a multivalent coronavirus pharmaceutical formulation. The other components may be included to induce a humoral response with antibodies to a different epitope than that presented in the instant adenoviral vector containing spike protein antigen. In other embodiments, the other component(s) may be included to induce a different arm of the immune system, such as cell-mediated or mucosal immune response to a coronavirus antigen.

In certain embodiments provided herein is a monovalent or multivalent coronavirus pharmaceutical formulation suitable for intranasal administration to a human subject, comprising: an effective amount of at least 10⁷ viral particles (vp) or infectious units (ifu) (e.g., at least 1×10⁷, or at least 1×10⁸, or at least 1×10⁹, or at least 1×10¹⁰, or at least 1×10¹¹ vp or ifu) of rdAd vector (i.e., one or more SARS-CoV-2 vectors); and, a pharmaceutically acceptable excipient, diluent, and/or carrier. In some embodiments, the rdAd vector contains and expresses SARS-CoV-2 spike antigen, or an immunogenic fragment thereof codon optimized for the human subject; and, a pharmaceutically acceptable excipient, diluent, and/or carrier. In certain embodiments, the effective amount induces a protective immune response configured to provide seroprotection, mucosal protection or cellular protection (e.g., based on a cellular immune response such as T cells) to the human subject for at least 1 month (e.g., 28 days or 4 weeks), at least 2 months, at least 3 months, at least 6 months, at least 8 months, at least 12 months, at least 13 months, or at least 14 months against SARS-CoV-2 infection. The period of at least one month to at least 14 months can be considered a duration of protection. In certain embodiments, the protective immune response comprises a combined mucosal, humoral and/or T cell response. In embodiments, the protective immune response is induced via a single intranasal dose. In alternative embodiments, the protective immune response is induced via two or more intranasal doses, for example a prime dose followed by a boost dose about 2 to 3 weeks later.

In exemplary embodiments provided herein is a SARS-CoV-2 immunogenic composition (e.g., vaccine) pharmaceutical formulation suitable for intranasal administration to a human subject, comprising: an effective amount of at least 10⁷ viral particles (vp) or infectious units (ifu) (e.g., at least 1×10⁷, or at least 1×10⁸, or at least 1×10⁹, or at least 1×10¹⁰, or at least 1×10¹¹ vp or ifu) of a replication defective adenoviral vector comprising an expression cassette comprising a coding sequence encoding at least SARS-CoV-2 spike (S) protein receptor binding domain (RBD), or at least one immunogenic fragment thereof codon optimized for a human subject, wherein the composition is configured to induce neutralizing antibody to the spike protein RBD, in the human subject; and, a pharmaceutically acceptable diluent or carrier.

With respect to dosages, routes of administration, formulations, adjuvants, and uses for recombinant viruses and expression products therefrom, compositions of the invention may be used for parenteral, topical, or mucosal administration, preferably by intradermal, subcutaneous, intranasal or intramuscular routes. When mucosal administration is used, it is possible to use oral, ocular or nasal routes. In exemplary embodiments, the present immunogenic compositions (e.g., vaccine) are administered intranasally. In exemplary embodiments, the present immunogenic compositions (e.g., vaccine) are administered intranasally to the mammalian subject. In some embodiments, the SARS-CoV-2 immunogenic composition can be administered using a device such as a microneedle (e.g., as in Kim et al. EBioMedicine, 00 (2020)).

The immunogenic compositions (e.g., formulations) which comprise the adenovirus vector of interest, can be prepared in accordance with standard techniques well known to those skilled in the pharmaceutical or veterinary art. See Example 1 and 2. Such formulations can be administered in dosages and by techniques well known to those skilled in the clinical arts taking into consideration such factors as the age, sex, weight, and the route of administration. The formulations can be administered alone (i.e., as the sole active agent(s)) or can be co-administered or sequentially administered with compositions, e.g., with “other” immunogenic compositions, or attenuated, inactivated, recombinant immunogenic compositions (e.g., vaccine) or therapeutic compositions thereby providing multivalent or “cocktail” or combination compositions of the invention and methods employing them. In some embodiments, the formulations may comprise sucrose as a cryoprotectant and polysorbate-80 as a non-ionic surfactant. In certain embodiments, the formulations further comprise free-radical oxidation inhibitors ethanol and histidine, the metal-ion chelator ethylenediaminetetraacetic acid (EDTA), or other agents with comparable activity (e.g., block or prevent metal-ion catalyzed free-radical oxidation).

The compositions (e.g., formulations) may be present in a liquid preparation for mucosal administration, e.g., oral, nasal, ocular, etc., formulations such as suspensions and, preparations for parenteral, subcutaneous, intradermal, intramuscular, intravenous (e.g., injectable administration) such as sterile suspensions or emulsions. In such formulations the adenoviral vector may be in admixture with a suitable carrier, diluent, or excipient such as sterile water, physiological saline, viscosity enhancing excipients or the like. Certain specialized formulations for mucosal administration can be used, including mucoadhesives, mucosal penetrants and mucosal disruptants. The formulations can also be lyophilized or frozen. The formulations can contain auxiliary substances such as wetting or emulsifying agents, pH buffering agents, adjuvants, preservatives, and the like, depending upon the route of administration and the preparation desired. The formulations can contain at least one adjuvant compound. In exemplary embodiments, the present immunogenic compositions (e.g., vaccines) are non-adjuvanted. Standard texts, such as “REMINGTON'S PHARMACEUTICAL SCIENCE”, 17th edition, 1985, incorporated herein by reference, may be consulted to prepare suitable preparations, without undue experimentation.

In some embodiments, an effective amount (e.g., an amount that induces a protective immune response) of the adenoviral vector is at least 10⁷ viral particle (vp) or infectious units (ifu) (e.g., at least 1×10⁷, or at least 1×10⁸, or at least 1×10⁹, or at least 1×10¹⁰, or at least 1×10¹¹ vp or ifu) of a replication deficient adenoviral vector containing and expressing coronavirus spike protein antigen, or fragment thereof, codon optimized for the human subject. As understood by one of skill in the art, codon optimization improves expression of heterologous genes in a host organism.

In preferred embodiments, an “effective amount” of adenoviral vector and/or immunogenic composition is one administered to a host in a form, dose, and/or administration regimen sufficient to induce an anti-SARS-CoV-2 immune response (e.g., humoral, mucosal and/or cell-mediated immune response) that in some embodiments can be protective from SARS-CoV-2 infection (and/or CoV disease progression). In some embodiments, such as described in the examples herein, a host to which the effective amount was administered can exhibit an induction of (e.g., the appearance of) and/or an increase in the number and/or function of anti-SARS-CoV-2 antibody-producing cells (e.g., B cells, plasma cells) that produce antibodies that bind to CoV and/or antigens (or immunogens) thereof, such as an SARS-CoV-2 specific immunoglobulin G (IgG) response. In some embodiments, such as described in the examples herein, a host to which the effective amount was administered can exhibit an induction of (e.g., the appearance of) and/or an increase in the number and/or function of cells forming an anti-SARS-CoV-2 cell-mediated response (e.g., T cells, granulocytes, natural killer (NK) cells, and the like). In some embodiments, a host to which the effective amount was administered can exhibit an induction of (e.g., the appearance of) and/or an increase in the number and/or function of anti-SARS-CoV-2 antibody-producing cells and cells forming an anti-SARS-CoV-2 cell-mediated response.

In order to determine whether a host to which the effective amount was administered exhibits exhibit an induction of (e.g., the appearance of) and/or an increase in the number and/or function of anti-SARS-CoV-2 antibody-producing cells, in some embodiments, a SARS-CoV-2-specific enzyme-linked immunosorbent assay (ELISA) can be used. As shown in the examples using a murine model, following administration of an immunogenic composition (e.g., 21 days after administration), mice can bled to provide samples for determining the presence of a systemic antibody response using a SARS-CoV-2-specific ELISA (e.g., to determine SARS-CoV-2 specific IgG response has occurred). Briefly, ELISA can be performed by coating polystyrene 96-well plates overnight at 4° C. with 1 μg/ml of SARS-CoV-2 S protein in sodium carbonate buffer (pH 9.3). Plates can be washed (e.g., three times in PBS with 0.02% Tween 20) and blocked (e.g., with non-fat dried milk) for a suitable amount of time and temperature (e.g., one hour at 37° C. with PBS, 2% BSA, and 0.02% Tween 20). Serum from hAd5-SARS-CoV-2 vaccinated mice can be serially diluted (e.g., in PBS) and incubated at an appropriate temperature and time (e.g., 37° C.), washed (e.g., four times with PBS with 0.02% Tween 20) and then incubated with a labeled secondary antibody (e.g., biotin-labeled goat anti-mouse secondary antibody) for an appropriate amount of time (e.g., one hour). The samples can then be washed and incubated with an appropriate reagent (e.g., HRP-conjugated streptavidin), and developed using an appropriate agent (e.g., tetramethylbenzidine substrate), the reaction being stopped with the addition of an appropriate reagent (e.g., 2 N H2504), and emission (450 nm) read using an microplate reader. In some embodiments, administration to a host of an effective amount of an immunogenic composition comprising an adenoviral vector encoding one or more CoV antigens (e.g. Spike protein) can result in the expression of SARS-CoV-2-specific (e.g., CoV S protein-specific) antibodies of a particular type (e.g., IgA, IgM, IgG) and/or amount (e.g., a particular reciprocal mean endpoint indicative of a response (e.g., as compared to naïve hosts). Other assay systems can also be used to determine whether an effective amount has been administered such as, for instance but without limitation, neutralizing antibody assays.

In order to determine whether a host to which the effective amount was administered exhibits exhibit an induction of (e.g., the appearance of) and/or an increase in the number and/or function of cells forming an anti-SARS-CoV-2 cell-mediated response, cell types and/or numbers and/or cytokine expression and/or functional assays can be used. For instance, in some embodiments, T cells of a host to which (or whom) an immunogenic composition was administered can be isolated and studied (e.g., physically isolated from other cells and/or as present within a biological sample such as blood). In some embodiments, an intracellular cytokine staining assay can be performed to determine the type and/or number of cells expressing a particular cytokine, and/or the level of such cytokine being expressed therein. Briefly, a biological sample (e.g., blood, spleen) of a host to which an immunogenic composition has been administered can be isolated at a particular point following administration (e.g., eight to 21 days post-administration). Cells (e.g., approximately 10⁶ cells in cell culture media (e.g., RPMI with 10% FBS and HEPES)) isolated from said biological sample(s) can then be plated in a culture plate(s) (e.g., round bottom 96 well plate), stimulated for an appropriate amount of time, temperature, etc. (e.g., 6 hours at 37° C., 5% CO₂) in the presence of stimulator(s) (e.g., 10 μg/ml brefeldin A and either α-CD3 (2C11 clone) or 10 μg of CoV peptide (e.g., the spike antigen SARS-CoV-2 peptide (SEQ ID NO 3) in 90% DMSO). Following CoV peptide stimulation, cells can be washed (e.g., once with phosphate-buffered saline (PBS)) and stained for the following cell surface markers indicating cell type (e.g., α-CD8-PerCP-Cy 5.5 (clone 53-6.7), α-CD3-AF700 (clone 500A2), and α-CD19-BV605 (clone 1D3)). Cells can then be fixed (e.g., using formalin), permeabilized, stained for intracellular cytokine markers (e.g., α-IFN-γ-APC (clone B27)), and analyzed by flow cytometry (e.g., using an Attune-NXT). In some embodiments, an effective amount can be an amount of immunogenic composition that raises the number of cells expressing the cytokine (e.g., IFN-γ) and/or the amount expressed by such cells.

In some embodiments, the anti-SARS-CoV-2 immune response is protective, meaning that it can protect a host from experiencing one or more of the symptoms of SARS-CoV-2 infection. In some embodiments, a protective immune response prevents SARS-CoV-2 infection, which can be demonstrated by challenge of a host to which (or whom) the effective amount was administered. In some embodiments, an immunogenic composition, and/or effective amount thereof, that is protective is a vaccine. To determine if an immunogenic composition is protective, a pre-clinical animal model can be used. For instance, in some embodiments, a SARS-CoV-2 immunogenic composition can be administered to mice susceptible to infection and disease and the mice can be challenged by live SARS-CoV-2 at a subsequent time (e.g., 7-21 days following administration) and monitored for survival and/or symptoms in comparison to the control group. Symptoms of SARS-CoV-2 infection can also be monitored, including clinical signs of disease (e.g., upper and lower respiratory symptoms). Thus, in some embodiments, in order to determine whether an hAd5-SARS-CoV-2 immunogenic composition is protective (i.e., is a vaccine), one of ordinary skill in the art can conduct an animal challenge study.

In some embodiments, an assay to determine the titer of neutralization antibody, following vaccination of an animal model, is performed such as a plaque reduction neutralization test (PRNT) or focus reduction neutralization test (FRNT) which will demonstrate induction of a protective immune response. In this instance, serum or other biological fluids is collected from post-vaccinated animals (e.g. with hAd5-SARS-CoV-2) and mixed with SARS-CoV-2 suspension and incubated for a time period to allow the serum antibodies to react with SARS-CoV-2. The serum antibody/SARS-CoV-2 mixture is poured over a confluent monolayer of host (i.e. SARS-CoV-2 permissive) cells. The surface of the cell layer is covered in a layer of agar or carboxymethyl cellulose to prevent the SARS-CoV-2 virus from spreading indiscriminately. The concentration of plaque forming units (pfu) can be estimated by the number of plaques (regions of infected cells) formed after a few days. The plaque forming units may be measured by microscopic observation, fluorescent antibodies or specific dyes that react with infected cells. The concentration of serum to reduce the number of plaques by 50% compared to the serum free virus gives the measure of how much neutralization antibody is present or how effective it is (i.e. protective). This measurement is denoted as the PRNT₅ value. Additionally, in an FRNT assay, virus may be visualized using antibody labeling to similarly calculate the FRNT₅₀. Other methods using a pseudo-virus neutralization assay or an ACE-2 binding inhibition assay can be used to quantity the presence of neutralizing antibodies against SARS-CoV-2.

In certain embodiments, the present immunogenic composition (e.g., vaccine) comprises an effective amount of about 10⁷ viral particles (vp) or infectious units (ifu) (e.g., at least 1×10⁷, or at least 1×10⁸, or at least 1×10⁹, or at least 1×10¹⁰, or at least 1×10¹¹ vp or ifu) of a replication deficient adenoviral vector. In exemplary embodiments, the present immunogenic composition (e.g., vaccine) comprises an effective amount of about 10⁸ viral particles (vp) of a replication deficient adenoviral vector. In certain other exemplary embodiments, the present immunogenic composition (e.g., vaccine) comprises an effective amount of about 10⁹ viral particles (vp) of a replication deficient adenoviral vector. In certain other exemplary embodiments, the present immunogenic composition (e.g., vaccine) comprises an effective amount of about 10¹⁰, or greater, viral particles (vp) of a replication deficient adenoviral vector. In certain other exemplary embodiments, the present immunogenic composition (e.g., vaccine) comprises an effective amount of about 10¹¹, or greater, viral particles (vp) of a replication deficient adenoviral vector. In some embodiments, the mammal is a companion or domesticated or food-producing or feed-producing or livestock or game or racing or sport animal such as a cow, a dog, a cat, a goat, a sheep, a rabbit, or a pig or a horse, or even fowl such as turkey, ducks or chicken. In exemplary embodiments the mammalian subject is a human.

Methods of Use

Provided herein is a method for inducing an immune response against coronavirus, the method comprising administering an effective amount of the SARS-CoV-2 immunogenic composition to a mammalian subject. In certain embodiments, is provided a method for transmucosal administration of a pharmaceutical dose of a present therapeutic/immunogenic composition (e.g., vaccine) configured to induce an immune response (e.g., a protective immune response as a vaccine) via intranasal administration. In certain embodiments the present immunogenic compositions comprise a replication defective adenoviral vector comprising an expression cassette comprising a coding sequence encoding at least one coronavirus antigen or at least one immunogenic fragment thereof, wherein the coding sequence encodes at least one or more B cell epitopes, one or more CD8+ T cell epitopes, and/or one or more CD4+ T cell epitopes. In exemplary embodiments the mammalian subject is a human being and the coronavirus antigen is from SARS-CoV-2. In some embodiments, the mammalian subject is a human being infected by SARS-CoV-2 (e.g., a hospitalized human being). In some embodiments, the SARS-CoV-2 immunogenic composition can be used to treat SARS-CoV-2 infection (e.g., in such an infected and/or hospitalized human being).

In some embodiments, the methods of this disclosure can include administration of one or more immunogenic compositions of this disclosure to, in a mammal, preferably a human being: induce an anti-SARS-CoV-2 immune response, preferably statistically significant anti-SARS-CoV-2 immune response; induce a protective and/or curative anti-SARS-CoV-2 immune response; induce an anti-SARS-CoV-2 immune response with an acceptable safety profile; confer prophylactic therapy against SARS-CoV-2; reduce the rates of intensive care unit (ICU) admission and mechanical ventilation in patients with early onset COVID-19; reduce the severity of COVID-19 in patients with early onset COVID-19 who require hospitalization; inhibit, suppress and/or prevent the development of a “cytokine storm” during infection by SARS-CoV-2 (in preferred embodiments, co-administering one or more of immunogenic compositions with one or more anti-cytokine reagents); induce significant decreases (e.g., as compared to placebo controls) in IL-1α, IL-6, and/or IL-12p70, and/or pulmonary interstitial inflammation in a patient having COVID-19 (i.e., a patient infected with SARS-CoV-2); accelerate the time to clinical improvement and/or recovery in patients (e.g., hospitalized patients) infected with SARS-CoV-2; induce anti-SARS-CoV-2 neutralizing antibodies (in preferred embodiments, IgG and/or IgA); induce anti-SARS-CoV-2 T cell immunity (e.g., systemic and/or mucosal); induce bone marrow and lung resident memory antibody secreting cells; induce an immune response (e.g., neutralizing antibodies and/or T cell immunity) that is effective and/or can be detected for at least 4 months, for at least about 5 months, at least about 6 months, at least about 7 months, at least about 8 months, at least about 9 months, at least about 10 months, at least about 11 months, or at least about 12 months; and/or, provide for repeated administration (e.g., as a seasonal vaccine administered about once every 11-14 months) without inducing a significant immune response against the adenoviral vector itself.

Dosage of the immunogenic composition (e.g., Ad-vector SARS-CoV-2 vaccine) when used with or without an adjuvant may range from about 10⁷ to about 10¹² infectious unit or plaque forming unit (ifu or pfu), or the dosage unit may be a viral particle (vp), wherein 1 vp equals about 1-100 ifu or pfu. In one embodiment the dose of Ad-vector SARS-CoV-2 immunogenic composition or vaccine administered to the mammalian subject is about, or at least about, 10⁷ vp or infectious units (ifu) (e.g., at least 1×10⁷, or at least 1×10⁸, or at least 1×10⁹, or at least 1×10¹⁰, or at least 1×10¹¹ vp or ifu). In another aspect the dose of Ad-vector SARS-CoV-2 immunogenic composition or vaccine administered to the mammalian subject is about, or at least about, 10⁸ vp. In yet another aspect, the dose of Ad-vector SARS-CoV-2 immunogenic composition or vaccine administered to the mammalian subject is about, or at least about, 10⁹ vp. In another aspect the dose of Ad-vector SARS-CoV-2 immunogenic composition or vaccine administered to the mammalian subject is about, or at least about, 10¹⁰ vp. In another aspect the dose of Ad-vector SARS-CoV-2 immunogenic composition or vaccine administered to the mammalian subject is about, or at least about, 10¹¹ vp. In another aspect the dose of Ad-vector SARS-CoV-2 immunogenic composition or vaccine administered to the mammalian subject is about, or at least about, 10¹² vp.

One of skill in the art understands that an effective dose in a mouse may be scaled for larger animals such as a human, dogs, pigs, non-human primates, minks, ferrets, cats, horses/equines, etc.; and, these larger animals are subjects for administration in accordance with this disclosure. In that way, through allometric scaling (also referred to as biological scaling) a dose in a larger animal may be extrapolated from a dose in a mouse to obtain an equivalent dose based on body weight or body surface area of the animal.

In certain embodiments, non-invasive administration of the Ad-vector SARS-CoV-2 immunogenic composition or vaccine includes, but is not limited to, topical application to the skin, and/or intranasal and/or mucosal and/or perlingual and/or buccal and/or oral and/or oral cavity administration. Dosage forms for the application of the Ad-vector SARS-CoV-2 immunogenic composition or vaccine may include liquids, ointments, powders and sprays. The active component may be admixed under sterile conditions with a physiologically acceptable carrier and any preservative, buffers, propellants, or absorption enhancers as may be needed.

In certain embodiments provided herein is a method for transmucosal administration of a therapeutic dose of a non-replicating viral vectored immunogenic composition (e.g., vaccine) to a human subject, wherein the method comprises administering intranasally to the human subject the immunogenic composition (e.g., vaccine) comprising an effective amount of at least 10⁷ viral particle (vp) or infectious units (ifu) (e.g., at least 1×10⁷, or at least 1×10⁸, or at least 1×10⁹, or at least 1×10¹⁰, or at least 1×10¹¹ vp or ifu) of replication deficient adenovirus vector that contains and expresses a heterologous SARS-CoV-2 antigen codon optimized for the mammalian subject; whereby the therapeutic dose administered transmucosally induces a protective immune response.

In embodiments, for (trans)mucosal administration compositions may be in a form and dispensed by a squeeze spray dispenser, pump dispenser, multi-dose dispenser, dropper-type dispenser or aerosol dispenser. Such dispensers may also be employed to deliver the composition to oral or oral cavity (e.g., buccal or perlingual) mucosa. Aerosols are usually under pressure by means of a hydrocarbon. Pump dispensers may preferably dispense a metered dose or, a dose having a particular particle size. The distribution of aerosol particle/droplet size can be expressed in terms of either: the mass median aerodynamic diameter (MMAD)—the droplet size at which half of the mass of the aerosol is contained in smaller droplets and half in larger droplets; volumetric mean diameter (VIVID); mass median diameter (MMD); or the fine particle fraction (FPF)—the percentage of particles that are <5 um in diameter. These measurements may be made by impaction (MMD and MMAD) or by laser (VIVID). For liquid particles, VMD, MMD and MMAD may be the same if environmental conditions are maintained, e.g., standard humidity. However, if humidity is not maintained, MMD and MMAD determinations will be smaller than VIVID due to dehydration during impactor measurements. For the purposes of this description, VMD, MMD and MMAD measurements are considered to be under standard conditions such that descriptions of VIVID, MMD and MMAD will be comparable. Particles having a mass median aerodynamic diameter (MMAD) of greater than about 5 microns generally do not reach the lung; instead, if administered orally they tend to impact the back of the throat and are swallowed and possibly orally absorbed; and, particles of this size administered nasally will lodge in the nasal mucosa. Particles having diameters of about 1 to about 5 microns are small enough to reach the upper-to mid-pulmonary region (conducting airways) but are too large to reach the alveoli. Smaller particles, i.e., about 0.5 to about 2 microns, are capable of reaching the alveolar region. Particles having diameters smaller than about 0.5 microns can also be deposited in the alveolar region by sedimentation, although very small particles may be exhaled. Depending on whether the desire is to mucosally administer to the nasal mucosa or into the upper-to-mid pulmonary region or into the alveoli, the skilled person can achieve any or all of these targets from this disclosure and the knowledge in the art. In addition, for intranasal administration an atomizer device, such as a LMA MAD300 from Teleflex LLC (Intranasal Mucosal Atomization Device LMA™ #MAD300 Nasal Device Without Syringe, 1.65 inch length, and 0.17 tip diameter) can advantageously be employed. An aerosol device or dispenser such as an atomizer that delivers a mist having a typical particle size of about 30 to about 100 microns to the olfactory mucosa or nasal mucosal membranes can advantageously be employed in the practice of the invention, and advantageous particle sizes of droplets of formulations of this disclosure for the practice of the invention can be from about 30 to about 100 microns, e.g., about 30 or about 40 or about 50 to about 60 or about 70 or about 80 or about 90 or about 100 microns, for intranasal administration.

In embodiments, the present SARS-CoV-2 pharmaceutical formulation is used to provide protection against seasonal coronavirus. In certain other embodiments, the present CoV pharmaceutical formulation is used to provide protection against pandemic SARS-CoV-2. In certain other embodiments, the present SARS-CoV-2 pharmaceutical formulation is used to provide protection against SARS-CoV-2. In embodiments, the seroprotection lasts at least about 1 month, 2 months, 4 months, 6 months, 8 months, 10 months, 12 month or at least about 13 months.

In some embodiments, is provided a method for inducing an immune response against coronavirus, the method comprising administering a single dose of a present immunogenic composition/formulation/dosage to a mammalian subject (e.g. human). In certain embodiments, the method comprises intranasal administration of an effective amount of the immunogenic composition to the mammalian subject, wherein the immune response provides protection against challenge with SARS-CoV-2. In certain embodiments, is provided a method of inducing a combined mucosal, humoral and/or T cell protective immune response in a human subject against coronavirus comprising administering intranasally to a human subject a single dose of the SARS-CoV-2 pharmaceutical formulation (immunogenic composition), or a pharmaceutical dosage thereof, wherein the administration induces serum antibodies, mucosal antibodies and T cells against coronavirus. In embodiments, the human subject is seroprotected at least about 1 month, 2 months, 4 months, 6 months, 8 months, 10 months, 12 month or at least about 13 months. In certain embodiments, the human subject is seroprotected for at least about 9 months.

In alternative embodiments, is provided a method for inducing an immune response against coronavirus wherein the method comprises administering at least a prime and boost dose of a present immunogenic composition/formulation/dosage. In certain embodiments, the boost dose is administered about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks or 52 weeks after administration of the prime dose.

In embodiments, the prime boost doses are homologous, meaning they are the same formulation. In certain embodiments, the methods and compositions provided include administering a heterologous immunogenic composition or vaccine prime dose and boost dose leading to an induction of an immune response (e.g., T cell, humoral and/or mucosal) where “heterologous” means a prime dose that is different than a boost dose, provided both comprise at least one, or more, of the SARS-Cov-2 antigen epitopes. In certain embodiments, that means a prime dose and boost dose wherein the antigen/immunogens/peptides are presented to the immune system via different delivery carriers and/or vectors. As used herein, a “heterologous dosing regimen”, means a prime dose and boost dose wherein the antigen/immunogens/peptides are presented to the immune system via different delivery carriers and/or vectors. For example, in the instant invention a first composition (as either a prime dose or a boost dose) comprises a viral vectored antigen or immunogen, and a second composition (as either a prime dose or a boost dose) comprises fluorocarbon-linked peptides, wherein the peptide comprises one or more T cell epitopes in common with the viral vectored antigen or immunogen. In other words, in certain embodiments, the present immunogenic composition or vaccine combination is two different T cell inducing immunogenic composition or vaccine compositions, wherein each composition induces antigen specific CD8+ T cells against the same antigen. In alternative embodiments, a first composition (as either a prime dose or a boost dose) comprises a viral vectored antigen or immunogen, and a second composition (as either a prime dose or a boost dose) comprises an RNA immunogenic composition or vaccine composition such as a composition comprising a modified mRNA, a unmodified mRNA or a self-amplifying mRNA formulated in liposomes or lipid nanoparticles., a DNA composition administered by electroporation or using a lipid based formulation, a live attenuated immunogenic composition or vaccine composition, a protein-based composition formulated or not with an adjuvant or delivery system, a killed immunogenic composition or vaccine composition, a different adenoviral vectored immunogenic composition or vaccine composition such as HAdV-1 to 57, a simian adenovirus or a non-adenoviral vector such as but not limited to an adeno-associated virus (AAV), a lentivirus or a poxvirus. The first composition can be administered first to prime the immune response locally or systemically and the second immunogenic composition or vaccine such as the adenovirus can be administered mucosally as a booster to “pull” the primed immune cells locally and re-stimulated them in an antigen specific manner.

In embodiments, the prime and boost dose are administered at least 7 days apart, at least 14 days apart, or longer. In embodiments, the prime dose and boost dose are administered about 7 days apart, about 14 days apart, about 20 days apart, about 25 days apart, about 30 days apart, about 35 days apart, about 40 days apart, about 45 days apart, about 50 days apart, about 55 days apart, about 60 days apart or about 65 days apart. Advantageously, the doses are administered about 40 days apart, about 41 days apart, about 42 days apart, about 43 days apart, about 44 days apart, about 45 days apart, about 46 days apart, about 47 days apart, about 48 days apart, about 49 days apart or about 50 days apart. In certain embodiments, the prime dose and boost dose are administered about 1 week apart, about 2 weeks apart, about 3 weeks apart, about 4 weeks apart, about 5 weeks apart, about 6 weeks apart, about 7 weeks apart, about 8 weeks apart, about 9 weeks apart, about 10 weeks apart, about 11 weeks apart or about 12 weeks apart. In certain other embodiments, the prime dose and boost dose are administered about 1 month apart, about 2 months apart, about 3 months apart, about 4 months apart, about 5 months apart, about 6 months apart, about 7 months apart, about 8 months apart, about 9 months apart, about 10 months apart, about 11 months apart, or about 12 months apart.

In embodiments, the first or second immunogenic composition or vaccine composition is administered as a prime and boost dose administered at least 7 days apart, at least 14 days apart, or longer. In embodiments, the prime dose and boost dose are administered about 7 days apart, about 14 days apart, about 20 days apart, about 25 days apart, about 30 days apart, about 35 days apart, about 40 days apart, about 45 days apart, about 50 days apart, about 55 days apart, about 60 days apart or about 65 days apart. Advantageously, the doses are administered about 40 days apart, about 41 days apart, about 42 days apart, about 43 days apart, about 44 days apart, about 45 days apart, about 46 days apart, about 47 days apart, about 48 days apart, about 49 days apart or about 50 days apart. In certain embodiments, the prime dose and boost dose are administered about 1 week apart, about 2 weeks apart, about 3 weeks apart, about 4 weeks apart, about 5 weeks apart, about 6 weeks apart, about 7 weeks apart, about 8 weeks apart, about 9 weeks apart, about 10 weeks apart, about 11 weeks apart or about 12 weeks apart. In certain other embodiments, the prime dose and boost dose are administered about 1 month apart, about 2 months apart, about 3 months apart, about 4 months apart, about 5 months apart, about 6 months apart, about 7 months apart, about 8 months apart, about 9 months apart, about 10 months apart, about 11 months apart, or about 12 months apart.

In some embodiments, rdAd anti-SARS-CoV-2 vectors (e.g., a SARS-CoV-2 immunogenic composition (which can include more than one type of rdAd anti-SARS-CoV-2 vector)) can be administered with one or more anti-cytokine reagents. As shown in Example 2, administration of AdE to mice was shown to decrease the expression of cytokines known to be involved in the progression and symptoms of infectious diseases caused by viruses such as influenza. For instance, AdE can cause an increase in the expression of monocyte chemoattractant protein (MCP-1 (CCL2)), interferon gamma (IFN-γ), and RANTES (CCL5) upon administration to non-infected mammals, which can be accompanied by a decrease in IL-12 expression. Three days after exposure to influenza, animals to which AdE was administered were found to exhibit decreased expression of IL-1α, IL-6, IL-12, MCP-1, with a significant decrease of IL-1α, and IL-12. Six (6) days after exposure to influenza, the animals exhibited decreased expression of IL-5, IL-6, IL-12, IL-17, MCP-1 and GM-CSF, and increased expression of macrophage inflammatory protein 1 alpha (MIP-la (CCL3)) and RANTES (CCL5). These results are consistent with the development of a “cytokine storm” during infection by SARS-CoV-2. In some embodiments, then, to prevent and/or treat SARS-CoV-2 infection, a SARS-CoV-2 immunogenic composition can be administered to a mammal, such as a human being, with one or more anti-cytokine reagent(s) (i.e., co-administered). Such co-administration can be carried out as single mixture (e.g., one or more anti-cytokine reagents can be included in the SARS-CoV-2 immunogenic composition), or as separate compositions administered essentially simultaneously and/or at or near the same anatomical site, or at different anatomical sites by an appropriate route (e.g., intranasal administration of the SARS-CoV-2 immunogenic composition and intradermal or intravenous administration of the one or more anti-cytokine reagent(s)), and in an effective amount that can vary for each type of anti-cytokine reagent (and as is known in the art). In some embodiments, the SARS-CoV-2 immunogenic composition can be administered as a single dose, as can the one or more anti-cytokine reagents. In some embodiments, the one or more anti-cytokine reagents can be administered multiple times (e.g., any of about 7, 14, 21 days, or any of about one, two or three months) after administration of the SARS-CoV-2 composition including, in some embodiments, an initial administration with the SARS-CoV-2 composition. Exemplary anti-cytokine reagents for administration to a mammal to prevent and/or treat SARS-CoV-2 can therefore include, but are not limited to, one or more anti-IL-1α reagent(s), one or more anti-IL5 reagent(s), one or more anti-IL-6 reagent(s), one or more anti-IL-12 reagent(s), one or more anti-IL-17 reagent(s), one or more anti-MCP-1 reagent(s), one or more anti-TNF-α reagent(s), one or more anti-GM-CSF reagent(s), and/or one or more anti-RANTES reagent(s). In some embodiments, the one or more anti-cytokine reagents would not include one or more anti-MIPα reagent(s) and/or one or more anti-RANTES reagent(s). Exemplary anti-cytokine reagents that can be used as described herein can include, for example, any of those shown in Table 10.

TABLE 10

| Cytokine | Exemplary Anti-Cytokine Reagents |
|---|---|
| IL-1α | anakinra (Kineret ®), canakinumab (Ilaris ®), |
| | rilonacept (Arcalyst ®) |
| IL-5 | mepolizumab (GlaxoSmithKline); |
| | benralizumab (Fasenra) |
| IL-6 | tocilzumab (Actemra), sarilumab (Kevzara), |
| | siltuximab (Sylvant) |
| IL-12 | briakinumab (ABT-874, Abbott); |
| | ustekinumab |
| IL-17 | brodalumab (Siliq; Amgen), ixekizumab |
| | (Taltz ®, Eli Lilly), |
| | secukinumab (Cosentyx; Novartis) |
| TNF-α | inflixibmab (Remicade), adalimumab |
| | (Humira), certolizumab pegol (Cimzia), |
| | golimumab (Simponi), etanercept (Enbrel), |
| | thalidomide (Immunoprin), lenalidomide |
| | (Revlimid), pomalidomide (Pomalyst, |
| | Imnovid), a xanthine derivative (e.g., |
| | pentoxifylline), bupropion |
| GM-CSF | otilimab (MOR103, GSK3196165) |

In some embodiments, one or more additional anti-SARS-CoV-2 agents can also be administered to the subject(s) before, essentially simultaneously, or after administration of SARS-CoV-2 immunogenic composition such as, for instance, chloroquine (e.g., pharmaceutical salt and/or derivative thereof; e.g., hydroxychloroquine 400 mg per day for 5 days or 200 mg three times per day for 10 days) and/or azithromycin (e.g., 500 mg on first day followed by four daily 250 mg doses) and/or remdesivir (e.g., 200 mg initial followed by 100 mg daily doses) and/or one or more anti-inflammatories (e.g., prednisone, dexamethasone) and/or any other suitable reagent. Other administration/dosing schemes, anti-cytokine reagents, combinations thereof, and combinations with other anti-SARS-CoV-2 agents as are available to those of ordinary skill in the art can be suitable for use as disclosed herein, as would be understood by those of ordinary skill in the art.

In some embodiments, a subject (e.g., human being) can be tested for coronavirus infection by a suitable technique (e.g., polymerase chain reaction (PCR), nasal swab to detect viral particles). An immunogenic composition comprising one or more rdAd anti-SARS-CoV-2 vectors (e.g., as viral particles; SARS-CoV-2 immunogenic composition) can then be administered to individuals that test positive for coronavirus infection. Preferably, such administration can be completed within seven to ten days after initial exposure to the coronavirus. In some embodiments, a SARS-CoV-2 immunogenic composition comprising one or more rdAd anti-SARS-CoV-2 vectors (e.g., as viral particles) can be administered to individuals at high risk for infection and/or symptoms (e.g., respiratory symptoms, death) such as immunocompromised individuals and/or suffering from another disease condition (e.g., kidney failure), and/or persons in high risk situations (e.g., travelers to pandemic areas, enclosed spaces such as cruise ships), whether or not such individuals have tested positive for coronavirus infection.

In some embodiments, the compositions disclosed herein can be administered to a host comprising nostrils, wherein such nostrils are tilted upwards (i.e., the dorsal position), to generate a strong immunogenic response via intranasal administration. Other administration and dosing strategies are also contemplated herein as would be understood by those of ordinary skill in the art.

In some embodiments, this disclosure provides an immunogenic composition comprising a replication defective adenoviral (rdAd) vector, the rdAd vector: a) lacking a coding sequence encoding an exogenous, non-adenoviral, antigen (e.g., AdE); b) comprises an expression cassette comprising a coding sequence encoding at least one SARS-CoV-2 antigen, optionally wherein said antigen comprises a SARS-CoV-2 spike (S) protein receptor binding domain (RBD); c) comprises an expression cassette comprising a coding sequence encoding at least one antigen of an infectious agent other than SARS-CoV-2 (e.g., AdD); d) a combination of the vectors of a) and b), wherein the vectors are administered together or separately; e) a combination of the rdAd vectors of b) and c), wherein the vectors are administered together or separately; f) a combination of any of the rdAd vectors of any of a), b), or c) wherein the rdAd vectors are administered together or separately; and/or, g) a combination of two different types of rdAd vectors of b), wherein each type of rdAd vector comprises an expression cassette encoding at least one SARS-CoV-2 antigen different from that encoded by the other types of rdAd vectors in the combination, wherein the rdAd vectors are administered together or separately; wherein said immunogenic composition is configured to induce neutralizing antibody and/or cellular immune response against SARS-CoV-2 in a mammalian subject to which said immunogenic composition is administered. In some embodiments, the immunogenic composition can comprise a combination of any two of the rdAd vectors of a)-c), such as in a two-part composition comprising at least one composition comprising rdAd vectors of a)-c) and at least a second composition comprising a different rdAd vector of a)-c). In some embodiments, the expression cassette comprises a coding sequence encoding at least one SARS-CoV-2 antigen selected from: the coding sequence for spike (S) protein or S1 domain of the spike protein; a sequence presented in SEQ ID NO: 3, or a sequence having at least 80% homology to SEQ ID NO: 3; comprises at least amino acids 331 to 527 of SEQ ID NO: 3; encodes a spike protein RBD sequence comprises one or more of the following residues (the numbering corresponding to SEQ ID NO: 3): L455, F486, Q493, S494 and/or N501, preferably in some embodiments Q493 and N501, preferably in some embodiments a residue selected from Y455, F455 or S455, preferably in some embodiments a residue selected from L486 or P486, preferably in some embodiments a residue selected from N493, R493 or K493, preferably in some embodiments a residue selected from D494 or G494, preferably in some embodiments a residue selected from T501 or S501; comprises an amino acid sequence of SEQ ID NO: 12, SEQ ID NO: 13, SEQ ID NO: 14, preferably SEQ ID NO: 15, SEQ ID NO: 16 or SEQ ID NO: 17, SEQ ID NO: 446; any of SEQ ID NOS: 412-417 and SEQ ID NOS: 438-445, and SEQ ID NOS: 475-476 or 460; or an immunogenic fragment thereof; comprises an amino acid sequence of SEQ ID NO: 16 or SEQ ID NO: 17, wherein amino acid 455 is selected from Y, F, L or S; amino acid 486 is selected from L, F or P; amino acid 493 is selected from N, Q, R or K; amino acid 494 is selected from D, G or S; and, amino acid 501 is selected from T, S or N; a coding sequence encoding one or more of SARS-CoV-2 structural proteins envelope (E), membrane (M) or nucleocapsid (N). In some embodiments, the expression cassette comprises a coding sequence for a modified version of SEQ ID NO: 411 comprising: one or more substitutions of any one or more of amino acids 333-388, 390-395, 397-399, 401-411, 413-415, 417-419, 424, 426-435, 437, 439-442, 444-446, 449, 450, 452, 453, 455-463, 465, 467-473, 475-479, 481-486, 490, 491, 493-495, 499-510, and/or 513-526; one or more substitutions of any one or more of amino acids 367, 403, 417, 439, 446, 449, 452, 453, 455, 456, 470, 473, 475, 476, 477, 478, 484, 486, 490, 493, 494, 495, 496, 499, 500, 501, 502, 503, 504, and/or 505; and/or, one or more substitutions selected from the group consisting of amino acid 367 (V) by F, I, L S or A, amino acid 403 (R) by K or S, 417 (K) by N or T; amino acid 439 (N) by K, amino acid 446 (G) by V, S or A; amino acid 449 (Y) by N; amino acid 452 (L) by L, M or Q, amino acid 453 (Y) by F; amino acid 455 (L) by F; amino acid 456 (F) by L; amino acid 470 (T) by I, A or N, amino acid 473 (Y) by V; amino acid 475 (A) by V; amino acid 476 (G) by S or A; amino acid 477 (S) by N, R, T, G, A or I; amino acid 476 (G) by S or A, amino acid 477 (S) by N, R, T, G, A or I, amino acid 478 (T) by I, K, R or A, amino acid 484 (E) by Q, K, D, A or R; amino acid 486 (F) by L or S; amino acid 490 (F) by L or S, amino acid 493 (Q) by L or R; amino acid 494 (S) by P or L, amino acid 495 (Y) by N or F; amino-acid 496 (G) by V or S, amino acid 499 (P) by H, S or R, amino acid 500 (T) by I; amino acid 501 (N) by Y, T or S; amino acid 502 (G) by R, D or C; amino acid 503 (V) by L, I or F; and, amino acid 504 (G) by V, D or S amino acid 505 (Y) by H, E, W or C. In some embodiments, the at least one antigen of an infectious agent other than SARS-CoV-2 is derived from an influenza virus. In some embodiments, the immunogenic composition is further configured to induce a combined mucosal, humoral and T cell protective immune response against SARS-CoV-2. In some embodiments, the coding sequence encodes at least one or more B cell epitopes, one or more CD8+ T cell epitopes, and/or one or more CD4+ T cell epitopes. In some embodiments, the coding sequence is codon optimized for a mammalian subject. In some embodiments, the immunogenic composition induces the production of neutralizing antibodies seroprotective against SARS-CoV-2 infection in a mammalian subject, optionally wherein the mammalian subject is a human being. In some embodiments, the replication defective adenoviral vector (rdAd) is a human adenovirus, optionally Ad5 or Ad26. In some embodiments, the rdAd is a primate adenovirus, a chicken adenovirus, or a porcine or swine adenovirus. In some embodiments, the rdAd is an E1, E3, and/or E4 deleted or disrupted adenovirus.

In some embodiments, this disclosure provides pharmaceutical formulations comprising an effective amount of such immunogenic composition (i.e., a composition comprising one or more rdAd anti-SARS-CoV-2 vectors) and, a pharmaceutically acceptable diluent or carrier, optionally wherein the diluent is phosphate-buffered saline. In some embodiments, the pharmaceutical formulation is configured for non-invasive administration, and/or for intranasal administration to the mammalian subject. In some embodiments, administration of the pharmaceutical formulation to the mammalian subject induces a protective immune response in the mammalian subject, optionally a combined mucosal, humoral and T cell protective immune response. In some embodiments, the pharmaceutically acceptable carrier is in a spray or aerosol form. In some embodiments, the effective amount is at least 10⁷ viral particles (vp), at least 10⁸ viral particles (vp), or at least 10⁹ viral particles (vp) (of the rdAd anti-SARS-CoV-2 vector(s)). In some embodiments, the pharmaceutical formulation is configured as a single intranasal dose. In some embodiments, the pharmaceutical formulation is configured as two or more intranasal doses. In some embodiments, this disclosure provides coronavirus pharmaceutical formulation suitable for a single dose intranasal administration to a human subject, comprising: an effective amount of at least 10⁷ viral particle (vp) or infectious units (ifu) (e.g., at least 1×10⁷, or at least 1×10⁸, or at least 1×10⁹, or at least 1×10¹⁰, or at least 1×10¹¹ vp or ifu) of the immunogenic composition comprising at least one replication defective adenoviral vector comprising an expression cassette comprising a coding sequence encoding at least SARS-CoV-2 spike (S) protein receptor binding domain (RBD), or at least one immunogenic fragment thereof, wherein the effective amount induces a combined mucosal, humoral and T cell protective immune response; and, a pharmaceutically acceptable diluent or carrier. In some embodiments, the formulation is configured to provide seroprotection to the human subject for at least 6 months or preferably 9 months against SARS-CoV-2. In some embodiments, this disclosure provides a pharmaceutical dosage for intranasal administration, comprising a pharmaceutical acceptable carrier in a spray or aerosol form admixed with an immunogenic composition disclosed herein, wherein the dosage is configured for intranasal administration to non-invasively induce a protective immune response against SARS-CoV-2. In some embodiments, the immunogenic composition comprises an effective amount of at least 10⁷ viral particles (vp), at least 10⁸ viral particles (vp), or at least 10⁹ viral particles (vp) of the rdAd anti-SARS-CoV-2 vector(s). In some embodiments, the effective amount induces a combined mucosal, humoral and T cell protective immune response against a coronavirus, preferably SARS-CoV-2. In some embodiments, the pharmaceutical dosage formulation is configured as two or more doses to induce a protective immune response against SARS-CoV-2.

In some embodiments, this disclosure provides methods for inducing an immune response against coronavirus, preferably SARS-CoV-2, the method comprising administering an effective amount of an immunogenic composition (or formulation or dosage form) disclosed herein to a mammalian subject, preferably wherein the immune response is protective against SARS-CoV-2. In some embodiments, the method comprises intranasal administration of an effective amount of an immunogenic composition disclosed herein to a mammalian subject, wherein the immune response provides protection against challenge with SARS-CoV-2. In some embodiments, this disclosure provides methods for inducing a combined mucosal, humoral and/or T cell protective immune response in a human subject against coronavirus comprising: administering intranasally to a human subject a single dose of a coronavirus, preferably SARS-CoV-2, pharmaceutical formulation or dosage disclosed herein, wherein the administration induces serum antibodies, mucosal antibodies and T cells against SARS-CoV-2, optionally whereby the human subject is seroprotected for at least about 6 months or preferably for at least about 9 months. Preferably, the seroprotection lasts for at least 12 months, at least 13 months or at least 14 months.

In preferred embodiments, the rdAd anti-SARS-CoV-2 vector comprises SEQ ID NO: 15 (or comprising a nucleic acid sequence encoding SEQ ID NO: 15), preferably within an expression cassette. In preferred embodiments, the rdAd anti-SARS-CoV-2 vector comprises an expression cassette comprising a SARS-CoV-2 spike protein Receptor Binding Domain (RBD) of the S1 domain, pTA signal sequence (italics) and long flanking sequences (underlined), as illustrated in FIG. 19. Variants of SEQ ID NO: 15 are also contemplated. For instance, any of the leader and flanking sequences shown in FIG. 19 can be deleted (i.e., not included) or substituted by other leader and/or flanking sequences. In preferred embodiments, the rdAd anti-SARS-CoV-2 vector comprising SEQ ID NO: 15 (or comprising a nucleic acid sequence encoding SEQ ID NO: 15), or a variant thereof, can be administered as a pharmaceutical composition comprising the effective amount is at least 10⁷ viral particles (vp), at least 10⁸ viral particles (vp), at least 10⁹ viral particles (vp), at least 10¹⁰ viral particles (vp), or at least 10¹¹ viral particles (vp); preferably administered intranasally (preferably wherein the mammal (preferably a human being) is in the supine position during administration); and preferably as a single administration (dose), but in some embodiments including at least two administrations (doses) separated from one another by time (e.g., 7-21 days). In preferred embodiments, the administration induces the production of anti-SARS-CoV-2 neutralizing antibodies and/or an anti-SARS-CoV-2 cellular response (e.g., T cells) that provides protection (e.g., in some preferred embodiments seroprotection) for at least about 6, 9, 10, 11, 12, 13, or months. In preferred embodiments, administration of the rdAd anti-SARS-CoV-2 vector comprising SEQ ID NO: 15 (or comprising a nucleic acid sequence encoding SEQ ID NO: 15) results in protection against infection by SARS-CoV-2, accelerates recovery from infection by SARS-CoV-2, slows and/or reverses clinical worsening in a patient infected by SARS-CoV-2, and/or reduces or eliminates the need for hospitalization and/or care in in ICU unit for a patient infected by SARS-CoV-2.

In some embodiments, this disclosure provides methods such as those above, further comprising administering one or more anti-cytokine reagents (see, e.g., Table 4) to the human being to prevent and/or treat SARS-CoV-2, optionally wherein the one or more anti-cytokine reagents include one or more anti-IL-1α reagent(s), one or more anti-IL5 reagent(s), one or more anti-IL-6 reagent(s), one or more anti-IL-12 reagent(s), one or more anti-IL-17 reagent(s), one or more anti-MCP-1 reagent(s), one or more anti-TNF-α reagent(s), one or more anti-GM-CSF reagent(s), and/or one or more anti-RANTES reagent(s). In some embodiments, the one or more anti-cytokine reagents does not include one or more anti-MIPα reagent(s) and/or one or more anti-RANTES reagent(s). In some embodiments, the one or more anti-cytokine reagent(s) are co-administered substantially with the effective amount of the immunogenic composition. In some embodiments, the one or more anti-cytokine reagent(s) are not administered substantially with the effective amount of the immunogenic composition. In some embodiments, the immunogenic composition is administered to the mammal once and the one or more anti-cytokine reagent(s) are administered multiple times. In some embodiments, the immunogenic composition is co-administered to the mammal with the one or more anti-cytokine reagent(s), and the one or more anti-cytokine reagent(s) are subsequently administered to the mammal. In some embodiments, this disclosure provides methods for treating and/or inhibiting (e.g., ameliorating) the symptoms of a respiratory viral infection in a mammal, said respiratory viral infection causing elevated expression of interleukin-6 (IL-6), interleukin-1-alpha (IL-la) and/or interleukin-12 (IL-12) in the lung of said mammal which can cause deleterious effects in a host. In some embodiments, such methods comprise intranasally administering an effective amount of an E1 and E3 deleted adenoviral vector to the subject, whereby expression of IL-6, IL-1α, and/or IL-12 in the lung is reduced thereby alleviating said symptoms for up to about 28 days following administration of the vector. In some embodiments, such methods cause the expression of monocyte chemoattractant protein 1 (MCP-1), tumor necrosis factor alpha (TNF-α), granulocyte macrophage colony stimulating factor (GM-CSF), RANTES, and/or IL-17 are reduced in the lung following administration of the vector. In some embodiments, the expression of macrophage inflammatory protein 1 alpha (MIP-1α) and/or RANTES are not reduced following administration of the vector. In some embodiments, this disclosure provides methods for inducing an anti-viral immune response in a mammalian subject in need thereof with, or at risk of, a respiratory viral infection, the method comprising: intranasal administration of an effective amount of an E1 and E3 deleted adenoviral vector to the subject, wherein the anti-viral immune response generates increased expression of monocyte chemoattractant protein 1 (MCP-1) and/or interferon alpha (IFN-γ) following the administration step. In some embodiments of such methods, the mammalian subject (e.g., human being) is infected by SARS-CoV-2 (e.g., in the hospital being treated for SARS-CoV-2 infection) prior to the administering of the pharmaceutical formulation thereto. In some embodiments, one or more additional anti-SARS-CoV-2 agents can be administered to the subject(s) before, essentially simultaneously, or after administration of SARS-CoV-2 immunogenic composition such as, for instance, chloroquine (e.g., pharmaceutical salt and/or derivative thereof; e.g., hydroxychloroquine 400 mg per day for 5 days or 200 mg three times per day for 10 days) and/or azithromycin (e.g., 500 mg on first day followed by four daily 250 mg doses) and/or remdesivir (e.g., 200 mg initial followed by 100 mg daily doses) and/or any other suitable reagent.

In some embodiments, this disclosure provides SARS-CoV-2 immunogenic compositions comprising one or more rdAd anti-SARS-CoV-2 vectors comprising one or more SARS-CoV-2 antigen coding sequences encoding one or more peptides comprising one or more T cell epitopes of Table 3A, one or more groups of T cell epitopes of Table 3B, or SEQ ID NOS: 27-282; and/or one or more B cell epitopes of SEQ ID NOS: 283-327; and/or one or more of SEQ ID NOS: 328-408 optionally wherein the peptides are concatenated, and optionally separated by a linker amino acid sequence of two to ten amino acids.

In some embodiments, this disclosure provides SARS-CoV-2 immunogenic compositions comprising one or more rdAd anti-SARS-CoV-2 vectors comprising at least one polynucleotide encoding at least one molecular adjuvant selected from the group consisting of: one or more polypeptides or peptides that functions as a co-stimulatory component; one or more cytokines; one or more chemokines; one or more immune inhibitory proteins; one or more TLR agonists, optionally wherein the one or more TLR agonists is selected from the group consisting of SEQ ID NOS: 463-474; and a combination thereof.

In some embodiments, this disclosure provides SARS-CoV-2 immunogenic compositions comprising one or more rdAd anti-SARS-CoV-2 vectors comprising at least one polynucleotide sequence encoding at least one SARS-CoV-2 blocking protein; wherein the at least one polynucleotide sequence encodes at least one peptide or polypeptide: that induces an immune response that interferes with the binding of the SARS-CoV-2 S protein to its cellular receptor, directly interferes with the binding of the SARS-CoV-2 S protein to its cellular receptor, is an RBD binding agent, is an ACE2 binding agent, and/or is both an RBD binding agent and an ACE2 binding agent.

In some embodiments, this disclosure provides one or more polynucleotide(s) encoding a rdAd anti-SARS-CoV-2 vector disclosed herein. In some embodiments, this disclosure provides one or more rdAd anti-SARS-CoV-2 vectors produced upon expression of such polynucleotide(s) in a host cell. In some embodiments, this disclosure provides one or more compositions comprising such polynucleotides and/or rdAd anti-SARS-CoV-2 vectors, which in some embodiments is a pharmaceutical composition or pharmaceutical dosage form.

### Preferred Aspects of the Disclosure

Preferred aspects of this disclosure include:

An immunogenic composition comprising a replication defective adenoviral (rdAd) vector, wherein the rdAd vector is selected from:
a) an rdAd vector lacking a coding sequence encoding an exogenous, non-adenoviral, antigen;
b) an rdAd vector comprising an expression cassette comprising a SARS-CoV-2 antigen coding sequence encoding at least one SARS-CoV-2 antigen, optionally wherein said antigen comprises a SARS-CoV-2 spike (S) protein receptor binding domain (RBD);
c) an rdAd vector comprising an expression cassette comprising a coding sequence encoding at least one exogenous antigen of an infectious agent other than SARS-CoV-2;
d) a combination of the vectors of a) and b), wherein the rdAd vectors are administered together or separately;
e) a combination of the vectors of b) and c), wherein the rdAd vectors are administered together or separately;
f) a combination of any of the rdAd vectors of any of a), b), or c), wherein the rdAd vectors are administered together or separately;
g) a combination of two different types of rdAd vectors of b), wherein each type of rdAd vector comprises an expression cassette encoding at least one SARS-CoV-2 antigen different from that encoded by the other types of rdAd vectors in the combination, wherein the rdAd vectors are administered together or separately;
said immunogenic composition being configured to induce neutralizing antibody and/or cellular immune response against SARS-CoV-2 in a mammalian subject to which said immunogenic composition is administered.

The immunogenic composition of the prior aspect, wherein the expression cassette comprises a SARS-CoV-2 antigen coding sequence for spike (S) protein or the S1 domain of the spike protein.

The immunogenic composition of any prior aspect, wherein the expression cassette comprises a SARS-CoV-2 antigen coding sequence selected from the group consisting of SEQ ID NO: 3; a sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) homology and/or identity to SEQ ID NO: 3; a sequence present in SEQ ID NO: 12; a sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) homology and/or identity to SEQ ID NO: 12; SEQ ID NO: 15; a sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) homology and/or identity to SEQ ID NO: 15; SEQ ID NO: 446; a sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) homology and/or identity to SEQ ID NO: 446; any of SEQ ID NOS: 412-417; any SEQ ID NOS: 438-445, and SEQ ID NOS: 475-476 and 460; a sequence having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) homology and/or identity to any of SEQ ID NOS: 412-417 and SEQ ID NOS: 438-445, and SEQ ID NOS: 475-476 and 460.

The immunogenic composition of any prior aspect, wherein the SARS-CoV-2 antigen coding sequence encodes a spike protein sequence comprising a sequence where the S1/S2 cleavable site and/or S2′ are resistant to proteomic degradation, and/or a sequence where the fusion peptide has been deleted or modified to prevent its fusogenic activity, and/or a sequence where the intracellular domain has been modified or partially to alter the endoplasmic reticulum retention motif. The immunogenic composition of any prior aspect, wherein the expression cassette comprises a SARS-CoV-2 antigen coding sequence selected from the group consisting and/or a sequence including at least one of NSPQQAQSVAS (SEQ ID NO: 451), NSPSGAGSVAS (SEQ ID NO: 456) or NSP-VAS (SEQ ID NO: 461) at the S1/S2 cleavage site, KRSFIADA (SEQ ID NO: 453), PSKPSKQSF (SEQ ID NO: 457), PSKPSKNSF (SEQ ID NO: 458), PSKPSNASF (SEQ ID NO: 459) at the S2′ cleavage site, or SRLDPPEAEV (SEQ ID NO: 455), and/or any sequence modification presented in Table 1 and/or Table 2.

The immunogenic composition of any prior aspect, wherein the SARS-CoV-2 antigen coding sequence is a sequence presented in SEQ ID NO: 18, SEQ ID NO: 19, SEQ ID NO: 20, or an immunogenic fragment thereof.

The immunogenic composition of any prior aspect, wherein the SARS-CoV-2 antigen coding sequence encodes at least amino acids 331 to 527 of SEQ ID NO: 3.

The immunogenic composition of any prior aspect, wherein the SARS-CoV-2 antigen coding sequence encodes a spike protein receptor binding domain (RBD) sequence comprises one or more of the following substitutions: K417N, K417T, R403K, N439K, G446V, G446S, L452R, G476A, S477N, T478K, E484D, T4781, E484K, F490S, Q493R, S494P, P499H and/or N501Y.

The immunogenic composition of any prior aspect, wherein the SARS-CoV-2 antigen coding sequence encodes a spike protein receptor binding domain (RBD) sequence, or immunogenic fragment thereof, and/or comprises an amino acid sequence of SEQ ID NO: 12, SEQ ID NO: 13, SEQ ID NO 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NOS: 412-417, SEQ ID NOS: 438-445, SEQ ID NO: 446, SEQ ID NO: 460, SEQ ID NO: 475 and SEQ ID NO: 476; or an immunogenic fragment thereof.

The immunogenic composition of any prior aspect, wherein the expression cassette of the replication defective adenoviral vector further comprises a coding sequence encoding one or more of SARS-CoV-2 structural proteins envelope (E), membrane (M) or nucleocapsid (N).

The immunogenic composition of any prior aspect, comprising an additional replication defective adenoviral vector comprises a coding sequence encoding one or more of SARS-CoV-2 structural proteins envelope (E), membrane (M) or nucleocapsid (N).

The immunogenic composition of any prior aspect, wherein the expression cassette of the replication defective adenoviral vector comprises a coding sequence for a modified version of SEQ ID NO: 411 comprising:
one or more substitutions of any one or more of amino acids 333-388, 390-395, 397-399, 401-411, 413-415, 417-419, 424, 426-435, 437, 439-442, 444-446, 449, 450, 452, 453, 455-463, 465, 467-473, 475-479, 481-486, 490, 491, 493-495, 499-510, and/or 513-526; one or more substitutions of any one or more of amino acids 367, 403, 417, 439, 446, 449, 452, 453, 455, 456, 470, 473, 475, 476, 477, 478, 484, 486, 490, 493, 494, 495, 496, 499, 500, 501, 502, 503, 504, and/or 505; one or more substitutions selected from the group consisting of amino acid 367 (V) by F, I, L S or A, amino acid 403 (R) by K or S, 417 (K) by N or T; amino acid 439 (N) by K, amino acid 446 (G) by V, S or A; amino acid 449 (Y) by N; amino acid 452 (L) by L, M or Q, amino acid 453 (Y) by F; amino acid 455 (L) by F; amino acid 456 (F) by L; amino acid 470 (T) by I, A or N, amino acid 473 (Y) by V; amino acid 475 (A) by V; amino acid 476 (G) by S or A; amino acid 477 (S) by N, R, T, G, A or I; amino acid 476 (G) by S or A, amino acid 477 (S) by N, R, T, G, A or I, amino acid 478 (T) by I, K, R or A, amino acid 484 (E) by Q, K, D, A or R; amino acid 486 (F) by L or S; amino acid 490 (F) by L or S, amino acid 493 (Q) by L or R; amino acid 494 (S) by P or L, amino acid 495 (Y) by N or F; amino-acid 496 (G) by V or S, amino acid 499 (P) by H, S or R, amino acid 500 (T) by I; amino acid 501 (N) by Y, T or S; amino acid 502 (G) by R, D or C; amino acid 503 (V) by L, I or F; and, amino acid 504 (G) by V, D or S amino acid 505 (Y) by H, E, W or C.

The immunogenic composition of any prior aspect wherein the at least one antigen of an infectious agent other than SARS-CoV-2 is derived from an influenza virus.

The immunogenic composition of any prior aspect comprising a combination of any two of the rdAd vectors of a)-c).

The immunogenic composition of any prior aspect, wherein said immunogenic composition is a two-part composition comprising at least one composition comprising rdAd vectors of a)-c) and at least a second composition comprising a different rdAd vector of a)-c).

The immunogenic composition of any prior aspect, further configured to induce a combined mucosal, humoral and T cell protective immune response against SARS-CoV-2.

The immunogenic composition of any prior aspect, wherein the coding sequence encodes at least one or more B cell epitopes, one or more CD8+ T cell epitopes, and/or one or more CD4+ T cell epitopes.

The immunogenic composition of any prior aspect, wherein the coding sequence is codon optimized for the mammalian subject.

The immunogenic composition of any prior aspect, wherein the neutralizing antibodies are seroprotective against SARS-CoV-2 infection in a mammalian subject, optionally wherein the mammalian subject is a human being.

The immunogenic composition of any prior aspect, wherein the replication defective adenoviral vector is a human adenovirus, optionally Ad5 or Ad26.

The immunogenic composition of any prior aspect, wherein the replication defective adenoviral vector is a bovine adenovirus, a canine adenovirus, a non-human primate adenovirus, a chicken adenovirus, or a porcine or swine adenovirus.

The immunogenic composition of any prior aspect, wherein the replication defective adenoviral vector is an E1, E3, and/or E4 deleted or disrupted adenovirus.

The immunogenic composition of any prior aspect, wherein the SARS-CoV-2 antigen coding sequence encodes one or more peptides comprising one or more T cell epitopes of Table 3A, one or more groups of T cell epitopes of Table 3B, or SEQ ID NOS: 27-282; and/or one or more B cell epitopes of SEQ ID NOS. 25-68; and/or one or more of SEQ ID NOS. 328-369; optionally wherein the peptides are concatenated, and optionally separated by a linker amino acid sequence of two to ten amino acids.

The immunogenic composition of any prior aspect wherein the rdAd vector further comprises at least one molecular adjuvant selected from the group consisting of: one or more polypeptides or peptides that functions as a co-stimulatory component; one or more cytokines; one or more chemokines; one or more immune inhibitory proteins; one or more TLR agonists, optionally wherein the one or more TLR agonists is selected from the group consisting of SEQ ID NOS: 463-474; and a combination thereof.

The immunogenic composition of any prior aspect, wherein the rdAd vector comprises at least one polynucleotide sequence encoding at least one SARS-CoV-2 blocking protein; wherein the at least one polynucleotide sequence encodes at least one peptide or polypeptide: that induces an immune response that interferes with the binding of the SARS-CoV-2 S protein to its cellular receptor, directly interferes with the binding of the SARS-CoV-2 S protein to its cellular receptor, is an RBD binding agent, is an ACE2 binding agent, and/or is both an RBD binding agent and an ACE2 binding agent.

A polynucleotide encoding a rdAd vector of an immunogenic composition of any prior aspect, an rdAd vector produced upon expression of the polynucleotide in a host cell, and a composition comprising the rdAd vector.

A pharmaceutical formulation, comprising an effective amount of the immunogenic composition of any prior aspect; and, a pharmaceutically acceptable diluent or carrier, optionally wherein the diluent is phosphate-buffered saline.

The pharmaceutical formulation of any prior aspect configured for non-invasive administration.

The pharmaceutical formulation of any prior aspect configured for intranasal administration to the mammalian subject.

The pharmaceutical formulation of any prior aspect, wherein administration of the pharmaceutical formulation to the mammalian subject induces a protective immune response in the mammalian subject, optionally a combined mucosal, humoral and T cell protective immune response.

The pharmaceutical formulation of any prior aspect wherein the pharmaceutically acceptable carrier is in a spray or aerosol form.

The pharmaceutical formulation of any prior aspect, wherein the effective amount is at least 10⁷ viral particles (vp), at least 10⁸ viral particles (vp), or at least 10⁹ viral particles (vp).

The pharmaceutical formulation of any prior aspect, configured as a single intranasal dose.

The pharmaceutical formulation of any prior aspect, configured as two or more intranasal doses.

A pharmaceutical formulation suitable for a single dose intranasal administration to a human subject, comprising:
an effective amount of at least 10⁷ viral particle (vp) of the immunogenic composition of any prior aspect comprising at least one replication defective adenoviral vector comprising an expression cassette comprising a coding sequence encoding at least SARS-CoV-2 spike (S) protein receptor binding domain (RBD), or at least one immunogenic fragment thereof, wherein the effective amount induces a combined mucosal, humoral and T cell protective immune response; and,
a pharmaceutically acceptable diluent or carrier.

The pharmaceutical formulation of any prior aspect, wherein the formulation is configured to provide seroprotection to the human subject for at least 6 months against SARS-CoV-2.

The pharmaceutical formulation of any prior aspect, wherein the coding sequence is codon optimized for the human subject.

A pharmaceutical dosage for intranasal administration, comprising:
a pharmaceutical acceptable carrier in a spray or aerosol form admixed with an immunogenic composition of any prior aspect, wherein the dosage is configured for intranasal administration to non-invasively induce a protective immune response against SARS-CoV-2.

The pharmaceutical dosage of any prior aspect, wherein the immunogenic composition comprises an effective amount of at least 10⁷ viral particles (vp), at least 10⁸ viral particles (vp), or at least 10⁹ viral particles (vp).

The pharmaceutical dosage of any prior aspect, wherein the effective amount induces a combined mucosal, humoral and T cell protective immune response.

The pharmaceutical dosage of any one of any prior aspect, configured as a single dose to induce a protective immune response against coronavirus.

The pharmaceutical dosage of any one of any prior aspect, configured as two or more doses to induce a protective immune response against SARS-CoV-2.

A method for inducing an immune response against coronavirus, the method comprising administering an effective amount of the immunogenic composition of any prior aspect to a mammalian subject.

The method of any prior aspect, wherein the immune response is protective against SARS-CoV-2.

A method for inducing an immune response against SARS-CoV-2, the method comprising administering a pharmaceutical formulation of any prior aspect or a pharmaceutical dosage of any prior aspect to a mammalian subject.

The method of any prior aspect wherein the immune response is protective against SARS-CoV-2.

The method of any prior aspect, the method comprising intranasal administration of an effective amount of the immunogenic composition to the mammalian subject, wherein the immune response provides protection against challenge with SARS-CoV-2.

A method of inducing a combined mucosal, humoral and/or T cell protective immune response in a human subject against coronavirus comprising:
administering intranasally to a human subject a single dose of the coronavirus (SARS-CoV-2) pharmaceutical formulation of any prior aspect or a pharmaceutical dosage of any prior aspect, wherein the administration induces serum antibodies, mucosal antibodies and T cells against SARS-CoV-2, optionally whereby the human subject is seroprotected for at least about 6 months or more preferably about 9 months.

The method of any prior aspect, wherein the seroprotection lasts for at least 12 months, at least 13 months or at least 14 months.

The method of any prior aspect further comprising administering one or more anti-cytokine reagents to the human being to prevent and/or treat SARS-CoV-2, optionally wherein the one or more anti-cytokine reagents include one or more anti-IL-1α reagent(s), one or more anti-IL5 reagent(s), one or more anti-IL-6 reagent(s), one or more anti-IL-12 reagent(s), one or more anti-IL-17 reagent(s), one or more anti-MCP-1 reagent(s), one or more anti-TNF-α reagent(s), one or more anti-GM-CSF reagent(s), and/or one or more anti-RANTES reagent(s).

The method of any prior aspect, wherein the one or more anti-cytokine reagents does not include one or more anti-MIPα reagent(s) and/or one or more anti-RANTES reagent(s).

The method of any prior aspect wherein:
the one or more anti-cytokine reagent(s) are co-administered substantially with the effective amount of the immunogenic composition;
the one or more anti-cytokine reagent(s) are not administered substantially with the effective amount of the immunogenic composition;
the immunogenic composition is administered to the mammal once and the one or more anti-cytokine reagent(s) are administered multiple times; or
the immunogenic composition is co-administered to the mammal with the one or more anti-cytokine reagent(s), and the one or more anti-cytokine reagent(s) are subsequently administered to the mammal.

A method of treating or inhibiting the symptoms of a respiratory viral infection in a mammal, said respiratory viral infection causing elevated expression of interleukin-6 (IL-6), interleukin-1-alpha (IL-la) and/or interleukin-12 (IL-12) in the lung of said mammal, the method comprising: intranasally administering an effective amount of an E1 and E3 deleted adenoviral vector, or formulation or composition comprising the same, of any prior aspect to the subject, whereby expression of IL-6, IL-1α, and/or IL-12 in the lung is reduced thereby alleviating said symptoms for up to about 28 days following administration of the vector.

The method of any prior aspect wherein expression of monocyte chemoattractant protein 1 (MCP-1), IFN-γ, and/or RANTES, are increased in the lung following administration of the vector.

The method of any prior aspect wherein the expression of macrophage inflammatory protein 1 alpha (MIP-1α) and/or RANTES are not reduced following administration of the vector.

A method of inducing an anti-viral immune response in a mammalian subject in need thereof with, or at risk of, a respiratory viral infection, the method comprising: intranasal administration of an effective amount of an E1 and E3 deleted adenoviral vector, or formulation or composition comprising the same, of any prior aspect to the subject, wherein the anti-viral immune response generates increased expression of monocyte chemoattractant protein 1 (MCP-1) and/or interferon alpha (IFN-γ) following the administration step.

The method of any prior aspect wherein one or more additional anti-SARS-CoV-2 agents is administered to subjects before, essentially simultaneously, or after administration of E1 and E3 deleted adenoviral vector, optionally wherein said one or more additional agents is selected from the group consisting of chloroquine, azithromycin, remdesivir, an anti-inflammatory agent, and a combination thereof.

The method of any prior aspect wherein the mammalian subject is infected by SARS-CoV-2 prior to the administering of the pharmaceutical formulation thereto.

The method of any prior aspect wherein the mammalian subject is a human being.

An immunogenic composition comprising a replication defective adenoviral (rdAd) vector, wherein the rdAd vector is selected from:

a) an rdAd vector lacking a coding sequence encoding an exogenous, non-adenoviral, antigen;
b) an rdAd vector comprising an expression cassette comprising a SARS-CoV-2 antigen coding sequence encoding at least one SARS-CoV-2 antigen, optionally wherein said antigen comprises a SARS-CoV-2 spike (S) protein receptor binding domain (RBD);
c) an rdAd vector comprising an expression cassette comprising a coding sequence encoding at least one exogenous antigen of an infectious agent other than SARS-CoV-2;
d) a combination of the vectors of a) and b), wherein the rdAd vectors are administered together or separately;
e) a combination of the vectors of b) and c), wherein the rdAd vectors are administered together or separately;
f) a combination of any of the rdAd vectors of any of a), b), or c), wherein the rdAd vectors are administered together or separately;
g) a combination of two different types of rdAd vectors of b), wherein each type of rdAd vector comprises an expression cassette encoding at least one SARS-CoV-2 antigen different from that encoded by the other types of rdAd vectors in the combination, wherein the rdAd vectors are administered together or separately;
said immunogenic composition being configured to induce neutralizing antibody and/or cellular immune response against SARS-CoV-2 in a mammalian subject to which said immunogenic composition is administered, for use in the treatment or prevention of SARS-CoV-2.

Use of an immunogenic composition comprising a replication defective adenoviral (rdAd) vector, wherein the rdAd vector is selected from:
a) an rdAd vector lacking a coding sequence encoding an exogenous, non-adenoviral, antigen;
b) an rdAd vector comprising an expression cassette comprising a SARS-CoV-2 antigen coding sequence encoding at least one SARS-CoV-2 antigen, optionally wherein said antigen comprises a SARS-CoV-2 spike (S) protein receptor binding domain (RBD);
c) an rdAd vector comprising an expression cassette comprising a coding sequence encoding at least one exogenous antigen of an infectious agent other than SARS-CoV-2;
d) a combination of the vectors of a) and b), wherein the rdAd vectors are administered together or separately;
e) a combination of the vectors of b) and c), wherein the rdAd vectors are administered together or separately;
f) a combination of any of the rdAd vectors of any of a), b), or c), wherein the rdAd vectors are administered together or separately;
g) a combination of two different types of rdAd vectors of b), wherein each type of rdAd vector comprises an expression cassette encoding at least one SARS-CoV-2 antigen different from that encoded by the other types of rdAd vectors in the combination, wherein the rdAd vectors are administered together or separately;
said immunogenic composition being configured to induce neutralizing antibody and/or cellular immune response against SARS-CoV-2 in a mammalian subject to which said immunogenic composition is administered, characterized by being in the manufacture of a medicament to provide treatment or prevention of SARS-CoV-2.

An immunogenic composition comprising a replication defective adenoviral (rdAd) vector comprising a nucleic acid sequence encoding SEQ ID NO: 446 or a variant comprising at least 90%, or at least 95% identity to SEQ ID NO: 446.

The immunogenic composition of the prior aspect, wherein the nucleic acid sequence encodes SEQ ID NO: 15.

The immunogenic composition of any prior aspect, wherein the nucleic acid sequence encodes SEQ ID NO: 13.

The immunogenic composition of any prior aspect, wherein the nucleic acid sequence encodes one or more of SEQ ID NOS: 412-417, SEQ ID NOS: 438-445, SEQ ID NOS: 475-476 and SEQ ID NO: 460.

The immunogenic composition of any prior aspect, wherein the nucleic acid sequence encodes a sequence comprising one or more point mutations of SEQ ID NO: 3.

The immunogenic composition of any prior aspect, wherein the nucleic acid sequence encodes a sequence comprising one or more mutations at positions 333-388, 390-395, 397-399, 401-411, 413-415, 417-419, 424, 426-435, 437, 439-442, 444-446, 449, 450, 452, 453, 455-463, 465, 467-473, 475-479, 481-486, 490, 491, 493-495, 499-510, or 513-526 wherein amino acid numbering corresponds to SEQ ID NO: 411.

The immunogenic composition of any prior aspect, wherein the nucleic acid sequence encodes a sequence comprising one or more mutations at amino acid positions 367, 403, 439, 417, 446, 447, 449, 452, 453, 455, 456, 470, 473, 475, 476, 477, 478, 484, 486, 487, 490, 493, 494, 496, 499, 500, 501, 502, 503, 504, and/or 505, wherein amino acid numbering corresponds to SEQ ID NO: 411.

The immunogenic composition of the prior aspect, wherein the one or more mutations are selected from substitution of amino acid 417 (K) by N; substitution of amino acid 446 (G) by V, S or A; substitution of amino acid 449 (Y) by N; substitution at amino acid 453 (Y) by F; substitution of amino acid 455 (L) by F; substitution of amino acid 456 (F) by L; substitution of amino acid 473 (Y) by V; substitution of amino acid 475 (A) by V; substitution of amino acid 476 (G) by S or A; substitution of amino acid 477 (S) by N, R, T, G, A or I; substitution at amino acid 484 (E) by Q, K, D, A or R; substitution of amino acid 486 (F) by L or S; substitution of amino acid 453 (Y) by F; substitution of amino acid 493 (Q) by L or R; substitution of amino acid 495 (Y) by N or F; substitution of amino acid 500 (T) by I; substitution of amino acid 501 (N) by Y, T or S; substitution of amino acid 502 (G) by R, D or C; substitution of amino acid 503 (V) by L, I or F; or, substitution of amino acid 505 (Y) by H, E, W or C, wherein amino acid numbering corresponds to SEQ ID NO: 411.

The immunogenic composition of any prior aspect, wherein the nucleic acid sequence encodes a sequence comprising one or more mutations selected from K417T, K417N, E484K, L452R and/or N501Y, wherein amino acid numbering corresponds to SEQ ID NO: 411.

The immunogenic composition of any prior aspect, wherein the nucleic acid sequence encodes one or more of SEQ ID NOS: 412-417.

The immunogenic composition of any prior aspect, wherein the nucleic acid sequence encodes one or more of SEQ ID NOS: 438-443 or 460.

The immunogenic composition of any prior aspect, wherein the nucleic acid sequence encoding SEQ ID NO: 446 further comprises a leader sequence encoded by a nucleic acid sequence encoding a sequence selected from SEQ ID NOS: 418 to 437.

The immunogenic composition of any prior aspect, wherein the coding sequence is codon optimized for a mammalian subject.

The immunogenic composition of any prior aspect, wherein the replication defective adenoviral vector is a bovine adenovirus, a canine adenovirus, a non-human primate adenovirus, a chicken adenovirus, a porcine or swine adenovirus, or a human adenovirus.

The immunogenic composition of the prior aspect, wherein the non-human primate adenovirus is a chimpanzee or gorilla adenovirus.

The immunogenic composition of any prior aspect, wherein the replication defective adenoviral vector is a human adenovirus.

The immunogenic composition of the prior aspect, wherein the human adenovirus is Ad5 or Ad26.

A pharmaceutical formulation, comprising an effective amount of the immunogenic composition of any prior aspects, the composition comprising at least one pharmaceutically acceptable diluent or carrier, optionally wherein the diluent is phosphate-buffered saline.

The pharmaceutical formulation of the prior aspect, configured for non-invasive or intranasal administration, optionally wherein the pharmaceutically acceptable carrier is in a spray or aerosol form.

A method for inducing an immune response against SARS-CoV-2, the method comprising administering an effective amount of the immunogenic composition of any prior aspect to a human being.

The method of the prior aspect, wherein the effective amount is at least 10⁸ viral particles (vp), at least 10⁹ viral particles (vp), or at least 10¹⁰ viral particles (vp).

The method of any prior aspect, wherein the immunogenic composition is administered intranasally.

The method of any prior aspect, wherein the immune response against SARS-CoV-2 persists for at least 6 months, at least 9 months or at least 12 months after administration to a human subject.

The method of any prior aspect, wherein the immune response against SARS-CoV-2 comprises a mucosal IgA and/or T cell response against SARS-CoV-2 induced after administration of the immunogenic composition.

The method of any prior aspect, wherein the effective amount of the immunogenic composition reduces incidence of mild or moderate COVID-19-related diseases after the administration to the human subject.

The method of any prior aspect, wherein the effective amount of the immunogenic composition reduces incidence of severe COVID-19-related diseases after the administration to the human subject.

The method of any prior aspect, wherein the effective amount of the immunogenic composition reduces severity of COVID-19-related diseases after the administration to the human subject.

The method of any prior aspect, wherein the effective amount of the immunogenic composition reduces incidence of infection with SARS-CoV-2 after the administration to the human subject.

The method of any prior aspect, wherein the effective amount of the immunogenic composition reduces incidence of asymptomatic COVID-19 after the administration to the human subject.

The method of any prior aspect, wherein the effective amount of the immunogenic composition reduces transmission of SARS-CoV-2 after the administration to the human subject.

Other embodiments are also contemplated herein as would be understood by those of ordinary skill in the art.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Materials and Methods

Recombinant replication deficient adenovirus type 5. Replication defective human Adenovirus 5 (hAd5) are generated using previously described methods (Miravet, et al. Methods Mol Biol 2014, 1089, 159-173). In brief, the defective hAd5 lacks E1 and E3 genes to allow genomic space for the transgene insert. In some embodiments, the hAd5 lacks coding sequences for any exogenous antigens (i.e., non adenoviral antigens as in the AdE vector). In some embodiments, the hAd5 encodes one or more SARS-CoV-2 antigen(s) (referred to herein as “hAd5-SARS-CoV-2”). In some embodiments, the hAd5 encodes one or more influenza antigen(s) (as in the AdD vector). The hAd5-SARS-CoV-2 vectors and AdD vectors can encode any SARS-CoV-2 or influenza antigen, respectively, that is immunogenic regarding such antigen(s) in a human being or non-human animal (e.g., a mammal). For instance, the SARS-CoV-2 antigen insert can encode any protein (and/or any one or more fragment(s) and/or derivative(s) thereof) encoded by SEQ ID NO:1 (FIGS. 1A-J), such as any one or more of SEQ ID Nos. 2-11, 13 or 15 and/or any one or more fragments and/or derivatives thereof (e.g., a peptide of at least 3, 6, 9 or 11 contiguous amino acids thereof). The expression cassette is a cytomegalovirus (CMV) immediate early driven transgene, optionally encoding a tissue plasminogen activator signal sequence (tPA), followed by a codon optimized SARS-CoV-2 spike (S) cassette, inserted into the E1 region of the Adenovirus vector. The present immunogenic compositions are manufactured by propagation of the RD-Ad5 vector in replication-permissive CAP cells (Wölfel, et al. BMC Proceedings, 2011, 5(Supp 8):P133; Cevec), followed by purification of the virus from the infected cell harvest, and the final product may include the following excipients Tris HCl (pH 7.4), histidine, sucrose, sodium chloride, magnesium chloride, polysorbate 80, ethylenediaminetetraacetic acid, and ethanol, the final product stored at −80° C.

For animal and/or clinical studies, the present pharmaceutical formulations are supplied in a single-use glass vials each containing a nominal volume of 0.7 mL of a sterile frozen suspension of immunogenic composition (e.g., vaccine) formulated to deliver the nominal dose of 1×10⁷, 1×10⁸, 1×10⁹, 1×10¹⁰ or 1×10¹¹ viral particles (vp). Alternatively, the present pharmaceutical formulations are supplied in a single-use (pre-filled) syringe, optionally with an atomizer, (e.g., BD Accuspray) containing a nominal volume of 0.5 mL of a sterile frozen suspension of immunogenic composition (e.g., vaccine) formulated to deliver the nominal dose of 1×10⁷, 1×10⁸, 1×10⁹, 1×10¹⁰ or 1×10¹¹ viral particles (vp).

Focus Forming Assay. hAd5 vector (e.g., AdE, hAd5-SARS-CoV-2, AdD) titration is performed by focus forming assay (FFA) or other suitable assay. Briefly, regarding the FFA, cells expressing the viral receptor (e.g., ACE2 receptor (angiotensin converting enzyme 2)) are plated the day before the assay in a 96-well plate then virus stocks, serially diluted, allowed to infect cells, and then, optionally overlayed with methylcellulose. The cells are incubated at 37° C. for 48 hours followed by fixation with paraformaldehyde. Immunostaining using a coronavirus monoclonal antibody and a secondary antibody are used to visualize the formation of foci for individual infected cells for the hAd5-SARS-CoV-2 stock.

Western Blot. To confirm protein expression from the hAd5 (e.g., recombinant hAd5-SARS-CoV-2 virus), 293 cells (e.g, 293, Calu-3, Caco-2 or Vero) are infected with hAd5-SARS-CoV-2. After an incubation period (e.g., 48 hours) at 37° C., 5% CO₂ the cells are rinsed once with PBS and harvested using 2× NuPage buffer. Samples are heated at 95° C. for 10 minutes, cooled, and loaded onto NuPage 4-12% gels. Protein are transferred to nitrocellulose membrane, blocked with non-fat milk, and probed with one or more types of primary antibodies in TBST, each having specificity for one or more SARS-CoV-2 antigens. After overnight incubation, membranes are washed 3 times with TBST and blotted with fluorescently conjugated secondary antibodies (molecular probes). After one hour in secondary, membranes are washed three times in TB ST and one time in PBS, then fluorescent signal are captured with an imager.

Intracellular Cytokine stain. Spleens are harvested from vaccinated mice eight days post vaccination. Spleens are ground over a 100 μm cell strainer and brought up in RPMI with 10% FBS and HEPES. Approximately 10⁶ cells are plated per well in a round bottom 96 well plate and stimulated for 6 hours at 37° C., 5% CO₂ in the presence of 10 μg/ml brefeldin A and either α-CD3 (2C11 clone) or 10 μg of peptide in 90% DMSO. Following peptide stimulation, cells are washed once with PBS and stained for surface markers. Cells are then fixed and permeablized and stained for intracellular markers (e.g. IFN-γ). The cells are analyzed by flow cytometry.

ELISA. Polystyrene 96-well plates are coated overnight at 4° C. with 1 μg/ml of SARS-CoV-2 antigen in sodium carbonate buffer (pH 9.3). Plates are washed three times in PBS with 0.02% Tween 20 and blocked with non-fat dried milk for one hour at 37° C. with PBS, 2% BSA, and 0.02% Tween 20. Serum from hAd5 (e.g., hAd5-SARS-CoV-2) vaccinated mice are serially diluted in PBS then incubated at 37° C. Plates are washed four times with PBS with 0.02% Tween 20 and incubated with labeled secondary antibody for one hour. After washing and incubation as needed the plates are read using a microplate reader.

Vaccination with Recombinant replication deficient adenovirus type 5. Mice are anesthetized using Ketamine/Xylazine (90 mg/kg: 10 mg/kg), and then vaccinated with an appropriate volume intranasally of 1×10⁷ particles of hAd5-SARS-CoV-2 diluted in PBS.

Animal Challenge. SARS-CoV-2 is diluted in sterile PBS pH 7.4 to obtain a suitable final concentration of SARS-CoV-2 per mouse (e.g., those expressing the viral receptor) in a final volume of 10-50 μL. Virus challenge is performed by intranasal administration 21 days post hAd5-SARS-CoV-2 vaccination. Mice are anesthetized during this procedure using Ketamine/Xylazine (90 mg/kg: 10 mg/kg). After challenge, each mouse is examined for visible trauma and, placed back into its cage for recovery.

Clinical Monitoring. Animals are observed for clinical outcomes daily after SARS-CoV-2 challenge. Body weight changes, symptoms of SARS-CoV-2 virus infection, and mortality are recorded for each animal daily.

### Example 2: AdE Compositions for Immunization Against Respiratory Infection

The studies described in this example evaluated the immune response following administration of AdE for prevention and/or treatment of respiratory infection (e.g., as may be caused by influenza), including measurement of serum antibody levels by hemagglutination inhibition assay and secretory IgA levels in lung lavage. Cellular immunity was evaluated by quantitation of cells, in lung lavage, releasing IFN-γ and IL-4 by ELISpot assay. In addition, adenovirus-specific immunity was evaluated by adenovirus neutralization using serum from vaccinated mice. This study determined the ability of the empty adenovirus vector (AdE) to provide protection against influenza A H1N1, H3N2, H5N1, and influenza B virus challenge infections in mice. In addition, cytokine levels in lung lavage were evaluated following vaccination and challenge in an attempt to determine the mechanism of protection afforded by the AdE vector.

Materials and Methods

Abbreviations used in this example include: IL—interleukin; MCP—monocyte chemoattractant protein; IFN—interferon; TNF—tumor necrosis factor, MIP—macrophage inflammatory protein; GM-C SF—granulocyte/macrophage colony stimulating factor; and RANTES—regulated upon activation, normal T cell expressed and secreted.

Animals: Female six week-old BALB/c mice were obtained from Charles River Laboratories. The mice were quarantined for 72 hours before use and maintained on Teklad Rodent Diet (Harlan Teklad) and tap water at the Laboratory Animal Research Center of Utah State University.

Virus: Influenza A/California/04/2009 (pandemic H1N1), strain designation 175190, was received from Dr. Elena Govorkova, Department of Infectious Diseases, St. Jude Children's Research Hospital, Memphis Tenn. The virus was adapted to replication in the lungs of BALB/c mice by 9 sequential passages through mouse lungs. Virus was plaque purified in Madin-Darby canine kidney (MDCK) cells (American Type Culture Collection, Manassas, Va.) and a virus stock was prepared by growth in embryonated chicken eggs and then MDCK cells. Influenza A/Victoria/3/75 (H3N2) virus was obtained from the American Type Culture Collection (Manassas, Va.). The virus became lethal to mice after seven serial passages in the lungs of infected animals. Following mouse-adaptation a virus stock was prepared by growth in MDCK cells. Influenza A/Vietnam/1203/2004 (H5N1) was obtained from the Centers for Disease Control (Atlanta, Ga.). Viral propagation and assays were done in MDCK cells. Parent virus was passaged once to prepare a challenge pool. Influenza B/Sichuan/379/99 virus was obtained from the Centers for Disease Control (Atlanta, Ga.). The virus was propagated twice in MDCK cells, and then passaged serially 10 times in mice. Following mouse-adaptation a virus stock was prepared by growth in MDCK cells.

AdE Composition: The virus titer for the AdE was 6.4×10⁹ infection forming units (ifu)/ml (3.2×10⁸ ifu/0.05 ml). The vaccine was administered by the intranasal route in a 50 μl volume on a single occasion (see experimental design).

Experimental design: Animal numbers and study groups are described in Tables 1 to 3. Groups of mice were vaccinated on study day 0 or 20 by the intranasal route. The placebo groups received 50 μl physiological sterile saline (PSS) by the same route. For influenza virus challenge, mice were anesthetized by i.p. injection of ketamine/xylazine (50 mg/kg//5 mg/kg) prior to intranasal challenge with 90 μl of influenza A/CA/04/2009 (H1N1p), A/Victoria/3/1975 (H3N2), B/Sichuan/379/1999 or 75 μl of influenza A/Vietnam/1203/2004 (H5N1). The challenge dose was approximately 3×LD50 CCID50 (cell culture infectious doses) of virus per mouse. All mice were administered virus challenge on study day 22. Following challenge all mice were observed for weight loss and mortality through day 21 post-challenge.

TABLE 11
Study Groups Observed for Morality Rates and Body Weight

| | | | Vaccine | | | | |
|---|---|---|---|---|---|---|---|
| No. | Group | Infected | Dosage | | Challenge | | Observations/ |
| Mice | No. | Y or N | (IFU/mouse) | Day/Route | Virus | Day | Testing |
| 10 | 1 | Yes | None (Placebo) | Day 0, IN | I^{A}/CA/04/2009 | Day 22 | Survival and |
| | | | | | (H1N1) | | weight |
| 10 | 3 | Yes | None (Placebo) | Day 0, IN | I^{A}/Vic/3/1975 | Day 22 | determination |
| | | | | | (H3N2) | | |
| 10 | 5 | Yes | None (Placebo) | Day 0, IN | I^{A}/Vietnam/1203/2004 | Day 22 | |
| | | | | | (H5N1) | | |
| 10 | 7 | Yes | None (Placebo) | Day 0, IN | I^{B}/Sichuan/379/1999 | Day 22 | |
| 10 | 9 | Yes | AdE (3.2 × 10⁸) | Day 0, IN | I^{A}/CA/04/2009 | Day 22 | |
| | | | | | (H1N1) | | |
| 10 | 11 | Yes | AdE (3.2 × 10⁸) | Day 0, IN | I^{A}/Vic/3/1975 | Day 22 | |
| | | | | | (H3N2) | | |
| 10 | 13 | Yes | AdE (3.2 × 10⁸) | Day 0, IN | I^{A}/Vietnam/1203/2004 | Day 22 | |
| | | | | | (H5N1) | | |
| 10 | 15 | Yes | AdE (3.2 × 10⁸) | Day 0, IN | I^{B}/Sichuan/379/1999 | Day 22 | |
| 10 | 17 | Yes | None (Placebo) | Day 20, IN | I^{A}/CA/04/2009 | Day 22 | |
| | | | | | (H1N1) | | |
| 10 | 19 | Yes | None (Placebo) | Day 20, IN | I^{A}/Vic/3/1975 | Day 22 | |
| | | | | | (H3N2) | | |
| 10 | 21 | Yes | None (Placebo) | Day 20, IN | I^{A}/Vietnam/1203/2004 | Day 22 | |
| | | | | | (H5N1) | | |
| 10 | 23 | Yes | None (Placebo) | Day 20, IN | I^{B}/Sichuan/379/1999 | Day 22 | |
| 10 | 25 | Yes | AdE (3.2 × 10⁸) | Day 20, IN | I^{A}/CA/04/2009 | Day 22 | |
| | | | | | (H1N1) | | |
| 10 | 27 | Yes | AdE (3.2 × 10⁸) | Day 20, IN | I^{A}/Vic/3/1975 | Day 22 | |
| | | | | | (H3N2) | | |
| 10 | 29 | Yes | AdE (3.2 × 10⁸) | Day 20, IN | I^{A}/Vietnam/1203/2004 | Day 22 | |
| | | | | | (H5N1) | | |
| 10 | 31 | Yes | AdE (3.2 × 10⁸) | Day 20, IN | I^{B}/Sichuan/379/1999 | Day 22 | |

| | | | |
|---|---|---|---|
| 5 | 2 | No | Normal controls observed for weight gain |

TABLE 12
Study Group Used for Cytokine Analysis

| | | | Vaccine | | | | |
|---|---|---|---|---|---|---|---|
| No. | Group | Infected | Dosage | | Challenge | | Observations/ |
| Mice | No. | Y or N | (IFU/mouse) | Day/Route | Virus | Day | Testing |
| 20 | 1a | Yes | None (Placebo) | Day 0, IN | I^{A}/CA/04/2009 | Day 22 | Sac 5 mice per |
| | | | | | (H1N1) | | group for lung |
| 20 | 5a | Yes | AdE (3.2 × 10⁸) | Day 0, IN | I^{A}/CA/04/2009 | Day 22 | lavage on day |
| | | | | | (H1N1) | | 3 and 6 post- |
| 20 | 9a | Yes | None (Placebo) | Day 20, IN | I^{A}/CA/04/2009 | Day 22 | vacc. |
| | | | | | (H1N1) | | Sac 5 mice per |
| 20 | 13a | Yes | AdE (3.2 × 10⁸) | Day 20, IN | I^{A}/CA/04/2009 | Day 22 | group for lung |
| | | | | | (H1N1) | | lavage on day |
| | | | | | | | 3 and 6 post- |
| | | | | | | | challenge. |

TABLE 13
Negative Controls for Cytokine Analysis

| | | | Vaccine | | |
|---|---|---|---|---|---|
| No. | Group | Infected | Dosage | | |
| Mice | No. | Y or N | (IFU/mouse) | Day/Route | Observations/Testing |
| 6 | 4 | No | None (Placebo) | Day 0, IN | Sac 3 mice per group for lung |
| 6 | 6 | No | AdE (3.2 × 10⁸) | Day 0, IN | lavage on days 25 and 28 post- |
| | | | | | vaccination. |

Groups of mice were vaccinated on study day 0 or 20 by the intranasal route. The placebo groups received 50 μl physiological sterile saline (PSS) by the same route. For influenza virus challenge, mice were anesthetized by i.p. injection of ketamine/xylazine (50 mg/kg//5 mg/kg) prior to intranasal challenge with 90 μl of influenza A/CA/04/2009 (H1N1p), A/Victoria/3/1975 (H3N2), B/Sichuan/379/1999 or 75 μl of influenza A/Vietnam/1203/2004 (H5N1). The challenge dose was approximately 3×LD50 CCID50 (cell culture infectious doses) of virus per mouse. All mice were administered virus challenge on study day 22. Following challenge all mice were observed for weight loss and mortality through day 21 post-challenge.

Statistical analysis: Kaplan-Meier survival curves were generated and compared by the Log-rank (Mantel-Cox) test followed by pairwise comparison using the Gehan-Breslow-Wilcoxon test in Prism 5.0f (GraphPad Software Inc., La Jolla, Calif.). The mean body weights were analyzed by analysis of variance (ANOVA) followed by Tukey's multiple comparison test using Prism 5.0f.

Bronchioalveolar lavage (BAL): The lavage procedure was begun immediately after blood collection and was completed within 5 to 10 min of each animal's death. A volume of 0.75 ml of phosphate buffered saline (PBS) was slowly delivered into the lung through the tracheal tube. Immediately after delivery the fluid was slowly withdrawn by gentle suction and the samples stored at −80. The procedure was repeated a total of three times and lavage fluids from each mouse were pooled.

Lung virus titer determination: BAL samples were centrifuged at 2000×g for 5 minutes. Varying 10-fold dilutions of BAL supernatants were assayed in triplicate for infectious virus in MDCK cells, with virus titers calculated as described previously (1, 2). Virus titer differences were evaluated by ANOVA on log-transformed values assuming equal variance and normal distribution. Following ANOVA, individual treatment values were compared to placebo control by Tukey's pair-wise comparison test using Prism 5.0f.

Lung cytokine/chemokine determinations: A sample (200 μl) from each lung lavage was tested for cytokines and chemokines using a chemiluminescent ELISA-based assay according to the manufacturer's instructions (Quansys Biosciences Q-Plex™ Array, Logan, Utah). The Quansys multiplex ELISA is a quantitative test in which 16 distinct capture antibodies have been applied to each well of a 96-well plate in a defined array. Each sample supernatant was tested at 2 dilutions for the following: IL-1α, IL-1β, IL-2, IL-3, IL-4, IL-5, IL-6, IL-10, IL-12p70, IL-17, MCP-1, IFN-g, TNF-α, MIP-1α, GM-CSF, and RANTES.

Cytokine and chemokine titers are reported in pg/ml of lung lavage fluid. Titer differences were evaluated by ANOVA on values assuming equal variance and normal distribution. In addition, treatment group mean values were evaluated by two-way ANOVA for effects based on the day post-infection using Prism 5.0f.

Results and Discussion

This study determined the ability of the empty adenovirus vector (AdE) to provide protection against influenza A H1N1, H3N2, H5N1, and influenza B virus challenge infections in mice. In addition, cytokine levels in lung lavage were evaluated following vaccination and challenge with influenza A/CA/04/2009 (pandemic H1N1) virus in an attempt to determine the mechanism of protection afforded by the AdE vector. Mice were vaccinated with 3.2×10⁸ ifu/50 μl of AdE by the intranasal route. A single vaccination was given three-weeks before challenge infection. In addition, this study evaluated the antiviral effects of the AdE-vector when administered 2 days before challenge infection. Following infection, all mice were observed for weight loss and mortality through day 21 post-challenge.

The AdE vector was found to provide 100% protection from challenge with influenza A/CA/04/2009 (pandemic H1N1) virus when administered 20 days before challenge, and provided 80% protection when administered two (2) days before challenge. The AdE vector provided 90% protection from influenza A/Victoria/3/75 (H3N2) virus when administered 20 days before challenge. However, the protection afforded by the AdE vector administered two (2) days before challenge was not significant. The AdE vector administered 20 days before influenza A/Vietnam/1203/2004 (H5N1) virus challenge did not provide protection from mortality, but did increase the mean day of death significantly. However, no protection from influenza A/Vietnam/1203/2004 (H5N1) was provided when the AdE vector was administered two (2) days before challenge. The AdE vector also provided 100% protection from influenza B/Sichuan/379/9 virus when administered 20 days before challenge. In addition, the AdE vector provided 90% protection when administered two (2) days before challenge with influenza B/Sichuan/379/9 virus. The AdE vector only provided significant protection from weight loss following challenge by the influenza B/Sichuan/379/9 virus. Both times of AdE administration, day 0 and day 20, provided protection from weight loss following challenge. The groups of mice receiving AdE 20 days before challenge showed a 1-2 log reduction in influenza A/CA/04/2009 (pandemic H1N1) virus titer compared to placebo controls on both days.

In an attempt to identify the immune mechanism of protection afforded by immunization with the AdE vector, the expression of cytokines and chemokines in lung lavage following influenza A/CA/04/2009 (pandemic H1N1) virus infection was determined. Expression of of IL-1α, IL-1β, IL-2, IL-3, IL-4, IL-5, IL-6, IL-10, IL-12p70, IL-17, MCP-1, IFN-γ, TNF-α, MIP-1α, GM-CSF, and RANTES were measured on days 3 and 6 post-vaccination, days 25 and 28 post-vaccination, and on days 3 and 6 post-challenge (which is the same as days 25 and 28 post-vaccination). Significant changes in cytokine and chemokine levels were observed for IL-1α, IL-6, IL-12p70, MCP-1, IFN-γ, and RANTES. Significant decreases, compared to placebo controls, were observed after challenge infection for IL-1α, IL-6, and IL-12p70. No significant changes were observed for IL-1b, IL-2, IL-3, IL-4, IL-5, IL-10, IL-17, TNF-α, MIP-1α, and GM-CSF. A significant decrease (p<0.01) in IL-1α expression was observed on day 3 post-challenge when the AdE was administered 20 days before challenge. A significant decrease (p<0.01) in IL-6 expression in lung lavage following vaccination with AdE and challenge was observed (e.g., on day 6 post-challenge when the AdE was administered 20 days before challenge)). A significant decrease (p<0.01) in IL-12p70 expression in lung lavage following vaccination with AdE and challenge was observed on day 3 post-challenge when the AdE was administered 20 days before challenge. Significant (p<0.01) changes in expression of MCP-1 and IFN-γ in lung lavage were observed after vaccination and after challenge infection. MCP-1 levels increased on days 3, 6, 25, and 28 for all AdE treated groups post-vaccination. However, the MCP-1 levels decreased on day 6 post-challenge when the AdE was administered 20 days before challenge. IFN-γ levels increased on day 6 post-vaccination when the AdE was administered two (2) days before challenge, and remained elevated until days 25 and 28 (p<0.001) post-vaccination when AdE was administered 20 days before challenge. In addition, IFNγ levels increased approximately 10-fold on day 6 post-challenge when AdE was administered two (2) days before challenge. Significant changes in levels of RANTES were observed on days 3 (p<0.0001), 6 (p<0.001) and 25 (p<0.01) post-vaccination). This data is summarized in Table 14.

TABLE 14

| | | | | |
|---|---|---|---|---|
| | | Lung Lavage | | |
| AdE | | Collection | | |
| Admin. | Infected? | Post-AdE | Decreased | Increased |

| | | | |
|---|---|---|---|
| Day 0 | No | Day 3 | MCP-1 |
| | | | RANTES (CCL5) |
| Day 0 | No | Day 6 | MCP-1 |
| | | | IFN-γ |
| | | | RANTES (CCL5) |
| Day 0 | No | Day 25 | MCP-1 |
| | | | IFN-γ |
| | | | RANTES (CCL5) |
| Day 0 | No | Day 28 | MCP-1 |
| | | | IFN-γ |

Challenge on Day 22 of Study

| | | | | |
|---|---|---|---|---|
| Day 0 | Yes | Day 25 | IL-1α | |
| | | (Day 3 post- | IL-12 | |
| | | challenge) | | |
| Day 0 | Yes | Day 28 Post-AdE | IL-6 | |
| | | (Day 6 post- | MCP-1 | |
| | | challenge) | | |
| Day 20 | Yes | Day 5 (Day 3 post- | | |
| | | challenge; same as | | |
| | | Day 25 of the study) | | |
| Day 20 | Yes | Day 8 (Day 6 post- | | IFN-γ |
| | | challenge; same as | | |
| | | Day 28 of the study) | | |

Conclusions

This example describes the use of an empty adenovirus vector (AdE) as a vaccine against influenza A H1N1, H3N2, H5N1, and influenza B virus challenge infections in mice. A single vaccination was given either three (3) weeks before challenge infection, or two (2) days before challenge infection. Remarkably, protection was provided against all challenge strains when the AdE was administered 20 days before challenge. The survival effects observed against the H5N1 virus was not actually from mortality, but rather an increase in mean day of death. In addition, protection was provided against the H1N1 and influenza B virus challenge by the AdE vector, when administered two (2) days before challenge. Protection observed two (2) days after vaccination suggests an innate immune mechanism. However, innate immunity is not expected to last longer than four (4) days post-infection. Therefore, the observation that mice can be protected from virus challenge after only two (2) days, in addition to three (3) weeks after vaccination, suggests more than one mechanism of action. One possible mechanism, suggested by the increased levels of MCP-1 and IFN-γ both post-vaccination and post-challenge, is that vaccination with AdE leads to an increase in MCP-1, which recruits monocytes, neutrophils, and/or lymphocytes, which then stimulates production of IFN-γ.

### Example 3: AdE Human Clinical Trial for SARS-CoV-2 Vaccination

In this example, the use of intranasal (i.n.) administration of AdE vectors (i.e., replication deficient ΔE1E3 adenovirus type 5 (Ad5)) viral particles without an exogenous non-Ad pathogen antigen encoded in the Ad5 genome) to confer prophylactic therapy against SARS-CoV-2 is described. To establish the immunogenic and/or protective capacity of an immunogenic composition comprising AdE vectors against SARS-CoV-2, an AdE immunogenic composition comprising AdE viral particles (vp), are administered to human being subjects and tested for its effect on the immune response against SARS-CoV-2 therein. To do so, a randomized, double-blind, placebo-controlled, dose-escalation clinical trial to evaluate the safety and immunogenicity of an AdE immunogenic composition in healthy adults 18 to 49 years of age can be carried out. Subjects are typically screened within 28 days of randomization (Day 1).

For instance, a study can comprise two parts; part A which evaluates safety, and part B which evaluates immunogenicity, of the AdE immunogenic composition. In part A, approximately 120 subjects who meet all inclusion and no exclusion criteria and provided written informed consent are enrolled into four sequential cohorts of 30 subjects each defined by the AdE dose (1×10⁸, 1×10⁹, 1×10¹⁰, and 1×10¹¹ vp). Within each cohort (and the sentinel group in the first dose cohort), subjects are randomized in a 4:1:1 ratio to receive one intranasal dose of the AdE immunogenic composition (Day 1) or one intranasal dose of placebo (normal saline) (Day 1). The AdE immunogenic composition and placebo are administered in a double-blind fashion. Reactogenicity can be ascertained by determining counts and percentages of subjects with local events including but not limited to nasal irritation, sneezing, nasal congestion, cough, sore throat, change in smell, change in taste, change in vision, eye pain, pain, tenderness, induration, erythema, regional lymphadenopathy, and systemic events (headache, fatigue, myalgia, nausea, vomiting, diarrhea, coughing, chills, fever) for 14 days after vaccination. Adverse Events (AEs) are determined as counts and percentages of subjects with AEs from Day 1 to Day 57; medically attended AEs (MAAEs), serious AEs (SAES), and new-onset chronic illnesses (NCIs) from Day 1 to Day 181 following administration of the AdE immunogenic composition. For instance, targeted and symptom-driven physical examinations including vital signs can be carried out on days 4, 8, 15, 22, 29, and 57; an electrocardiogram can be carried out on day 57; safety laboratory tests can be carried out on days 8 and 57; and serum samples taken for immunogenicity testing at days 8, 15, 22, 29, 57, 91, 181, and 361. The primary endpoint for evaluation of the safety profile in Part A is the number and percentage (95% confidence interval (CI)) of subjects with solicited and unsolicited AEs recorded postvaccination. Safety analyses is performed using the Safety Population. The number (percentage, 95% CI) of subjects with local events and systemic events is summarized by group, as is reactogenicity. The number (percentage, 95% CI) of subjects with AEs from Day 1 to Day 57 (including MAAEs, NCIs, SAES) is summarized for each Medical Dictionary for Regulatory Activities system organ class (SOC) by preferred term (PT) and group. The number (percentage) of subjects with MAAEs, with NCIs, and with SAES from Day 1 to Day 181 is summarized in a similar fashion. The number (percentage, 95% CI) of subjects with AEs by severity and by relationship to investigational product (IP) is also summarized. Listings of AEs, MAAEs, NCIs, and SAES are provided.

In part B, the immunogenicity of the AdE immunogenic composition is determined. Following administration of the AdE immunogenic composition by intranasal spray as a single dose of 1×10⁸, 1×10⁹, 1×10¹⁰, and 1×10¹¹ vp and as two doses (3 weeks apart) of the highest well tolerated of these doses to subjects, the immune response can be measured by ELISA of serum to measure anti-SARS-CoV-2 antigen antibodies, and the GMT, geometric mean ratio (GMR) (the ratio of postvaccination and pre-vaccination GMTs within the same dose group), and responder rate (≥four-fold rise in IgG post dose) determined. For instance, approximately 25 subjects who meet all inclusion and no exclusion criteria and provided written informed consent are randomized in a 4:1 ratio to receive two intranasal doses of the AdE immunogenic composition at the highest well tolerated dose from Part A or placebo 21 days apart (Days 1 and 22). The AdE immunogenic composition and placebo are administered in a double-blind fashion. Intranasal doses of the AdE immunogenic composition and placebo are administered to subjects in a sitting or reclined position. In part B, targeted and symptom-driven physical examination including vital signs can be carried out on days 8, 15, 22, 29, 36, 43, 50, and 57; an electrocardiogram can be carried out on day 57; safety laboratory tests on days 8, 29, and 57; serum samples taken for immunogenicity testing at days 8, 15, 22, 29, 57, 91, 181, and 361; and nasopharyngeal swabs collected on days 8, 15, 29, 36, 43, 50, 57, and 91. Nasopharyngeal samples collected at Screening and on Days 29 and 57 can also be subsequently tested for evaluation of mucosal immune response.

In some embodiments, the clinical trial can be carried out using patients already infected by SARS-CoV-2 and time to clinical improvement and/or recovery determined (or, in some embodiments, a cohort of the patients tested). A primary outcome measure is Time to Clinical Improvement (TTCI) and/or Time to Clinical Recovery (TTCR) which are determined for up to 28 days following administration of the AdE composition as described above. TTCI is defined as the time (in days) from initiation of study treatment (active or placebo) until a decline of two categories from status at randomization on a six-category ordinal scale of clinical status which ranges from 1 (discharged) to 6 (death). The six-category ordinal scale is as follows: 6. Death; 5. ICU, requiring extracorporeal membrane oxygenation (ECMO) and/or invasive mechanical ventilation (IMV); 4. Intensive care unit (ICU)/hospitalization, requiring non-invasive mechanical ventilation (NIV)/high-flow nasal cannula (HFNC) therapy; 3. Hospitalization, requiring supplemental oxygen (but not NIV/HFNC); 2. Hospitalization, not requiring supplemental oxygen; and, 1. Hospital discharge or meet discharge criteria (discharge criteria are defined as clinical recovery, i.e. fever, respiratory rate, oxygen saturation return to normal, and cough relief). Secondary outcome TTCI measures include all cause mortality (baseline SpO2 during screening, PaO2/FiO2<300 mmHg or a respiratory rate ≥24 breaths per min without supplemental oxygen); frequency of respiratory progression (SPO2≤94% on room air or PaO2/FiO2<300 mmHg and requirement for supplemental oxygen or more advanced ventilator support); time to defervescence (in those with fever at enrolment); time to cough reported as mild or absent (in those with cough at enrolment rated severe or moderate); time to dyspnea reported as mild or absent (on a scale of severe, moderate, mild absent, in those with dyspnoea at enrolment rated as severe or moderate,); frequency of requirement for supplemental oxygen or non-invasive ventilation; time to SARS-CoV-2 RT-PCR negative in throat swab, sputum, lower respiratory tract specimen, and/or upper respiratory tract specimen; change (reduction) in SARS-CoV-2 viral load in throat swab, sputum, lower respiratory tract specimen, and/or upper respiratory tract specimen as assessed by area under viral load curve (e.g., as determined using polymerase chain reaction (PCR)); frequency of requirement for mechanical ventilation; and, frequency of serious adverse events. TTCI is defined as the time (in hours) from initiation of study treatment (active or placebo) until normalization of fever, respiratory rate, and oxygen saturation, and alleviation of cough, sustained for at least 72 hours. The primary TTCR outcome measures include normalization and alleviation criteria; fever—≤36.9° C. or -axilla, ≤37.2° C. oral; respiratory rate—≤24/minute on room air; oxygen saturation—>94% on room air; and, cough—mild or absent on a patient reported scale of severe, moderate, mild, absent. The secondary TTCR outcome measures are the same as the TTCI secondary outcomes listed above.

Standard clinical trial design and statistical methods are used in the analyses thereof. For instance, the sample size for this study is selected as adequate and reasonable for an initial review of the safety and immunogenicity profile of the AdE immunogenic composition at doses to be well tolerated, rather than for statistical power (e.g., 120 subjects as described above). The sample size permits initial estimates of reactogenicity. For example, given a total of 100 subjects receiving AdE immunogenic composition, the study is designed to have an 80% probability of detecting at least one AE that occurred at a rate of 1.6%. If no SAEs were observed among the 100 subjects who received AdE immunogenic composition, an approximation to the 1-sided upper bound of the 95% confidence interval (CI) on the rate of SAE occurrence would be 3%. Immunology analyses are conducted using the Evaluable and Per-protocol (PP) Populations with primary conclusions drawn from the PP Population. Analyses based on the Evaluable Population are undertaken and presented only if >1 subject in any one group were excluded from the PP Population. With the exception responder analyses, as described below, no imputation for missing data is performed. Data is transformed as appropriate prior to analysis. Baseline is defined as the sample collected prior to AdE immunogenic composition administration on Day 1. The primary variables of interest for assessment of humoral and cellular immune response to SARS-CoV-2 (e.g., cell mediated responses) are determined. In some embodiments, comparisons of responders in each AdE immunogenic composition dose group against the placebo group can also be conducted using Fisher's exact test. To determine the effect of pre-dose Ad5 serum antibody levels on immunogenicity of AdE immunological composition on Day 29 (Part A) or Day 50 (Part B), analyses are performed using ANCOVA with baseline Ad5 titer as a covariate. Mucosal immunogenicity analyses are conducted using the Evaluable and PP Populations. No imputation for missing data is performed. Endpoints analyzed are GMT and GMR for IgA antibody level measured by ELISA. Summary statistics for continuous parameters (safety laboratory tests and vital signs) are presented by group as follows: pre-vaccination, postvaccination, and change from pre-vaccination to postvaccination assessment. The number and percentage of subjects with postvaccination safety laboratory values or vital sign values recorded as newly abnormal (i.e., an event with an increase in the toxicity grade relative to the baseline value and with a severity grade of moderate or higher) after study vaccination are tabulated. Shift tables that cross-tabulate the pre-vaccination and postvaccination safety laboratory values of each subject by severity grade are prepared. Summaries of the number and percentage of subjects with normal, abnormal not clinically significant, and abnormal clinically significant ECG interpretations are presented. For shedding of the Ad5 vector, data are summarized by count and percent positive by time point, along with median copy number. The median duration of Ad5 shedding, interquartile range, minimum and maximum duration of Ad5 shedding are presented for each AdE immunogenic composition group and all immunological composition dose groups combined. Viral culture results for evaluation of adenovirus infection are also listed.

These studies will show that the AdE composition can be used to induce an anti-SARS-CoV-2 immune response in human beings (e.g., it is an immunogenic composition), and exhibits an acceptable safety profile. It is preferred that the immune response is statistically significant, and even more preferably, a protective immune response (i.e., it is a SARS-CoV-2 vaccine). In preferred embodiments, the data shows the AdE composition can be used to treat subjects infected by SARS-CoV-2 (e.g., hospitalized patients).

### Example 4

A. Generation of Recombinant human Adenovirus type 5 SARS-CoV-2 (hAd5-SARS-CoV-2)

An E1/E3 deleted, replication defective hAd5 that uses optionally the tissue plasminogen activator (tPA) leader sequence followed by a codon optimized nucleotide sequence encoding at least one SARS-CoV-2 protein(s) (e.g., any one or more of SEQ ID NOS. 2-11, and/or one or more fragment(s) and/or derivative(s) thereof). The expression cassette containing the cytomegalovirus (CMV) promoter and SARS-CoV-2 coding sequence are inserted into the E1 region of the hAd5. This vector is referred to herein as “hAd5-SARS-CoV-2”.

To investigate the expression level of SARS-CoV-2 protein(s) from the hAd5-SARS-CoV-2 vaccine, the expression of the SARS-CoV-2 protein(s) are compared from SARS-CoV-2-infected Vero cells by western blot with an anti-SARS-CoV-2 rabbit polyclonal specific to S. The western blot shows that the hAd5-SARS-CoV-2 vaccine expresses S in infected cells. The expressed S antigen is also sequenced to verify the native sequence.

B. Immunogenicity of hAd5-SARS-CoV-2 vaccine.

To understand the immunogenicity of the hAd5-SARS-CoV-2 vaccine, eight-week old mice (e.g., wild type or transgenic provided they expresses the receptor for SARS-CoV-2) are intranasally (i.n.) immunized with sufficient number of (e.g., 1×10⁷) hAd5-SARS-CoV-2 viral particles. On day eight, the peak of the T cell adaptive immune response, a subset of vaccinated mice are euthanized and SARS-CoV-2 antigen specific CD8⁺ T cell responses evaluated by intracellular cytokine staining and/or IFN-γ ELISpot. Splenic or PBMCs T cells are stimulated with peptide pools covering the antigen and/or an immunodominant SARS-CoV-2 peptide and the frequency of IFN-γ-producing T cells are determined. Twenty-one days after vaccination mice are bled to evaluate the serum antibody response. A SARS-CoV-2 specific ELISA is used to determine SARS-CoV-2 S specific immunoglobulin (IgG) responses. A single hAd5-SARS-CoV-2 vaccination may generate a SARS-CoV-2 S specific IgG response, e.g., with a higher reciprocal mean endpoint versus naïve animals with a reciprocal mean endpoint titer below the limit of detection. To determine the quantity of neutralizing antibodies, focus reduction neutralization tests are completed.

C. Neutralization of SARS-CoV-2

The virus neutralizing capacity of the vaccine will be determined using sera from immunized mice as described in section B. of this example, using the method of plaque reduction, or infected foci reduction. Either wild type, attenuated or VSV-pseudo-typed with the SARS-CoV-2 S will be mixed with various dilutions of vaccinated mouse serum and incubated for 30 min at room temperature followed by infection of Vero or other appropriate cell line. Virus neutralization then is quantified as the highest antibody dilution capable of reducing the number of plaque or infectious foci by a predetermined value, for example 50%.

D. Protective Capacity of hAd5-SARS-CoV-2 immunogenic compositions

To establish the protective capacity of the SARS-CoV-2 immunogenic composition, a sufficient number of hAd5-SARS-CoV-2 viral particles (e.g., 1×10⁷) are administered i.n. to mice or other appropriate rodent (e.g. transgenic rodent expressing the virus receptor). Twenty-one days later the animals are challenged with SARS-CoV-2, along with a phosphate buffered saline (PBS)-vaccinated control group and monitored for survival. After stringent challenge all PBS-vaccinated control mice are found to exhibit symptoms and/or succumbed to SARS-CoV-2 infection, while the majority of hAd5-SARS-CoV-2 vaccinated mice survive. Clinical signs of disease are scored for twelve days and found improved in hAd5-SARS-CoV-2 vaccinated mice compared to the non-vaccinated controls. Such results will demonstrate that the hAd5-SARS-CoV-2 vaccine is able to induce a protective response leading to reduced disease severity in this animal model.

E. Other

The tests described above can also be carried out using one or more other rdAd anti-SARS-CoV-2 vectors (e.g., AdE, AdD) by substituting hAd5-SARS-CoV-2 with another such vector (e.g., AdE, AdD), as would be understood by those of ordinary skill in the art. The generation and interpretation of results would be adjusted according to the particular rdAd anti-SARS-CoV-2 vector(s) being tested, as would be understood by those of ordinary skill in the art.

### Example 5: hAd5-SARS-CoV-2 Human Clinical Trial for SARS-CoV-2 Vaccination

To establish the immunogenic and/or protective capacity of a hAdv5-SARS-CoV-2 immunogenic composition comprising hAd5-SARS-CoV-2 viral particles (vp) and at least one pharmaceutically acceptable excipient, the same is administered to human subjects and tested for its effect on the immune response against SARS-CoV-2 in such subjects and, for its effect on time to clinical improvement and/or recovery on patients (e.g., hospitalized patients) infected with SARS-CoV-2. To do so, a randomized, double-blind, placebo-controlled, dose-escalation clinical trial to evaluate the safety and immunogenicity of hAdv5-SARS-CoV-2 composition in healthy adults 18 to 49 years of age is carried out. Subjects are typically screened within 28 days of randomization (Day 1).

For instance, a study can comprise two parts; part A which evaluates safety and part B which evaluates immunogenicity of the hAdv5-SARS-CoV-2 immunogenic composition. In part A, approximately 120 subjects who meet all inclusion and no exclusion criteria and provided written informed consent are enrolled into four sequential cohorts of 30 subjects each defined by the hAdv5 SARS-CoV-2 dose (1×10⁸, 1×10⁹, 1×10¹⁰, and 1×10¹¹ vp). Within each cohort (and the sentinel group in the first dose cohort), subjects are randomized in a 4:1:1 ratio to receive one intranasal dose of the hAdv5-SARS-CoV-2 immunogenic composition (Day 1) or one intranasal dose of placebo (normal saline) (Day 1). The hAdv5-SARS-CoV-2 immunogenic composition and placebo are administered in a double-blind fashion. Reactogenicity can be ascertained by determining counts and percentages of subjects with local events including but not limited to nasal irritation, sneezing, nasal congestion, cough, sore throat, change in smell, change in taste, change in vision, eye pain, pain, tenderness, induration, erythema, regional lymphadenopathy, and systemic events (headache, fatigue, myalgia, nausea, vomiting, diarrhea, coughing, chills, fever) for 14 days after vaccination. Adverse Events (AEs) are determined as counts and percentages of subjects with AEs from Day 1 to Day 57; medically attended AEs (MAAEs), serious AEs (SAEs), and new-onset chronic illnesses (NCIs) from Day 1 to Day 181 following administration of the hAdv5-SARS-CoV-2 immunogenic composition. For instance, targeted and symptom-driven physical examinations including vital signs can be carried out on days 4, 8, 15, 22, 29, and 57; an electrocardiogram can be carried out on day 57; safety laboratory tests can be carried out on days 8 and 57; and serum samples taken for immunogenicity testing at days 8, 15, 22, 29, 57, 91, 181, and 361 (e.g., enzyme-linked immunosorbent assay (ELISA) of serum to measure anti-SARS-CoV-2 antigen antibodies, geometric mean titer (GMT) as compared to day 0 (baseline)). Responder rates are also determined as described below (e.g., ≥four-fold rise in IgG post dose). The primary endpoint for evaluation of the safety profile in Part A is the number and percentage (95% confidence interval (CI)) of subjects with solicited and unsolicited AEs recorded postvaccination. Safety analyses is performed using the Safety Population. The number (percentage, 95% CI) of subjects with local events and systemic events is summarized by group, as is reactogenicity. The number (percentage, 95% CI) of subjects with AEs from Day 1 to Day 57 (including MAAEs, NCIs, SAEs) is summarized for each Medical Dictionary for Regulatory Activities system organ class (SOC) by preferred term (PT) and group. The number (percentage) of subjects with MAAEs, with NCIs, and with SAEs from Day 1 to Day 181 is summarized in a similar fashion. The number (percentage, 95% CI) of subjects with AEs by severity and by relationship to investigational product (IP) is also summarized. Listings of AEs, MAAEs, NCIs, and SAEs are provided.

In part B, the immunogenicity of hAdv5-SARS-CoV-2 immunogenic composition is determined. Following administration of the hAdv5-SARS-CoV-2 immunogenic composition by intranasal spray as a single dose of 1×10⁸, 1×10⁹, 1×10¹⁰, and 1×10¹¹ vp and as two doses (3 weeks apart) of the highest well tolerated of these doses to subjects, the immune response can be measured by ELISA of serum to measure anti-SARS-CoV-2 antigen antibodies, and the GMT, geometric mean ratio (GMR) (the ratio of postvaccination and pre-vaccination GMTs within the same dose group), and responder rate (≥four-fold rise in IgG post dose) determined. For instance, approximately 25 subjects who meet all inclusion and no exclusion criteria and provided written informed consent are randomized in a 4:1 ratio to receive two intranasal doses of the hAdv5-SARS-CoV-2 immunogenic composition at the highest well tolerated dose from Part A or placebo 21 days apart (Days 1 and 22). The hAdv5-SARS-CoV-2 immunogenic composition and placebo are administered in a double-blind fashion. Intranasal doses of the hAdv5-SARS-CoV-2 immunogenic composition and placebo are administered to subjects in a sitting position. In part B, targeted and symptom-driven physical examination including vital signs can be carried out on days 8, 15, 22, 29, 36, 43, 50, and 57; an electrocardiogram can be carried out on day 57; safety laboratory tests on days 8, 29, and 57; serum samples taken for immunogenicity testing at days 8, 15, 22, 29, 57, 91, 181, and 361; and nasopharyngeal swabs collected on days 8, 15, 29, 36, 43, 50, 57, and 91. Nasopharyngeal samples collected at Screening and on Days 29 and 57 can also be subsequently tested for evaluation of mucosal immune response.

The clinical trial can be carried out in patients already infected by SARS-CoV-2 and time to clinical improvement and/or recovery determined (or, in some embodiments, a cohort of the patients tested). A primary outcome measure is Time to Clinical Improvement (TTCI) and/or Time to Clinical Recovery (TTCR) which are determined for up to 28 days following administration of the hAdv5-SARS-CoV-2 composition as described above. TTCI is defined as the time (in days) from initiation of study treatment (active or placebo) until a decline of two categories from status at randomization on a six-category ordinal scale of clinical status which ranges from 1 (discharged) to 6 (death). The six-category ordinal scale is as follows: 6. Death; 5. ICU, requiring extracorporeal membrane oxygenation (ECMO) and/or invasive mechanical ventilation (IMV); 4. Intensive care unit (ICU)/hospitalization, requiring non-invasive mechanical ventilation (NIV)/high-flow nasal cannula (HFNC) therapy; 3. Hospitalization, requiring supplemental oxygen (but not NIV/HFNC); 2. Hospitalization, not requiring supplemental oxygen; and, 1. Hospital discharge or meet discharge criteria (discharge criteria are defined as clinical recovery, i.e. fever, respiratory rate, oxygen saturation return to normal, and cough relief). Secondary outcome TTCI measures include all-cause mortality (baseline SpO2 during screening, PaO2/FiO2<300 mmHg or a respiratory rate 24 breaths per min without supplemental oxygen); frequency of respiratory progression (SPO2≤94% on room air or PaO2/FiO2 <300 mmHg and requirement for supplemental oxygen or more advanced ventilator support); time to defervescence (in those with fever at enrolment); time to cough reported as mild or absent (in those with cough at enrollment rated severe or moderate); time to dyspnea reported as mild or absent (on a scale of severe, moderate, mild absent, in those with dyspnea at enrollment rated as severe or moderate,); frequency of requirement for supplemental oxygen or non-invasive ventilation; time to 2019-nCoV-2 RT-PCR negative in throat swab, sputum, lower respiratory tract specimen, and/or upper respiratory tract specimen; change (reduction) in SARS-CoV-2 viral load in throat swab, sputum, lower respiratory tract specimen, and/or upper respiratory tract specimen; change (reduction) in 2019-nCoV-2 viral load in in throat swab, sputum, lower respiratory tract specimen, and/or upper respiratory tract specimen; change (reduction) in SARS-CoV-2 viral load in throat swab, sputum, lower respiratory tract specimen, and/or upper respiratory tract specimen as assessed by area under viral load curve (e.g., as determined using polymerase chain reaction (PCR)); frequency of requirement for mechanical ventilation; and, frequency of serious adverse events. TTCI is defined as the time (in hours) from initiation of study treatment (active or placebo) until normalization of fever, respiratory rate, and oxygen saturation, and alleviation of cough, sustained for at least 72 hours. The primary TTCR outcome measures include normalization and alleviation criteria; fever≤36.9° C. or -axilla, ≤37.2° C. oral; respiratory rate—≤24/minute on room air; oxygen saturation—>94% on room air; and, cough—mild or absent on a patient reported scale of severe, moderate, mild, absent. The secondary TTCR outcome measures are the same as the TTCI secondary outcomes listed above.

Standard clinical trial design and statistical methods are used in the analyses of the data obtained from the trial. For instance, the sample size for this study is selected as adequate and reasonable for an initial review of the safety and immunogenicity profile of the hAdv5-SARS-CoV-2 composition at doses to be well tolerated, rather than for statistical power (e.g., 120 subjects as described above). The sample size permits initial estimates of reactogenicity. For example, given a total of 100 subjects receiving hAdv5-SARS-CoV-2 composition, the study is designed to have an 80% probability of detecting at least one AE that occurred at a rate of 1.6%. If no SAEs were observed among the 100 subjects who received hAdv5-SARS-CoV-2 immunological composition, an approximation to the 1-sided upper bound of the 95% confidence interval (CI) on the rate of SAE occurrence would be 3%. Immunology analyses are conducted using the Evaluable and Per-protocol (PP) Populations with primary conclusions drawn from the PP Population. Analyses based on the Evaluable Population are undertaken and presented only if >1 subject in any one group were excluded from the PP Population. With the exception responder analyses, as described below, no imputation for missing data is performed. Data is transformed as appropriate prior to analysis. Baseline is defined as the sample collected prior to hAdv5-SARS-CoV-2 composition administration on Day 1. The primary variables of interest for assessment of humoral immune response to SARS-CoV-2 are anti-SARS-CoV-2 antigen IgG titers. GMTs are determined at Baseline and postvaccination on Days 8, 15, 22, 29, 57, 91, and 181 (Part A) and Days 8, 15, 22, 29, 36, 43, 50, 91, and 181 (Part B) and summarized by dose group. Comparisons between hAdv5-SARS-CoV-2 composition doses and placebo are evaluated by analysis of covariance (ANCOVA) with treatment as a fixed effect and baseline log-transformed level as a covariate on the post-baseline log-transformed level of anti-SARS-CoV-2 IgG as a dependent variable. From these analyses, least-square (LS) means, LS treatment differences, and 95% CIs for the treatment differences on log-scale are obtained. The results are transformed back to the original scale by exponentiation to provide treatment geometric LS means, point estimates of the geometric LS mean ratios, and 95% CI for these ratios on each study day. A “responder” is defined as a subject with a 4-fold rise in anti-SARS-CoV-2 antigen titer from baseline on Days 8, 15, 22, 29, 57, 91, and 181 (Part A) and Days 8, 15, 22, 29, 36, 43, 50, 91, and 181 (Part B). Fold change for determination of responder status is computed using the post-imputation values without the +1 transformation, i.e., fold change=current imputed value/baseline imputed value. Responder rates are tabulated by percentages per dose group and the 95% Clopper-Pearson exact CI of the percentage. Differences of 95% CIs are presented to compare the response rate of each hAdv5-SARS-CoV-2 composition dose group to the and placebo group. In some embodiments, comparisons of responders in each hAdv5-SARS-CoV-2 composition dose group against the against the placebo group can also be conducted using Fisher's exact test. To determine the effect of pre-dose Ad5 serum antibody levels on immunogenicity of hAdv5-SARS-CoV-2 composition on Day 29 (Part A) or Day 50 (Part B), analyses are performed using ANCOVA with baseline Ad5 titer as a covariate. Mucosal immunogenicity analyses are conducted using the Evaluable and PP Populations. No imputation for missing data is performed. Endpoints analyzed are GMT and GMR for IgA antibody level measured by ELISA. Methods used are the same as for humoral immunogenicity analyses. Summary statistics for continuous parameters (safety laboratory tests and vital signs) are presented by group as follows: pre-vaccination, postvaccination, and change from pre-vaccination to postvaccination assessment. The number and percentage of subjects with postvaccination safety laboratory values or vital sign values recorded as newly abnormal (ie, an event with an increase in the toxicity grade relative to the baseline value and with a severity grade of moderate or higher) after study vaccination are tabulated. Shift tables that cross-tabulate the pre-vaccination and postvaccination safety laboratory values of each subject by severity grade are prepared. Summaries of the number and percentage of subjects with normal, abnormal not clinically significant, and abnormal clinically significant ECG interpretations are presented. For shedding of the RD-Ad5 vector, data are summarized by count and percent positive by time point, along with median copy number. The median duration of Ad5 shedding, interquartile range, minimum and maximum duration of Ad5 shedding are presented for each hAdv5-SARS-CoV-2 composition group and all hAdv5-SARS-CoV-2 composition dose groups combined. Viral culture results for evaluation of adenovirus infection are also listed.

These studies will show that the hAdv5-SARS-CoV-2 composition can be used to induce an anti-SARS-CoV-2 immune response in human beings (i.e., it is an immunogenic composition), and exhibits an acceptable safety profile. It is preferred that that immune response be statistically significant, and even more preferably, a protective immune response (i.e., it is a SARS-CoV-2 vaccine). In preferred embodiments, the data shows the hAdv5-SARS-CoV-2 vaccine composition can be used to treat subjects infected by SARS-CoV-2 (e.g., hospitalized patients).

### Example 6: AdD Vectors Human Clinical Trial for SARS-CoV-2 Vaccination

In this example, the use of intranasal (i.n.) administration of AdD vectors (i.e., replication deficient ΔE1E3 adenovirus type 5 (Ad5) viral particles encoding a pathogen antigen derived from an infectious agent other than SARS-CoV-2, e.g. influenza such as NasoVAX which is an AdVector (Ad5) expressing influenza hemagglutinin (HA) antigen, described in, e.g., Ser. No. 62/830,444 filed on 6 Apr. 2019 which is incorporated herein by reference and discloses preparation of NasoVAX) to confer prophylactic therapy against SARS-CoV-2 is described. In embodiments, the AdD vector induces an immune response, preferably a protective immune response, against both SARS-CoV-2 and the pathogen associated with the expressed exogenous antigen of the AdD vector. For example, NasoVAX will induce an immune response against both influenza and coronavirus including SARS-CoV-2. In this way, AdD is a dual vaccine inducing an immune response against two respiratory infectious agents.

To establish the immunogenic and/or protective capacity of a composition comprising AdD vectors against SARS-CoV-2, an AdD composition comprising AdD viral particles (vp), are administered to human being subjects and tested for its effect on the immune response against SARS-CoV-2 therein. To do so, a randomized, double-blind, placebo-controlled, dose-escalation clinical trial to evaluate the safety and immunogenicity of a AdD immunological composition in healthy adults 18 to 49 years of age can be carried out. Subjects are typically screened within 28 days of randomization (Day 1).

For instance, a study can comprise two parts; part A which evaluates safety, and part B which evaluates immunogenicity, of the AdD composition (e.g., NasoVax). In part A, approximately 120 subjects who meet all inclusion and no exclusion criteria and provided written informed consent are enrolled into four sequential cohorts of 30 subjects each defined by the AdD dose (1×10⁸, 1×10⁹, 1×10¹⁰, and 1×10¹¹ vp), each subject being administered a single intranasal dose in a total volume of 0.5 ml split evenly between nostrils as a nasal spray (“placebo” subjects receive 0.5 ml of normal saline not including AdD, the does also being split evenly between nostrils as a nasal spray). Within each cohort (and the sentinel group in the first dose cohort), subjects are randomized in a 4:1:1 ratio to receive one intranasal dose of the AdD composition (Day 1) or one intranasal dose of placebo (normal saline) (Day 1). The AdD composition and placebo are administered in a double-blind fashion. Reactogenicity can be ascertained by determining counts and percentages of subjects with local events including but not limited to nasal irritation, sneezing, nasal congestion, cough, sore throat, change in smell, change in taste, change in vision, eye pain, pain, tenderness, induration, erythema, regional lymphadenopathy, and systemic events (headache, fatigue, myalgia, nausea, vomiting, diarrhea, coughing, chills, fever) for 14 days after vaccination. Adverse Events (AEs) are determined as counts and percentages of subjects with AEs from Day 1 to Day 57; medically attended AEs (MAAEs), serious AEs (SAES), and new-onset chronic illnesses (NCIs) from Day 1 to Day 181 following administration of the AdD composition. For instance, targeted and symptom-driven physical examinations including vital signs can be carried out on days 4, 8, 15, 22, 29, and 57; an electrocardiogram can be carried out on day 57; safety laboratory tests can be carried out on days 8 and 57; and serum samples taken for immunogenicity testing at days 8, 15, 22, 29, 57, 91, 181, and 361 (e.g., enzyme-linked immunosorbent assay (ELISA) of serum to measure anti-SARS-CoV-2 antigen and/or anti-D antigen antibodies, geometric mean titer (GMT) as compared to day 0 (baseline)), and/or anti-SARS-CoV-2 and/or anti-AdD immune cellular responses (e.g., T cell response). Responder rates are also determined as described below (e.g., ≥four-fold rise in IgG post dose). The primary endpoint for evaluation of the safety profile in Part A is the number and percentage (95% confidence interval (CI)) of subjects with solicited and unsolicited AEs recorded postvaccination. Safety analyses is performed using the Safety Population. The number (percentage, 95% CI) of subjects with local events and systemic events is summarized by group, as is reactogenicity. The number (percentage, 95% CI) of subjects with AEs from Day 1 to Day 57 (including MAAEs, NCIs, SAEs) is summarized for each Medical Dictionary for Regulatory Activities system organ class (SOC) by preferred term (PT) and group. The number (percentage) of subjects with MAAEs, with NCIs, and with SAEs from Day 1 to Day 181 is summarized in a similar fashion. The number (percentage, 95% CI) of subjects with AEs by severity and by relationship to investigational product (IP) is also summarized. Listings of AEs, MAAEs, NCIs, and SAEs are provided.

In part B, the immunogenicity of the AdD composition is determined. Following administration of the AdD composition by intranasal spray as a single dose of 1×10⁸, 1×10⁹, 1×10¹⁰, and 1×10¹¹ vp in a 0.5 ml volume split evenly between nostrils as a nasal spray (and in some embodiments as two doses (3 weeks apart) of the highest well tolerated of these doses) to subjects, the immune response can be measured by ELISA of serum to measure anti-SARS-CoV-2 antigen antibodies and/or anti-AdD antibodies, and the GMT, geometric mean ratio (GMR) (the ratio of postvaccination and pre-vaccination GMTs within the same dose group), and responder rate (≥four-fold rise in IgG post dose), as well as immune cellular responses (e.g., T cell responses), are determined. For instance, approximately 25 subjects who meet all inclusion and no exclusion criteria and provided written informed consent are randomized in a 4:1 ratio to receive two intranasal doses of the AdD composition at the highest well tolerated dose from Part A or placebo 21 days apart (Days 1 and 22). The AdD composition and placebo are administered in a double-blind fashion. Intranasal doses of the AdD composition and placebo are administered to subjects in a sitting or reclined position. In part B, targeted and symptom-driven physical examination including vital signs can be carried out on days 8, 15, 22, 29, 36, 43, 50, and 57; an electrocardiogram can be carried out on day 57; safety laboratory tests on days 8, 29, and 57; serum samples taken for immunogenicity testing at days 8, 15, 22, 29, 57, 91, 181, and 361; and nasopharyngeal swabs collected on days 8, 15, 29, 36, 43, 50, 57, and 91. Nasopharyngeal samples collected at Screening and on Days 29 and 57 can also be subsequently tested for evaluation of mucosal immune response.

In some embodiments, the clinical trial can be carried out using patients already infected by SARS-CoV-2 and time to clinical improvement and/or recovery determined (or, in some embodiments, a cohort of the patients tested). A primary outcome measure is Time to Clinical Improvement (TTCI) and/or Time to Clinical Recovery (TTCR) which are determined for up to 28 days following administration of the AdD composition as described above. TTCI is defined as the time (in days) from initiation of study treatment (active or placebo) until a decline of two categories from status at randomization on a six-category ordinal scale of clinical status which ranges from 1 (discharged) to 6 (death). The six-category ordinal scale is as follows: 6. Death; 5. ICU, requiring extracorporeal membrane oxygenation (ECMO) and/or invasive mechanical ventilation (IMV); 4. Intensive care unit (ICU)/hospitalization, requiring non-invasive mechanical ventilation (NIV)/high-flow nasal cannula (HFNC) therapy; 3. Hospitalization, requiring supplemental oxygen (but not NIV/HFNC); 2. Hospitalization, not requiring supplemental oxygen; and, 1. Hospital discharge or meet discharge criteria (discharge criteria are defined as clinical recovery, i.e. fever, respiratory rate, oxygen saturation return to normal, and cough relief). Secondary outcome TTCI measures include all-cause mortality (baseline SpO2 during screening, PaO2/FiO2<300 mmHg or a respiratory rate ≥24 breaths per min without supplemental oxygen); frequency of respiratory progression (SPO2≤94% on room air or PaO2/FiO2<300 mmHg and requirement for supplemental oxygen or more advanced ventilator support); time to defervescence (in those with fever at enrolment); time to cough reported as mild or absent (in those with cough at enrolment rated severe or moderate); time to dyspnea reported as mild or absent (on a scale of severe, moderate, mild absent, in those with dyspnoea at enrollment rated as severe or moderate,); frequency of requirement for supplemental oxygen or non-invasive ventilation; time to SARS-CoV-2 RT-PCR negative in upper respiratory tract specimen; change (reduction) in SARS-CoV-2 viral load in upper respiratory tract specimen as assessed by area under viral load curve (e.g., as determined using polymerase chain reaction (PCR)); frequency of requirement for mechanical ventilation; and, frequency of serious adverse events. TTCI is defined as the time (in hours) from initiation of study treatment (active or placebo) until normalization of fever, respiratory rate, and oxygen saturation, and alleviation of cough, sustained for at least 72 hours. The primary TTCR outcome measures include normalization and alleviation criteria; fever—≤36.9° C. or -axilla, ≤37.2° C. oral; respiratory rate—≤24/minute on room air; oxygen saturation—>94% on room air; and, cough—mild or absent on a patient reported scale of severe, moderate, mild, absent. The secondary TTCR outcome measures are the same as the TTCI secondary outcomes listed above.

In some embodiments, a clinical trial can be carried out on approximately 120 subjects testing positive for SARS-CoV-2 aged over 50 years randomized into placebo and treatment groups. The placebo group receives 0.5 ml normal saline not including any AdD vector, administered as a single intranasal dose split evenly between the nostrils as a nasal spray. The treatment group is administered 0.5 ml normal saline including any AdD vector (i.e., NasoVax), administered as a single intranasal dose split evenly between the nostrils as a nasal spray. The primary efficacy endpoints are the proportion of subjects developing acute respiratory distress symptoms (ARDS) and maximum severity of COVID-19 (i.e., the symptoms of SARS-CoV-2 infection) by forced expiratory volume (FEV-1) and radiographic criteria. Secondary endpoints include viral shedding, days on mechanical ventilation and length of hospital stay.

Standard clinical trial design and statistical methods are used in the analyses thereof. For instance, the sample size for this study is selected as adequate and reasonable for an initial review of the safety and immunogenicity profile of the AdD composition at doses to be well tolerated, rather than for statistical power (e.g., 120 subjects as described above). The sample size permits initial estimates of reactogenicity. For example, given a total of 100 subjects receiving AdD composition, the study is designed to have an 80% probability of detecting at least one AE that occurred at a rate of 1.6%. If no SAEs were observed among the 100 subjects who received hAdv5-D (AdD) composition, an approximation to the 1-sided upper bound of the 95% confidence interval (CI) on the rate of SAE occurrence would be 3%. Immunology analyses are conducted using the Evaluable and Per-protocol (PP) Populations with primary conclusions drawn from the PP Population. Analyses based on the Evaluable Population are undertaken and presented only if >1 subject in any one group were excluded from the PP Population. With the exception responder analyses, as described below, no imputation for missing data is performed. Data is transformed as appropriate prior to analysis. Baseline was defined as the sample collected prior to AdD composition administration on Day 1. The primary variables of interest for assessment of humoral and cellular immune response to SARS-CoV-2 (e.g., anti-SARS-CoV-2 and/or anti-AdD antigen IgG titers, T cell responses) are determined. GMTs are determined at Baseline and postvaccination on Days 8, 15, 22, 29, 57, 91, and 181 (Part A) and Days 8, 15, 22, 29, 36, 43, 50, 91, and 181 (Part B) and summarized by dose group. Comparisons between AdD composition doses and placebo were evaluated by analysis of covariance (ANCOVA) with treatment as a fixed effect and baseline log-transformed level as a covariate on, e.g., the post-baseline log-transformed level of anti-SARS-CoV-2 IgG as a dependent variable. From these analyses, least-square (LS) means, LS treatment differences, and 95% CIs for the treatment differences on log-scale are obtained. The results are transformed back to the original scale by exponentiation to provide treatment geometric LS means, point estimates of the geometric LS mean ratios, and 95% CI for these ratios on each study day. A “responder” is defined as a subject with a 4-fold rise in anti-SARS-CoV-2 and/or anti-D antigen titer from baseline on Days 8, 15, 22, 29, 57, 91, and 181 (Part A) and Days 8, 15, 22, 29, 36, 43, 50, 91, and 181 (Part B). Fold change for determination of responder status is computed using the post-imputation values without the +1 transformation, i.e., fold change=current imputed value/baseline imputed value. Responder rates are tabulated by percentages per dose group and the 95% Clopper-Pearson exact CI of the percentage. Differences of 95% CIs are presented to compare the response rate of each AdD composition dose group to the and placebo group. In some embodiments, comparisons of responders in each AdD composition dose group against the against the placebo group can also be conducted using Fisher's exact test. To determine the effect of pre-dose Ad5 serum antibody levels on immunogenicity of AdD composition on Day 29 (Part A) or Day 50 (Part B), analyses are performed using ANCOVA with baseline Ad5 titer as a covariate. Mucosal immunogenicity analyses are conducted using the Evaluable and PP Populations. No imputation for missing data is performed. Endpoints analyzed are GMT and GMR for IgA antibody level measured by ELISA. Methods used are the same as for humoral immunogenicity analyses. Summary statistics for continuous parameters (safety laboratory tests and vital signs) are presented by group as follows: pre-vaccination, postvaccination, and change from pre-vaccination to postvaccination assessment. The number and percentage of subjects with postvaccination safety laboratory values or vital sign values recorded as newly abnormal (ie, an event with an increase in the toxicity grade relative to the baseline value and with a severity grade of moderate or higher) after study vaccination are tabulated. Shift tables that cross-tabulate the pre-vaccination and postvaccination safety laboratory values of each subject by severity grade are prepared. Summaries of the number and percentage of subjects with normal, abnormal not clinically significant, and abnormal clinically significant ECG interpretations are presented. For shedding of the Ad5 vector, data are summarized by count and percent positive by time point, along with median copy number. The median duration of Ad5 shedding, interquartile range, minimum and maximum duration of Ad5 shedding are presented for each AdD composition group and all immunological composition dose groups combined. Viral culture results for evaluation of adenovirus infection are also listed.

These studies will show that the AdD composition can be used to induce an anti-SARS-CoV-2 immune response in human beings (e.g., it is an immunogenic composition), and exhibits an acceptable safety profile. It is preferred that that immune response be statistically significant, and even more preferably, a protective immune response (i.e., it is a SARS-CoV-2 vaccine). In preferred embodiments, the data shows the AdD composition can be used to treat subjects infected by SARS-CoV-2 (e.g., hospitalized patients).

### Example 7: Double-Blind, Randomized, Placebo-Controlled Study of NasoVAX in the Prevention of Clinical Worsening in Patients with Early Onset COVID-19

In this example, NasoVAX is used as therapy for the early phases of infection or as a concomitant therapy with direct antiviral agents. Pertaining to the treatment of COVID-19, NasoVAX (and AdE) were demonstrated in preclinical mouse models to provide protection from lethal challenge with a respiratory virus, an effect that occurred in as little as 2 days and lasted 3 or more weeks. See U.S. Pat. No. 9,175,310. In Example 2 above, use of AdE (no transgene expression) was associated with down-regulation of IL-6, IL-1α and IL-12. Those are cytokines that have been demonstrated to mediate pulmonary interstitial inflammation in COVID-19. The protection afforded by NasoVAX and AdE in the pre-clinical mouse models may be attributed to the adenovirus vector, as the vector had commensurate effects in the presence or absence of a transgene expressing the influenza hemagglutinin (HA) antigen. The protective effects of NasoVAX can be be viewed as a biologic response modulating innate immunity that dampens the excessive and pathogenic immune response to a respiratory pathogen. This would be analogous to the use of IL-6 inhibitors Kevzara (sarilumab) and Actemra (tocilizumab) to modulate lung inflammation associated with COVID-19. In embodiments, an adenoviral vector (with or without expressing a transgene from a respiratory pathogen) when administered intranasally ameliorates COVID-19 disease symptoms.

The strength of the vector approach is that unlike other agents that can be used to modulate lung inflammation associated with COVID-19 (e.g., Kevzara (sarilumab) and Actemra (tocilizumab)), NasoVAX is administered intranasally, leaving IV access ports free for other medication; not limited to a single cytokine; not having a hematological side-effect profile (e.g., neutropenia); and exhibits a prolonged duration of action, which would be useful in very early stages in the disease process to modulate subsequent cytokine damage. Moreover, as shown in Example 9 below, when an adenoviral vectored immunogenic composition is administered intranasally bypasses adenovirus immunity of the subject (e.g., those that are seropositive for the viral vector, i.e. Ad5), allowing for repeated dosing and/or doing to adenovirus seropositive subjects.

Thus, in some embodiments, NasoVAX could be used as therapy for the early phases of infection or as a concomitant therapy for COVID-19, in some embodiments in combination with direct antiviral agents (e.g., chloroquine, azithromycin). At some juncture, the drug substance could transition into a product in which the vector alone (e.g., sans transgene as in AdE) is administered.

NasoVAX is prepared by isolation of bacterial colony harboring the large recombinant adenovirus plasmid bearing the human codon-optimized hemagglutinin cDNA from influenza A/California/04/2009 (pAdcoCA09.HA). AdcoCA09.HA recombinant vector was recovered from PER.C6 cells in suspension following large scale transfection of pAdcoCA09.HA plasmid into approximately 4×10⁹ cells in a single operation using flow-cell electroporation technology (Model STX-100, Maxcyte Inc. Gaithersburg, Md.). At the time of harvest, the infected cells and growth media containing any released vector were subjected to three cycles of freeze-thaw followed by isolation and purification of the vector by CsCl isopycnic centrifugation and dialysis against final product formation buffer. The purified vector was amplified in PER.C6 cells, released from the infected cell pellet by three cycles of freeze-thaw to create an infected cell lysate that was clarified by centrifugation and sterile filtered as the Pre-Master Virus Seed. The NasoVAX Pre-MVS was released following testing. The manufacture of AdcoCA09.HA includes a vector expansion step whereby vector pre-MVS is amplified under cGMP to increase the available seed stock for infection of the production run. The product of this intermediate expansion step is the AdcoCA09.HA MVS. Production of the AdcoCA09.HA MVS starts with cGMP vector expansion of the pre-MVS followed by derivation, production, and characterization of the MVS. Manufacturing of the drug substance includes AdcoCA09.HA infection of PER.C6 cells in suspension followed by concentration of the vector in the cell pellet, release of the vector from the cell pellet, clarification of the lysate and purification of the vector using two sequential anion exchange chromatography resins. The product eluate is diafiltered against formulation buffer, concentrated if necessary, and sterile filtered to create the bulk drug substance (BDS). Final drug product (FDP) is obtained from BDS following dilution to the appropriate strength with formulation buffer and sterile filtration before filling into the final container. The test product to be used in this example is NasoVAX supplied (e.g., 1×10⁹, 10¹⁰ and/or 10¹¹ vp) in single-use glass syringes containing 500 μL of a sterile, frozen suspension in A195 buffer (10 mM Tris, 10 mM histidine, 5% (w/v) sucrose, 75 mM NaCl, 1 mM MgCl₂, 0.02% (w/v) polysorbate-80, 0.1 mM EDTA, 0.5% (v/v) ethanol, pH 7.4). The syringes (BD, Accuspray™) are designed to deliver 250 μL of an intranasal spray to each nostril. Alternatively, NasoVAX is supplied in 2 mL glass vials, wherein the dose is removed to a tuberculin syringe and affixed to a LMA MAD300 atomizer device (Teleflex, Israel) before intranasal administration. Stability samples were packaged in the same dosage form and container (BD Accuspray™) as the test product and tested using stability indicating assays including physical stability of the virus particles (viral particles test, HPLC), infectivity of the virus (infectious titer, Adenovirus Fluorescent Focus Unit (FFU) Assay), functionality (transgene expression) of the adenovirus vector (potency), the physical stability of the formulation (appearance and pH), and sterility.

In this example, a clinical trial including approximately 120 patients with early onset COVID-19 randomized 1:1 to NasoVAX (1×10¹¹ vp dose) or placebo and stratified by age is described. Each patient will participate in the study up to approximately 6 weeks (a 2-week Treatment Period and a 1-month follow-up phone call). Patients are included in this study are selected using the following non-limiting criteria: able and willing to provide informed consent; men and women 35 years of age and older; early onset COVID-19, defined as oral temperature ≥38.0° Celsius, onset of symptoms within 48 hours, and confirmation of COVID-19 by a PCR-based diagnostic within 24 hours of randomization; saturated 02 (SaO₂) ≥96.0% at rest for 5 minutes on two successive measurements; women of childbearing potential (women who are not permanently sterile [documented hysterectomy, bilateral tubal ligation, salpingectomy, or oophorectomy] or postmenopausal [12 months with no menses without an alternative medical cause]) exhibit negative urine pregnancy test at screening and willingness to practice a highly effective method of contraception that includes, but is not limited to, abstinence, sex only with persons of the same sex, monogamous relationship with a postmenopausal partner, monogamous relationship with vasectomized partner, vasectomy, licensed hormonal methods, intrauterine device, or consistent use of a barrier method (e.g., condom, diaphragm) with spermicide for 28 days after the last dose of study medication; men with sexual partners of childbearing potential have a willingness to practice a highly effective method of contraception, as defined above, for 45 days after the last dose of study medication; and the ability and willingness to comply with all aspects of the study through the entire study period. Exclusion criteria include: pregnant or lactating women; moderate or severe shortness of breath at rest; findings on physical examination suggesting rapid disease progression, need for immediate hospitalization, obstructive airway diseases, including chronic obstructive pulmonary disease (COPD) and asthma, or other respiratory diseases that could exacerbate independent of those caused by COVID-19; nasal conditions that might affect the suitability of intranasal medication, such as a history of chronic rhinitis, nasal septal defect, cleft palate, nasal polyps, or nasal surgery other than cosmetic rhinoplasty; use of chloroquine and hydroxychloroquine and other investigational agents for COVID-19 within the past 30 days; history of conditions associated with immunocompromise, or treatments known to affect the immune system, including but not limited to oral or intravenous corticosteroids, alkylating drugs, antimetabolites, cytotoxic drugs, radiation, immune-modulating biologics, within 30 days of screening; and, any medical, psychiatric, or social condition or occupational or other responsibility that in the judgment of the Investigator would interfere with or serve as a contraindication to protocol adherence, assessment of safety (including reactogenicity), or a patient's ability to give informed consent.

Patients are randomized 1:1 to NasoVAX or placebo administered as a single intranasal dose of 0.5 mL (0.25 mL each nostril) within 24 hours of COVID-19 diagnosis. The first 20 study participants (approximately 10 treated with NasoVAX and 10 treated with placebo) consist of a sentinel cohort of patients ages 35 to 49 years. Patients are provided disposable finger-tip oximeters and digital thermometers and return home for the duration of the trial. SaO₂, oral temperature, and pulse respiratory rate is monitored remotely at rest for 2 minutes twice daily for 14 days using mobile/smart phone pulse oximetry and web-based diaries to record oral temperature, symptoms and concomitant medications and telephoned daily to document clinical status and adverse events (AEs). Patients are called approximately every 7±2 days after the last day of remote monitoring to document final outcome and adverse events. SAEs, hospital and ICU lengths of stays, and mortality in hospitalized patients will be documented. No in-person visits are expected during the study.

With respect to efficacy, the Primary Objective of this study is to assess the effectiveness of NasoVAX in preventing clinical worsening in patients with early onset COVID-19; and the Secondary Objectives are to assess the effectiveness of NasoVAX in reducing rates of ICU admission and mechanical ventilation in patients with early onset COVID-19 and the severity of COVID-19 in patients with early onset COVID-19 who require hospitalization. The primary endpoint is the proportion of patients with clinical worsening, defined as a 4% decrease in mean SaO₂ to a level of 94% or less by mobile pulse oximetry at any measurement during home follow-up, or hospitalization. In ambulatory patients, secondary efficacy endpoints include severity of COVID-19, assessed by maximum decrease in SaO₂ and spontaneous ventilation rate by outpatient pulse oximetry during home follow-up and the proportion of patients requiring mechanical ventilation. In hospitalized patients, the secondary efficacy endpoints include ventilator-free days, defined as one point (day) for each day between the dose of study medication on Day 1 and Day 30 (relative to the first dose of study drug) that a patient is both alive and free of mechanical ventilation and the ratio of arterial oxygen partial pressure to fractional inspired oxygen (P/F ratio).

With respect to safety, the Primary Objective is to assess the safety and tolerability of NasoVAX in preventing clinical worsening in patients with early onset COVID-19. These include incidence and severity of adverse events (AEs), mortality, hospital length of stay, and ICU length of stay. Safety endpoints are categorized separately between AEs reported at home vs. those reported during hospitalization for medical care.

Statistical methods include the Power and Sample Size Assumptions that 10% of patients receiving NasoVAX develop clinical worsening vs. 29% receiving placebo, 60 patients per treatment arm provides 77% power at a one-sided a of 0.05 to achieve statistical significance on the primary efficacy variable. Population definitions include: “Safety Analysis Set”: all patients who receive any study medication; “Intent to treat” (ITT): all randomized patients who receive any amount of study medication, have a baseline and at least one post-baseline SaO₂ measurement. Subjects are analyzed according to the treatment that they receive; and, “Per Protocol” (PP): all randomized patients who receive any amount of study medication according to the correct treatment assignment and who have twice daily results from SaO₂ measurements through Day 14 or hospitalization. Baseline is defined as data collected closest to randomization prior to any study medication dosing. All analyses and summary statistics are presented by treatment group (NasoVAX, placebo). Descriptive statistics, including the numbers and percentages for categorical variables and the numbers, means, standard deviations, medians, minimums and maximums for continuous variables are provided by treatment. Patients are randomized 1:1 to NasoVAX or placebo and stratified by age group (35-49 years vs. 50 years and older). To assure a 1:1 distribution of NasoVax and placebo (10 patients each of 20 patients) in the sentinel cohort, randomization in this group are not stratified. For the Efficacy Analyses, descriptive statistics are used to evaluate differences in demographic and baseline characteristics. For the primary analysis, proportions of patients with clinical worsening, defined as a 4% decrease in mean SaO₂ to a level of 94% or less by mobile pulse oximetry at any measurement during home follow-up, or hospitalization, are compared between NasoVAX and placebo groups using the Cochrane Mantel Haenszel test at a 0.05% one-sided level of significance. The same approach is applied for secondary or exploratory endpoints that are categorical in nature. Subjects who discontinue prematurely or have missing data are considered non-responders for that endpoint. Sensitivity analyses are performed to assess the effect of site on the response to study medication. Linear and logistic regression are employed to examine the effects of baseline factors, such as age, sex, medications and medical co-morbidities on response. Quantitative safety data are summarized using descriptive statistics and frequency distributions. All summaries are presented by treatment arms. AEs are coded using Medical Dictionary for Regulatory Activities)(MedDR®), Concomitant medications are coded using World Health Organization (WHO) drug dictionary. Changes from baseline in Severity of COVID-19, assessed by maximum decrease in SaO₂ and spontaneous ventilation rate by outpatient pulse oximetry during home follow-up, are analyzed using a mixed model for repeated measures (MMRM) model. The model will include the fixed effects of treatment, stratification factor, week, and treatment-by-visit interaction as well as the continuous, covariate of baseline level. The model will employ an unstructured within patient covariance matrix and a restricted maximum likelihood (ReML) estimation method. Ventilator-free days are analyzed using a t-test or Mann-Whitney for continuous data. A Kaplan-Meier model is developed to compare changes between treatment groups in SaO₂ over time. No multiplicity adjustments are made for secondary or exploratory endpoints.

These studies will show that the NasoVAX can be used to induce an anti-SARS-CoV-2 immune response in human beings (e.g., it is an immunogenic composition), and exhibits an acceptable safety profile. The data will also show that NasoVAX is effective in reducing rates of ICU admission and mechanical ventilation in patients with early onset COVID-19 and the severity of COVID-19 in patients with early onset COVID-19 who require hospitalization. In some embodiments, a decrease in expression of inflammatory cytokines such as IL-1α, IL-5, IL-6, IL-12, IL-17, MCP-1, tumor necrosis factor alpha (TNF-α), granulocyte macrophage colony stimulating factor (GM-CSF), and/or RANTES (CCL5) (see, e.g., Example 2) following administration of NasoVAX to subjects can occur, and can in some embodiments be used to diagnose COVID-19, and/or predict recovery therefrom and used to adjust treatment protocols (e.g., non-NasoVAX treatments) accordingly. In some embodiments, an increase in MCP1 and/or RANTES shortly after administration of NasoVAX, can be used to predict (e.g., as a marker) recovery from COVID-19 and amelioration of symptoms. It is preferred that that immune response be statistically significant, and even more preferably, a protective immune response (i.e., it is a SARS-CoV-2 vaccine). In preferred embodiments, the data shows that NasoVAX can be used to treat subjects infected by SARS-CoV-2 (e.g., hospitalized patients).

### Example 8. NasoVAX Stability at Room Temperature

This example describes the long-term stability of NasoVAX in a liquid formulation at room temperature. Long-term stability at room temperature is desire feature of vaccines that can be used in situations in which refrigeration or other means for stabilizing a formulation may not be available. This would be important in epidemic or pandemic situations during which vaccines need to be shipped to remote areas that may lack the equipment to maintain formulations at a cooler temperature, or shipped directly to end-users, such as individual self-isolating at home or in quarantine. As shown in Tables 9 and 10 below, low dose (2×10⁹ vp/mL dose) and high dose (2×10¹¹ vp/mL dose) formulations, respectively, were prepared and maintained at room temperature in glass vials for one, three and six months. Viability of the NasoVAX vectors was determined using the Adenovirus Fluorescent Focus Unit (FFU) assay. Briefly, the FFU assay is carried out by infecting cell monolayers with the appropriate NasoVAX dilution and incubated for 24-48 hours. The cells were then washed, inspected, fixed (e.g., ice-cold 90% methanol for four minutes), and washed again. Anti-Ad5 antibody was then added at various dilutions (antibody omitted in control samples), followed by a detection agent (e.g., NCL-Adeno (Novocastra, Newcastle, UK)) under appropriate conditions (e.g., ten minutes at room temperature with shaking). The cells are then washed, and the total number of infectious particles determined (e.g., by digital light scattering (DLS)). As shown in Tables 15 and 16, the low-dose and high-dose NasoVAX formulations were stable for at least three months at room temperature.

This study shows an adenoviral vectored vaccine composition (e.g., NasoVAX) is stable for about 3 months at an ambient temperature, such as room temperature (e.g., 15 to 30° C., preferably 20-25° C.). In embodiments, an adenoviral vectored vaccine composition can be stored, or shipped, without the need for refrigeration or specific storage conditions. In certain embodiments, the present intranasal adenoviral vectored vaccine is configured to induce an immune response against SARS-CoV-2 virus (a pandemic coronavirus strain) infection and/or to ameliorate COVID-19 disease symptoms and may be shipped directly to the user for intranasal administration.

TABLE 15
Stability Data for NasoVAX (2 × 10⁹ vp/mL dose)

| | Stability Time Point | | | | | | | |
|---|---|---|---|---|---|---|---|---|
| Analysis | T = 0 M | | T = 1 M | | T = 3 M | | T = 6 M | |
| Appearance | Liquid, Colorless; | | Liquid, Colorless, | | Liquid, Colorless; | | Liquid, Colorless; | |
| | Translucent; | | Translucent; | | Clear; | | Transparent; | |
| | No visible | | No visible | | No visible | | No visible | |
| | particulate | | particulate | | particulate | | particulate | |
| | matter observed | | matter observed | | matter observed | | observed | |
| pH | 7.5 | | 7.5 | | 7.7 | | 7.5 | |
| vp by HPLC | 1.2 × 10⁹ | vp/mL | 1.1 × 10⁹ | vp/mL | 0.9 × 10⁹ | vp/mL | 1.2 × 10⁹ | vp/mL |
| Adenovirus | 1.1 × 10⁸ | FFU/mL | 2.3 × 10⁸ | FFU/mL | 0.7 × 10⁸ | FFU/mL | 0.1 × 10⁸ | FFU/mL |
| Fluorescent | | | | | | | | |
| Focus Unit | | | | | | | | |
| (FFU) Assay | | | | | | | | |

| % Infectious | 9% | | 21% | 8% | 0.4% |
|---|---|---|---|---|---|
| Particles | | | | | |
| Aggregation | 66.7 | nm | 139.7 nm (23% PD) | 91.9 nm (14% PD) | 107.5 nm (8% PD) |
| by DLS | | | | | |

TABLE 16
Stability Data for NasoVAX (2 × 10¹¹ vp/mL dose)

| | Stability Time Point | | | | | | | |
|---|---|---|---|---|---|---|---|---|
| Analysis | T = 0 M | | T = 1 M | | T = 3 M | | T = 6 M | |
| Appearance | Liquid, Colorless; | | Liquid, Colorless; | | Liquid, Colorless; | | Liquid, Colorless; | |
| | Translucent; | | Translucent; | | Translucent; | | Translucent; | |
| | No visible | | No visible | | No visible | | No visible | |
| | particulate | | particulate | | particulate | | particulate | |
| | matter observed | | matter observed | | matter observed | | observed | |
| pH | 7.6 | | 7.5 | | 7.5 | | 7.6 | |
| vp by HPLC | 1.3 × 10¹¹ | vp/mL | 1.0 × 10¹¹ | vp/mL | 0.4 × 10¹¹ | vp/mL | 1.2 × 10¹¹ | vp/mL |
| Adenovirus | 0.9 × 10¹⁰ | FFU/mL | 0.9 × 10¹⁰ | FFU/mL | 0.5 × 10¹⁰ | FFU/mL | 0.1 × 10¹⁰ | FFU/mL |
| Fluorescent | | | | | | | | |
| Focus Unit | | | | | | | | |
| (FFU) Assay | | | | | | | | |

| % Infectious | 7% | | 9% | 12% | 0.5% |
|---|---|---|---|---|---|
| Particles | | | | | |
| Aggregation | 122 | nm | 118.2 nm (19% PD) | 116.9 nm (13% PD) | 115.5 nm (14% PD) |

by DLS

### Example 9: NasoVAX Shedding and Anti-NasoVAX Vector Antibodies

NasoVAX was previously evaluated in a Phase 2a, randomized, double-blind, placebo-controlled trial to evaluate the safety and immunogenicity of NasoVAX (monovalent Adco.CA.HA), in healthy adults 18 to 49 years of age. The subjects were randomized and given a single dose of 1×10⁹, 1×10¹⁰, and 1×10¹¹ viral particles (vp) or saline placebo, all given as a 0.5 mL dose split approximately as 0.25 ml nasal spray in each nostril. The protocol was described in U.S. Ser. No. 62/830,442 filed 6 Apr. 2019.

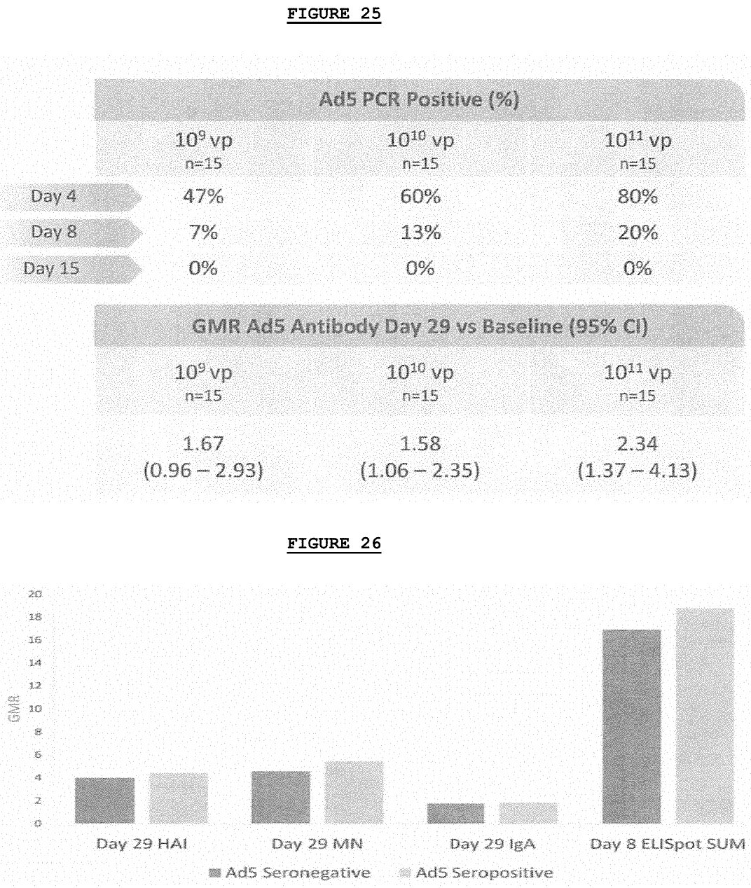

A secondary objective of that study was to evaluate the immune response against the adenoviral vector (Ad5) for subjects that were seropositive for Ad5 (as compared to subjects that were seronegative) at the time of administration of NasoVAX. At four, eight and 15 days post-dose, nasopharyngeal swab samples were collected from each subject and the concentration of the Ad5 vector shed by each quantified by polymerase chain reaction (PCR) assay. As shown in FIG. 25, dose-dependent shedding of NasoVAX vector was detected until day 8 post-dose and was not detected at day 15. No replication-competent virus was detected.

FIG. 25 also illustrates the GMR of antibodies against the Ad5 vector component of NasoVAX following administration of a single intranasal dose of 10⁹ vp, 10¹⁰ vp, or 10¹¹ vp of NasoVAX. As shown therein, administration of the highest dose (10¹¹ vp) surprisingly only resulted in about a 2.3-fold induction of anti-Ad5 vector antibodies in subjects as compared to control. This is an important finding as it indicates the intranasal route of administration can be used for repeated dosing of NasoVAX, or potentially other adenoviral vectored immunogenic compositions, including Ad5-based vectors.

FIG. 26 shows the effect of pre-existing anti-Ad5 immunity on Ad5 serostatus following administration of a single intranasal dose (10¹¹ vp) of NasoVAX to subjects. As shown therein, pre-existing anti-Ad5 immunity (“Ad5 Seropositive” (median titer being 22-fold above the lower limit of quantitation (“LLOQ”)) had little effect on humoral (HAI), microneutralization (MN), mucosal (IgA), or cellular (ELISpot) anti-Ad5 immunity following administration of the intranasal dose of NasoVAX. This is another important finding as it indicates that NasoVAX can be administered intranasally even to a subject with pre-existing immunity to Ad5. In embodiments, the present intranasal adenoviral vectored vaccine can be administered repeatedly (e.g., as a seasonal vaccine administered about once every 11-14 months) without inducing a significant immune response against the viral vector.

### Example 10: Combination of rdAd Anti-SARS-CoV-2 Vectors Human Clinical Trial as SARS-CoV-2 Vaccine

In this example, intranasal (i.n.) administration of a combination of rdAd anti-SARS-CoV-2 vectors (e.g., a “combined SARS-CoV-2 composition”) to confer prophylactic therapy against SARS-CoV-2 is described. To establish the immunogenic and/or protective capacity of such immunogenic composition(s), a composition comprising AdE is first administered to a human being, followed seven days later by administration of a composition comprising hAd5-SARS-CoV-2, which is followed by testing of the effect of this combination on the immune response against SARS-CoV-2 in the human being(s). The sequential administration of the composition AdE and then the composition comprising hAd5-SARS-CoV-2 is referred to herein as the “combined SARS-CoV-2 composition”. To do so, a randomized, double-blind, placebo-controlled, dose-escalation clinical trial to evaluate the safety and immunogenicity of combined SARS-CoV-2 composition in healthy adults 18 to 49 years of age can be carried out. Subjects are typically screened within 28 days of randomization (Day 1).

For instance, a study can comprise two parts; part A which evaluates safety, and part B which evaluates immunogenicity, of the combined SARS-CoV-2 composition. In part A, approximately 120 subjects who meet all inclusion and no exclusion criteria and provided written informed consent are enrolled into four sequential cohorts of 30 subjects each defined by the combined SARS-CoV-2 composition doses (1×10⁸, 1×10⁹, 1×10¹⁰, and 1×10¹¹ vp in each dose). Within each cohort (and the sentinel group in the first dose cohort), subjects are randomized in a 4:1:1 ratio to receive one intranasal dose of the combined SARS-CoV-2 composition (AdE composition on day 1 followed by hAd5-SARS-CoV-2 composition on day 7) or intranasal doses of placebo (normal saline) (on days 1 and 7). The combined SARS-CoV-2 composition and placebo are administered in a double-blind fashion. Reactogenicity is ascertained by determining counts and percentages of subjects with local events including but not limited to nasal irritation, sneezing, nasal congestion, cough, sore throat, change in smell, change in taste, change in vision, eye pain, pain, tenderness, induration, erythema, regional lymphadenopathy, and systemic events (headache, fatigue, myalgia, nausea, vomiting, diarrhea, coughing, chills, fever) for 14 days after vaccination. Adverse Events (AEs) are determined as counts and percentages of subjects with AEs from Day 1 to Day 57; medically attended AEs (MAAEs), serious AEs (SAES), and new-onset chronic illnesses (NCIs) from Day 1 to Day 181 following administration of the combined SARS-CoV-2 composition or placebo. For instance, targeted and symptom-driven physical examinations including vital signs can be carried out on days 4, 8, 15, 22, 29, and 57; an electrocardiogram can be carried out on day 57; safety laboratory tests can be carried out on days 8 and 57; and serum samples taken for immunogenicity testing at days 8, 15, 22, 29, 57, 91, 181, and 361 (e.g., enzyme-linked immunosorbent assay (ELISA) of serum to measure anti-SARS-CoV-2 antigen antibodies, geometric mean titer (GMT) as compared to day 0 (baseline)), and/or anti-SARS-CoV-2 immune cellular responses (e.g., T cell response). Responder rates are also determined as described below (e.g., ≥four-fold rise in IgG post dose). The primary endpoint for evaluation of the safety profile in Part A is the number and percentage (95% confidence interval (CI)) of subjects with solicited and unsolicited AEs recorded postvaccination. Safety analyses is performed using the Safety Population. The number (percentage, 95% CI) of subjects with local events and systemic events is summarized by group, as is reactogenicity. The number (percentage, 95% CI) of subjects with AEs from Day 1 to Day 57 (including MAAEs, NCIs, SAEs) is summarized for each Medical Dictionary for Regulatory Activities system organ class (SOC) by preferred term (PT) and group. The number (percentage) of subjects with MAAEs, with NCIs, and with SAEs from Day 1 to Day 181 is summarized in a similar fashion. The number (percentage, 95% CI) of subjects with AEs by severity and by relationship to investigational product (IP) is also summarized. Listings of AEs, MAAEs, NCIs, and SAEs are provided.

In part B, the immunogenicity of the combined SARS-CoV-2 composition is determined. Following administration of the combined SARS-CoV-2 composition by intranasal spray as a single dose of 1×10⁸, 1×10⁹, 1×10¹⁰, and 1×10¹¹ vp (i.e., combined SARS-CoV-2 composition (AdE composition on day 1 followed by hAd5-SARS-CoV-2 composition on day 7) of the highest well tolerated of these doses to subjects, the immune response can be measured by ELISA of serum to measure anti-SARS-CoV-2 antigen antibodies, and the GMT, geometric mean ratio (GMR) (the ratio of postvaccination and pre-vaccination GMTs within the same dose group), and responder rate (≥four-fold rise in IgG post dose), as well as immune cellular responses (e.g., T cell responses). For instance, approximately 25 subjects who meet all inclusion and no exclusion criteria and provided written informed consent are randomized in a 4:1 ratio to receive two intranasal doses of the combined SARS-CoV-2 composition at the highest well tolerated dose from Part A or placebo 21 days apart (Days 1 and 22). The combined SARS-CoV-2 composition and placebo are administered in a double-blind fashion. Intranasal doses of the combined SARS-CoV-2 composition immunogenic composition and placebo are administered to subjects in a sitting or reclined position. In part B, targeted and symptom-driven physical examination including vital signs can be carried out on days 8, 15, 22, 29, 36, 43, 50, and 57; an electrocardiogram can be carried out on day 57; safety laboratory tests on days 8, 29, and 57; serum samples taken for immunogenicity testing at days 8, 15, 22, 29, 57, 91, 181, and 361; and nasopharyngeal swabs collected on days 8, 15, 29, 36, 43, 50, 57, and 91. Nasopharyngeal samples collected at screening and on Days 29 and 57 can also be subsequently tested for evaluation of mucosal immune response.

In some embodiments, the clinical trial can be carried out using patients already infected by SARS-CoV-2 and time to clinical improvement and/or recovery determined (or, in some embodiments, a cohort of the patients tested). A primary outcome measure is Time to Clinical Improvement (TTCI) and/or Time to Clinical Recovery (TTCR) which are determined for up to 28 days following administration of the combined SARS-CoV-2 composition as described above. TTCI is defined as the time (in days) from initiation of study treatment (active or placebo) until a decline of two categories from status at randomization on a six-category ordinal scale of clinical status which ranges from 1 (discharged) to 6 (death). The six-category ordinal scale is as follows: 6. Death; 5. ICU, requiring extracorporeal membrane oxygenation (ECMO) and/or invasive mechanical ventilation (IMV); 4. Intensive care unit (ICU)/hospitalization, requiring non-invasive mechanical ventilation (NIV)/high-flow nasal cannula (HFNC) therapy; 3. Hospitalization, requiring supplemental oxygen (but not NIV/HFNC); 2. Hospitalization, not requiring supplemental oxygen; and, 1. Hospital discharge or meet discharge criteria (discharge criteria are defined as clinical recovery, i.e. fever, respiratory rate, oxygen saturation return to normal, and cough relief). Secondary outcome TTCI measures include all cause mortality (baseline SpO2 during screening, PaO2/FiO2<300 mmHg or a respiratory rate ≥24 breaths per min without supplemental oxygen); frequency of respiratory progression (SPO2≤94% on room air or PaO2/FiO2<300 mmHg and requirement for supplemental oxygen or more advanced ventilator support); time to defervescence (in those with fever at enrolment); time to cough reported as mild or absent (in those with cough at enrollment rated severe or moderate); time to dyspnea reported as mild or absent (on a scale of severe, moderate, mild absent, in those with dyspnoea at enrolment rated as severe or moderate,); frequency of requirement for supplemental oxygen or non-invasive ventilation; time to 2019-nCoV RT-PCR negative in in throat swab, sputum, lower respiratory tract specimen, and/or upper respiratory tract specimen; change (reduction) in SARS-CoV-2 viral load in throat swab, sputum, lower respiratory tract specimen, and/or upper respiratory tract specimen; change (reduction) in 2019-nCoV viral load in in throat swab, sputum, lower respiratory tract specimen, and/or upper respiratory tract specimen; change (reduction) in SARS-CoV-2 viral load in throat swab, sputum, lower respiratory tract specimen, and/or upper respiratory tract specimen as assessed by area under viral load curve (e.g., as determined using polymerase chain reaction (PCR)); frequency of requirement for mechanical ventilation; and, frequency of serious adverse events. TTCI is defined as the time (in hours) from initiation of study treatment (active or placebo) until normalization of fever, respiratory rate, and oxygen saturation, and alleviation of cough, sustained for at least 72 hours. The primary TTCR outcome measures include normalization and alleviation criteria; fever—≤36.9° C. or -axilla, ≤37.2° C. oral; respiratory rate—≤24/minute on room air; oxygen saturation—>94% on room air; and, cough—mild or absent on a patient reported scale of severe, moderate, mild, absent. The secondary TTCR outcome measures are the same as the TTCI secondary outcomes listed above.

Standard clinical trial design and statistical methods are used in the analyses thereof. For instance, the sample size for this study is selected as adequate and reasonable for an initial review of the safety and immunogenicity profile of the combined SARS-CoV-2 composition at doses to be well tolerated, rather than for statistical power (e.g., 120 subjects as described above). The sample size permits initial estimates of reactogenicity. For example, given a total of 100 subjects receiving the combined SARS-CoV-2 composition, the study is designed to have an 80% probability of detecting at least one AE that occurred at a rate of 1.6%. If no SAEs were observed among the 100 subjects who received hAdv5-SARS-CoV-2 composition, an approximation to the 1-sided upper bound of the 95% confidence interval (CI) on the rate of SAE occurrence would be 3%. Immunology analyses are conducted using the Evaluable and Per-protocol (PP) Populations with primary conclusions drawn from the PP Population. Analyses based on the Evaluable Population are undertaken and presented only if >1 subject in any one group were excluded from the PP Population. With the exception responder analyses, as described below, no imputation for missing data is performed. Data are transformed as appropriate prior to analysis. Baseline is defined as the sample collected prior to combined SARS-CoV-2 composition administration on days 1 (for the AdE composition) and 7 (for the hAdv5-SARS-CoV-2 composition). The primary variables of interest for assessment of humoral and cellular immune response to SARS-CoV-2 (e.g., anti-SARS-CoV-2 antigen IgG titers, T cell responses) are determined. GMTs are determined at Baseline and postvaccination on Days 8, 15, 22, 29, 57, 91, and 181 (Part A) and Days 8, 15, 22, 29, 36, 43, 50, 91, and 181 (Part B) and summarized by dose group. Comparisons between combined SARS-CoV-2 composition doses and placebo are evaluated by analysis of covariance (ANCOVA) with treatment as a fixed effect and baseline log-transformed level as a covariate on, e.g., the post-baseline log-transformed level of anti-SARS-CoV-2 IgG as a dependent variable. From these analyses, least-square (LS) means, LS treatment differences, and 95% CIs for the treatment differences on log-scale are obtained. The results are transformed back to the original scale by exponentiation to provide treatment geometric LS means, point estimates of the geometric LS mean ratios, and 95% CI for these ratios on each study day. A “responder” is defined as a subject with a 4-fold rise in anti-SARS-CoV-2 antigen titer from baseline on Days 8, 15, 22, 29, 57, 91, and 181 (Part A) and Days 8, 15, 22, 29, 36, 43, 50, 91, and 181 (Part B). Fold change for determination of responder status is computed using the post-imputation values without the +1 transformation, i.e., fold change=current imputed value /baseline imputed value. Responder rates are tabulated by percentages per dose group and the 95% Clopper-Pearson exact CI of the percentage. Differences of 95% CIs are presented to compare the response rate of each combined SARS-CoV2 composition dose group to the and placebo group. In some embodiments, comparisons of responders in each combined SARS-CoV-2 composition dose group against the against the placebo group can also be conducted using Fisher's exact test. To determine the effect of pre-dose Ad5 serum antibody levels on immunogenicity of combined SARS-CoV-2 composition on Day 29 (Part A) or Day 50 (Part B), analyses are performed using ANCOVA with baseline Ad5 titer as a covariate. Mucosal immunogenicity analyses are conducted using the Evaluable and PP Populations. No imputation for missing data is performed. Endpoints analyzed are GMT and GMR for IgA antibody level measured by ELISA. Methods used are the same as for humoral immunogenicity analyses. Summary statistics for continuous parameters (safety laboratory tests and vital signs) are presented by group as follows: pre-vaccination, postvaccination, and change from pre-vaccination to postvaccination assessment. The number and percentage of subjects with postvaccination safety laboratory values or vital sign values recorded as newly abnormal (ie, an event with an increase in the toxicity grade relative to the baseline value and with a severity grade of moderate or higher) after study vaccination are tabulated. Shift tables that cross-tabulate the pre-vaccination and postvaccination safety laboratory values of each subject by severity grade are prepared. Summaries of the number and percentage of subjects with normal, abnormal not clinically significant, and abnormal clinically significant ECG interpretations are presented. For shedding of the Ad5 vector, data are summarized by count and percent positive by time point, along with median copy number. The median duration of Ad5 shedding, interquartile range, minimum and maximum duration of Ad5 shedding are presented for each combined SARS-CoV-2 composition group and all immunogenic composition dose groups combined. Viral culture results for evaluation of adenovirus infection are also listed.

These studies will show that the combined SARS-CoV-2 composition can be used to induce an anti-SARS-CoV-2 immune response in human beings (e.g., it is an immunogenic composition), and with an acceptable safety profile. It is preferred that that immune response be statistically significant, and even more preferably, a protective immune response (i.e., it is a SARS-CoV-2 vaccine). In preferred embodiments, the data shows the combined SARS-CoV-2 composition can be used to treat subjects infected by SARS-CoV-2 (e.g., hospitalized patients).

### Example 11: Anti-SARS-CoV-2 Human Clinical Trial Using Cytokine Inhibition

As shown in Example 2, administration of AdE to mice was shown to decrease the expression of certain cytokines known to be involved in the progression and symptoms of infectious diseases caused by viruses such as influenza. For instance, it was shown that non-infected mice (by influenza), 25 days after administration of AdE, exhibited an increase in expression of monocyte chemoattractant protein (MCP-1 (CCL2)), interferon gamma (IFN-γ), and RANTES (CCL5). At 28 days post-administration of AdE, such non-infected mice exhibited increased expression of MCP-1 and IFN-γ but also a decrease in IL-12 expression. Mice challenged with influenza at day 3 post-administration of AdE, mice were found to exhibit decreased expression of IL-1α, IL-6, IL-12, MCP-1, tumor necrosis factor alpha (TNF-α), granulocyte macrophage colony stimulating factor (GM-CSF), and RANTES. At day six (6) post-administration of AdE, the infected mice exhibited decreased expression of IL-5, IL-6, IL-12, IL-17, MCP-1 and GM-CSF, and increased expression of macrophage inflammatory protein 1 alpha (MIP-la (CCL3)) and RANTES (CCL5). These results are consistent with the development of a “cytokine storm” during infection by SARS-CoV-2. In some embodiments, then, to prevent and/or treat SARS-CoV-2 infection by, for instance, inhibiting the development of or suppressing a cytokine storm, aSARS-CoV-2 immunogenic composition is administered to a human being with one or more anti-cytokine reagent(s) (e.g., one or more anti-IL-1α reagent(s), one or more anti-IL5 reagent(s), one or more anti-IL-6 reagent(s), one or more anti-IL-12 reagent(s), one or more anti-IL-17 reagent(s), one or more anti-MCP-1 reagent(s), one or more anti-TNF-α reagent(s), one or more anti-GM-CSF reagent(s), and/or one or more anti-RANTES reagent(s). In some embodiments, the one or more anti-cytokine reagents would not include one or more anti-MIPα reagent(s) and/or one or more anti-RANTES reagent(s). Exemplary anti-cytokine reagents that can be used as described herein can include, for example, any of those shown in Table 10.

Such anti-cytokine reagents are administered with the SARS-CoV-2 immunogenic composition at the same time (i.e., simultaneously), or essentially the same time, by a suitable route appropriate for each reagent (e.g., intranasal administration of the SARS-CoV-2 immunogenic composition and subcutaneous injection for the anti-cytokine reagent(s) in effective amounts. In some embodiments, the one or more anti-cytokine reagent(s) are co-administered with the SARS-CoV-2 composition and, in some embodiments, the one or more anti-cytokine reagents are subsequently administered as the sole active agents. These studies will show that the combination of SARS-CoV-2 composition(s) and one or more anti-cytokine reagent(s) are useful for inducing an anti-SARS-CoV-2 immune response in human beings (e.g., it is an immunogenic composition), with an acceptable safety profile, and with alleviation of symptoms related to the deleterious effects of cytokines experienced by some patients (e.g., the aforementioned cytokine storm). It is preferred that that immune response be statistically significant, and even more preferably, that it is a protective and/or curative immune response (i.e., it is a SARS-CoV-2 vaccine). In preferred embodiments, the data shows the combination of SARS-CoV-2 composition(s) and one or more anti-cytokine reagent(s) can be used to treat subjects infected by SARS-CoV-2 (e.g., hospitalized patients).

### Example 12. Animal Study Dosing Strategies

In some embodiments, one or more anti-SARS-CoV-2 vectors and/or combination(s) of anti-SARS-CoV-2 vectors (i.e., “SARS-CoV-2 vaccine”) are administered to animals in various dosages to assess pre-clinical validation of the same. In some embodiments, the mechanisms of vaccine-induced protection in animals are determined. In some embodiments, the mechanisms of vaccine-induced protection in animals with pre-existing immunity to the more common circulating coronaviruses (as is the case for most humans) are determined. These studies provide: (i) an assessment of SARS-CoV-2 vaccine-induced pulmonary inflammation; (ii) SARS-CoV-2 vaccine Ab titers with isotype and breadth of reactivity to day 28; (iii) anti-SARS-CoV-2 antibody (Ab) neutralization titers to day 28 post-administration; and, (iv) identification of optimal SARS-CoV-2 vaccine dose/administration schedule.

In some embodiments, the immunity of the animals can be studied using the flow cytometric techniques described by Yu, et al. (PLOS ONE DOI:10.1371/journal.pone.0150606 Mar. 3, 2016) which has been shown to be useful for accurately quantifying eleven distinct immune cell types, including T cells, B cells, natural killer (NK) cells, neutrophils, eosinophils, inflammatory monocytes, resident monocytes, macrophages (e.g., tissue specific macrophages, resident/interstitial macrophages, alveolar macrophages, microglia), mast cells, basophils, and/or plasmacytoid DCs, and/or to perform detailed phenotyping of specific cell types. In some embodiments, for instance, a comparison of SSC vs. MHC Class II expression can be used to separate NK cells and monocytes from mature myeloid cells; and/or, a comparison of CD64 vs. CD24 expression can be used to distinguish macrophages from dendritic cells as can CD11c vs. MHC class two expression (although the former may be more accurate). Other markers may also be studied as is known in the art (e.g., any one or more of CD11b, CD14, CD24, CD68, CD103, CD169, CD206, CX₃CR1, CCR2, F4/80, Ly6C, and/or MerTK). In some embodiments, anti-SARS-2-CoV-2 specific antibody titers in the lung airways and serum are determined (e.g., using bead arrays); inflammatory cell infiltrate into the lungs is measured (e.g., using flow cytometry); anti-SARS-CoV-2 neutralization titers are determined (e.g., using SARS-2 microneutralization assays); local and systemic cytokine levels are assessed; the number and functional attributes of anti-SARS-2 specific B cells (e.g., and subsets), T cells (e.g., (using ICCS or cytokine ELISPOTs) and plasma cells (e.g., using antibody ELISPOT) from early (7-14 days) to memory (1-4 months) timepoints following vaccination in naïve mice and those with pre-existing immunity to related endemic coronaviruses are determined. Other types of analyses may also be used as is known in the art, or would otherwise be understood by those of ordinary skill in the art to be applicable.

In some embodiments, the mechanisms of vaccine-induced protection in animals are determined studying animals to which the SARS-CoV-2 vaccine is administered following the dosing scheme shown in Table 17.

TABLE 17
Experiments 1 and 2 (Vaccine Candidate #1 and #2)*

| | N strain | | | | |
|---|---|---|---|---|---|
| | (C57BL/6 | 7 days | 14 days | 21 days | 28 days |
| Vaccine | or CD1) | (n = 10) | (n = 10) | (n = 10) | (n = 10) |
| High dose | 30 | BAL^{a} | BAL^{a} | Not done | BAL^{a} |
| Single admin** | | tissue | tissue | | tissue |
| | | phenotype^{b} | phenotype^{b} | | phenotype^{b} |
| | | Serum^{c,d} | Serum^{c,d} | | Serum^{c,d} |
| Mid dose | 30 | BAL^{a} | BAL^{a} | Not done | BAL^{a} |
| Single admin | | tissue | tissue | | tissue |
| | | phenotype^{b} | phenotype^{b} | | phenotype^{b} |
| | | Serum^{c,d} | Serum^{c,d} | | Serum^{c,d} |
| Low dose | 30 | BAL^{a} | BAL^{a} | Not done | BAL^{a} |
| Single admin | | tissue | tissue | | tissue |
| | | phenotype^{b} | phenotype^{b} | | phenotype^{b} |
| | | Serum^{c,d} | Serum^{c,d} | | Serum^{c,d} |
| High dose | 20 | Not done | Not done | BAL^{a} | BAL^{a} |
| Two admin. | | | | tissue | tissue |
| (day 0 & day | | | | phenotype^{b} | phenotype^{b} |
| 14) | | | | Serum^{c,d} | Serum^{c,d} |
| Mock | 10 | BAL^{a} | NA | NA | NA |
| | | tissue | | | |
| | | phenotype^{b} | | | |
| | | Serum^{c,d} | | | |

*Vaccine Candidate #1: S1 vector (SEQ ID NO: 13); Vaccine Candidate #2: RBD vector (SEQ ID NO: 15)
admin” represents “administration
^{a}Abs in BAL by Bead Array
^{b}BAL, lung tissue, medLN, spleen, blood T cell, B cell NK and myeloid lineage subsets (T cells, B cells, NK cells, neutrophils, eosinophils, inflammatory monocytes, resident monocytes, alveolar macrophages, resident/interstitial macrophages, CD11b− DC, and CD11b+ DC) https://www.ncbi.nlm.nih.gov/pmc/articles/PMC4777539/pdf/pone.0150606.pdf
^{c}serum Ab COVID specific, domain specific and cross reactivity to endemic coronaviruses by bead array (include isotype and subisotypes)
^{d}serum neutralizing or virus reduction assays (selecting most relevant timepoints and vaccine candidate based on binding assays from the serum samples collected)

In some embodiments, such as to evaluate B and T cell responses to vaccine candidates to determine best dosing and administration schedule, the dosing regimen shown in Table 17 is used. In some embodiments, the animals receive the SARS-CoV-2 vaccine (or two different SARS-CoV-2 vaccines as shown in Table 17) using the optimal dosing and administration schedule determined by the scheme presented in Table 18. Data is collected at four timepoints (e.g., n=7/group/timepoint/functional assay, timepoints between 0-120 days). SARS-CoV-2 vaccine-induced pulmonary inflammation and Ab responses are measured to day 28, anti-SARS-CoV-2 Ab titers with isotype and breadth of reactivity are determined to day 120, anti-SARS-CoV-2 Ab neutralization titers are determined to day 120, SARS-CoV-2 vaccine-induced T cell responses are determined to day 28, and SARS-CoV-2 vaccine-induced ASC and memory B cell responses are determined to day 120.

TABLE 18
Anti- SARS-CoV-2 functional and antigen specific assays post-vaccination

| Optimal | N | 7-14 days | 14-28 days | 60 days | 120 days |
|---|---|---|---|---|---|
| Vaccine/dose/time | strain | (n = 20) | (n = 20) | (n = 10) | (n = 10) |
| SARS-CoV-2 | 60 | BAL^{f,g,h} | BAL^{f,g,h} | BAL^{g} | BAL^{g} |
| Vaccine #1 | (inbred) | Tissues^{i,j,k} | Tissues^{i,j,k} | Tissues^{i,k} | Tissues^{i,k} |
| (optimal dosing) | | Serum^{l} | Serum^{l,m} | Serum^{l,m} | Serum^{l,m} |
| SARS-CoV-2 | 60 | BAL^{f,g,h} | BAL^{f,g,h} | BAL^{g} | BAL^{g} |
| Vaccine #2 | (inbred) | Tissues^{i,j,k} | Tissues^{i,j,k} | Tissues^{i,k} | Tissues^{i,k} |
| (optimal dosing) | | Serum^{l} | Serum^{l,m} | Serum^{l,m} | Serum^{l,m} |
| Mock | 20 | BAL^{f,g,h} | NA | NA | NA |
| | (inbred) | Tissues^{i,j,k} | | | |
| | | Serum^{l} | | | |

^{f}Cytokines/Abs in BAL supernatant (e.g. 5 cytokines from this list IL-6, IL-10, TNF-α, IL-5, IFN-α, IFN-β, IFN-γ, MIP-1a and MIP-2)
^{g}BAL cells -ELISPOTs (SARS2 specific IgA, M and G).
^{h}BAL cells T cell recall IFNg ICCS (CD4 and CD8 restim anti-CD3 and/or EL4 cells expressing SARS2 S protein)
ⁱlung tissue, medLN, Spleen, BM ELISPOTs (SARS2 specific IgA, M and G)
^{j}lung tissue, medLN, Spleen Antigen-specific T cell recall IFNγ ICCS (CD4 and CD8 restim anti-CD3 and/or EL4 cells expressing SARS2 S protein)
^{k}BAL, lung, medLN, Spleen SARS2 specific B cell responses flow cytometry
^{l}serum Ab repeats as needed
^{m}serum neutralizing or virus reduction assays as needed

In some embodiments, the following protocol is used whether pre-existing immunity to endemic β-coronavirus affects vaccine responses to SARS2. In this experiment (table 3), impact of pre-existing anti-coronavirus Spike protein memory B cells against endemic 13-coronaviruses is assessed. Inbred mice (BALB/c or B6) are vaccinated with recombinant OC43 or HKU1 Spike protein using two different adjuvants (CFA or alum) to establish a memory B cell response to the endemic coronavirus Spike protein (i.e., “memory mice”). Immunized mice and control naïve mice then receive the optimal SARS-CoV-2 vaccine(s). Pulmonary inflammation, anti-SARS-CoV-2 Ab and B cell responses, anti-SARS-CoV-2 Ab quality are assessed in the memory mice. Additional details regarding the administration scheme is provided in Table 19.

TABLE 19
Expt #4 - Pre-existing immunity to related Coronavirus
S proteins - impact on vaccination with SARS2 Spike protein

| Vaccination #1 | Vaccination | N | 7 days post | 14 days | 28 days |
|---|---|---|---|---|---|
| Day 0&14 | #2 Day 60 | strain | Vac2 (n = 10) | (n = 10) | (n = 10) |
| HKU1 or OC43 | nil | 30 | BAL^{n,o} | BAL^{n,o} | Serum^{s,t} |
| recombinant S | | (inbred) | Tissue^{p,q,r} | Tissue^{p,q,r} | |
| in adjuvant 1 | | | Serum^{s} | Serum^{s} | |
| HKU1 or OC43 | nil | 30 | BAL^{n,o} | BAL^{n,o} | Serum^{s,t} |
| recombinant S | | (inbred) | Tissue^{p,q,r} | Tissue^{p,q,r} | |
| in adjuvant 2 | | | Serum^{s} | Serum^{s} | |
| HKU1 or OC43 | SARS2 Ad5 | 30 | BAL^{n,o} | BAL^{n,o} | Serum^{s,t} |
| recombinant S | best | (inbred) | Tissue^{p,q,r} | Tissue^{p,q,r} | |
| in adjuvant 1 | | | Serum^{s} | Serum^{s} | |
| HKU1 or OC43 | SARS2 Ad5 | 30 | BAL^{n,o} | BAL^{n,o} | Serum^{s,t} |
| recombinant S | best | (inbred) | Tissue^{p,q,r} | Tissue^{p,q,r} | |
| in adjuvant 2 | | | Serum^{s} | Serum^{s} | |
| Mock | SARS2 Ad5 | 30 | BAL^{n,o} | BAL^{n,o} | Serum^{s,t} |
| | best | (inbred) | Tissue^{p,q,r} | Tissue^{p,q,r} | |
| | | | Serum^{s} | Serum^{s} | |

ⁿCytokines in BAL by Luminex (IL-6, IL-10, TNF-α, IL-5, IFN-α, IFN-β and IFN-γ)
^{o}Abs in BAL by Bead Array
^{p}BAL, lung tissue, medLN, spleen, blood T cell, B cell NK and myeloid lineage subsets (T cells, B cells, NK cells, neutrophils, eosinophils, inflammatory monocytes, resident monocytes, alveolar macrophages, resident/interstitial macrophages, CD11b− DC, and CD11b+ DC) https://www.ncbi.nlm.nih.gov/pmc/articles/PMC4777539/pdf/pone.0150606.pdf
^{q}BAL, lung, medLN, Spleen SARS2 specific B cell responses flow cytometry
^{r}cloning SARS2 specific B cell receptors and looking at breadth and depth of reactivity (and affinity/avidity)
^{s}serum Ab COVID specific, domain specific and cross reactivity to endemic coronaviruses by bead array (include isotype and subisotypes)
^{t}serum neutralizing or virus reduction assays (pseudotyped virus first and primary SARS2 second)

### Example 13A. Preparation of the Adenoviral Vectors (S1 and RBD) for Preclinical Testing

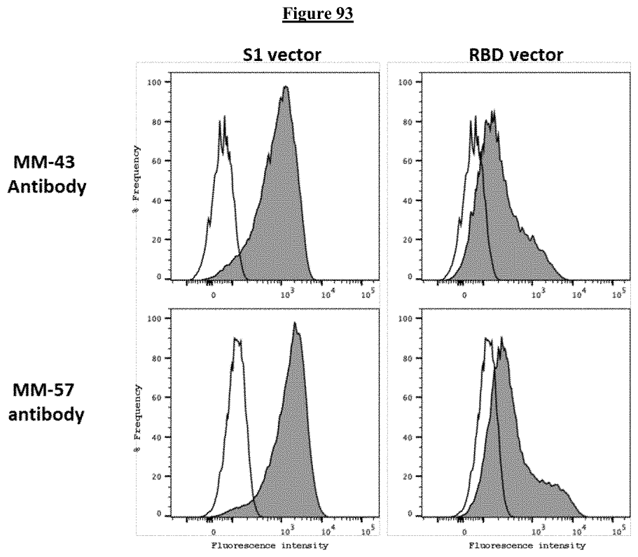

Vaccine candidates utilized in example 13A to 18 were prepared using a replication-deficient, E1- and E3-deleted adenovirus type 5 vector platform (Tang et al 2009) to express the human codon-optimized gene for the S1 domain (residues 16 to 685) or RBD domain (residues 302 to 543) of SARS-CoV-2 spike protein (accession number QHD43416.1). The Ad5-vectored S1 and RBD transgenes included a human tissue plasminogen activator leader sequence and were expressed under the control of the cytomegalovirus immediate early promoter/enhancer, SEQ ID NO: 13 and SEQ ID NO: 15, respectively. Initial seed stocks were obtained from a large-scale transfection of recombinant vector plasmid into E1-complementing PER.C6 cells using a scalable transfection system (Maxcyte STX-100). Cell transfection was performed by a static electroporation using the CL1.1 Processing Assembly procedure (Li L H, Shivakumar R, Feller S, Allen C, Weiss J M, Dzekunov S, Singh V, Holaday J, Fratantoni J, Liu LN. Highly efficient, large volume flow electroporation. Technol Cancer Res Treat. 2002 October; 1(5):341-50.). Following further expansion, infected cells were collected 70 hours post-infection and virus was released from pelleted cells by freeze-thaw cycles. Resulting cell lysate was clarified by centrifugation, then filtered using a 0.22 μm cutoff. RBD and S1 Ad5 vectors were purified over a CsCl gradient, dialyzed against a formulation buffer (A195) containing 10 mM Tris at a pH of 7.4, 75 mM NaCl, 1 mM MgCl₂, 10 mM histidine, 5% (wt/vol) sucrose, 0.02% polysorbate-80 (wt/vol), 0.1 mM EDTA, and 0.5% (vol/vol) ethanol and were then frozen and stored at −65° C. To confirm that the S1 and RBD transgenes were expressed and antigenically intact, PerC6 cells were infected with S1 vector or RBD vectors before intracellular staining using two different neutralizing monoclonal antibodies against SARS-CoV-2 S1 (SinoBiologicals Cat: 40591-MM43) and SARS-CoV-2 RBD (SinoBiologicals, Cat: 40592-MM57) and analysis by flow cytometry (FIG. 93).

### Example 13B. Intranasal Administration of Ad5 Vector Expressing RDB Domain in C57BL/6 Mice

Replication-deficient Ad5 vector expressing the RBD domain from the spike antigen of SARS-CoV-2 (SEQ ID NO: 15) was administered intranasally to C57BL/6 mice to evaluate the induction of systemic and mucosal immunity against SARS-CoV-2. C57BL/6 mice received one or two intranasal administration of the Ad5 vector at three different doses in a volume of 50 μl as indicated in table 18. High dose was 6.7E+09 ifu/ml (3.35E+08 ifu in 50 μL), mid-dose 1.2E+09 ifu/ml (6E+07 ifu in 50 μL) and Low dose 1.2E+08 ifu/ml (6E+06 ifu in 50 μL) in A195 buffer. The control group received an intranasal administration of 50 μl of the A195 buffer alone. At day 7, day 14, day 21 or day 28 post-vaccine administration, sera, bronchoalveolar lavages (BAL) and tissues including lungs, mediastinal lymph nodes and spleens were collected from 10 animals per group according to the table below. Immunological readouts included the measurement of SARS-CoV-2 spike antigen-specific-IgG in the serum and BAL, SARS-CoV-2 spike antigen-specific IgA in the BAL, neutralizing antibody responses against SARS-CoV-2 in the serum and the numeration of immune cells in the lung, lymph nodes, BALs and spleens at different time-points. These parameters are summarized in Table 20.

TABLE 20

| | | Number of | | Sample collection |
|---|---|---|---|---|
| Vaccine/ | | animals | | (10 animals per |
| Control | Intranasal dose | per group | Immunization | time point) |
| RBD Ad5 | 3.35E+08 ifu in 50 μL | 30 | Day 0 | Day 7, 14, 28 |
| RBD Ad5 | 6E+07 ifu in 50 μL | 30 | Day 0 | Day 7, 14, 28 |
| RBD Ad5 | 6E+06 ifu in 50 μL | 30 | Day 0 | Day 7, 14, 28 |
| RBD Ad5 | 3.35E+08 ifu in 50 μL | 20 | Day 0 & 14 | Day 21, 28 |
| A195 buffer | 50 μL | 10 | Day 0 | Day 7 |

The quantification of SARS-CoV-2 spike IgG and IgA was performed in serum or BAL samples obtained from immunized animals using a cytometric bead array conjugated a recombinant SARS-CoV-2 ectodomain spike protein. To produce recombinant SARS-CoV-2 S ectodomain protein, two codon-optimized constructs were generated with linear sequence order encoding: a human IgG leader sequence, the SARS-CoV-2 S ectodomain (amino acids 14-1211), a GGSG linker, T4 Fibritin Foldon sequence, a GS linker, and finally an AviTag (construct 1) or 6×-HisTag (construct 2). Each construct was engineered with two sets of mutations to stabilize the protein in a pre-fusion conformation. These included substitution of RRAR>SGAG (residues 682 to 685, as in Walls et al 2020) at the S1/S2 cleavage site and the introduction of two proline residues; K983P, V984P, as in Walls et al 2020 and Wrapp et al 2020. Avi/His-tagged trimers were produced by co-transfecting plasmid constructs 1 and 2 (1:2 ratio) into FreeStyle 293-F Cells. Cells were grown for three days and the supernatant (media) was recovered by centrifugation. Recombinant S trimers were purified from media by FPLC using a HisTrap HP Column (GE) and elution with 250 mM of imidazole. After exchanging into either 10 mM Tris-HCl, pH 8.0 or 50 mM Bicine, pH 8.3, purified spike ectodomain trimers were biotinylated by addition of biotin-protein ligase (Avidity, Aurora, Colo.). Biotinylated spike ectodomain trimers were buffer exchanged into PBS, sterile filtered, aliquoted, then stored at −80° C. until used. Following affinity purification of his-tagged protein and enzymatic biotinylation, the resulting recombinant SARS-CoV-2 trimers were passively absorbed onto streptavidin functionalized fluorescent microparticles (Spherotech 3.6 um cat #CPAK-3567-4K, peak 4). 500 μg of biotinylated SARS2-CoV-2 was incubated with 2×1e7 Streptavidin functionalized fluorescent microparticles in 400 ul of 1% BSA PBS. Following coupling, the SARS-CoV-2 spike conjugated beads were washed twice in 1 ml of 1% BSA, PBS, 0.05% NaN3 prior to final resuspension to a concentration of 1×10⁸ beads/mL. SARS-CoV-2 coupled beads were stored at 4° C. The loading of recombinant SARS2-CoV-2 spike onto the beads was evaluated by staining 1×10⁵ beads with dilutions ranging from 1 ug to 2 ng/ml of the recombinant anti-SARS spike antibody CR3022 and visualized with an anti-human IgG secondary. IgG and IgA standards were obtained by covalent coupling of isotype specific polyclonal antibodies to fluorescent particles. Briefly, 0.2 mg of goat polyclonal anti-mouse IgG (southern Biotech cat #1013-01), anti-IgM (cat #1 022-01), and anti-IgA (cat #1040-01) antibodies in PBS were mixed with 5×1e7 fluorescent microparticles each with a unique fluorescent intensity in the far red channels (Spherotech 3.6 um cat #CPAK-3567-4K, peaks 1-3) resuspended in 0.1 M MES buffer pH 5.0. An equal volume of EDC (1-Ethyl-3-(3-dimethylaminopropyl)-carbodiimide), 10 mg/mL, in 0.1 M IVIES (2-(N-morpholino) ethanesulfonic acid) buffer pH 5.0, and the mixture was incubated overnight at room temperature. The beads were washed twice by pelleting by centrifugation and resuspension in PBS. Following washing, beads were resuspended in 1% BSA, PBS with 0.005% NaN3 as a preservative. BAL samples, diluted 1/4-8, or serum samples were diluted to 1/1000-5000 in 50 μl of PBS were arrayed in 96 well u-bottom polystyrene plates along with 50 ul of standards consisting of either mouse IgG, IgM, or IgA ranging from 1 μg/ml to 2 ng/ml at 0.75× dilutions (southern biotech IgM: cat #0106-01, IgG: cat #0107-01, IgA cat #0106-01). 5 μl of a suspension containing 5×1e5 of each SARS-CoV-2 spike, anti-IgM, anti-IgA, and anti-IgG beads was added to the diluted samples. The suspensions were mixed by pipetting and incubated for 15 mins at room temperature. The beads were washed by the addition of 200 μl of PBS and centrifugation at 3000 g for 5 min at room temperature. The CBA particles were resuspended in a secondary staining solution consisting of poly-clonal anti-IgG 488 (southern Biotech cat #1010-30), and either a goat polyclonal anti-IgM (southern Biotech cat #1020-09) or anti-IgA (southern Biotech cat #1040-09) conjugated to PE diluted 1/400 in 1% BSA in PBS. The suspension was incubated for 15 min in the dark at room temperature. The beads were washed by the addition of 200 μl of PBS and pelleted by centrifugation at 3000 g for 5 min at room temperature. The particles were resuspended in 75 μl of PBS and directly analyzed on a BD Cytoflex flow cytometer in plate mode at sample rate of 100 ul per minute. Sample collection was stopped following the acquisition of 75 μL. Following acquisition, the resulting FCS files were analyzed in flowJo (treestar). Briefly, the beads were identified by gating on singlet 3.6 um particles in log scale in the forward scatter and side scatter parameters. APC-Cy7 channel fluorescence gates were used to segregate the particles by bead identity. Geometric mean fluorescent intensity was calculated in the PE and 488 channels. Best fit power curves were generated from the Ig capture beads using the know concertation of standards on a plate by plate basis. This formula was applied to the MFI of the SARS-COV-2 spike particles for all samples of the corresponding assay converting MFI to ng/ml or μg/ml. These calculated values were corrected for the dilution factor.

A foci reduction neutralization test (FNRT) was used to quantify the titer of neutralizing antibodies against SARS-CoV-2 isolate USA-WA1/2020. Vero E6 cells were grown on 96-well plates to confluence. On the day of the infection phase of the assay, serial dilutions (1:20-1:2560) of antisera were made and combined and incubated with an equal volume of viral stock, at a specified dilution for 30 min at RT, such that the final dilutions of antisera ranged from (1:40-1:5120). The viral stock was diluted from a concentrated working stock to produce an estimated 30 viral focal units per well. After incubation, the sera:virus mixtures were added to the wells (100 μL), and infection allowed to proceed for 1 hour on the Vero cells at 35° C. At the completion of the 1-hour incubation, a viscous overlay of Eagle's MEM with 4% FBS and antibiotics and 1.2% Avicell were added to sera:virus mixture on the cell monolayers such that the final volume was 200 μL per well. The infection was allowed to proceed for 24 hr. The next day, each plate was fixed by submerging the entire plate and contents in 10% formalin/PBS for 24 h. Detection of virus foci reduction was performed on fixed 96 well plates. Briefly, plates were rinsed in H₂O, and methanol:hydrogen peroxide added to the wells for 30 min with rocking to quench endogenous peroxidase activity. After quenching, plates were rinsed in H₂O to remove methanol and 5% Blotto was added to the wells as a blocking solution for 1 hour. For primary antibody detection, a SARS-CoV-2 Spike/RBD antibody (Rabbit, Polyclonal, SinoBiologicals Ct No. 40592-T62) was added to 5% Blotto and incubated on the monolayers overnight. Plates are rinsed in 5 washes with PBS, and further incubated with a secondary antibody of goat anti-rabbit IgG conjugated to horseradish peroxidase (Boster Biological Technology Co., #BA1054-0.5) in 5% Blotto for 1 hour. Plates were rinsed once with 0.05% tween in 1×PBS followed by 5 washes in 1×PBS. Detection of peroxidase activity was by use of Impact DAB detection kit (Vector Labs #SK-4105) per manufacturer's instructions. Brown foci are counted manually from the scanned image of each well, recorded, and the reduction of foci as compared to equivalent naïve mouse sera controls was determined. FRNT₅₀ titers were also calculated using a 4PL curve fit.

The analysis of bronchoalveolar lavage (BAL) cells by flow cytometry was performed as follows. BAL cells present were obtained by centrifugation at 700×g for 5 min at 4° C. of the BAL fluids. Cells were resuspended in 500 μl Red Blood Cell Lysis Buffer [ACK buffer (10 mM KHCO3, pH 7.2-7.4, 150 mM NH4Cl and 0.1 mM EDTA)]. After 1 min, 2 ml of staining media (PBS+2% Fetal Bovine Serum) with 2 mM EDTA. This media is referred to as SME. The sample were then filtered by passing it through a 70 um Nitex® Nylon filter membrane and into a clean 15-ml conical tube. Following centrifugation at 700×g for 5 min at 4° C., the cells were resuspended in 225 μl SME. 25 ul of each sample are then transferred into a 96-well plate for cell counting by flow cytometry using Fluoresbrite Carboxylate YG 10 μm microspheres. The remaining of the cells were transferred into a separate V-bottom 96-well plate for antibody staining for flow cytometric analysis. The BAL cell samples were incubated for 10 min at 4° C. in the dark with Fc-Block (1:1000 dilution), and then stained with the following BAL staining panel: Autofluorescence (empty FITC channel), Ly6G-PE (clone 1A8; 1:200 dilution), CD64-PerCP-Cy5.5 (clone X54-5/7.1; 1:150 dilution), CD8a-APC (clone 53-6.7; 1:200 dilution), CD11c-PE-Cy7 (clone N418; 1:200 dilution), CD19-APC-Fire750 (clone 6D5; 1:200 dilution), CD4-eFluor450 (clone GK1.5; 1:200 dilution) and Aqua LIVE/DEAD (1:1000 dilution). After incubation with antibody mix (50 μl total volume) for 20 min at 4° C. in the dark, cells were washed with 200 ul SME. Cells were then resuspension in 200 μl 10% formalin before analysis on FACSCanto II within 2 days.

The analysis of mediastinal lymph node by flow cytometry was performed as follows. Mediastinal lymph node (mLN) were collected and placed into separate wells of a 24-well plate containing 1 ml Staining Media (PBS+2% Fetal Bovine Serum) with added 2 mM EDTA. This media is referred to as SME. The mLN were gently grinded and crushed by rubbing in-between two microscope slides, then rinsed with 1 ml SME before transfer in a 15-ml conical tube. The volume was brought to 10 ml using SME. The cell suspension were then filtered by passing it through a 70 um Nitex® Nylon filter membrane and into a clean 15-ml conical tube before rinsing the filter membrane with an additional 2 ml SME. After centrifugation at 1800 rpm at 4° C. for 5 min, cells were resuspended with 1 ml SME. 50 ul of each sample were transferred into a 96-well plate for cell counting by flow cytometry using Fluoresbrite Carboxylate YG 10 μm microspheres. 200-250 μl of each sample were transferred into 3 separate V-bottom 96-well plates for antibody staining for flow cytometric analysis. mLN tissue were stained with 3 different flow panels. The mLN samples were incubated for 10 min at 4° C. in the dark with Fc-Block (1:1000 dilution), washed with 200 ul SME, and then stained with the following 3 panels. The myeloid panel consisted of B220/CD45R-FITC (clone RA3-6B2; 1:200 dilution), Ly6G-PE (clone 1A8; 1:200 dilution), CD64-PerCP-Cy5.5 (clone X54-5/7.1; 1:150 dilution), CD11b-APC (clone M1/70; 1:200 dilution), CD11c-PE-Cy7 (clone N418; 1:300 dilution), Ly6C-APC-Cy7 (clone AL-21; 1:200 dilution), MHCII-PB (clone M5/114.15.4; 1:600 dilution), CD3-BV510 (clone 17A2; 1:200 dilution), CD19-BV510 (clone HIB19; 1:200 dilution) and Aqua LIVE/DEAD (1:1000 dilution). For this myeloid panel staining, cells are incubated with the antibody mix (50 ul total volume) for 20 min at 4° C. in the dark. Cells were then washed with 200 μl SME and resuspended in 200 μl 10% formalin (fixative) before analysis on FACSCanto II within 2 days. The T cell panel consisted of CXCR5/CD185-FITC (clone J252D4; 1:50 dilution), PD1/CD279-PE (clone J43; 1:200 dilution), CD8a-PerCP-Cy5.5 (clone 53-6.7; 1:200 dilution), CD25-PE-Cy7 (clone PC61; 1:300 dilution), CD4-AF647 (clone GK1.5; 1:200 dilution), CD3-APC-eFluor780 (clone 17A2; 1:200 dilution), NK1.1-eFlour450 (clone PK136; 1:200 dilution) and Aqua LIVE/DEAD (1:1000 dilution). For the T cell panel staining, cells were incubated with anti-CXCR5-FITC alone (30 ul total volume; 1:50 dilution) for 30 min at 4° C. in the dark and then washed with 200 μl SME. Cell were then incubated with the rest of the antibody mix (50 μl total volume) for 15 min at 4° C. in the dark and then washed with 200 ul SME. Cells were resuspended in 200 ul 10% formalin (fixative) before analysis on FACSCanto II within 2 days. The B cell panel consisted of CD95/FAS-FITC (clone Jo2; 1:200 dilution), SPIKE (Covid-19)-PE (1:50 dilution) 7-AAD (1:1000 dilution), F4/80-PerCP-Cy5.5 (clone BM8; 1:200 dilution), CD3-PerCP-Cy5.5 (clone 17A2; 1:200 dilution), SPIKE (Covid-19)-APC (1:50 dilution) CD38-PE-Cy7 (clone 90; 1:400 dilution), CD19-APC-Fire750 (clone 6D5; 1:200 dilution), CD138-BV421 (clone 281-2; 1:200 dilution) and IgD-BV510 (clone 11-26c.2a; 1:500 dilution). For the B cell panel staining, cells were incubated with antibody mix (50 ul total volume) for 20 min at 4° C. in the dark and then washed with 200 ul SME. Cell were resuspended in 200 ul SME (no fixative) and immediately analyzed on FACSCanto II.

The analysis of lung tissues by flow cytometry was performed as follows. Excised lungs were cut into very small pieces using scissors before addition Collagenase/DNase. After incubation at 37° C. for 30 min, lung homogenates were diluted with lml SME. Digested tissues were gently grinded and crushed by using the flat end of a syringe plunger onto a strainer before rinsing with 10-15 ml SME. After centrifugation at 1800 rpm and 4° C. for 5 min, pellets were resuspended in 3 ml Red Blood Cell Lysis Buffer [ACK buffer (10 mM KHCO3, pH 7.2-7.4, 150 mM NH4C1 and 0.1 mM EDTA)]. After incubation at room temperature for 5 min. Samples were diluted 1:2 by adding 3 ml SME to inactivate the lysis buffer. Cells were filtered each lysate by passing it through a Nitex® Nylon filter membrane and into a clean 50-ml conical tube. After rinsing with additional 5-10 ml SME, cells were centrifuged before resuspending the cell pellets in 1 ml SME. 50 μl of each sample were placed into a 96-well plate for cell counting by flow cytometry using Fluoresbrite Carboxylate YG 10 μm microspheres. 200 μl of each sample were placed into 3 separate V-bottom 96-well plates for antibody staining for flow cytometric analysis. Samples were incubated for 10 min at 4° C. in the dark with Fc-Block (1:1000 dilution) and then washed with 200 μl SME before staining with the same 3 panels used for the lymph nodes.

The analysis of splenocytes by flow cytometry was performed as follows. Spleen were transferred into a 70-um cell strainer fitted on a 50-ml conical tube and gently grinded and crushed by using the flat end of a syringe plunger before rinsing the strainer with 10-15 ml SME. After centrifugation at 1800 rpm and 4° C. for 5 min, pellets are resuspended in 5 ml Red Blood Cell Lysis Buffer [ACK buffer (10 mM KHCO3, pH 7.2-7.4, 150 mM NH4C1 and 0.1 mM EDTA)]. After incubation at room temperature for 5 min, 20 ml SME was added to each lysate to dilute the ACK buffer. Cells were then filtered by passing it through a 70 um Nitex® Nylon filter membrane. Filtered were rinse with an additional 5 ml SME. After centrifugation, cell pellets were resuspended in 20 ml SME. 50 μl of each sample were placed into a 96-well plate for cell counting by flow cytometry using Fluoresbrite Carboxylate YG 10 μm microspheres. 200 μl of each sample were placed into 3 separate V-bottom 96-well plates for antibody staining for flow cytometric analysis. Samples were incubated for 10 min at 4° C. in the dark with Fc-Block (1:1000 dilution) and then washed with 200 μl SME before staining with the same 3 panels used for the lymph nodes.

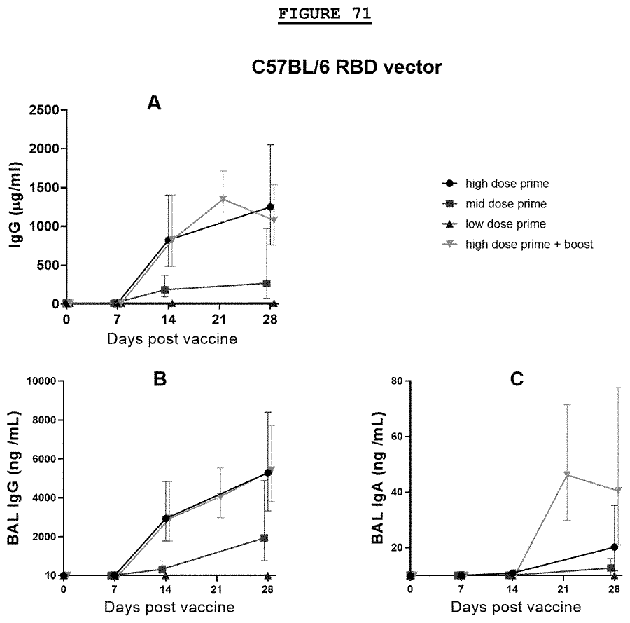

Following a single intranasal administration, the replication-deficient Ad5 vector expressing the RBD domain of Spike (SEQ ID NO: 15) was demonstrated to stimulate the production of IgG antibodies in the serum indicating the induction of systemic responses as well as the production of IgG and IgA antibodies in bronchoalveolar lavages indicating the induction of a mucosal responses as shown in FIGS. 71A-71C. A dose response was observed across the different antibody markers with the high dose vaccine leading the highest responses and the low dose leading to a marginal response while the mid-dose generated an intermediate response. It is worth noting that at day 14 and day 28 with a single high dose vaccine, spike-specific IgG response in the serum reached very high level, exceeding 1 milligram per ml representing approximately 5% of the total serum IgG. After an intranasal boost at day 14 with the same vector, the IgA production in the BAL increased significantly above the response induced by a single dose of the vaccine IgG response at day 21 and day 28 while the impact on the IgG responses was limited.

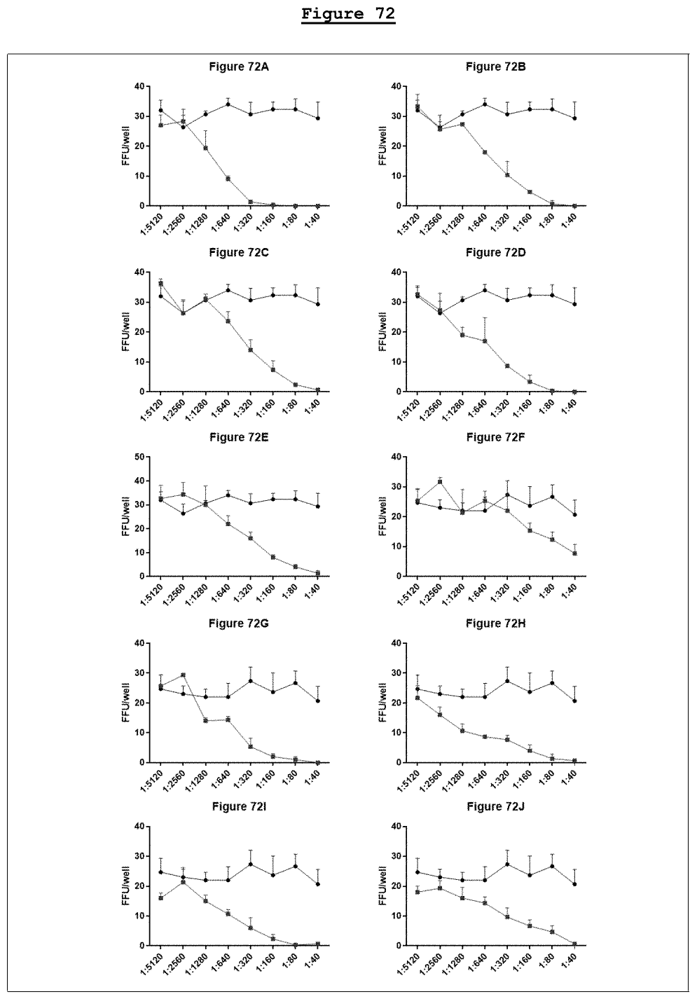

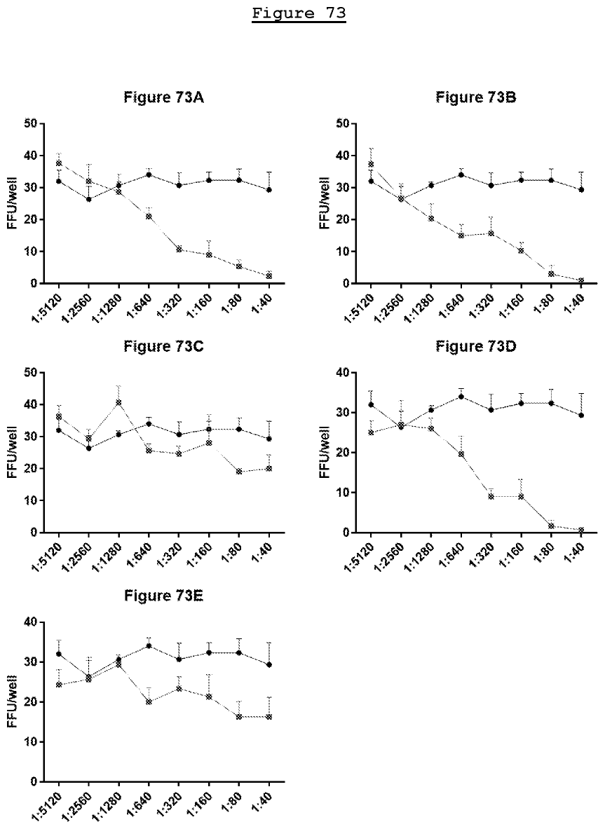

All ten animals (100%) tested from the group that received a single administration of the high dose vaccine showed the presence of neutralizing antibodies against SARS-CoV-2 as measured by focus reduction neutralization test (FRNT) (FIGS. 72A-72J) while only three out of five animals (60%) show significant neutralizing response in the group that has received a single administration of the mid-dose vaccine (FIGS. 73A-73E).

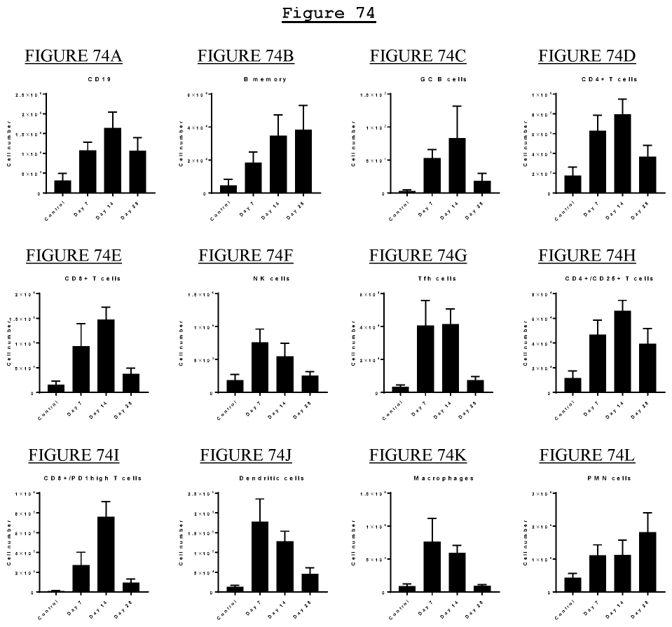

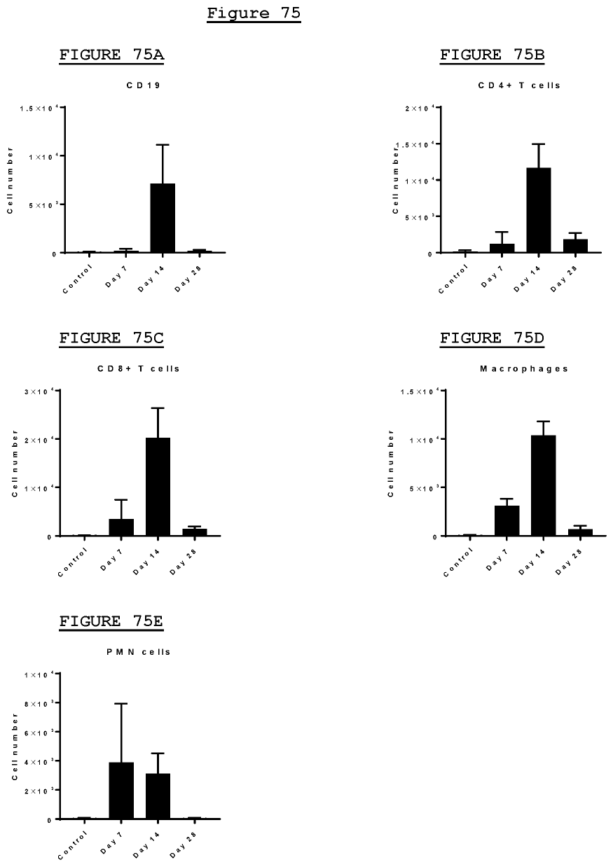

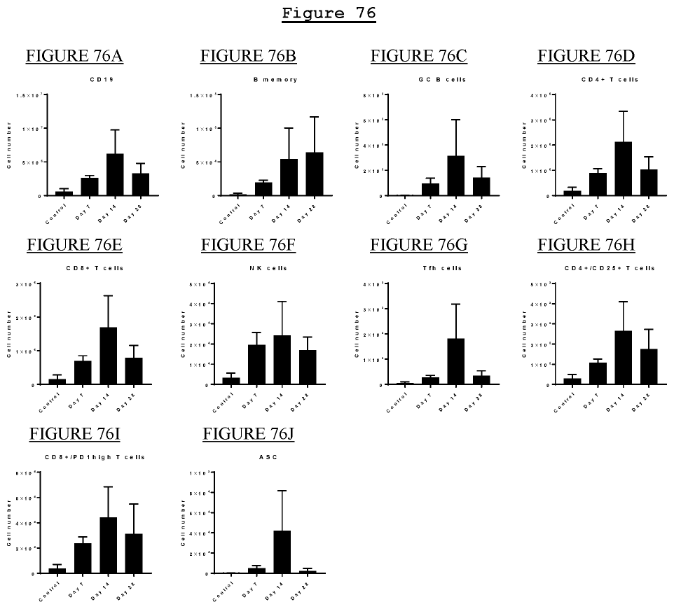

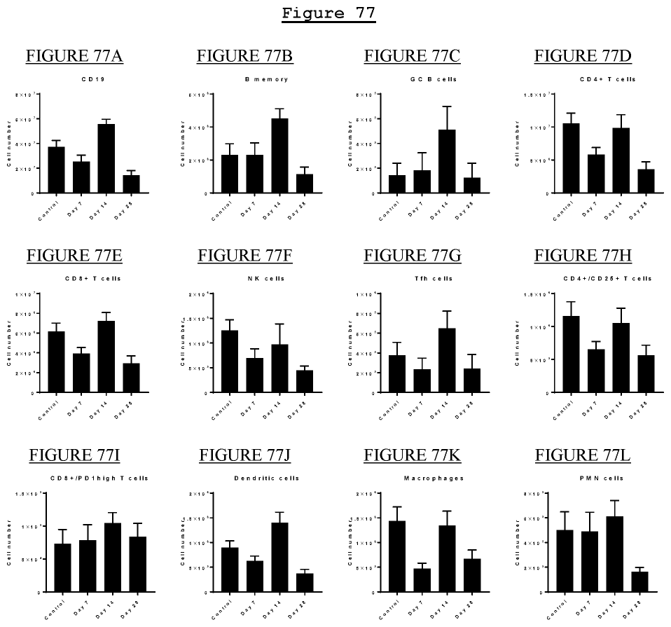

Intranasal administration of the vaccine induces the recruitment and/or proliferation of innate and adaptive immune cells in different immune compartments. FIGS. 74A-74L, 75A-75E, 76A-76J, and 77A-L present the variations in the number of immune cells respectively in the lung, BAL, mediastinal lymph nodes and the spleen following the single intranasal administration of the high dose vaccine. Importantly, elevation on the number of CD19 cells, memory B cells, Germinal Center (GC) B cells and/or T follicular helper (hf) cells observed in the lung, BAL and lymph nodes and to a lesser extend in the spleen are indicative of the ability of the vaccine to stimulate a long-lived humoral and mucosal antibody response. In addition, the elevation of macrophages, dendritic cells, CD4+ and/or CD8+ T cells observed in the lung, BAL and lymph nodes and to a lesser extend in the spleen are indicatives of the ability of the vaccine to stimulate mucosal and systemic, innate and cell-mediated immune responses that are anticipated to be of benefit in the context of SARS-CoV-2 infection. Results obtained with the lower vaccine doses were not presented as they show a similar trend but at a lower level.

### Example 14. Intranasal Administration of Ad5 Vector Expressing S1 Domain in C57BL/6 Mice

Replication-deficient Ad5 vector expressing the S1 domain from the spike antigen of SARS-CoV-2 (SEQ ID NO: 13) was administered intranasally to C57BL/6 mice to evaluate the induction of systemic and mucosal immunity against SARS-CoV-2. C57BL/6 mice received one or two intranasal administration of the Ad5 vector at three different doses in a volume of 50 μl as indicated in Table 19. High dose was 1.2E+09 ifu/ml (6E+08 ifu in 50 μL), mid-dose 1.2E+09 ifu/ml (6E+07 ifu in 50 μL) and Low dose 1.2E+08 ifu/ml (6E+06 ifu in 50 μL) in A195 buffer. The control group received an intranasal administration of 50 μl of the A195 buffer alone. At day 7, day 14, day 21 or day 28 post-vaccine administration, sera, bronchoalveolar lavages (BAL) and tissues including lungs, mediastinal lymph nodes and spleens were collected from 10 animals per group according to the table below. Immunological readouts included the measurement of SARS-CoV-2 spike antigen-specific-IgG in the serum and BAL, SARS-CoV-2 spike antigen-specific IgA in the BAL, neutralizing antibody responses against SARS-CoV-2 in the serum and the numeration of immune cells in the lung, lymph nodes, BALs and spleens at different time-points. These parameters are summarized in Table 21.

TABLE 21

| | | Number of | | Sample collection |
|---|---|---|---|---|
| Vaccine/ | | animals | | (10 animals per |
| Control | Intranasal dose | per group | Immunization | time point) |
| S1 Ad5 | 6E+08 ifu in 50 μL | 30 | Day 0 | Day 7, 14, 28 |
| S1 Ad5 | 6E+07 ifu in 50 μL | 30 | Day 0 | Day 7, 14, 28 |
| S1 Ad5 | 6E+06 ifu in 50 μL | 30 | Day 0 | Day 7, 14, 28 |
| S1 Ad5 | 6E+08 ifu in 50 μL | 20 | Day 0 & 14 | Day 21, 28 |
| A195 buffer | 50 μL | 10 | Day 0 | Day 7 |

Methods for the quantification of SARS-CoV-2 spike IgG and IgA in serum or BAL samples, focus reduction neutralization tests as well as the flow cytometric analysis of bronchoalveolar lavage (BAL) cells, mediastinal lymph nodes, lung tissues and splenocytes are described in Example 13B.

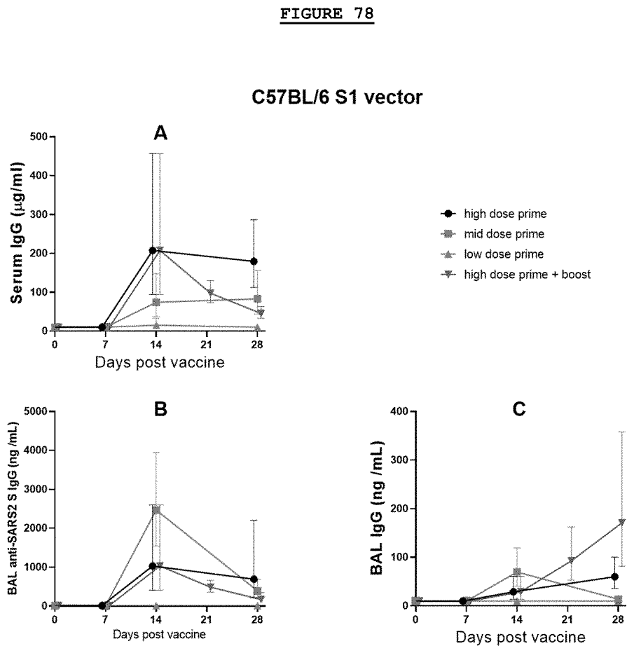

Following a single intranasal administration, the replication-deficient Ad5 vector expressing the S1 domain of Spike (SEQ ID NO: 13) was demonstrated to stimulate the production of IgG antibodies in the serum indicating the induction of systemic responses as well as the production of IgG and IgA antibodies in bronchoalveolar lavages indicating the induction of a mucosal responses as shown in FIGS. 78A-78C. A dose response was observed across the different antibody markers with the high dose vaccine leading to the highest responses and the low dose leading to a marginal response while the mid-dose generated an intermediate response. Overall, IgG responses in the serum and BALs were lower with the S1 vector compared to the results obtained with the RBD vector as presented in Example 13B. After an intranasal boost at day 14 with the same vector, the IgA production in the BAL increased significantly above the response induced by a single dose of the vaccine IgG response at day 21 and day 28 while the impact on the IgG responses was limited.

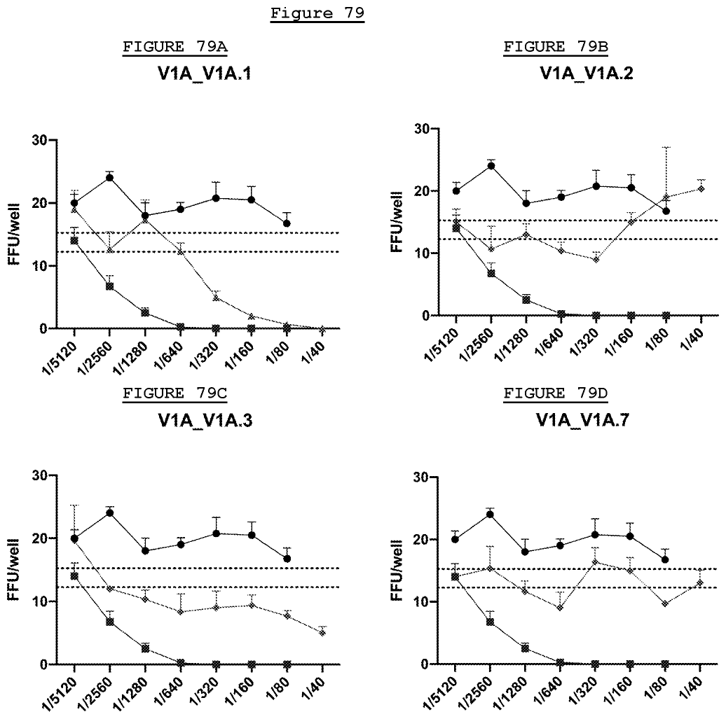

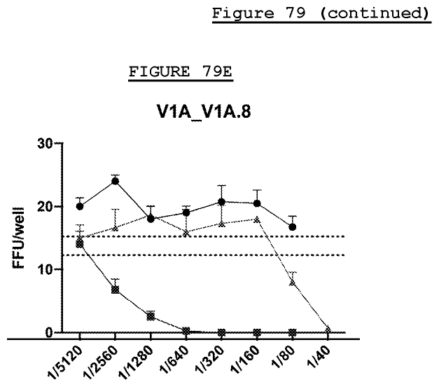

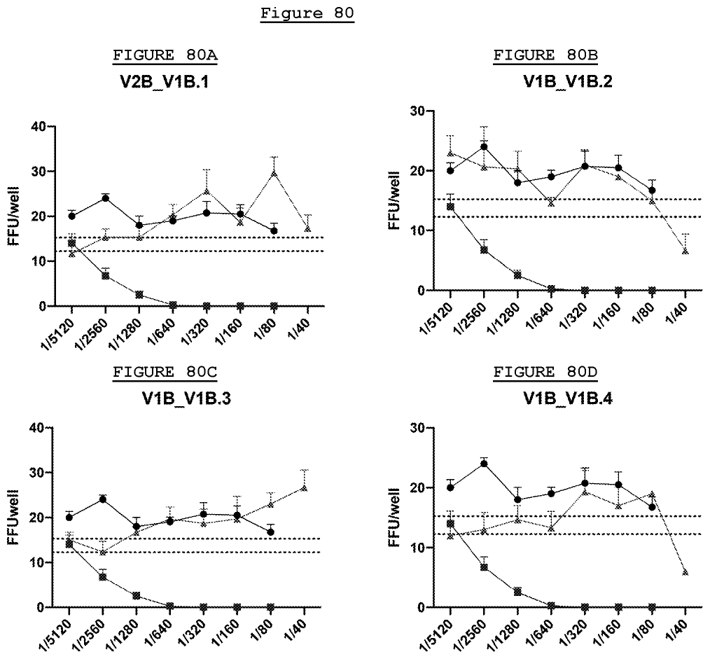

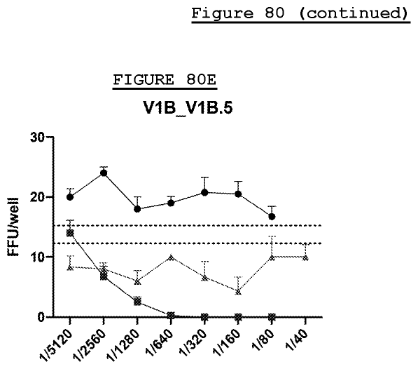

Three out of five animals tested from the group that received a single administration of the high dose vaccine showed significant induction of neutralizing antibodies against SARS-CoV-2 as measured by focus reduction neutralization test (FRNT) (FIGS. 79A-79E). In the single mid-dose group, two out of five animals (FIGS. 80B and D) show low neutralizing response (FIGS. 80A-80E).

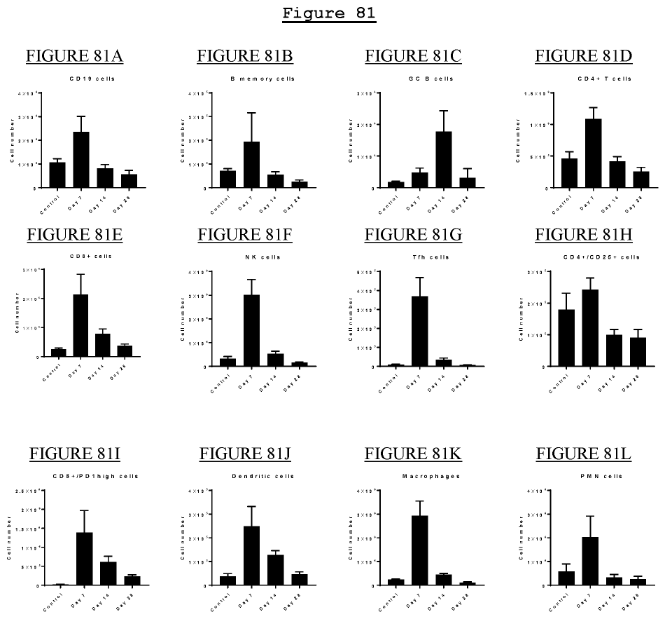

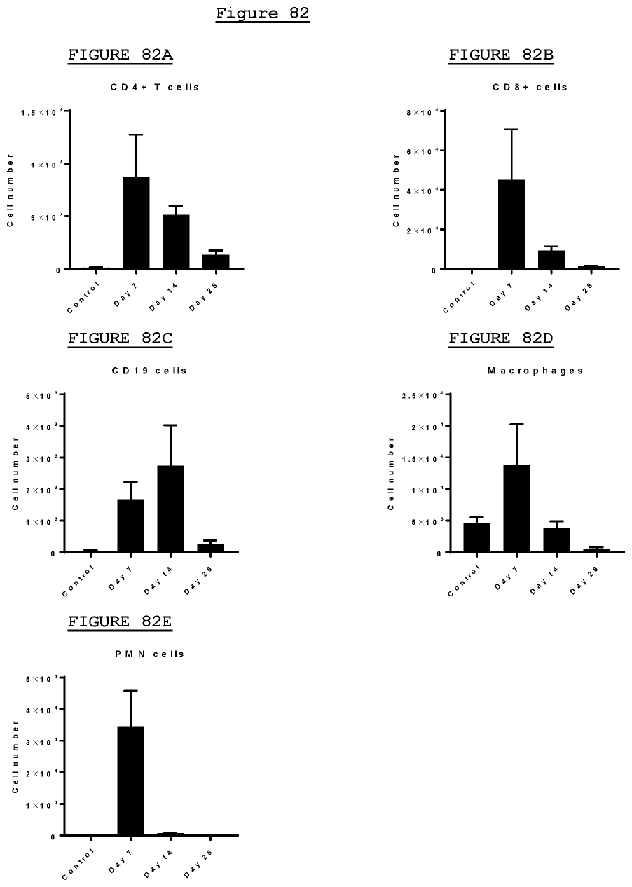

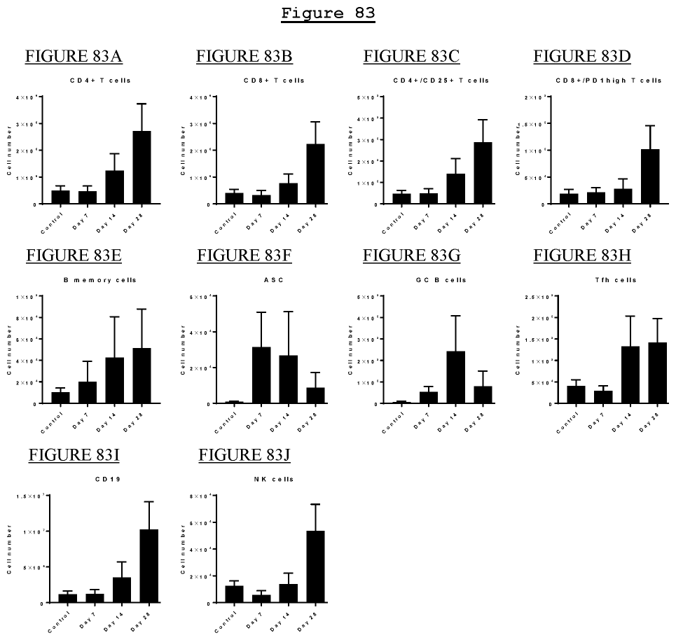

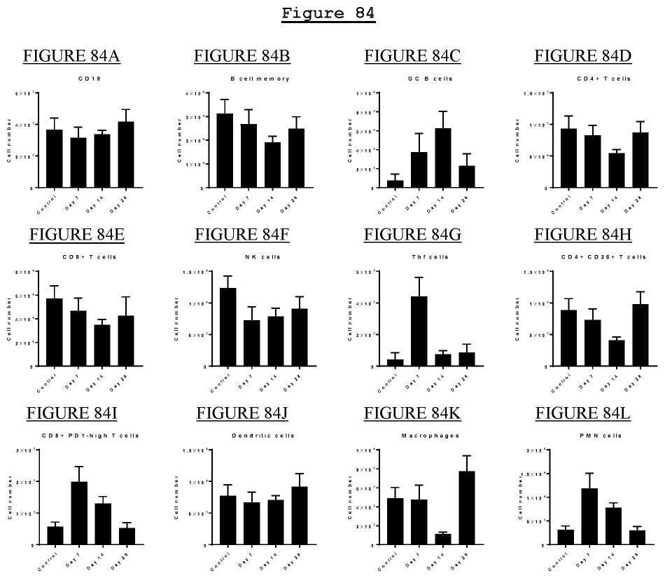

Intranasal administration of the vaccine induces the recruitment and/or proliferation of innate and adaptive immune cells in different immune compartments. FIGS. 81A-81L, 82A-82E, 83A-83J, and 84A-L present the variations in the number of immune cells respectively in the lung, BAL, mediastinal lymph nodes and the spleen following the single intranasal administration of the high dose vaccine. Importantly, elevation on the number of CD19 cells, memory B cells, Germinal Center (GC) B cells and/or T follicular helper (hf) cells observed in the lung, BAL and lymph nodes and to a lesser extend in the spleen are indicative of the ability of the vaccine to stimulate a long-lived humoral and mucosal antibody response. In addition, the elevation of macrophages, dendritic cells, CD4+ and/or CD8+ T cells observed in the lung, BAL and lymph nodes and to a lesser extend in the spleen are indicatives of the ability of the vaccine to stimulate mucosal and systemic, innate and cell-mediated immune responses that are anticipated to be of benefit in the context of SARS-CoV-2 infection. Results obtained with the lower vaccine doses were not presented as they show a similar trend but at a lower level.

### Example 15. Intranasal Administration of Ad5 Vector Expressing RBD Domain in CD-1 Mice

Replication-deficient Ad5 vector expressing the RBD domain from the spike antigen of SARS-CoV-2 (SEQ ID NO: 15) was administered intranasally to CD-1 mice to evaluate the induction of systemic and mucosal immunity against SARS-CoV-2. CD-1 mice received one or two intranasal administration of the Ad5 vector at three different doses in a volume of 50 μl. High dose was 6.7E+09 ifu/ml (3.35E+08 ifu in 50 μL), mid-dose 1.2E+09 ifu/ml (6E+07 ifu in 50 μL) and Low dose 1.2E+08 ifu/ml (6E+06 ifu in 50 μL) in A195 buffer. The control group received an intranasal administration of 50 μl of the A195 buffer alone. At day 7, day 14 and/or day 21 post-vaccine administration, sera and bronchoalveolar lavages (BAL) were collected from 10 animals per group according to Table 22. Immunological readouts included the measurement of SARS-CoV-2 spike antigen-specific-IgG in the serum and BAL, SARS-CoV-2 spike antigen-specific IgA in the BAL, and neutralizing antibody responses against SARS-CoV-2 in the serum. These parameters are summarized in Table 22.

TABLE 22

| | | Number of | | Sample collection |
|---|---|---|---|---|
| Vaccine/ | | animals | | (10 animals per |
| Control | Intranasal dose | per group | Immunization | time point) |
| RBD Ad5 | 3.35E+08 ifu in 50 μL | 30 | Day 0 | Day 7, 14, 21 |
| RBD Ad5 | 6E+07 ifu in 50 μL | 30 | Day 0 | Day 7, 14, 21 |
| RBD Ad5 | 6E+06 ifu in 50 μL | 30 | Day 0 | Day 7, 14, 21 |
| RBD Ad5 | 3.35E+08 ifu in 50 μL | 20 | Day 0 & 14 | Day 21 |
| A195 buffer | 50 μL | 10 | Day 0 | Day 7 |

Methods for the quantification of SARS-CoV-2 spike IgG and IgA in serum or BAL samples and focus reduction neutralization tests are described in Example 13B.

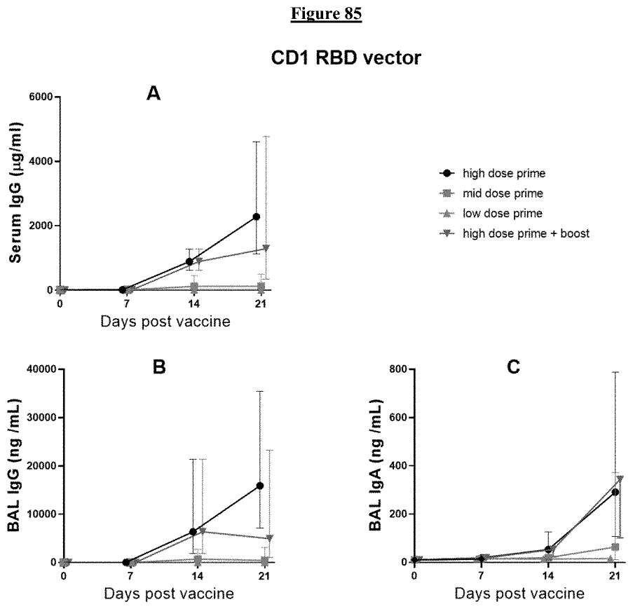

Following a single intranasal administration, the replication-deficient Ad5 vector expressing the RBD of the S1 domain of Spike (SEQ ID NO: 15) was demonstrated to stimulate the production of IgG antibodies in the serum indicating the induction of systemic responses as well as the production of IgG and IgA antibodies in bronchoalveolar lavages indicating the induction of a mucosal responses as shown in FIGS. 85A-85C. A dose response was observed across the different antibody markers with the high dose vaccine leading to the highest responses and the low dose leading to a marginal response while the mid-dose generated an intermediate response. After an intranasal boost at day 14 with the same vector, impact on the IgG and IgA responses were limited compared to the single administration at the same dose.

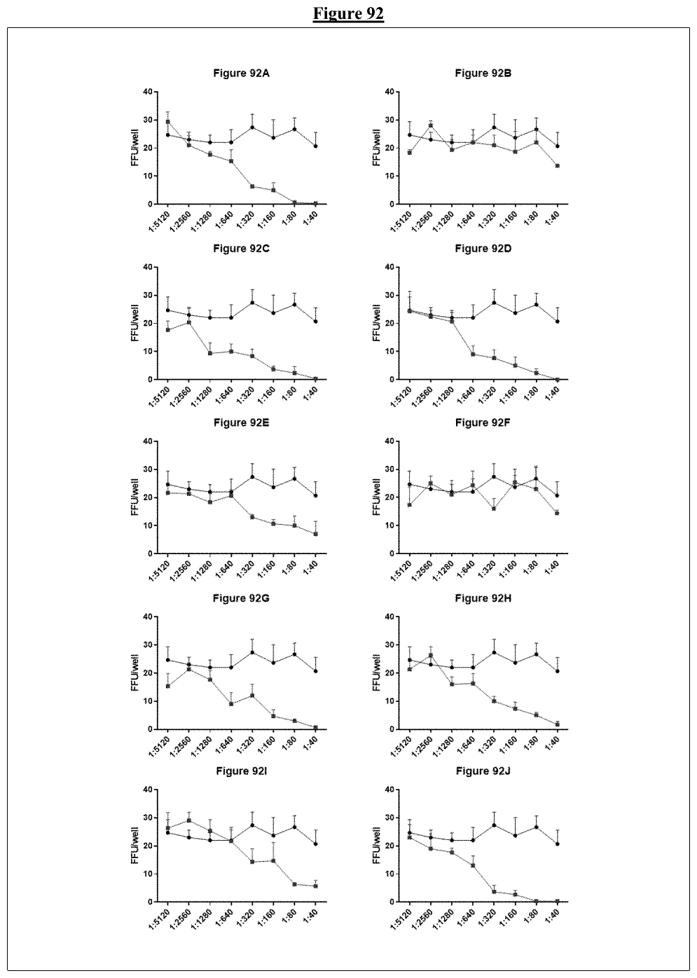

Eight out of 10 animals tested (FIGS. 92A, 92C-92E and 92G to 92J) from the group that received a single administration of the high dose vaccine showed significant induction of neutralizing antibodies against SARS-CoV-2 as measured by focus reduction neutralization test (FRNT) (FIGS. 92A-92J).

### Example 16. Intranasal Administration of Ad5 Vector Expressing S1 Domain in CD-1 Mice

Replication-deficient Ad5 vector expressing the S1 domain from the spike antigen of SARS-CoV-2 (SEQ ID NO: 13) was administered intranasally to CD-1 mice to evaluate the induction of systemic and mucosal immunity against SARS-CoV-2. CD-1 mice received a single intranasal administration of the Ad5 vector at three different doses in a volume of 50 μl. High dose was 6.7E±09 ifu/ml (3.35E±08 ifu in 50 μL), mid-dose 1.2E±09 ifu/ml (6E±07 ifu in 50 μL) and Low dose 1.2E±08 ifu/ml (6E±06 ifu in 50 μL) in A195 buffer. The control group received an intranasal administration of 50 μl of the A195 buffer alone. At day 7, day 14 and day 21 post-vaccine administration, sera and bronchoalveolar lavages (BAL) were collected from 10 animals per group according to Table 23. Immunological readouts included the measurement of SARS-CoV-2 spike antigen-specific-IgG in the serum and BAL, SARS-CoV-2 spike antigen-specific IgA in the BAL, neutralizing antibody responses against SARS-CoV-2 in the serum. These parameters are summarized in Table 23.

TABLE 23

| | | Number of | | Sample collection |
|---|---|---|---|---|
| Vaccine/ | | animals | | (10 animals per |
| Control | Intranasal dose | per group | Immunization | time point) |
| S1 Ad5 | 3.35E+08 ifu in 50 μL | 30 | Day 0 | Day 7, 14, 21 |
| S1 Ad5 | 6E+07 ifu in 50 μL | 30 | Day 0 | Day 7, 14, 21 |
| S1 Ad5 | 6E+06 ifu in 50 μL | 30 | Day 0 | Day 7, 14, 21 |
| A195 buffer | 50 μL | 10 | Day 0 | Day 7 |

Methods for the quantification of SARS-CoV-2 spike IgG and IgA in serum or BAL samples and focus reduction neutralization tests are described in Example 13B.

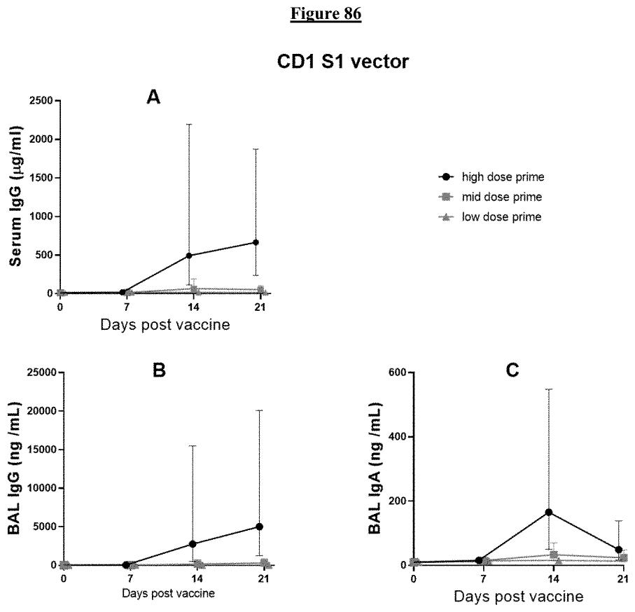

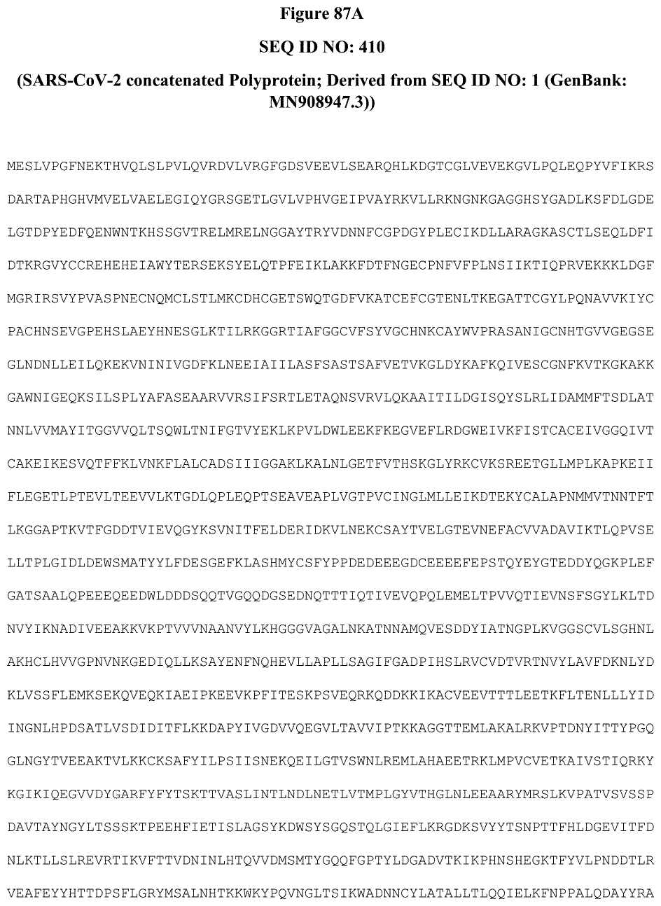

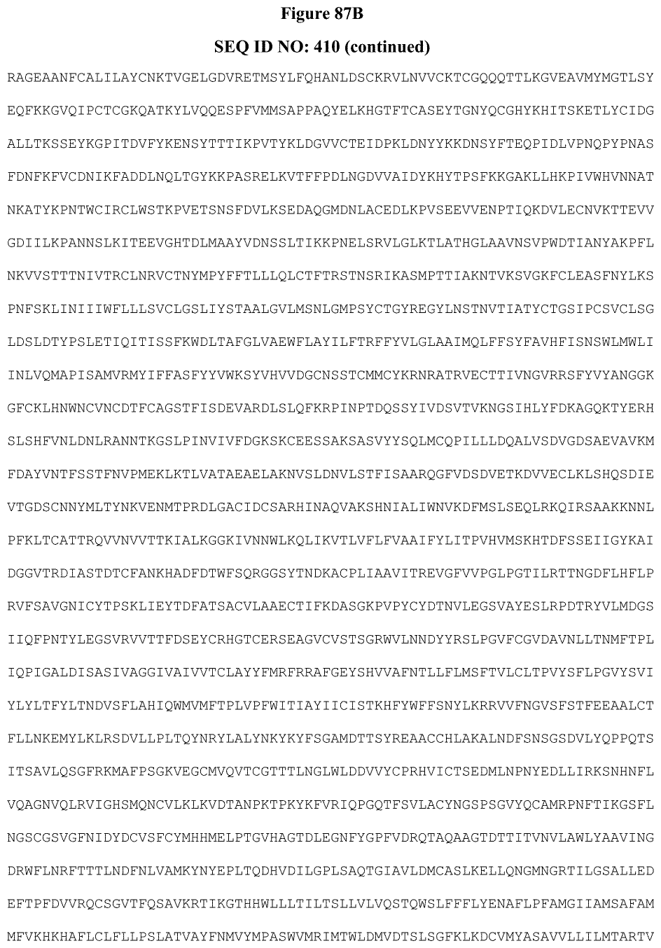

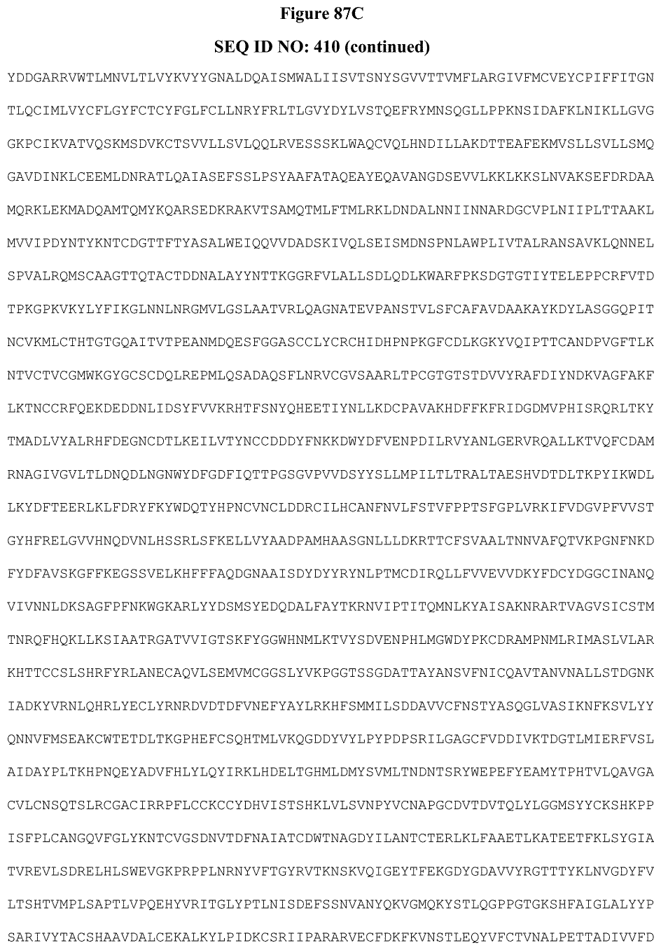

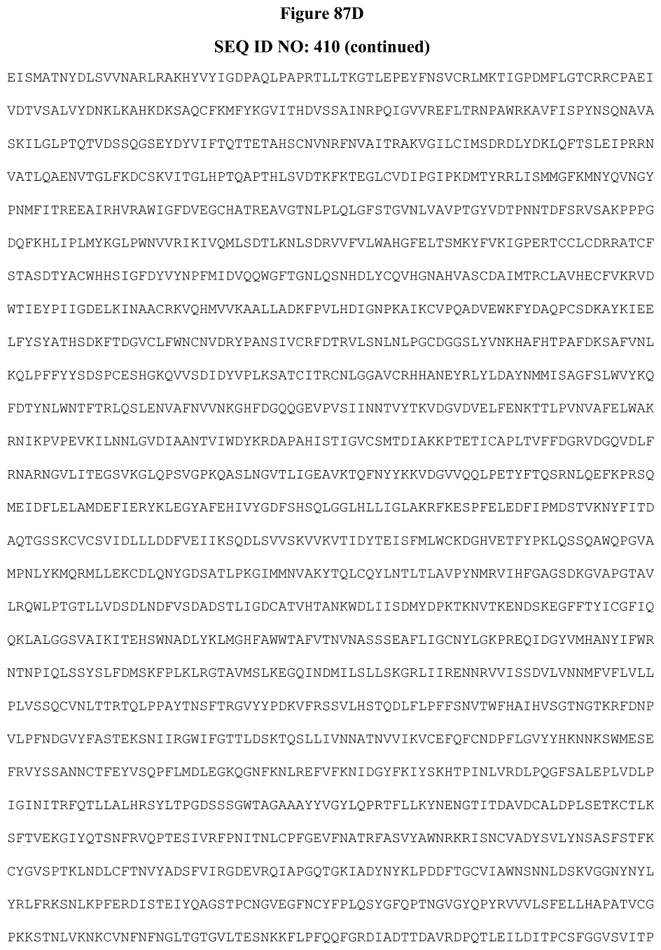

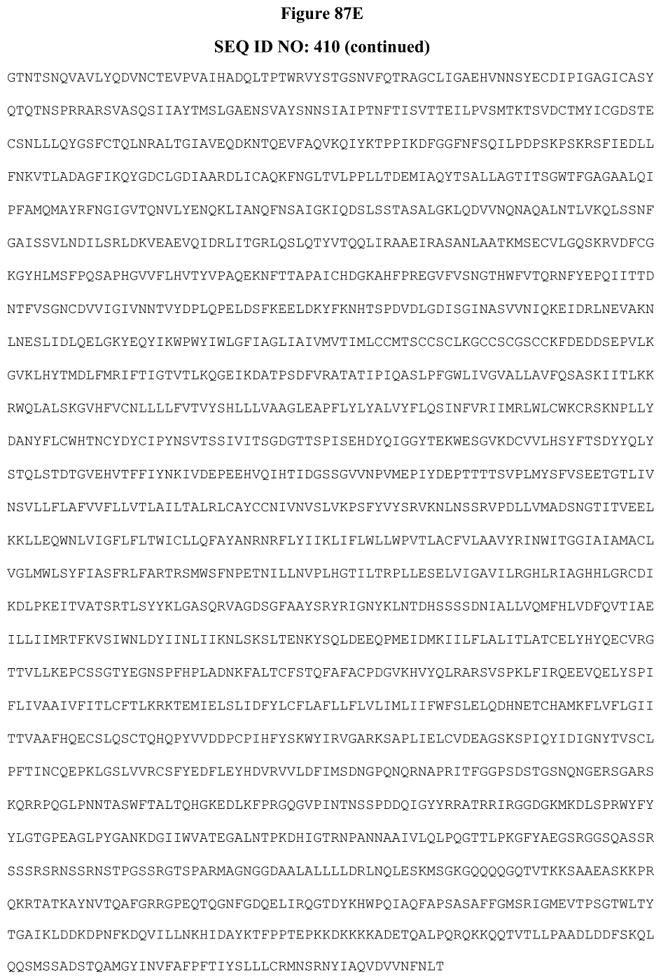

Following a single intranasal administration, the replication-deficient Ad5 vector expressing the S1 domain of Spike (SEQ ID NO: 13) was demonstrated to stimulate the production of IgG antibodies in the serum indicating the induction of systemic responses as well as the production of IgG and IgA antibodies in bronchoalveolar lavages indicating the induction of a mucosal responses as shown in FIGS. 86A-86C. A dose response was observed across the different antibody markers with the high dose vaccine leading to the highest responses and the low dose and mid dose leading to a marginal immune response.

### Example 17. Intranasal Administration of Ad5 Vector Expressing RBD Domain in CD-1 Mice

Replication-deficient Ad5 vector expressing the RBD domain from the spike antigen of SARS-CoV-2 (SEQ ID NO: 15) was administered intranasally to CD-1 mice to evaluate the induction of systemic and mucosal T cell immunity against SARS-CoV-2. CD-1 mice received a single intranasal administration of the Ad5 vector at 6.7E±09 ifu/ml (3.35E±08 ifu in 50 μL) in A195 buffer. The control group received an intranasal administration of 50 μl of the A195 buffer alone. At day 10, day 14 and day 28 post-vaccine administration, lungs and spleens from 10 mice in the vaccine group and 3 mice from the control group were collected according to Table 24. Immunological readouts included the measurement of CD4+ and CD8+ T cell responses in the lungs and spleens by flow cytometry, the measurement of T cell response in the lungs and spleens by an IFN-gamma ELISpot assay as well as the measurement of T cell cytokines following in vitro recall with RBD-derived peptides.

TABLE 24

| | | Number of | | Sample collection |
|---|---|---|---|---|
| Vaccine/ | | animals | | (10 animals per |
| Control | Intranasal dose | per group | Immunization | time point) |
| RBD Ad5 | 3.35E+08 ifu in 50 μL | 30 | Day 0 | Day 7, 14, 28 |
| A195 buffer | 50 μL | 9 | Day 0 | Day 7, 14, 28 |

The analysis of RBD-specific T cell responses by IFN-gamma ELISpot in the spleens and lungs was performed as follows. Spleens were transferred into a 70-um cell strainer fitted on a 50-ml conical tube and gently grinded and crushed by using the flat end of a syringe plunger before rinsing the strainer with 10-15 ml SME. After centrifugation at 1800 rpm and 4° C. for 5 min, pellets are resuspended in 5 ml Red Blood Cell Lysis Buffer [ACK buffer (10 mM KHCO3, pH 7.2-7.4, 150 mM NH4C1 and 0.1 mM EDTA)]. After incubation at room temperature for 5 min, 20 ml SME was added to each lysate to dilute the ACK buffer. Cells were then filtered by passing through a 70 um Nitex® Nylon filter membrane. Filtered cells were rinsed with an additional 5 ml SME. After centrifugation, cell pellets were resuspended in 20 ml SME. 50 μl of each sample were placed into a 96-well plate for cell counting by flow cytometry using Fluoresbrite Carboxylate YG 10 μm microspheres.

Excised lungs were cut into very small pieces using scissors before addition of Collagenase/DNase. After incubation at 37° C. for 30 min, lung homogenates were diluted with 1 ml SME. Digested tissues were gently grinded and crushed by using the flat end of a syringe plunger onto a strainer before rinsing with 10-15 ml SME. After centrifugation at 1800 rpm and 4° C. for 5 min, pellets were resuspended in 3 ml Red Blood Cell Lysis Buffer [ACK buffer (10 mM KHCO3, pH 7.2-7.4, 150 mM NH4C1 and 0.1 mM EDTA)]. After incubation at room temperature for 5 min. Samples were diluted 1:2 by adding 3 ml SME to inactivate the lysis buffer. Cells were filtered by passing it through a Nitex® Nylon filter membrane and into a clean 50-ml conical tube. After rinsing with additional 5-10 ml SME, cells were centrifuged before resuspending the cell pellets in 1 ml SME. 50 μl of each sample were placed into a 96-well plate for cell counting by flow cytometry using Fluoresbrite Carboxylate YG 10 μm microspheres.

For analysis of T cell responses, a pool of 53 peptides derived from a peptide scan through RBD of Spike Glycoprotein of SARS-CoV-2 (319-541) was designed and synthesized by JPT (JPT Peptide technologies, Berlin, Germany). Peptides were designed with a length of 15 a.a. and an overlap of 11a.a. Before use, each vial containing 15 nmol (appr. 25 μg) of each peptide per vial was reconstituted in 50 μl of DMSO before dilution into complete culture media.

Spleen and lung cell suspensions (150,000 cells/well) were placed in individual wells of ELIspot plates (Millipore-Sigma) that were pre-coated with anti-IFN-γ (AN18, (5 μg/ml)). Cells were stimulated with the RBD peptide pool described above at 0.5 to 2.0 μg/peptide/ml. Following 24 hr stimulation, plates were stained with biotinylated anti-IFN-γ (R4-6A2), followed by washing steps, and incubation with streptavidin-ALP. Secreted IFN-γ was detected following incubation with NBT/NCPI substrate for 7-10 min. The number of IFN-γ spot-forming cells were manually counted from digital images of each well. Statistical analysis was performed in GraphPad Prism using a Mann-Whitney test.

The analysis of CD4+ and CD8+ T cell responses in lung tissues and spleens by flow cytometry was performed as follows. Spleen and lung single cell suspensions were stimulated with the RBD peptide pool for 5 hrs in the presence of Brefeldin A (5 hrs, 12.5 ug/mL concentration). Cells were then incubated on ice with a combination of fluorescent dye-labelled antibodies including anti-CD4-V500 (clone GK1.5; 1:200 dilution), anti-CD8a-APC-Fire750 (clone 53-6.7; 1:200 dilution), anti-CD11a/CD18-Pacific Blue (H155-78; 1:200 dilution), anti-CD103-PE (M290; 1:200 dilution), anti-CD69-FITC (H1-2F3; 1:200 dilution), anti-Ly6G-PerCP-Cy5.5 (clone 1A8; 1:200 dilution), anti-CD64-PerCP-Cy5.5 (clone X54-5/7.1; 1:200 dilution), anti-B220/CD45R-PerCP (clone RA3-6B2; 1:200 dilution), and Red LIVE/DEAD (1:1000 dilution). Following surface staining, cells were permeabilized using BD Biosciences Cytofix/Cytoperm kit, and stained with anti-IFN-γ-PE-Cy7 (XMG1.2; 1:200 dilution) and anti-TNF-α-APC (MP6-XT22; 1:200 dilution). Following incubation with the antibodies, cells were washed and resuspended before analysis on FACSCanto II within 12 hours. Statistical analysis was performed in GraphPad Prism using a Mann-Whitney test.

Protein levels of IFNγ, IL-2, IL-4, IL-5, IL-10, IL-13, IL-17A and TNFα were quantified in culture supernatants using the mouse-specific Milliplex® multi-analyte panel kit MT17MAG-47K (Millipore; Sigma) and the MagPix® instrument platform with related xPONENT® software (Luminex Corporation). The readouts were analyzed with the standard version of EMD Millipore's Milliplex® Analyst software. Statistical analysis was performed in GraphPad Prism using a Mann-Whitney test.

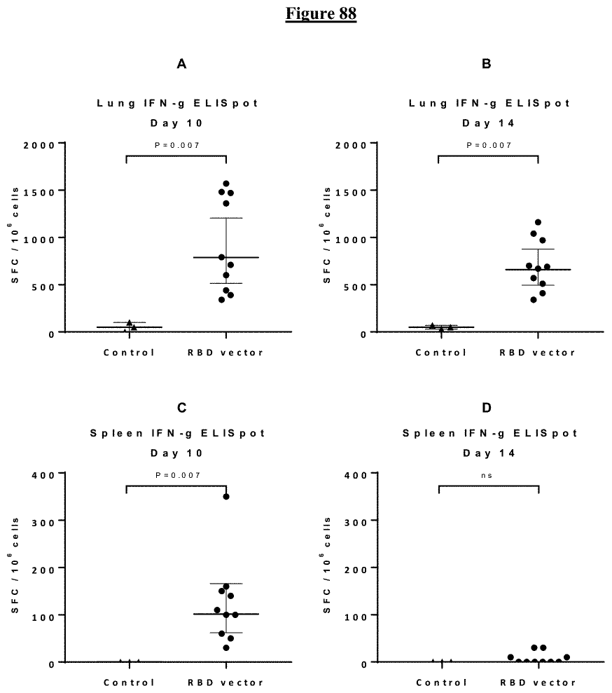

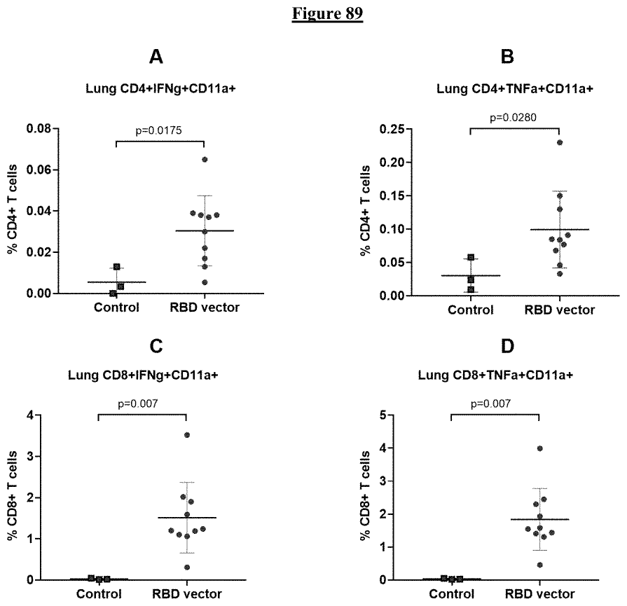

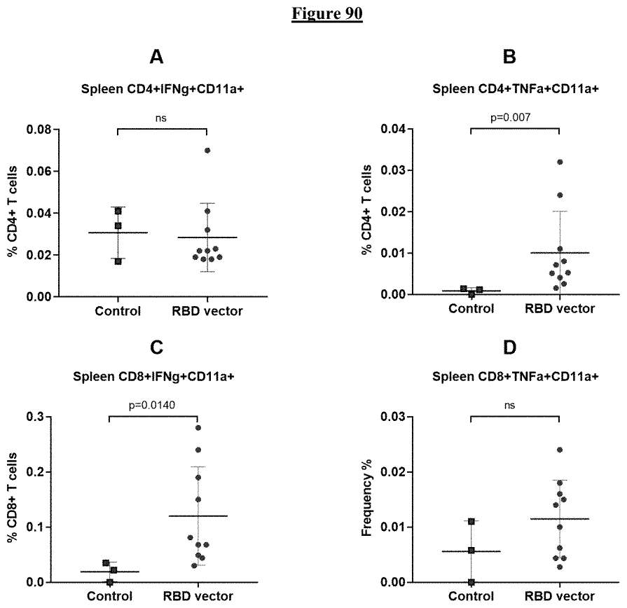

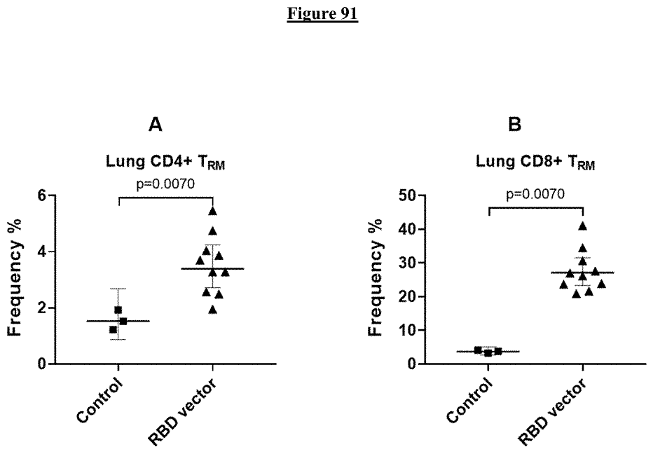

Following a single intranasal administration, the replication-deficient Ad5 vector expressing the RBD domain of Spike (SEQ ID NO: 15) was demonstrated to induce a significant production of IFN-gamma producing T cells in the lung and spleen as shown in FIGS. 88 A to D. A high frequency of IFN-γ-producing RBD-specific T cells were detected in the lung at 10- and 14-days post-vaccination, reaching a mean response of 915 and 706 spot per million input cells respectively. See FIG. 88. IFN-γ producing RBD-specific T cells were also detected by ELISpot in the spleen—albeit at lower frequency compared to the lungs. This suggests that functional effector T cells primed in response to mucosal-delivered antigens can migrate to peripheral lymphoid tissues. In addition, RBD-specific CD4+ and CD8+ T cells expressing the early activation homing markers CD11a and IFN-gamma and/or TNF-alpha were also found at significant levels in the lungs (FIGS. 89 A to D) and spleens (FIGS. 90 A to D). The expression of the integrin CD11a, which is only upregulated in recently activated T cells and is required for optimal vascular adhesion in the tissue and retention within the respiratory tract (Thatte J, Dabak V, Williams M B, Braciale T J, Ley K. LFA-1 is required for retention of effector CD8 T cells in mouse lungs. Blood. 2003 Jun. 15; 101(12):4916-22.), supporting the hypothesis that these cells were recently recruited to the lung. The T cell responses were found at higher levels in the lung and dominated by CD8+ T cells. Intranasal RBD vector also induced high level tissue-resident memory CD4+ and CD8+ T cells (TRM) in the lungs (FIGS. 91 A and B).

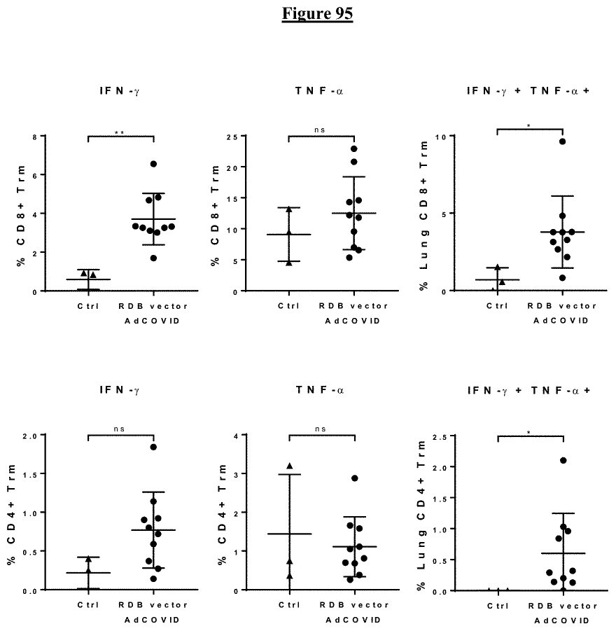

To assess whether vaccine-induced T cells might represent resident memory T cells (Trm), the expression of the Trm markers CD103 and CD69 (Takamura S. Persistence in Temporary Lung Niches: A Survival Strategy of Lung-Resident Memory CD8+ T Cells. Viral Immunol. 2017 July/August; 30(6):438-450. doi: 10.1089/vim.2017.0016.) was assessed on the lung CD4⁺ and CD8⁺ cells. Consistent with the intranasal administration route, induction of lung RDB-specific CD4⁺ and CD8⁺ Trm expressing either IFN-γ, TNF-α or both cytokines were observed (FIG. 95).

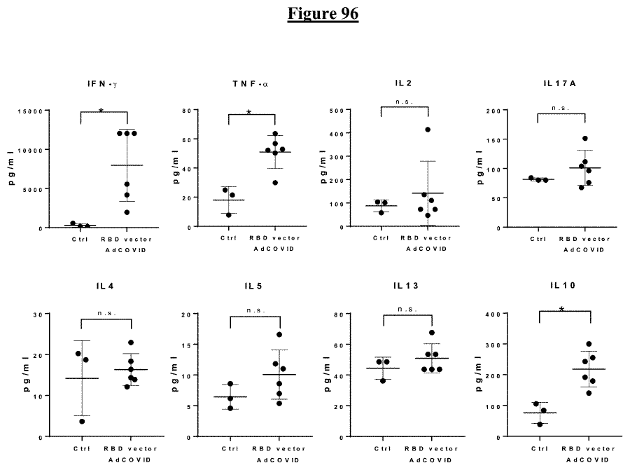

The data showed that intranasal administration of the RBD vector vaccine induced T cells competent to produce IFN-γ and TNF-α cytokines that are associated with Th-1 biased cellular response. In addition, we observed that the vaccine elicited high frequencies of antigen-specific CD8⁺ T cells that generally correlate with an IFN-regulated T cell response that is important for control of viral infection. To further assess the cytokine producing potential of the T cells from vaccinated mice, we restimulated the splenic T cells with RBD peptides for 48 hours and then used cytokine bead arrays to measure cytokine levels in the supernatant. As expected, we observed induction of IFN-γ and TNF-α by the T cells, Moreover, we found that the T cells from the vaccinated animals produced moderate levels of IL-10 compared to T cells from the vehicle control treated mice. Importantly, Interleukin (IL)-4, IL-5, IL-13 and IL-17a levels in the supernatant from re-stimulated cells derived from the vaccinated mice were equivalent to that seen in cultures containing peptide-stimulated cells from the vehicle control animals (FIG. 96).

### Example 18. Intranasal Administration of Ad5 Vector Expressing RBD Domain in C57BL/6 Mice Elicits Persistent Antibody Responses

The immunogenicity of an intranasal replication-defective Ad5 vector encoding the RBD domain residues 302 to 543 (SEQ ID NO: 15) of SARS-Cov-2 was assessed in inbred C57BL/6 mice by measuring spike-specific serum IgG responses over time. At Day 0 prevaccination and Days 15, 30, 63 and 120 post-vaccination (e.g., about 2 weeks, about 1 month, about 2 months and about 4 months), sera were collected after a single intranasal vaccination as described in Table 25. Methods for the quantification of SARS-CoV-2 spike IgG in serum is described in Example 13B.

TABLE 25

| | | Number of | | |
|---|---|---|---|---|
| Vaccine/ | Intranasal | animals | | Sample |
| Vehicle control | dose | per group | Immunization | collection |
| AdtPAWHSRBD | 3.78E+08 | 20 C57BL/6 | Day 0 | Day 0 (pre- |
| | ifu in 30 μL | | | vaccination), 15, |
| | | | | 30, 63 and 120 |

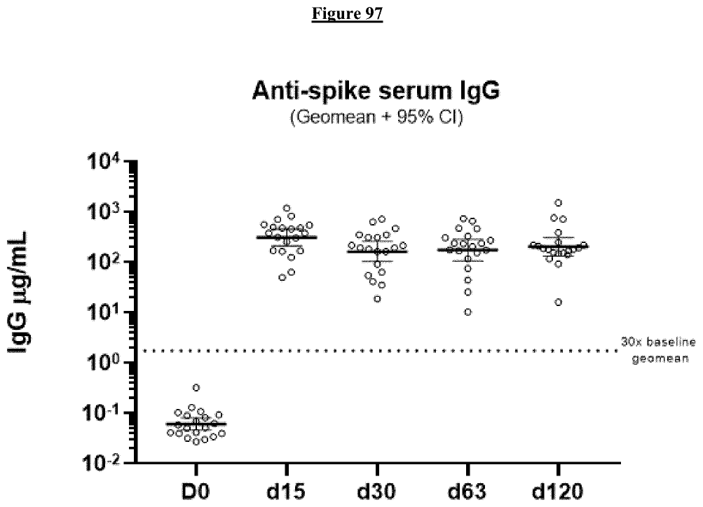

Results are presented in FIG. 97 wherein the 30× baseline is indicated with the dotted line. After detection of a spike-specific IgG response detected in all 20 vaccinated animals at day 15, responses remain stable up to day 120 with no statistical difference across the different post-vaccination timepoints based on a Wilcoxon matched-pairs signed rank test. This example demonstrated no significant decay in the measured anti-spike IgG from serum between 30 days and 120 days post vaccination. In embodiments, provided herein is a vaccine or RBD vector, that when administered as a single intranasal dose to a mammal induces an antibody response against the spike protein that is durable for at least 4 months. In some embodiments provided herein is a vaccine or RBD vector, that when administered as a single intranasal dose to a mammal induces an antibody response against the spike protein that is durable (given the absence of decay as measured at about 4 months) for at least about 5 months, at least about 6 months, at least about 7 months, at least about 8 months, at least about 9 months, at least about 10 months, at least about 11 months or at least about 12 months (on year).

### Example 19. Intranasal Administration of Ad5 Vector Expressing RBD Domain in CD1 Mice Elicits Long-Lived Antibody Secreting Cells in the Bone Marrow and Lung

The immunogenicity of an intranasal replication-defective Ad5 vector encoding the RBD domain residues 302 to 543 (SEQ ID NO: 15) of SARS-Cov-2 was assessed in outbred CD-1 mice by measuring RBD-specific plasma cells (antibody secreting cells; ASCs) in the bone marrow (BM) and lung that produce spike-specific IgG and IgA. At Days 69 post-vaccination, bone marrows and lungs were collected after a single intranasal vaccination as described in Table 26.

TABLE 26

| | | Number of | | |
|---|---|---|---|---|
| Vaccine/ | Intranasal | animals | | Sample |
| Vehicle control | dose | per group | Immunization | collection |
| RBD vector | 3.78E+08 | 5 CD-1 | Day 0 | Day 69 |
| | ifu in 30 μL | | | |

The detection of RBD-specific ASCs in the bone marrow (BM) and lung that produce spike-specific IgG and IgA by ELISPOT is summarized as follows. Single cell suspensions from bone marrow (2 tibia+2 femur/mouse) and lung cells were prepared from vaccinated mice. Cells were serially diluted in duplicate in complete media and incubated for 5 hours at 37° C. on multiscreen cellulose filter ELISPOT plates (Millipore) that were previously coated with purified recombinant RBD protein (Sino Biological). RBD-specific antibodies secreted by plasma cells present in these tissues were detected using AP-conjugated goat anti-mouse IgG Ab (Jackson ImmunoResearch) or AP-conjugated goat anti-mouse IgA (Jackson ImmunoResearch). ELISPOTS were imaged and counted using S6 Ultra-V Analyzer (Cellular Technology Limited).

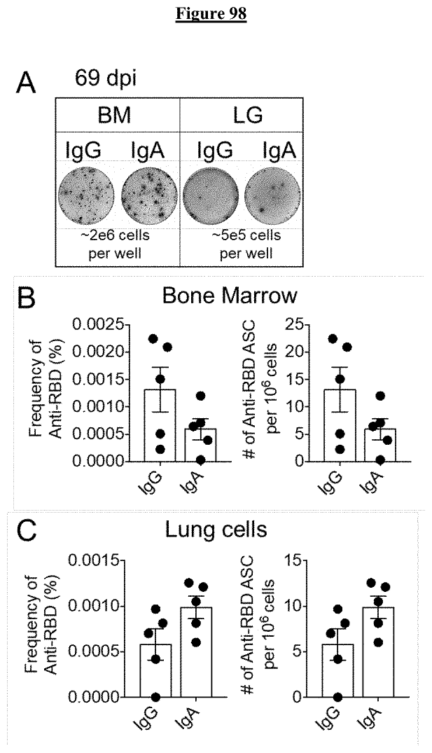

Results are presented in FIG. 98. RBD-specific ASCs were identified in the bone marrow of day 69 vaccinated mice. As these ASCs are located in a niche that specifically supports the long-term survival of ASCs, we predict that the systemic RBD-specific antibody titers induced by the RBD vector will be highly durable. Moreover, intranasal vaccination also resulted in the establishment of a durable ASC population in the lung that included IgA ASCs that can provide mucosal protection at the site of future infection. It is known that infection with respiratory viruses can also establish long-lived ASCs within the lung and that these cells are important in early protection from reinfection. Therefore, it is a reasonable supposition that the presence of these intranasal vaccine-induced ASCs may also provide local protection to the vaccinated animals. In embodiments provided herein are compositions (e.g., RBD vector) and methods for inducing bone marrow and lung resident memory antibody secreting cells. In some embodiments provided herein is a vaccine or RBD vector, that when administered as a single intranasal dose to a mammal induces bone marrow and lung resident memory antibody secreting cells that secrete both anti-spike IgG and IgA.

### Example 20. Intranasal Administration of Ad5 Vector Expressing RBD Domain in C57BL/6 Mice Elicits Long-Lived RBD-Specific B Cell Memory

The immunogenicity of an intranasal replication-defective Ad5 vector encoding the RBD domain residues 302 to 543 (SEQ ID NO: 15) of SARS-Cov-2 was assessed in inbred C57BL/6 mice by measuring long-lived RBD-specific B cell memory in the mediastinal lymph nodes and RBD-specific plasma cells (antibody secreting cells; ASCs) in the bone marrow (BM). At Day 168 post-vaccination (e.g., 24 weeks or 6 months), mediastinal lymph nodes and bone marrows were collected after a single intranasal vaccination as described in Table 27. Naïve C57BL/6 mice (n=5) were used negative controls.

TABLE 27

| | | Number of | | |
|---|---|---|---|---|
| Vaccine/ | Intranasal | animals | | Sample |
| Vehicle control | dose | per group | Immunization | collection |
| RBD vector | 3.78E+08 | 5 C57BL/6 | Day 0 | Day 168 |
| | ifu in 30 μL | | | |

For the flow cytometry analysis of RBD-specific B cell memory, the B cell antibody panel consisted of CD95/FAS-FITC (clone Jo2; 1:200 dilution), SARS-CoV-2 RBD-PE (1:200 dilution), SARS-CoV-2 RBD-APC (1:200 dilution), 7-AAD (1:1000 dilution), CD3-PerCP-Cy5.5 (clone 17A2; 1:200 dilution), CD38-PE-Cy7 (clone 90; 1:400 dilution), CD19-APC-Fire750 (clone 6D5; 1:200 dilution), CD138-BV421 (clone 281-2; 1:200 dilution) and IgD-BV510 (clone 11-26c.2a; 1:500 dilution). Cells were incubated with antibody mix (50 ul total volume) for 20 min at 4° C. in the dark and then washed with 200 ul SME. Cell were resuspended in 200 ul SME (no fixative) and immediately analyzed on FACSCanto II.

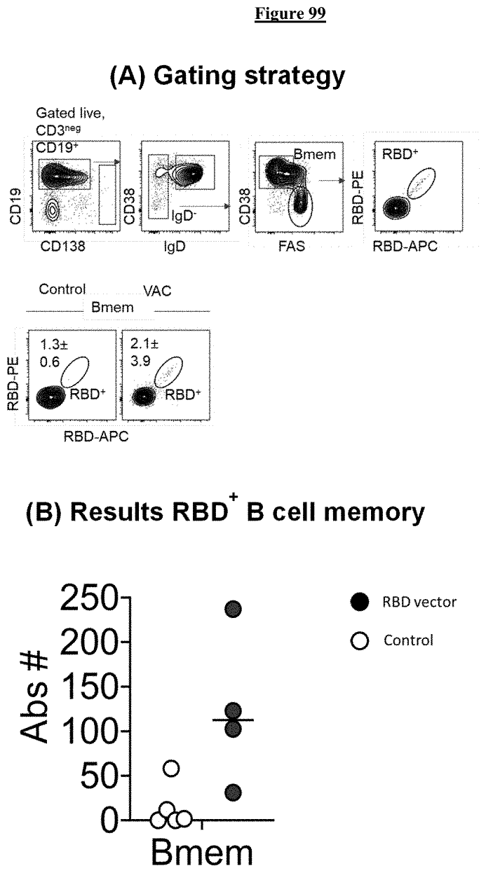

Results for RBD-specific memory B cells are presented in FIG. 99. FIG. 99B shows a higher absolute number of RBD-specific memory B cells present in the mediastinal lymph nodes of vaccinated animals compared to naïve animals. Detecting the presence of antigen-specific memory B cells 168 days after a single intranasal administration of the RBD vector provide a strong indication of the long-live nature of RBD-specific B induced by the intranasal vaccine. RBD-specific memory B cell present in mediastinal lymph nodes would be well-suited to be rapidly respond to respiratory infection such as SARS-CoV-2.

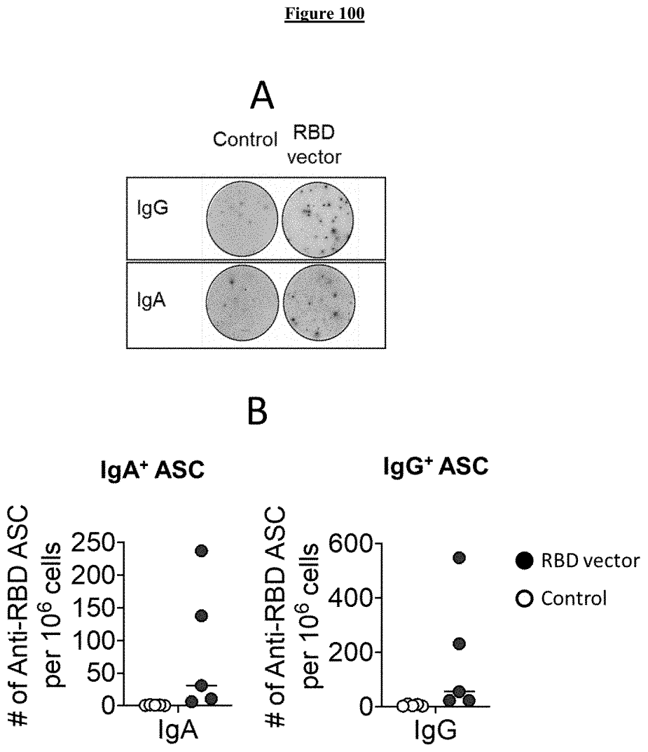

Measurement of ASCs in bone marrow followed the same method as described in example 19. Results are presented in FIG. 100. Long lived RBD-specific ASCs produced IgG and IgA antibodies were detected in the bone marrow of most animals 168 days after single intranasal administration of the RBD vector.

Discussion—Single-Dose Intranasal Vaccination with S1 and RBD Adenoviral Vector Elicits Rapid and Durable Systemic and Mucosal Immunity Against SARS-CoV-2 in Mice

The immunogenicity of the S1 and RBD vectors following a single administration of a replication-defective Ad5 vector encoding the S1 domain (residues 16 to 685) (SEQ ID NO: 13) or the RBD domain (residues 302 to 543) (SEQ ID NO: 15) from the Wuhan-1 strain of SARS-CoV-2 (accession number QHD43416.1) to inbred C57BL/6 and outbred CD-1 mice were assessed by measuring the induction of spike-specific antibody levels in sera and bronchoalveolar lavage (BAL) fluids. Each vaccine was evaluated at three (3) different dose levels (high, medium or low) as described above. Following single vaccine administration on day 0, sera, bronchoalveolar lavage (BAL) samples were collected between days 7-28 (C57BL/6) or days 7-21 (CD-1). IgG and IgA antibodies specific for SARS-CoV-2 spike were measured in serum or BAL samples using a spike cytometric bead array. The functionality of these vaccine-elicited antibodies was measured in live virus neutralization assays. In addition to the induction of robust neutralizing antibody responses and mucosal IgA against SARS-CoV-2, RBD stimulated systemic and mucosal cell-mediated immune responses characterized by a T-helper 1 (Th1) type cytokine profile and through the induction of cytokine-producing CD4⁺ and CD8⁺ T cells, including lung-resident memory T (Trm) cells.

Systemic spike-specific IgG antibody responses were detected in both strains of mice receiving a single intranasal administration of either the S1 vector or RBD vector vaccine. FIGS. 78 and 85. At the medium and high vaccine dose, the RBD vector induced a modestly larger serum spike-specific IgG response compared to the S1 vector—an effect that was more pronounced in the C57BL/6 mice compared to CD-1 mice. Moreover, intranasal administration of either S1 or RBD vector led to a rapid elevation of spike-specific IgG in the BAL of both strains of mice. Induction of IgA following a single intranasal dose of S1 or RBD was also demonstrated. Systemic spike-specific IgG antibody responses induced by the RBD vector (encoding SEQ ID NO: 15) following intranasal administration were also demonstrated to be durable with no evidence of decline over time for up to 120 days post-vaccination as presented in example 18. See FIG. 97. Moreover, intranasal vaccination also resulted in the establishment of a durable RBD-specific ASC and long-lived B cell memory population in the lung environment that included IgA ASCs that can provide local protection to the vaccinated animals. See FIGS. 98-100.

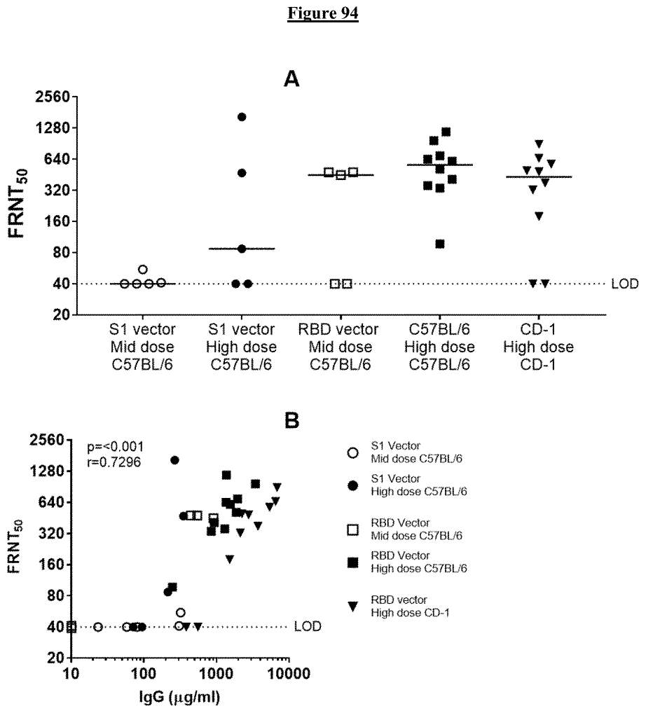

Intranasal administration of S1 vector or RBD vector elicited neutralizing antibody responses against SARS-CoV-2 were measured in a focus neutralization reduction test (FNRT) (similar as the plaque reduction neutralization test (PRNT)) in infection permissive Vero E6 cells using the wild-type SARS-CoV-2 isolate USA-WA1/2020. The analysis included samples from C57BL/6 mice 4-weeks after vaccination with S1 and RBD using either the mid or high vaccine dose, and samples from CD1 mice 3-weeks after vaccination with the high vaccine dose of RBD vector (FIG. 94A). Intranasal administration of RBD vector elicited significantly greater neutralizing titers compared to use of S1 vector under all conditions evaluated. At the highest dose, intranasal vaccination with RBD vector induced neutralizing antibody responses above background in 10/10 C57BL/6 and 8/10 CD-1 mice with a median titer of 563 and 431 respectively (FIG. 94A). The level of the neutralizing antibody response correlated tightly with magnitude of the spike-specific serum IgG response measured in individual animals (FIG. 94B), indicating that robust antibody responses to administration with RBD vector were associated with the generation of potentially protective neutralizing antibodies.

Examples 13, 15 and 17 demonstrate that a present vaccine composition, e.g., an adenovirus-vectored vaccine encoding the RBD sequence (SEQ ID NO: 15) of the SARS-CoV-2 spike protein configured for intranasal administration, is highly immunogenic in both inbred and outbred mice and elicits robust systemic and local mucosal antibody and T cell responses. Following a single intranasal vaccination, the RBD vector composition elicited a strong and focused immune response against SARS-CoV-2 Spike through the induction of functional antibodies that neutralize wild-type SARS-CoV-2 infection of permissive cells as well as mucosal IgA and cytokine-producing pulmonary CD4⁺ and CD8⁺ T cells. Cell-mediated responses induced by the RBD vector composition were biased toward an anti-viral response as demonstrated by the high rates of antigen-specific CD8⁺ T cells and cytokine expression that included IFN-γ and TNF-α. The establishment of a resident memory CD8+ T cell population in the lungs complements the robust induction of mucosal IgA antibody against the spike protein and represents an important addition to the overall immune response to the RBD vector composition. These data also indicate that intranasal administration of the RBD vector vaccine composition did not initiate a potentially deleterious Th2 response but rather induced the expected anti-viral T cell responses due to the anti-viral response induced with use of an adenoviral vector administered intranasally. See U.S. Pat. No. 9,605,275. Taken altogether, the data show that intranasal administration of the replication incompetent Ad5 vector expressing SARS-CoV-2 spike RBD sequence (SEQ ID NO: 15) generates humoral and cellular immune responses in both systemic and mucosal sites, particularly within the lung, which represents a major site for infection and clinical disease.

While certain embodiments have been described in terms of the preferred embodiments, it is understood that variations and modifications will occur to those skilled in the art. Therefore, it is intended that the appended claims cover all such equivalent variations that come within the scope of the following claims.

1-62. (canceled)

63. An immunogenic composition comprising a replication defective adenoviral (rdAd) vector comprising a nucleic acid sequence encoding SEQ ID NO: 446 or a variant comprising at least 95% identity to SEQ ID NO: 446.

64. The immunogenic composition of claim 63, wherein the nucleic acid sequence encodes SEQ ID NO: 15.

65. (canceled)

66. (canceled)

67. (canceled)

68. (canceled)

69. (canceled)

70. (canceled)

71. The immunogenic composition of claim 63, wherein the nucleic acid sequence encodes a sequence comprising one or more mutations selected from K417T, K417N, E484K, L452R and/or N501Y, wherein amino acid numbering corresponds to SEQ ID NO: 411.

72. (canceled)

73. (canceled)

74. The immunogenic composition of claim 63, wherein the nucleic acid sequence encoding SEQ ID NO: 446 further comprises a leader sequence encoded by a nucleic acid sequence encoding a sequence selected from SEQ ID NOS: 418 to 437.

75. The immunogenic composition of claim 63, wherein the coding sequence is codon optimized for a mammalian subject.

76. The immunogenic composition of claim 63, wherein the replication defective adenoviral vector is a bovine adenovirus, a canine adenovirus, a non-human primate adenovirus, a chicken adenovirus, a porcine or swine adenovirus, or a human adenovirus.

77. The immunogenic composition of claim 76, wherein the non-human primate adenovirus is a chimpanzee or gorilla adenovirus.

78. The immunogenic composition of claim 63, wherein the replication defective adenoviral vector is a human adenovirus.

79. The immunogenic composition of claim 78, wherein the human adenovirus is Ad5 or Ad26.

80. A pharmaceutical formulation, comprising an effective amount of the immunogenic composition of claim 63, the composition comprising at least one pharmaceutically acceptable diluent or carrier.

81. The pharmaceutical formulation of claim 80, configured for non-invasive or intranasal administration, wherein the pharmaceutically acceptable carrier is in a spray or aerosol form.

82. A method for inducing an immune response against SARS-CoV-2, the method comprising administering an effective amount of the immunogenic composition of claim 63 to a human being.

83. The method of claim 82, wherein the effective amount is at least 10⁸ viral particles (vp), at least 10⁹ viral particles (vp), or at least 10¹⁰ viral particles (vp).

84. The method of claim 82, wherein the immunogenic composition is administered intranasally.

85. (canceled)

86. The method of claim 82, wherein the immune response against SARS-CoV-2 comprises a mucosal IgA and/or T cell response against SARS-CoV-2 induced after administration of the immunogenic composition.

87. The method of claim 82, wherein the effective amount of the immunogenic composition reduces incidence of mild or moderate COVID-19-related diseases after the administration to the human subject.

88. (canceled)

89. (canceled)

90. The method of claim 82, wherein the effective amount of the immunogenic composition reduces incidence of infection with SARS-CoV-2 after the administration to the human subject.

91. (canceled)

92. The method of claim 82, wherein the effective amount of the immunogenic composition reduces transmission of SARS-CoV-2 after the administration to the human subject.

93. An immunogenic composition comprising a replication defective adenoviral (rdAd) vector comprising a nucleic acid sequence encoding SEQ ID NO: 15 or a variant comprising at least 95% identity to SEQ ID NO: 15.

94. An immunogenic composition comprising an Ad5 human adenovirus replication defective adenoviral (rdAd) vector comprising a nucleic acid sequence encoding SEQ ID NO: 15 or a variant comprising at least 95% identity to SEQ ID NO: 15.
